(19) United States
(12) Patent Application Publication
(10) Pub. No.: US 2024/0209061 A1
(43) Pub. Date: Jun. 27, 2024

(54) FUSION PROTEINS AND USES THEREOF

(71) Applicant: Pfizer Inc., New York, NY (US)

(72) Inventors: Eric Matthew Bennett, Arlington, MA (US); Christina Hwei-Lin Eng, Leonia, NJ (US); Laura Lin Lohse, Weston, MA (US); Lidia Mosyak, Newton, MA (US); Jeremy Shawn Myers, Orange, CT (US); Mohosin Sarkar, Woodbridge, CT (US); Hayretin Rafet Yumerefendi, Belmont, MA (US)

(21) Appl. No.: 17/908,822

(22) PCT Filed: Mar. 5, 2021
(86) PCT No.: PCT/IB2021/051865

§ 371 Date: Sep. 1, 2022

Related U.S. Application Data
(60) Provisional application No. 62/986,900, filed on Mar. 9, 2020.

(51) Int. Cl. C07K 14/705; A61K 31/5025; A61K 39/395; A61P 35/00; C07K 16/28

(52) U.S. Cl. CPC C07K 14/70532; A61K 31/5025; A61K 39/3955; A61P 35/00; C07K 16/2818; C07K 2317/94; C07K 2319/30

(57) ABSTRACT

The present invention provides for CD80-Fc fusion proteins that have therapeutic and diagnostic use, and methods for making thereof. The present invention further relates to variant CD80 polypeptides. The present invention also provides for CD80-Fc fusion proteins for use in the treatment of cancer.

### FIELD OF THE INVENTION

The present invention relates to CD80-Fc fusion proteins that have therapeutic and diagnostic use, and methods for making thereof. The present invention provides for CD80-Fc fusion proteins comprising an antibody Fc region and variant CD80 polypeptides. Also provided are methods for promoting T cell function and improving anti-tumor immunity, and methods for treating disorders (e.g. cancer) using the CD80-Fc fusion proteins alone or in combination with one or more additional agents.

### BACKGROUND OF THE INVENTION

CD80 (cluster of differentiation 80), also known as B7-1, is a type I membrane protein which is a member of the surface immunoglobulin superfamily that is expressed by activated B cells, macrophages, and dendritic cells. CD80 binds to the CD28 receptor and provides T cell co-stimulation after antigen recognition and prevents the formation of dysfunctional T cells. CD80 also binds CTLA-4 receptor, with higher affinity than CD28, which inhibits CD80-dependent co-stimulation.

Approved oncology agents that target this pathway include high dose IL-2 (Aldesleukin) and anti-CTLA-4 antibody (Ipilimumab). IL-2 was the first cytokine approved for cancer therapy, but efficacy is limited by systemic toxicity. Anti-CTLA-4 therapies prevent CTLA-4 from engaging CD80/CD86, allowing CD80/CD86 to stimulate CD28 and promote T cell priming. Anti-CTLA-4 therapies show clinical activity, however immune-related adverse effects occur due to systemic immune activation. Further, developing anti-CD28 antibodies has been challenging to date. For example, TGN1412 (a CD28 agonist monoclonal antibody) entered Phase I clinical trials in 2006 and acute cytokine release syndrome (CRS) was observed in patients. This was determined to be due to CD28 superagonism where CD28 activation occurs in the absence of T cell receptor (TCR) stimulation.

Therapeutics directed to the above described pathway have experienced some success clinically, however, there remains a significant clinical need for the development of optimized immune-modulating drugs having improved therapeutic and safety characteristics.

### BRIEF SUMMARY OF THE INVENTION

The invention disclosed herein is directed to CD80-Fc fusion proteins and variant CD80 polypeptides for use therein. In some aspects, provided herein is a CD80-Fc fusion protein comprising (i) an antibody Fc region and (ii) a variant CD80 polypeptide comprising a substitution of one or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In some aspects, the substitution is at position K36, K89, D90, and/or A91. In some aspects, the substitution at position K36 is K36R, the substitution at position K89 is K89D, K89E or K89Q, the substitution at position D90 is D90K, D90N or D90Q, and the substitution at position A91 is A91S. In another aspect, the substitution comprises K36R, K89D, K89E, K89Q, D90K, D90N, D90Q, A91S, K36R-K89D, K36R-K89E, K36R-K89Q, K36R-D90K, K36R-D90N, K36R-D90Q, K36R-A91S, K89D-D90K, K89D-D90N, K89D-D90Q, K89D-A91S, K89E-D90K, K89E-D90N, K89E-D90Q, K89E-A91S, K89Q-D90K, K89Q-D90N, K89Q-D90Q, K89Q-A91S, D90K-A91S, D90N-A91S, D90Q-A91S, K36R-K89D-D90K, K36R-K89D-D90N, K36R-K89D-D90Q, K36R-K89D-A91S, K36R-K89E-D90K, K36R-K89E-D90N, K36R-K89E-D90Q, K36R-K89E-A91S, K36R-K89Q-D90K, K36R-K89Q-D90N, K36R-K89Q-D90Q, K36R-K89Q-A91S. K36R-D90K-A91S, K36R-D90N-A91S, K36R-D90Q-A91S, K89D-D90K-A91S, K89D-D90N-A91S, K89D-D90Q-A91S, K89E-D90K-A91S, K89E-D90N-A91S, K89E-D90Q-A91S, K89Q-D90K-A91S, K89Q-D90N-A91S, K89Q-D90Q-A91S, K36R-K89D-D90K-A91S, K36R-K89D-D90N-A91S, K36R-K89D-D90Q-A91S, K36R-K89E-D90K-A91S, K36R-K89E-D90N-A91S, K36R-K89E-D90Q-A91S, K36R-K89Q-D90K-A91S, K36R-K89Q-D90N-A91S, or K36R-K89Q-D90Q-A91S. In a further aspect, the substitution comprises K89D, K89E, K89Q, D90K, D90N, D90Q, A91S, K89D-D90N, K89D-D90Q, K89D-D90K, or K89Q-D90Q.

In some aspects, the substitution increases the binding affinity of a CD80-Fc fusion protein to CD28 compared to the binding affinity of a wild-type CD80-Fc fusion protein to CD28.

In another aspect, the substitution is at position V11, V22, T28, E23, A26, Y31, Q33, G45, K54, T57, D60, I61, T62, N63 and/or N64. In some aspects, the substitution at position V11 is V11L; the substitution at position V22 is V22C, V22F or V22M; the substitution at position T28 is T28V; the substitution at position E23 is E23C; the substitution at position A26 is A26C; the substitution at position Y31 is Y31Q; the substitution at position Q33 is Q33E; the substitution at position G45 is G45C; the substitution at position K54 is K54E; the substitution at position T57 is T57V; the substitution at position D60 is D60F, D60Q, D60R, D60T or D60Y; the substitution at position I61 is I61C; the substitution at position T62 is T62F, T62I, T62L or T62Y; the substitution at position N63 is N63D or N63E; and the substitution at position N64 is N64D or N64E.

In some aspects, the substitution comprises V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D, N64E, V11L-V22C, V11L-V22F, V11L-V22M, V11L-T28V, V11L-E23C, V11L-A26C, V11L-Y31Q, V11L-Q33E, V11L-G45C, V11L-K54E, V11L-T57V, V11L-D60F, V11L-D60Q, V11L-D60R, V11L-D60T, V11L-D60Y, V11L-I61C, V11L-T62F, V11L-T62I, V11L-T62L, V11L-T62Y, V11L-N63D, V11L-N63E, V11L-N64D, V11L-N64E, V22C-T28V, V22C-E23C, V22C-A26C, V22C-Y31Q, V22C-Q33E, V22C-G45C, V22C-K54E, V22C-T57V, V22C-I61C, V22C-T62F, V22C-T62I, V22C-T62L, V22C-T62Y, V22C-N63D, V22C-N63E, V22C-N64D, V22C-N64E, V22F-T28V, V22F-E23C, V22F-A26C, V22F-Y31Q, V22F-Q33E, V22F-G45C, V22F-K54E, V22F-T57V, V22F-I61C, V22F-T62F, V22F-T62I, V22F-T62L, V22F-T62Y, V22F-N63D, V22F-N63E, V22F-N64D, V22F-N64E, V22M-T28V, V22M-E23C, V22M-A26C, V22M-Y31Q, V22M-Q33E, V22M-G45C, V22M-K54E, V22M-T57V, V22M-I61C, V22M-T62F, V22M-T62I, V22M-T62L, V22M-T62Y, V22M-N63D, V22M-N63E, V22M-N64D, V22M-N64E, T28V-E23C, T28V-A26C, T28V-Y31Q, T28V-Q33E, T28V-G45C, T28V-K54E, T28V-T57V, T28V-D60F, T28V-D60Q, T28V-D60R, T28V-D60T, T28V-D60Y, T28V-I61C, T28V-T62F, T28V-T62I, T28V-T62L, T28V-T62Y, T28V-N63D, T28V-N63E, T28V-N64D, T28V-N64E, E23C-A26C, E23C-Y31Q, E23C-Q33E, E23C-G45C, E23C-K54E, E23C-T57V, E23C-D60F, E23C-D60Q, E23C-D60R, E23C-D60T, E23C-D60Y, E23C-I61C, E23C-T62F, E23C-T62I, E23C-T62L, E23C-T62Y, E23C-N63D, E23C-N63E, E23C-N64D, E23C-N64E, A26C-Y31Q, A26C-Q33E, A26C-G45C, A26C-K54E, A26C-T57V, A26C-D60F, A26C-D60Q, A26C-D60R, A26C-D60T, A26C-D60Y, A26C-I61C, A26C-T62F, A26C-T62I, A26C-T62L, A26C-T62Y, A26C-N63D, A26C-N63E, A26C-N64D, A26C-N64E, Y31Q-Q33E, Y31Q-G45C, Y31Q-K54E, Y31Q-T57V, Y31Q-D60F, Y31Q-D60Q, Y31Q-D60R, Y31Q-D60T, Y31Q-D60Y, Y31Q-I61C, Y31Q-T62F, Y31Q-T62I, Y31Q-T62L, Y31Q-T62Y, Y31Q-N63D, Y31Q-N63E, Y31Q-N64D, Y31Q-N64E, Q33E-G45C, Q33E-K54E, Q33E-T57V, Q33E-D60F, Q33E-D60Q, Q33E-D60R, Q33E-D60T, Q33E-D60Y, Q33E-I61C, Q33E-T62F, Q33E-T62I, Q33E-T62L, Q33E-T62Y, Q33E-N63D, Q33E-N63E, Q33E-N64D, Q33E-N64E, G45C-K54E, G45C-T57V, G45C-D60F, G45C-D60Q, G45C-D60R, G45C-D60T, G45C-D60Y, G45C-I61C, G45C-T62F, G45C-T62I, G45C-T62L, G45C-T62Y, G45C-N63D, G45C-N63E, G45C-N64D, G45C-N64E, K54E-T57V, K54E-D60F, K54E-D60Q, K54E-D60R, K54E-D60T, K54E-D60Y, K54E-I61C, K54E-T62F, K54E-T62I, K54E-T62L, K54E-T62Y, K54E-N63D, K54E-N63E, K54E-N64D, K54E-N64E, T57V-D60F, T57V-D60Q, T57V-D60R, T57V-D60T, T57V-D60Y, T57V-I61C, T57V-T62F, T57V-T62I, T57V-T62L, T57V-T62Y, T57V-N63D, T57V-N63E, T57V-N64D, T57V-N64E, D60F-I61C, D60F-T62F, D60F-T62I, D60F-T62L, D60F-T62Y, D60F-N63D, D60F-N63E, D60F-N64D, D60F-N64E, D60E-I61C, D60E-T62F, D60E-T62I, D60E-T62L, D60E-T62Y, D60E-N63D, D60E-N63E, D60E-N64D, D60E-N64E, D60R-I61C, D60R-T62F, D60R-T62I, D60R-T62L, D60R-T62Y, D60R-N63D, D60R-N63E, D60R-N64D, D60R-N64E, D60T-I61C, D60T-T62F, D60T-T62I, D60T-T62L, D60T-T62Y, D60T-N63D, D60T-N63E, D60T-N64D, D60T-N64E, D60Y-I61C, D60Y-T62F, D60Y-T62I, D60Y-T62L, D60Y-T62Y, D60Y-N63D, D60Y-N63E, D60Y-N64D, D60Y-N64E, T62F-N63D, T62F-N63E, T62F-N64D, T62F-N64E, T62I-N63D, T62I-N63E, T62I-N64D, T62I-N64E, T62L-N63D, T62L-N63E, T62L-N64D, T62L-N64E, T62Y-N63D, T62Y-N63E, T62Y-N64D, T62Y-N64E, N63D-N64D, N63D-N64E, N63E-N64D, N63E-N64E, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, V22F-T28V-T57V-Y31Q-Q33E-K54E or any other combination of V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D and/or N64E.

In another aspect, the substitution comprises D60Y, I61C, V11L-V22F, V11L-T62Y, V22C-G45C, V22F-D60Y, V22F-T62L, E23C-A26C, T28V-T57V, D60F T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E.

In some aspects, the substitution increases stability of a CD80-Fc fusion protein compared to the stability of a wild-type CD80-Fc fusion protein. In some aspects, the increased stability provides for enhanced thermal stability, reduced thermal forced aggregation and/or reduced viscosity.

In another aspect, the substitution comprises K89E-I61C, K89E-D60Y, K89E-E23C-A26C, K89E-V22C-G45C, K89E-T28V-T57V, K89E-V11L-V22F, K89E-V11L-T62Y, K89E-V22F-T62L, K89E-D60Y-T62L, K89E-V22F-K89E-D60Y, K89E-D60F-T62I, K89E-D60R-T62Y, K89E-D60Y-V11L, K89E-D60Y-V22M, K89E-D60T-T62Y, K89E-D60Q-T62F, K89E-V22F-T28V-T57V, K89E-V11L-T62Y-N63D, K89E-D60Y-V11L-N63D, K89E-V22F-T62L-N64E, K89E-D60Y-T62L-N63D, K89E-D60Y-K54E-N63E-N64D, K89E-D60Y-T62L-N63D-N64E, K89E-V22F-D60Y-K54E-N64E, K89E-V22F-T62L-N63D-N64E, K89E-T28V-T57V-Y31Q-Q33E-K54E, K89E-V22F-T28V-T57V-Y31Q-Q33E-K54E, K89Q-I61C, K89Q-D60Y, K89Q-E23C-A26C, K89Q-V22C-G45C, K89Q-T28V-T57V, K89Q-V11L-V22F, K89Q-V11L-T62Y, K89Q-V22F-T62L, K89Q-D60Y-T62L, K89Q-V22F-D60Y, K89Q-D60F-T62I, K89Q-D60R-T62Y, K89Q-D60Y-V11L, K89Q-D60Y-V22M, K89Q-D60T-T62Y, K89Q-D60Q-T62F, K89Q-V22F-T28V-T57V, K89Q-V11L-T62Y-N63D, K89Q-D60Y-V11L-N63D, K89Q-V22F-T62L-N64E, K89Q-D60Y-T62L-N63D, K89Q-D60Y-K54E-N63E-N64D, K89Q-D60Y-T62L-N63D-N64E, K89Q-V22F-D60Y-K54E-N64E, K89Q-V22F-T62L-N63D-N64E, K89Q-T28V-T57V-Y31Q-Q33E-K54E, K89Q-V22F-T28V-T57V-Y31Q-Q33E-K54E, K89D-I61C, K89D-D60Y, K89D-E23C-A26C, K89D-V22C-G45C, K89D-T28V-T57V, K89D-V11L-V22F, K89D-V11L-T62Y, K89D-V22F-T62L, K89D-D60Y-T62L, K89D-V22F-D60Y, K89D-D60F-T62I, K89D-D60R-T62Y, K89D-D60Y-V11L, K89D-D60Y-V22M, K89D-D60T-T62Y, K89D-D60Q-T62F, K89D-V22F-T28V-T57V, K89D-V11L-T62Y-N63D, K89D-D60Y-V11L-N63D, K89D-V22F-T62L-N64E, K89D-D60Y-T62L-N63D, K89D-D60Y-K54E-N63E-N64D, K89D-D60Y-T62L-N63D-N64E, K89D-V22F-D60Y-K54E-N64E, K89D-V22F-T62L-N63D-N64E, K89D-T28V-T57V-Y31Q-Q33E-K54E, K89D-V22F-T28V-T57V-Y31Q-Q33E-K54E, D90K-I61C, D90K-D60Y, D90K-E23C-A26C, D90K-V22C-G45C, D90K-T28V-T57V, D90K-V11L-V22F, D90K-V11L-T62Y, D90K-V22F-T62L, D90K-D60Y-T62L, D90K-V22F-D60Y, D90K-D60F-T62I, D90K-D60R-T62Y, D90K-D60Y-V11L, D90K-D60Y-V22M, D90K-D60T-T62Y, D90K-D60Q-T62F, D90K-V22F-T28V-T57V, D90K-V11L-T62Y-N63D, D90K-D60Y-V11L-N63D, D90K-V22F-T62L-N64E, D90K-D60Y-T62L-N63D, D90K-D60Y-K54E-N63E-N64D, D90K-D60Y-T62L-N63D-N64E, D90K-V22F-D60Y-K54E-N64E, D90K-V22F-T62L-N63D-N64E, D90K-T28V-T57V-Y31Q-Q33E-K54E, D90K-V22F-T28V-T57V-Y31Q-Q33E-K54E, D90N-I61C, D90N-D60Y, D90N-E23C-A26C, D90N-V22C-G45C, D90N-T28V-T57V, D90N-V11L-V22F, D90N-V11L-T62Y, D90N-V22F-T62L, D90N-D60Y-T62L, D90N-V22F-D60Y, D90N-D60F-T62I, D90N-D60R-T62Y, D90N-D60Y-V11L, D90N-D60Y-V22M, D90N-D60T-T62Y, D90N-D60Q-T62F. D90N-V22F-T28V-T57V, D90N-V11L-T62Y-N63D, D90N-D60Y-V11L-N63D, D90N-V22F-T62L-N64E, D90N-D60Y-T62L-N63D, D90N-D60Y-K54E-N63E-N64D, D90N-D60Y-T62L-N63D-N64E, D90N-V22F-D60Y-K54E-N64E, D90N-V22F-T62L-N63D-N64E, D90N-T28V-T57V-Y31Q-Q33E-K54E, D90N-V22F-T28V-T57V-Y31Q-Q33E-K54E, D90Q-I61C, D90Q-D60Y, D90Q-E23C-A26C, D90Q-V22C-G45C, D90Q-T28V-T57V. D90Q-V11L-V22F, D90Q-V11L-T62Y, D90Q-V22F-T62L, D90Q-D60Y-T62L, D90Q-V22F-D60Y, D90Q-D60F-T62I, D90Q-D60R-T62Y, D90Q-D60Y-V11L, D90Q-D60Y-V22M, D90Q-D60T-T62Y, D90Q-D60Q-T62F, D90Q-V22F-T28V-T57V, D90Q-V11L-T62Y-N63D, D90Q-D60Y-V11L-N63D, D90Q-V22F-T62L-N64E, D90Q-D60Y-T62L-N63D, D90Q-D60Y-K54E-N63E-N64D, D90Q-D60Y-T62L-N63D-N64E, D90Q-V22F-D60Y-K54E-N64E, D90Q-V22F-T62L-N63D-N64E, D90Q-T28V-T57V-Y31Q-Q33E-K54E, D90Q-V22F-T28V-T57V-Y31Q-Q33E-K54E, K89Q-D90Q-I61C, K89Q-D90Q-D60Y, K89Q-D90Q-E23C-A26C, K89Q-D90Q-V22C-G45C, K89Q-D90Q-T28V-T57V, K89Q-D90Q-V11L-V22F, K89Q-D90Q-V11L-T62Y, K89Q-D90Q-V22F-T62L, K89Q-D90Q-D60Y-T62L, K89Q-D90Q-V22F-D60Y, K89Q-D90Q-D60F-T62I, K89Q-D90Q-D60R-T62Y, K89Q-D90Q-D60Y-V11L, K89Q-D90Q-D60Y-V22M, K89Q-D90Q-D60T-T62Y, K89Q-D90Q-D60Q-T62F, K89Q-D90Q-V22F-T28V-T57V, K89Q-D90Q-V11L-T62Y-N63D, K89Q-D90Q-D60Y-V11L-N63D, K89Q-D90Q-V22F-T62L-N64E, K89Q-D90Q-D60Y-T62L-N63D, K89Q-D90Q-D60Y-K54E-N63E-N64D, K89Q-D90Q-D60Y-T62L-N63D-N64E, K89Q-D90Q-V22F-D60Y-K54E-N64E, K89Q-D90Q-V22F-T62L-N63D-N64E, K89Q-D90Q-T28V-T57V-Y31Q-Q33E-K54E. K89Q-D90Q-V22F-T28V-T57V-Y31Q-Q33E-K54E, K89D-D90N-I61C, K89D-D90N-D60Y, K89D-D90N-E23C-A26C, K89D-D90N-V22C-G45C, K89D-D90N-T28V-T57V, K89D-D90N-V11L-V22F, K89D-D90N-V11L-T62Y, K89D-D90N-V22F-T62L, K89D-D90N-D60Y-T62L, K89D-D90N-V22F-D60Y, K89D-D90N-D60F-T62I, K89D-D90N-D60R-T62Y, K89D-D90N-D60Y-V11L, K89D-D90N-D60Y-V22M, K89D-D90N-D60T-T62Y, K89D-D90N-D60Q-T62F, K89D-D90N-V22F-T28V-T57V, K89D-D90N-V11L-T62Y-N63D, K89D-D90N-D60Y-V11L-N63D, K89D-D90N-V22F-T62L-N64E, K89D-D90N-D60Y-T62L-N63D, K89D-D90N-D60Y-K54E-N63E-N64D, K89D-D90N-D60Y-T62L-N63D-N64E, K89D-D90N-V22F-D60Y-K54E-N64E, K89D-D90N-V22F-T62L-N63D-N64E, K89D-D90N-T28V-T57V-Y31Q-Q33E-K54E, K89D-D90N-V22F-T28V-T57V-Y31Q-Q33E-K54E, K89D-D90Q-I61C, K89D-D90Q-D60Y, K89D-D90Q-E23C-A26C, K89D-D90Q-V22C-G45C, K89D-D90Q-T28V-T57V, K89D-D90Q-V11L-V22F, K89D-D90Q-V11L-T62Y, K89D-D90Q-V22F-T62L, K89D-D90Q-D60Y-T62L, K89D-D90Q-V22F-D60Y, K89D-D90Q-D60F-T62I, K89D-D90Q-D60R-T62Y, K89D-D90Q-D60Y-V11L, K89D-D90Q-D60Y-V22M, K89D-D90Q-D60T-T62Y, K89D-D90Q-D60Q-T62F, K89D-D90Q-V22F-T28V-T57V, K89D-D90Q-V11L-T62Y-N63D, K89D-D90Q-D60Y-V11L-N63D, K89D-D90Q-V22F-T62L-N64E, K89D-D90Q-D60Y-T62L-N63D, K89D-D90Q-D60Y-K54E-N63E-N64D, K89D-D90Q-D60Y-T62L-N63D-N64E, K89D-D90Q-V22F-D60Y-K54E-N64E, K89D-D90Q-V22F-T62L-N63D-N64E, K89D-D90Q-T28V-T57V-Y31Q-Q33E-K54E, K89D-D90Q-V22F-T28V-T57V-Y31Q-Q33E-K54E, K89D-D90K-I61C, K89D-D90K-D60Y, K89D-D90K-E23C-A26C, K89D-D90K-V22C-G45C, K89D-D90K-T28V-T57V, K89D-D90K-V11L-V22F, K89D-D90K-V11L-T62Y, K89D-D90K-V22F-T62L, K89D-D90K-D60Y-T62L, K89D-D90K-V22F-D60Y, K89D-D90K-D60F-T62I, K89D-D90K-D60R-T62Y, K89D-D90K-D60Y-V11L, K89D-D90K-D60Y-V22M, K89D-D90K-D60T-T62Y, K89D-D90K-D60Q-T62F, K89D-D90K-V22F-T28V-T57V, K89D-D90K-V11L-T62Y-N63D, K89D-D90K-D60Y-V11L-N63D, K89D-D90K-V22F-T62L-N64E, K89D-D90K-D60Y-T62L-N63D, K89D-D90K-D60Y-K54E-N63E-N64D, K89D-D90K-D60Y-T62L-N63D-N64E, K89D-D90K-V22F-D60Y-K54E-N64E, K89D-D90K-V22F-T62L-N63D-N64E, K89D-D90K-T28V-T57V-Y31Q-Q33E-K54E, K89D-D90K-V22F-T28V-T57V-Y31Q-Q33E-K54E, A91S-I61C, A91S-D60Y, A91S-E23C-A26C, A91S-V22C-G45C, A91S-T28V-T57V, A91S-V11L-V22F, A91S-V11L-T62Y, A91S-V22F-T62L, A91S-D60Y-T62L, A91S-V22F-D60Y, A91S-D60F-T62I, A91S-D60R-T62Y, A91S-D60Y-V11L, A91S-D60Y-V22M, A91S-D60T-T62Y, A91S-D60Q-T62F, A91S-V22F-T28V-T57V, A91S-V11L-T62Y-N63D, A91S-D60Y-V11L-N63D, A91S-V22F-T62L-N64E, A91S-D60Y-T62L-N63D, A91S-D60Y-K54E-N63E-N64D, A91S-D60Y-T62L-N63D-N64E, A91S-V22F-D60Y-K54E-N64E, A91S-V22F-T62L-N63D-N64E, A91S-T28V-T57V-Y31Q-Q33E-K54E, or A91S-V22F-T28V-T57V-Y31Q-Q33E-K54E.

In some aspects, the substitution comprises D90Q. In another aspect, the substitution comprises K89Q-D90Q. In another aspect, the substitution comprises K89Q-D90Q-E23C-A26C. In another aspect, the substitution comprises K89D-D90K-T28V-T57V.

Further provided herein is a CD80-Fc fusion protein comprising (i) an antibody Fc region and (ii) a variant CD80 polypeptide comprising a variant CD80 polypeptide comprises i) a first substitution at position K36, K89, D90, and/or A91 of the amino acid sequence of SEQ ID NO: 2, and ii) a second substitution at position V11, V22, T28, E23, A26, Y31, Q33, G45, K54, T57, D60, I61, T62, N63 and/or N64 of the amino acid sequence of SEQ ID NO: 2.

In some aspects, i) the first substitution at position K36 is K36R, the first substitution at position K89 is K89D, K89E or K89Q, and the first substitution at position D90 is D90K or D90Q, and the first substitution at position A91 is A91S, and ii) the second substitution at position V11 is V11L; the second substitution at position V22 is V22C, V22F or V22M; the second substitution at position T28 is T28V; the second substitution at position E23 is E23C; the second substitution at position A26 is A26C; the second substitution at position Y31 is Y31Q; the second substitution at position Q33 is Q33E; the second substitution at position G45 is G45C; the second substitution at position K54 is K54E; the second substitution at position T57 is T57V; the second substitution at position D60 is D60F, D60Q, D60R, D60T or D60Y; the second substitution at position I61 is I61C; the second substitution at position T62 is T62F, T62I, T62L or T62Y; the second substitution at position N63 is N63D or N63E; and the second substitution at position N64 is N64D or N64E.

In some aspects, i) the first substitution comprises K36R, K89D, K89E, K89Q, D90K, D90N, D90Q, A91S, K36R-K89D, K36R-K89E, K36R-K89Q, K36R-D90K, K36R-D90N, K36R-D90Q, K36R-A91S, K89D-D90K, K89D-D90N, K89D-D90Q, K89D-A91S, K89E-D90K, K89E-D90N, K89E-D90Q, K89E-A91S, K89Q-D90K, K89Q-D90N, K89Q-D90Q, K89Q-A91S, D90K-A91S, D90N-A91S, D90Q-A91S, K36R-K89D-D90K, K36R-K89D-D90N, K36R-K89D-D90Q, K36R-K89D-A91S, K36R-K89E-D90K, K36R-K89E-D90N, K36R-K89E-D90Q, K36R-K89E-A91S, K36R-K89Q-D90K, K36R-K89Q-D90N, K36R-K89Q-D90Q, K36R-K89Q-A91S, K36R-D90K-A91S, K36R-D90N-A91S, K36R-D90Q-A91S, K89D-D90K-A91S, K89D-D90N-A91S, K89D-D90Q-A91S, K89E-D90K-A91S, K89E-D90N-A91S, K89E-D90Q-A91S, K89Q-D90K-A91S, K89Q-D90N-A91S, K89Q-D90Q-A91S, K36R-K89D-D90K-A91S, K36R-K89D-D90N-A91S, K36R-K89D-D90Q-A91S, K36R-K89E-D90K-A91S, K36R-K89E-D90N-A91S, K36R-K89E-D90Q-A91S, K36R-K89Q-D90K-A91S, K36R-K89Q-D90N-A91S, or K36R-K89Q-D90Q-A91S, and ii) the second substitution comprises V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D, N64E, V11L-V22C, V11L-V22F, V11L-V22M, V11L-T28V, V11L-E23C, V11L-A26C, V11L-Y31Q, V11L-Q33E, V11L-G45C, V11L-K54E, V11L-T57V, V11L-D60F, V11L-D60Q, V11L-D60R, V11L-D60T, V11L-D60Y, V11L-I61C, V11L-T62F, V11L-T62I, V11L-T62L, V11L-T62Y, V11L-N63D, V11L-N63E, V11L-N64D, V11L-N64E, V22C-T28V, V22C-E23C, V22C-A26C, V22C-Y31Q, V22C-Q33E, V22C-G45C, V22C-K54E, V22C-T57V, V22C-I61C, V22C-T62F, V22C-T62I, V22C-T62L, V22C-T62Y, V22C-N63D, V22C-N63E, V22C-N64D, V22C-N64E, V22F-T28V, V22F-E23C, V22F-A26C, V22F-Y31Q, V22F-Q33E, V22F-G45C, V22F-K54E, V22F-T57V, V22F-I61C, V22F-T62F, V22F-T62I, V22F-T62L, V22F-T62Y, V22F-N63D, V22F-N63E, V22F-N64D, V22F-N64E, V22M-T28V, V22M-E23C, V22M-A26C, V22M-Y31Q, V22M-Q33E, V22M-G45C, V22M-K54E, V22M-T57V, V22M-I61C, V22M-T62F, V22M-T62I, V22M-T62L, V22M-T62Y, V22M-N63D, V22M-N63E, V22M-N64D, V22M-N64E, T28V-E23C, T28V-A26C, T28V-Y31Q, T28V-Q33E, T28V-G45C, T28V-K54E, T28V-T57V, T28V-D60F, T28V-D60Q, T28V-D60R, T28V-D60T, T28V-D60Y, T28V-I61C, T28V-T62F, T28V-T62I, T28V-T62L, T28V-T62Y, T28V-N63D, T28V-N63E, T28V-N64D, T28V-N64E, E23C-A26C, E23C-Y31Q, E23C-Q33E, E23C-G45C, E23C-K54E, E23C-T57V, E23C-D60F, E23C-D60Q, E23C-D60R, E23C-D60T, E23C-D60Y, E23C-I61C, E23C-T62F, E23C-T62I, E23C-T62L, E23C-T62Y, E23C-N63D, E23C-N63E, E23C-N64D, E23C-N64E, A26C-Y31Q, A26C-Q33E, A26C-G45C, A26C-K54E, A26C-T57V, A26C-D60F, A26C-D60Q, A26C-D60R, A26C-D60T, A26C-D60Y, A26C-I61C, A26C-T62F, A26C-T62I, A26C-T62L, A26C-T62Y, A26C-N63D, A26C-N63E, A26C-N64D, A26C-N64E, Y31Q-Q33E, Y31Q-G45C, Y31Q-K54E, Y31Q-T57V, Y31Q-D60F, Y31Q-D60Q, Y31Q-D60R, Y31Q-D60T, Y31Q-D60Y, Y31Q-I61C, Y31Q-T62F, Y31Q-T62I, Y31Q-T62L, Y31Q-T62Y, Y31Q-N63D, Y31Q-N63E, Y31Q-N64D, Y31Q-N64E, Q33E-G45C, Q33E-K54E, Q33E-T57V, Q33E-D60F, Q33E-D60Q, Q33E-D60R, Q33E-D60T, Q33E-D60Y, Q33E-I61C, Q33E-T62F, Q33E-T62I, Q33E-T62L, Q33E-T62Y, Q33E-N63D, Q33E-N63E, Q33E-N64D, Q33E-N64E, G45C-K54E, G45C-T57V, G45C-D60F, G45C-D60Q, G45C-D60R, G45C-D60T, G45C-D60Y, G45C-I61C, G45C-T62F, G45C-T62I, G45C-T62L, G45C-T62Y, G45C-N63D, G45C-N63E, G45C-N64D, G45C-N64E, K54E-T57V, K54E-D60F, K54E-D60Q, K54E-D60R, K54E-D60T, K54E-D60Y, K54E-I61C, K54E-T62F, K54E-T62I, K54E-T62L, K54E-T62Y, K54E-N63D, K54E-N63E, K54E-N64D, K54E-N64E, T57V-D60F, T57V-D60Q, T57V-D60R, T57V-D60T, T57V-D60Y, T57V-I61C, T57V-T62F, T57V-T62I, T57V-T62L, T57V-T62Y, T57V-N63D, T57V-N63E, T57V-N64D, T57V-N64E, D60F-I61C, D60F-T62F, D60F-T62I, D60F-T62L, D60F-T62Y, D60F-N63D, D60F-N63E, D60F-N64D, D60F-N64E, D60E-I61C, D60E-T62F, D60E-T62I, D60E-T62L, D60E-T62Y, D60E-N63D, D60E-N63E, D60E-N64D, D60E-N64E, D60R-I61C, D60R-T62F, D60R-T62I, D60R-T62L, D60R-T62Y, D60R-N63D, D60R-N63E, D60R-N64D, D60R-N64E, D60T-I61C, D60T-T62F, D60T-T62I, D60T-T62L, D60T-T62Y, D60T-N63D, D60T-N63E, D60T-N64D, D60T-N64E, D60Y-I61C, D60Y-T62F, D60Y-T62I, D60Y-T62L, D60Y-T62Y, D60Y-N63D, D60Y-N63E, D60Y-N64D, D60Y-N64E, T62F-N63D, T62F-N63E, T62F-N64D, T62F-N64E, T62I-N63D, T62I-N63E, T62|-N64D, T62I-N64E, T62L-N63D, T62L-N63E, T62L-N64D, T62L-N64E, T62Y-N63D, T62Y-N63E, T62Y-N64D, T62Y-N64E, N63D-N64D, N63D-N64E, N63E-N64D, N63E-N64E, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, V22F-T28V-T57V-Y31Q-Q33E-K54E or any other combination of V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D and/or N64E.

In another aspect, i) the first substitution comprises K89D K89E, K89Q, D90K, D90N, D90Q, A91S, K89D-D90N, K89D-D90Q, K89D-D90K, or K89Q-D90Q, and ii) the second substitution comprises D60Y, I61C, V11L-V22F, V11L-T62Y, V22C-G45C, V22F-D60Y, V22F-T62L, E23C-A26C, T28V-T57V, D60F T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E.

In some aspects, i) the first substitution comprises K89Q-D90Q and ii) the second substitution comprises E23C-A26C. In another aspect, i) the first substitution comprises K89D-D90K and ii) the second substitution comprises T28V-T57V.

In another aspect, i) the first substitution increases the binding affinity of a CD80-Fc fusion protein to CD28 compared to the binding affinity of a wild-type CD80-Fc fusion protein to CD28, and ii) the second substitution increases stability of a CD80-Fc fusion protein compared to the stability of a wild-type CD80-Fc fusion protein. In some aspects, the increased stability provides for enhanced thermal stability, reduced thermal forced aggregation and/or reduced viscosity.

Further provided herein is a CD80-Fc fusion protein that i) does not increase or enhance binding to PD-L1, or ii) demonstrates minimal or no detectable binding to PD-L1.

In some aspects, the variant CD80 polypeptide comprises the amino acid sequence of any of SEQ ID NO: 20-63. In some aspects, the antibody Fc region is derived from IgG1, IgG2 or IgG4. In some aspects, the antibody Fc region comprises the amino acid sequence of any of SEQ ID NO. 13-18. In some aspects, the antibody Fc region is linked to the variant CD80 polypeptide. In some aspects, the CD80-Fc fusion protein comprises the amino acid sequence of any of SEQ ID NO: 64-114.

In some aspects, the invention provides an isolated cell line that produces the CD80-Fc fusion protein described here. In another aspect, the invention provides an isolated nucleic acid encoding the CD80-Fc fusion protein described herein. In another aspect, the invention provides a vector comprising the nucleic described herein. In another aspect, the invention provides a host cell comprising the nucleic acid or the vector described herein

Further provided herein is a method of producing a CD80-Fc fusion protein, comprising culturing the host cell described herein under conditions that result in the production of the CD80-Fc fusion protein described herein, and purifying the produced CD80-Fc fusion protein.

In some aspects, the invention provides for a pharmaceutical composition comprising the CD80-Fc fusion protein of any one of claims 1-31 and a pharmaceutically acceptable carrier.

In another aspect, the invention provides for a method for treating cancer in a subject in need thereof, the method comprising administering to the subject an effective amount of the CD80-Fc fusion protein described herein or the pharmaceutical composition described herein.

In some aspects, the cancer is gastric cancer, small intestine cancer, sarcoma, lymphoma, Hodgkin's lymphoma, leukemia, multiple myeloma, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer (e.g., non-small-cell lung cancer), mesothelioma, ovarian cancer, breast cancer, prostate cancer, esophageal cancer, pancreatic cancer, glioma, renal cancer (e.g., renal cell carcinoma), bladder cancer, cervical cancer, uterine cancer, vulvar cancer, penile cancer, testicular cancer, anal cancer, choriocarcinoma, colon cancer, colorectal cancer, oral cancer, skin cancer, Merkel cell carcinoma, glioblastoma, brain tumor, bone cancer, eye cancer, melanoma, or cancer with high microsatellite instability (MSI-H). In another aspect, the cancer is relapsed, resistant, refractory, and/or metastatic. In another aspect, the cancer is resistant and/or refractory to anti-PD-1 and/or anti-PD-L1 therapies

In some aspects, the invention provides for a method of enhancing an immune response in a subject in need thereof, the method comprising administering to the subject an effective amount of the CD80-Fc fusion protein described herein, or the pharmaceutical composition described herein.

In another aspect, the method further comprises administering an effective amount of one or more additional agents. In some aspects, the one or more additional agents is an antibody selected from the group consisting of an anti-CTLA-4 antibody, an anti-CD3 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, and an anti-GITR antibody.

In some aspects, the one or more additional agents is a cytokine, an immunocytokine, a targeted cytokine, TNFα, a PARP inhibitor, an oncolytic virus, a kinase inhibitor, an ALK inhibitor, a MEK inhibitor, an IDO inhibitor, a GLS1 inhibitor, a tyrosine kinase inhibitor, a CART cell or T cell therapy, a TLR agonist, cancer vaccine, KRAS inhibitor, BRAF inhibitor, PI3K inhibitor, EGFR inhibitor, HPK1 inhibitor, CDK or other cell cycle inhibitor, EZH2 inhibitor or other epigenetic modifier, anti-estrogen or anti-androgen therapy, radiation therapy, chemotherapy, a PRR agonist, a bispecific or multispecific antibody, an antibody-drug conjugate or other innate immune modulator.

In some aspects, the one or more additional agents is an anti-PD-1 antibody, a bispecific antibody, a CDK inhibitor and/or chemotherapy.

In some aspects, the anti-PD-1 antibody is PF-06801591/RN888. In some aspects, the anti-PD-1 antibody comprises a VH CDR1, VH CDR2, and VH CDR3 of a heavy chain variable region set forth as SEQ ID NO: 123 and/or a VL CDR1, VL CDR2, and VL CDR3 of a light chain variable region set forth as SEQ ID NO: 127. In some aspects, the anti-PD-1 antibody comprises a VH CDR1 of SEQ ID NO: 120, a VH CDR2 of SEQ ID NO: 121, and a VH CDR3 of SEQ ID NO: 122, and/or a VL CDR1 of SEQ ID NO: 124, a VL CDR2 of SEQ ID NO: 125, and/a VL CDR3 of SEQ ID NO: 126. In some aspects, the anti-PD-1 antibody comprises a heavy chain variable region set forth as SEQ ID NO: 123 and/or a light chain variable region set forth as SEQ ID NO: 127. In some aspects, the CDK inhibitor is palbociclib, PF-06873600, abemaciclib or ribociclib.

Further provided is the use of the CD80-Fc fusion protein described herein, or the pharmaceutical composition, the isolate nucleic acid, the vector, or the host cell described herein in the manufacture of a medicament. In some aspects, the CD80-Fc fusion protein described herein or the pharmaceutical composition described herein, is for use as a medicament. In some aspects, the medicament is for use in the treatment of cancer. In some aspects the invention provides for any of CD80-Fc fusion proteins disclosed herein for use in a therapy.

Further provided herein is a variant CD80 polypeptide comprising a substitution of one or more amino acids at position V11, V22, T28, E23, A26, Y31, Q33, K36, G45, K54, T57, D60, I61, T62, N63, N64, K89, D90, or A91 of the amino acid sequence of SEQ ID NO: 2. In some aspects, the substitution at position V11 is V11L; the substitution at position V22 is V22C, V22F or V22M; the substitution at position T28 is T28V; the substitution at position E23 is E23C; the substitution at position A26 is A26C; the substitution at position Y31 is Y31Q; the substitution at position Q33 is Q33E; the substitution at position K36 is K36R, the substitution at position G45 is G45C; the substitution at position K54 is K54E; the substitution at position T57 is T57V; the substitution at position D60 is D60F, D60Q, D60R, D60T or D60Y; the substitution at position I61 is I61C; the substitution at position T62 is T62F, T62I, T62L or T62Y; the substitution at position N63 is N63D or N63E; and the substitution at position N64 is N64D or N64E; the substitution at position K89 is K89D, K89E or K89Q; the substitution at position D90 is D90K, D90N or D90Q; and the substitution at position A91 is A91S.

In some aspects, the substitution comprises K89D, K89E, K89Q, D90K, D90N, D90Q, A91S, K89D-D90N, K89D-D90Q, K89D-D90K, K89Q-D90Q, D60Y, I61C, V11L-V22F, V11L-T62Y, V22C-G45C, V22F-D60Y, V22F-T62L, E23C-A26C, T28V-T57V, D60F-T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E, K89Q-D90Q-I61C, D90Q-E23C-A26C, K89Q-D90Q-E23C-A26C, or K89Q-D90Q-V22C-G45C, or K89D-D90K-T28V-T57V of the amino acid sequence of SEQ ID NO: 2.

### BRIEF DESCRIPTION OF THE DRAWINGS

FIGS. 1A and 1B depict A) the regulation of T cell function by the interaction of CD80 (B7-1)/CD86 (B7-2) with CD28 and CTLA-4 and B) the mechanism of CD80-Fc fusion proteins.

FIGS. 2A and 2B depict binding activity of WT and variant CD80-Fc fusion proteins against recombinant soluble A) CD28 and B) CTLA-4 proteins using standard ELISA.

FIG. 3 depicts the binding affinity of WT and variant CD80-Fc fusion proteins against CD28 expressed on Jurkat cells measured by flow cytometry.

FIG. 4 depicts IL-2 production levels from a primary T cell and HCT116-CD64 cell co-stimulation assay with WT and variant CD80-Fc fusion proteins.

FIG. 5 depicts the normalized responses for luciferase reporter activity from a Jurkat-IL-2-Luc and HCT116-CD63 co-stimulation assay with WT and variant CD80-Fc fusion proteins.

FIG. 6 depicts the normalized responses for luciferase reporter activity from a Jurkat-NYESO1-IL-2-Luc and A375-CD64 co-stimulation assay with WT and variant CD80-Fc fusion proteins.

FIG. 7 depicts luciferase reporter activity IL-2 from a Jurkat-CTLA-4-IL-2-Luc and HCT116-CD64 co-stimulation assay with WT and variant CD80-Fc fusion proteins.

FIGS. 8A and 8B depict thermal forced aggregation of WT and variant CD80-Fc fusion proteins.

FIG. 9A-9D depict binding of varying concentrations of WT and variant CD80-Fc fusion proteins at A-B) 60 ug/ml and C-D) 75 ug/ml concentrations of immobilized human PD-L1 measured by surface plasmon resonance (Biacore).

FIG. 10 depicts the viscosity of WT and variant CD80-Fc fusion proteins assessed at various concentrations.

FIG. 11 depicts the IL-2 reporter activity of WT and variant CD80-Fc fusion proteins using a co-culture HCT116-CD64-Jurkat IL-2 reporter assay.

FIG. 12A-12C depict IL-2 production levels from a human PBMC assay with WT and variant CD80-Fc fusion proteins.

FIG. 13A-13C depicts tumor growth inhibition after treatment with WT and variant CD80-Fc fusion proteins at a dose of A) 0.3 mg/kg B) 1 mg/kg and C) 3 mg/kg in a Renca murine renal carcinoma model.

FIGS. 14A and 14B depict tumor growth inhibition after treatment with variant CD80-Fc fusion proteins at a dose of 0.1 mg/kg and 1 mg/kg, respectively, in a CT26 murine colorectal carcinoma model.

FIGS. 15A and 15B depict tumor growth inhibition after treatment with variant CD80-Fc fusion proteins at various doses in an EMT6 murine breast cancer model.

FIGS. 16A and 16B depict tumor growth inhibition after treatment with variant CD80-Fc fusion proteins (cysteine stabilized) administered intravenously (IV) and subcutaneously (SC) at various doses in MC38 murine colorectal carcinoma model.

FIGS. 17A and 17B depict tumor growth inhibition after treatment with variant CD80-Fc fusion proteins (non-cysteine stabilized) administered intravenously (IV) and subcutaneously (SC) at various doses in MC38 murine colorectal carcinoma model.

FIG. 18 depicts the level of tumor-infiltrating CD8+ T cells expressed as a percentage of total immune cells (CD45+) after treatment with WT and variant CD80-Fc fusion proteins.

FIG. 19 depicts the plasma concentrations of WT and variant CD80-Fc fusion proteins at various timepoints following administration in cynomolgus monkeys.

FIG. 20 depicts the plasma concentration of WT and variant CD80-Fc fusion proteins at various timepoints following administration in transgenic mice expressing the human neonatal Fc receptor (huFcRn) a-chain transgene under the control of its natural human promoter.

FIG. 21 depicts tumor growth inhibition after treatment with variant CD80-Fc fusion proteins or αPD1 antibody alone, and a combination treatment of variant CD80-Fc fusion protein and αPD1 antibody in a CT26 murine colorectal carcinoma model.

FIG. 22 depicts tumor growth inhibition after treatment with variant CD80-Fc fusion protein or αPD1 antibody alone, and a combination treatment of variant CD80-Fc fusion protein and αPD1 antibody in a B16F10 murine melanoma model.

FIG. 23 depicts tumor growth inhibition after treatment with variant CD80-Fc fusion proteins or talazoparib alone, and a combination treatment of variant CD80-Fc fusion protein and talazoparib in an EMT6 breast cancer model.

FIG. 24A-24E depict A) IL-2 production, B) CD25 expression, C) Ki-67 expression, D) IFNγ expression, and E) relative abundance (%) of IFNγ+CD8+ T cells after treatment with WT and variant CD80-Fc fusion proteins in primary human T cells.

FIG. 25A-25E depict gene expression levels of A) IL-2, IL-21 and lymphotoxin alpha (LTA), B) BCL-XL and CASP8, C) OX-40, D) IL-7Ra, and E) TIGIT after treatment with WT and variant CD80-Fc fusion proteins in primary human T cells. In each graph, A=anti-human CD3 antibody, B=CD80-WT-Fc, C=CD80-K89D-D90K-T28V-T57V-Fc, and D=anti-human CD28 antibody.

### DETAILED DESCRIPTION OF THE INVENTION

The present invention disclosed herein provides for CD80-Fc fusion proteins and variant CD80 polypeptides. The CD80-Fc fusion proteins described herein have an antibody Fc region (e.g. IgG1) and a variant CD80 polypeptide (e.g. extracellular domain (ECD) of human CD80). The CD80-Fc fusion proteins of the present invention demonstrated improved properties, including but not limited to, increased or enhanced binding affinity to CD28 and increased or enhanced stability, as compared to wild-type CD80-Fc fusion proteins (i.e., fusion proteins comprising wild-type CD80). Further, it is demonstrated that the CD80-Fc fusion proteins described herein did not increase or enhance binding affinity to PD-L1, instead no detectable binding to PD-L1 was observed.

The CD80-Fc fusion proteins described herein demonstrated increased or enhanced co-stimulation, increased or enhanced production of IL-2, significant tumor growth inhibition and tumor growth regression, increased levels of tumor reactive T cells in the spleen and tumor-draining lymph nodes (TDLNs) and enhanced efficacy in combination with one or more additional agents. Further, the CD80-Fc fusion proteins of the present invention demonstrated enhanced thermal stability, decreased or reduced aggregation, decreased or reduced viscosity and improved manufacturability. The invention also provides for processes for modifying, expressing and producing CD80-Fc fusion proteins. The CD80-Fc fusion proteins described herein are useful for the preparation and manufacture of compositions, such as medicaments, that may be used in the enhancement of anti-tumor immunity and treatment of cancer, along with the diagnosis, prophylaxis and/or treatment of disorders. The invention further provides for nucleic acids encoding the CD80-Fc fusion proteins and components thereof.

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, analytical chemistry, biochemistry, molecular biology, immunology, microbiology, genetics and protein and nucleic acid chemistry, medicinal and pharmaceutical chemistry and hybridization described herein are those well-known and commonly used in the art.

The practice of the present invention will employ, unless otherwise indicated, conventional techniques of molecular biology (including recombinant techniques), microbiology, cell biology, biochemistry and immunology, which are within the skill of the art. Such standard methods are explained in the literature, such as, described Sambrook, Fritsch and Maniatis (1982 & 1989 2nd ed., 2001 3rd ed.) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY: Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. NY, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

Throughout this specification and claims, the word “comprise,” or variations such as “comprises” or “comprising,” will be understood to imply the inclusion of a stated integer or group of integers but not the exclusion of any other integer or group of integers. It is understood that wherever aspects are described herein with the language “comprising,” otherwise analogous aspects described in terms of “consisting of” and/or “consisting essentially of” are also provided.

The articles “a”, “an” and “the” are used herein to refer to one or to more than one (i.e., to at least one) of the grammatical object of the article. Unless otherwise required by context, singular terms shall include pluralities and plural terms shall include the singular. Any example(s) following the term “e.g.” or “for example” is not meant to be exhaustive or limiting.

As used herein, the terms “CD80”, “B7-1”, “B7.1” or “B7/BB1”, which are used interchangeably, refer to any form of CD80 and variants thereof that retain at least part of the activity of CD80. Unless indicated differently, such as by specific reference to human CD80, CD80 includes all species of CD80. Exemplary wild-type human CD80 sequences include without limitation: UniProtKB: P33681-1; isoform 1 (SEQ ID NO: 1), UniProtKB: P33681-2; isoform 2 and UniProtKB: P33681-3; isoform 3. An exemplary mouse CD80 sequence is found as UniProtKB: Q3U4B5. An exemplary cynomolgus monkey CD80 sequence is found as UniProtKB: G7NXN7. Exemplary wild-type human CD80 proteins included, but are not limited to, the sequences listed below.

Wild-type human CD80
(UniProtKB: P33681-1; isoform 1):
(SEQ ID NO: 1)
MGHTRRQGTSPSKCPYLNFFQLLVLAGLSHFCSGVIHVTKEVKEV
ATLSCGHNVSVEELAQTRIYWQKEKKMVLTMMSGDMNIWPEYKNR
TIFDITNNLSIVILALRPSDEGTYECVVLKYEKDAFKREHLAEVT
LSVKADFPTPSISDFEIPTSNIRRIICSTSGGFPEPHLSWLENGE
ELNAINTTVSQDPETELYAVSSKLDFNMTTNHSFMCLIKYGHLRV
NQTFNWNTTKQEHFPDNLLPSWAITLISVNGIFVICCLTYCFAPR
CRERRRNERLRRESVRPV
Wild-type human CD80 extracellular
domain (ECD):
(SEQ ID NO: 2)
VIHVTKEVKEVATLSCGHNVSVEELAQTRIYWQKEKKMVLTMMSG
DMNIWPEYKNRTIFDITNNLSIVILALRPSDEGTYECVVLKYEKD
AFKREHLAEVTLSVKADFPTPSISDFEIPTSNIRRIICSTSGGFP
EPHLSWLENGEELNAINTTVSQDPETELYAVSSKLDFNMTTNHSF
MCLIKYGHLRVNQTFNWNTTKQEHFPDN

As used herein, the terms “CD28”, “TP44”, or “T cell-specific surface glycoprotein CD28”, which are used interchangeable, refer to any form of CD28 and variants thereof and retain at least part of the activity of CD28. Unless indicated differently, such as by specific reference to human CD28, CD28 includes all species of CD28. Exemplary human CD28 sequences are as found as UniProtKB: P10747, including isoforms 1-7. Exemplary mouse CD28 sequences are found as UniProtKB: P31041, including isoforms 1-7. An exemplary cynomolgus monkey CD28 sequence is found as UniProtKB: Q0ODN3.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

An “antibody” or “Ab” as used herein refers to an immunoglobulin molecule capable of recognizing and binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding portion or fragments thereof (for example Fab, Fab′, F(ab′)₂, Fd, Fv), domain antibodies (dAbs, e.g., shark and camelid antibodies), fragments including complementarity determining regions (CDRs), single chain variable fragment antibodies (scFv), bispecific single chain fragment (bis-scFv), disluside-linked Fv fragment (dsFv), anti-idiotypic (anti-id) antibodies, bispecific antibodies, heteroconjugate antibodies, fusion proteins having an antibody, maxibodies, minibodies, intrabodies, diabodies, triabodies, tetrabodies, v-NAR and polypeptides that contain at least a portion of an immunoglobulin that is sufficient to confer specific antigen binding to the polypeptide.

An antibody includes an antibody of any class (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgA₁, IgA₂, IgG₁, IgG₂, IgG₃ and IgG₄. The heavy-chain constant regions that correspond to the different classes of immunoglobulins are called alpha, delta, epsilon, gamma, and mu, respectively. The subunit structures and three-dimensional configurations of different classes of immunoglobulins are well known. The invention also includes “antibody analog(s),” other non-antibody molecule protein-based scaffolds, e.g., fusion proteins and/or immunoconjugates that use CDRs to provide specific antigen binding. The antibodies of the invention can be derived from any species including, but not limited to mouse, human, camel, llama, fish, shark, goat, rabbit, chicken, and bovine. The term “antibody” or “Ab” further includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (VH) and a heavy chain constant region. The heavy chain constant region comprises three domains, CH1, CH2 and CH3. The CH1 and CH2 domains are connected by a hinge region. Each light chain comprises a light chain variable region (VL) and a light chain constant region. The light chain constant region comprises one domain (CL1).

As used herein, “variable region” of an antibody refers to the variable region of the antibody light chain (VL) or the variable region of the antibody heavy chain (VH), either alone or in combination. As known in the art, the variable regions of the heavy and light chains each consist of four framework regions (FRs) connected by three complementarity determining regions (CDRs), also known as hypervariable regions, and contribute to the formation of the antigen binding site of antibodies. The CDRs in each chain are held together in close proximity by the FRs and, with the CDRs from the other chain, contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J. Mol. Biol. 196(4): 901-917, 1987). In certain aspects, definitive delineation of a CDR and identification of residues comprising the binding site of an antibody is accomplished by solving the structure of the antibody and/or solving the structure of the antibody-ligand complex. This may be accomplished by any of a variety of techniques known to those skilled in the art, such as X-ray crystallography.

A “CDR” of a variable region are amino acid residues within the variable region that are identified in accordance with the definitions of the Kabat, Chothia, the accumulation of both Kabat and Chothia, AbM, contact, North and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1986, J. Mol. Biol., 196: 901-17; Chothia et al., 1989, Nature, 342: 877-83. The AbM definition of CDRs is a compromise between Kabat and Chothia and uses Oxford Molecular's AbM antibody modeling software (Accelrys®). The “contact” definition of CDRs is based on observed antigen contacts, set forth in MacCallum et al., J. Mol. Biol., 262:732-745, 1996. The “conformational” definition of CDRs is based on residues that make enthalpic contributions to antigen binding (see, e.g., Makabe et al., J. Biol. Chem., 283:1156-1166, 2008). North has identified canonical CDR conformations using a different preferred set of CDR definitions (North et al., J. Mol. Biol. 406: 228-256, 2011). Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, North and/or conformational definitions, or any method of CDR determination well known in the art. Antibodies, or antigen-binding fragments thereof, of the present invention include one or more CDR(s) (such as one, two, three, four, five, or all six CDRs).

The term “percent identical” in the context of amino acid sequences means the number of residues in two sequences that are the same when aligned for maximum correspondence. There are a number of different algorithms known in the art which can be used to measure amino acid percent identity (i.e., the Basic Local Alignment Tool or BLAST®). Unless otherwise specified, default parameters for a particular program or algorithm are used.

As known in the art, a “constant region” of an antibody refers to the constant region of the antibody light chain or the constant region of the antibody heavy chain, either alone or in combination.

The term “fusion protein” refers to a protein or polypeptide that has an amino acid sequence derived from two or proteins. The fusion protein may also include linking regions of amino acids between the two or more proteins. For example, a fusion protein may comprise a protein (e.g., CD80 or variant thereof) and an antibody or antibody fragment (e.g., an antibody Fc region), or a protein (e.g., CD80 or variant thereof) and human serum albumin (HSA).

The terms “polypeptide”, “oligopeptide”, “peptide” and “protein” are used interchangeably herein to refer to chains of amino acids of any length (e.g., CD80 or variant thereof). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As used herein, the term “Fc region,” “Fc domain,” “Fc chain” or analogous terms are used to define a C-terminal region of an immunoglobulin heavy chain. The Fc region interacts with cell receptors (e.g. Fc receptors) and complement proteins. The Fc region of an immunoglobulin generally comprises two constant domains, CH2 and CH3. As is known in the art, an Fc region can be present in monomeric or multimeric (e.g. dimer) form. The Fc region may be a native sequence Fc region or a variant Fc sequence. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991. In certain aspects, an Fc chain begins in the hinge region just upstream of the papain cleavage site and ends at the C-terminus of the antibody.

Accordingly, a Fc chain may comprise at least a hinge domain, a CH2 domain, and a CH3 domain. In certain aspects, an Fc chain comprises at least one of: a hinge (e.g., upper, middle, and/or lower hinge region) domain, a CH2 domain, a CH3 domain, a CH4 domain, or a variant, portion, or fragment thereof. In certain aspects, an Fc domain comprises a complete Fc chain (i.e., a hinge domain, a CH2 domain, and a CH3 domain). In certain aspects, an Fc chain comprises a hinge domain (or portion thereof) fused to a CH3 domain (or portion thereof). In certain aspects, an Fc chain comprises a CH2 domain (or portion thereof) fused to a CH3 domain (or portion thereof). In certain aspects, an Fc chain consists of a CH3 domain or portion thereof. In certain aspects, an Fc chain consists of a CH2 domain (or portion thereof) and a CH3 domain. In certain aspects, an Fc chain consists of a hinge domain (or portion thereof) and a CH2 domain (or portion thereof). The Fc chain may be derived from an immunoglobulin of any species and/or any subtype, including, but not limited to, a human IgG1, IgG2, IgG3, IgG4, IgD, IgA, IgE, or IgM antibody. In some embodiment, the Fc chain comprises the carboxy-terminal portions of both heavy chains held together by disulfides.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, Ann. Rev. Immunol., 9:457-92, 1991; Capel et al., Immunomethods, 4:25-34, 1994; and de Haas et al., J. Lab. Clin. Med., 126:330-41, 1995. “FcR” also includes the neonatal receptor, FcRn, which is responsible for the transfer of maternal IgGs to the fetus (Guyer et al., J. Immunol., 117:587, 1976; and Kim et al., J. Immunol., 24:249, 1994).

A “native sequence Fc region” or “wild-type Fc region” comprises an amino acid sequence identical to the amino acid sequence of an Fc region found in nature.

A “variant Fc region” or “variant Fc chain” comprises an amino acid sequence which differs from that of a native sequence Fc region by virtue of at least one amino acid modification yet retains at least one effector function of the native sequence Fc region. In some aspects, the variant Fc chain has at least one amino acid substitution compared to a native sequence Fc chain or to the Fc region of a parent polypeptide, e.g., from about one to about ten amino acid substitutions, or from about one to about five amino acid substitutions in a native sequence Fc chain or in the Fc chain of the parent polypeptide. A variant Fc chain herein may possess at least about 80% sequence identity with a native sequence Fc chain and/or with an Fc chain of a parent polypeptide, or may be at least about 90%, at least about 95%, at least about 96%, at least about 97%, at least about 98%, or at least about 99% sequence identity therewith.

A “functional Fc region” possesses at least one effector function of a native sequence Fc region. The term “effector function” refers to the biological activities attributable to the Fc region (a native sequence Fc chain or variant Fc chain) of an antibody, and vary with the antibody isotype. Examples of antibody effector functions include, but are not limited to, antibody-dependent cell-mediated cytotoxicity (ADCC), Fc receptor binding, complement dependent cytotoxicity (CDC), phagocytosis, C1q binding, down regulation of cell surface receptors (e.g., B cell receptor; BCR) and B cell activation. Such effector functions generally require the Fc region to be combined with a binding domain (e.g., an antibody variable domain) and can be assessed using various assays known in the art for evaluating such antibody effector functions. An exemplary measurement of effector function is through Fcγ3 and/or C1q binding.

As used herein “antibody-dependent cell-mediated cytotoxicity” or “ADCC” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” or “CDC” refers to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202: 163 (1996), may be performed.

As used herein, the term “binding affinity,” generally refers to the strength of the sum total of noncovalent interactions between a single binding site of a molecule and its binding partner (e.g. polypeptide-receptor or antibody-antigen interaction). For example, the interaction between CD80 and T-cell receptors CD28 and CTLA-4. The binding affinity of a molecule for its binding partner may be represented by the equilibrium dissociation constant (K_{D}). The K_{D} is the ratio of the rate of dissociation, also called the “off-rate” or “k_{d}”, to the rate of association, or “on-rate” or “kₐ”. Thus, K_{D} equals kₐ/kₐ and is expressed as a molar concentration (M). It follows that the smaller the K_{D}, the greater the binding affinity of a molecule for its binding partner. Therefore, a K_{D} of 1 μM indicates weak binding affinity compared to a K_{D} of 1 nM. K_{D} values can be determined using methods well established in the art, including those described herein. One method for determining the binding affinity and K_{D} is by using surface plasmon resonance, typically using a biosensor system such as a BIAcore® system. Other standard assays to evaluate the binding ability of polypeptides to ligands are known in the art, including for example, ELISAs, Western blots, RIAs, and flow cytometry analysis.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, sialylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “target antigen,” a “tumor antigen,” or a “tumor-associated antigen,” as used herein refers to an antigenic determinant presented on the surface of a target cell, for example a cell in a tumor such as a cancer cell or a cell of the tumor stroma.

As used herein, the term “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond. In the context of this invention, the cysteines which form the disulfide bond are within the framework regions of the single chain antibody and serve to stabilize the conformation of the antibody or fragment thereof. Cysteine residues can be introduced, e.g., by site directed mutagenesis, so that stabilizing disulfide bonds can be made within the molecule.

A polypeptide or antibody that “specifically binds” or “preferentially binds” (used interchangeably herein) to a receptor or antigen (e.g., CD28 protein) is a term well understood in the art, and methods to determine such specific or preferential binding are also well known in the art. A molecule is said to exhibit “specific binding” or “preferential binding” if it reacts or associates with greater affinity, avidity, more readily, and/or with greater duration with a particular cell or substance than it does with alternative cells or substances. For example, a polypeptide (e.g., CD80) that specifically or preferentially binds to a target receptor (e.g., CD28) binds this receptor with greater affinity, avidity, more readily, and/or with greater duration than it binds to other target receptors or non-target receptors.

The term “monoclonal antibody” or “mAb” refers to an antibody obtained from a population of substantially homogeneous antibodies, i.e., the individual antibodies comprising the population are identical except for possible naturally-occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, “humanized” antibody refers to forms of antibodies that contain sequences of non-human (e.g., mouse, rat, rabbit, non-human primate or other mammal) antibodies as well as human antibodies. Preferably, humanized antibodies are human immunoglobulins (recipient antibody) in which residues from one or more CDRs of the recipient are replaced by residues from one or more CDRs of a non-human species (donor antibody) such as mouse, rat, rabbit, non-human primate or other mammal having the desired specificity, affinity, capacity or other biological activity. The humanized antibody optionally also will comprise at least a portion of an immunoglobulin constant region (Fc), typically that of a human immunoglobulin.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chains is identical with or homologous to corresponding sequences in an antibody derived from another species (e.g., mouse) or belonging to another antibody class or subclass, as well as fragments of such antibodies, so long as they exhibit the desired biological activity.

As used herein, “human antibody” means an antibody having an amino acid sequence corresponding to that of an antibody produced by a human and/or which has been made using any of the techniques for making human antibodies known to those skilled in the art or disclosed herein. Accordingly, human antibody is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. This definition of a human antibody includes antibodies comprising at least one human heavy chain polypeptide or at least one human light chain polypeptide.

As used herein, the term “isolated” refers to material that is removed from its original environment (e.g., the natural environment if it is naturally occurring). For example, a naturally occurring polynucleotide or polypeptide present in a living animal is not isolated, but the same polynucleotide or polypeptide that is separated from some or all of the coexisting materials in the natural system is isolated. Such polynucleotide could be part of a vector and/or such polynucleotide or polypeptide could be part of a composition, e.g., a mixture, solution or suspension or comprising an isolated cell or a cultured cell which comprises the polynucleotide or polypeptide, and still be isolated in that the vector or composition is not part of its natural environment.

As known in the art, the terms “nucleic acid” and “polynucleotide” as used interchangeably refer to polymeric forms of nucleotides of any length, either deoxyribonucleotides or ribonucleotides, analogs thereof, or any substrate that can be incorporated into a chain by DNA or RNA polymerase. Polynucleotides may have any three-dimensional structure, and may perform any function, known or unknown. Polynucleotides may be naturally-occurring, synthetic, recombinant or any combination thereof. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. Nucleic acids and polynucleotide encoding the polypeptides and antibodies of the invention can be cloned into a vector for expression or propagation. The present invention also includes polynucleotides that encode the polypeptides and antibodies of the invention, including binding regions of the polypeptides and antibodies. The polynucleotides encoding the molecules of the invention may be obtained, and the nucleotide sequence of the polynucleotides determined, by any method known in the art. The sequence encoding the polypeptides and antibodies of interest may be maintained in a vector in a host cell and the host cell may then be expanded and frozen for future use. Production of recombinant polypeptides and/or antibodies in cell culture can be carried out through cloning of genes from B cells by means known in the art. See, e.g. Tiller et al., J. Immunol. Methods 329:112-124, 2008; U.S. Pat. No. 7,314,622.

As used herein, the term “vector” refers to a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, the term “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The term “biomarker” as used herein refers to an indicator molecule or set of molecules (e.g., predictive, diagnostic, and/or prognostic indicator), which can be detected in a sample. The biomarker may be a predictive biomarker and serve as an indicator of the likelihood of sensitivity or benefit of a patient having a particular disease or disorder (e.g., a proliferative cell disorder (e.g., cancer)) to a particular treatment (e.g. treatment with a CD80-Fc fusion protein). Biomarkers include, but are not limited to, polynucleotides (e.g., DNA and/or RNA (e.g., mRNA)), polynucleotide copy number alterations (e.g., DNA copy numbers), polynucleotide sequence alterations (e.g. gene mutations or gene variants), polypeptides, polypeptide and polynucleotide modifications (e.g., post-translational modifications), carbohydrates, and/or glycolipid-based molecular markers. In some aspects, a biomarker is a gene.

The term “neoplastic disorder” refers to a condition in which cells proliferate at an abnormally high and uncontrolled rate, the rate exceeding and uncoordinated with that of the surrounding normal tissues. It usually results in a solid lesion or lump known as “tumor.” This term encompasses benign and malignant neoplastic disorders. The term “malignant neoplastic disorder”, which is used interchangeably with the term “cancer” in the present disclosure, refers to a neoplastic disorder characterized by the ability of the tumor cells to spread to other locations in the body (known as “metastasis”). The term “benign neoplastic disorder” refers to a neoplastic disorder in which the tumor cells lack the ability to metastasize.

As used herein, the term “cancer”, “cancerous” or “malignant” refers to or describes a physiological condition in mammals that is typically characterized by unregulated cell growth, a neoplasm or a tumor resulting from abnormal uncontrolled growth of cells. In some aspects, cancer refers to a malignant primary tumor without metastasis, which has remained localized. In other aspects, cancer refers to a malignant tumor, which has invaded and destroyed neighboring body structures and spread to distant sites. In some aspects, the cancer is associated with a specific cancer antigen. Examples of cancer include but are not limited to, gastric cancer, small intestine cancer, sarcoma, lymphoma, Hodgkin's lymphoma, leukemia, multiple myeloma, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer (e.g., non-small-cell lung cancer), mesothelioma, ovarian cancer, breast cancer, prostate cancer, esophageal cancer, pancreatic cancer, glioma, renal cancer (e.g., renal cell carcinoma), bladder cancer, cervical cancer, uterine cancer, vulvar cancer, penile cancer, testicular cancer, anal cancer, choriocarcinoma, colon cancer, colorectal cancer, oral cancer, skin cancer, Merkel cell carcinoma, glioblastoma, brain tumor, bone cancer, eye cancer, melanoma, and cancer with high microsatellite instability (MSI-H).

As used herein, “treat,” “treating” or “treatment” is an approach for obtaining beneficial or desired clinical results. For purposes of the present invention, treatment is defined as the administration of a CD80-Fc fusion protein to a subject, e.g., a patient. Such administration can be e.g., by direct administration to the subject or by application to an isolated tissue or cell from a subject which is returned to the subject. The CD80-Fc fusion protein be administered alone or in combination with one or more additional agents. The treatment can be to cure, heal, alleviate, relieve, alter, remedy, ameliorate, palliate, improve or affect the disorder, the symptoms of the disorder or the predisposition toward the disorder, e.g., a cancer. In some aspects treatment includes, but is not limited to, one or more of the following: reducing the proliferation of (or destroying) neoplastic or cancerous cells, inhibiting metastasis of neoplastic cells, shrinking or decreasing the size of a tumor, remission of cancer, decreasing symptoms resulting from cancer, increasing the quality of life of those suffering from cancer, decreasing the dose of other medications required to treat cancer, delaying the progression of cancer, curing a cancer, and/or prolong survival of patients having cancer.

As used herein, the term “ameliorating” means a lessening or improvement of one or more symptoms as compared to not administering a CD80-Fc fusion protein as described herein. “Ameliorating” also includes shortening or reduction in duration of a symptom.

As used herein, the terms “prevent”, “preventing” and “prevention” refer to the prevention of the recurrence or onset of a disorder or one or more symptoms of a disorder in a subject as result of the administration of a prophylactic or therapeutic agent.

As used herein, “inhibiting the growth” of the tumor or cancer refers to slowing, interrupting, arresting or stopping its growth and/or metastases and does not necessarily indicate a total elimination of the tumor growth.

The term “immune-effector-cell enhancer” or “IEC enhancer” refers to a substance capable of increasing or enhancing the number, quality, or function of one or more types of immune effector cells of a mammal. Examples of immune effector cells include cytolytic CD8 T cells, CD4 T cells, NK cells, and B cells.

The term “immune modulator” refers to a substance capable of altering (e.g., inhibiting, decreasing, increasing, enhancing, or stimulating) the immune response (as defined herein) or the working of any component of the innate, humoral or cellular immune system of a host mammal. Thus, the term “immune modulator” encompasses the “immune-effector-cell enhancer” as defined herein and the “immune-suppressive-cell inhibitor” as defined herein, as well as substance that affects other components of the immune system of a mammal.

The term “immune response” refers to any detectable response to a particular substance (such as an antigen or immunogen) by the immune system of a host mammal, such as innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids).

The term “immunogenic” refers to the ability of a substance to cause, elicit, stimulate, or induce an immune response, or to improve, enhance, increase or prolong a pre-existing immune response, against a particular antigen, whether alone or when linked to a carrier, in the presence or absence of an adjuvant.

The term “immune-suppressive-cell inhibitor” or “ISC inhibitor” refers to a substance capable of reducing or suppressing the number or function of immune suppressive cells of a mammal. Examples of immune suppressive cells include regulatory T cells (“Treg”), myeloid-derived suppressor cells, and tumor-associated macrophages.

As used herein, the term “subject” is intended to include any animal (e.g., a mammal), including, but not limited to, humans, non-human primates, rodents, and the like, which is to be the recipient of a particular treatment. For example, a subject can be a patient (e.g., a human patient or a veterinary patient), having a cancer. Typically, the terms “subject,” “individual” and “patient” are used interchangeably herein in reference to a human subject.

As used herein, the term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, P A, 1990; and Remington, The Science and Practice of Pharmacy 20th Ed. Mack Publishing, 2000).

As used herein, an “effective amount,” “therapeutically effective amount.” “therapeutically sufficient amount,” or “effective dosage” refers to any amount of a therapeutic agent which is effective or sufficient, upon single or multiple dose administration to a subject, in preventing, healing, ameliorating, treating or managing a disease, disorder or side effect, or decreasing the rate of advancement of a disease or disorder, or in prolonging curing, alleviating, relieving, or improving the condition of a subject with a disorder as described herein beyond that expected in the absence of such treatment. The term also includes within its scope amounts effective to enhance normal physiological function. An effective amount may be considered in the context of administering one or more therapeutic agents, and a single agent may be considered to be given in an effective amount if, in conjunction with one or more other agents, a desirable result may be or is achieved.

Potency is a measure of the activity of a therapeutic agent expressed in terms of the amount required to produce an effect of given intensity. A highly potent agent evokes a greater response at low concentrations compared to an agent of lower potency that evokes a smaller response at low concentrations. Potency is a function of affinity and efficacy. Efficacy refers to the ability of therapeutic agent to produce a biological response upon binding to a target ligand and the quantitative magnitude of this response. As used herein, the term “half maximal effective concentration (EC50)” refers to the concentration of a therapeutic agent which causes a response halfway between the baseline and maximum after a specified exposure time. The therapeutic agent may cause inhibition or stimulation. The EC50 value is commonly used, and is used herein, as a measure of potency.

Amino acid modifications can be made by any method known in the art and many such methods are well known and routine for the skilled artisan, e.g. mutations, substitutions, deletions, and/or additions. For example, but not by way of limitation, amino acid substitutions, deletions and insertions may be accomplished using any well-known PCR-based technique. Amino acid substitutions may be made by site-directed mutagenesis (see, for example, Zoller and Smith, 1982, Nucl. Acids Res. 10:6487-6500; and Kunkel, 1985, PNAS 82:488).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest, DNA, RNA or virus-like particles. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164). Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2nd ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

Enzymatic reactions and purification techniques are performed according to manufacturer's specifications, as commonly accomplished in the art or as described herein. Standard techniques are used for chemical syntheses, chemical analyses, pharmaceutical preparation, formulation, and delivery, and treatment of patients.

Reference to “about” a value or parameter herein includes (and describes) aspects that are directed to that value or parameter per se. For example, description referring to “about X” includes description of “X.” Numeric ranges are inclusive of the numbers defining the range. Generally, the term “about” refers to the indicated value of the variable and to all values of the variable that are within the experimental error of the indicated value (e.g. within the 95% confidence interval for the mean) or within percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

Where aspects or aspects of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control.

Exemplary methods and materials are described herein, although methods and materials similar or equivalent to those described herein can also be used in the practice or testing of the present invention. The materials, methods, and examples are illustrative only and not intended to be limiting.

### CD80-Fc Fusion Proteins

CD80 (B7-1, B.7 or B7/BB1) and CD86 (B7-2) are costimulatory molecules on the surface of antigen presenting cells (APCs) that bind to CD28 and cytotoxic T-lymphocyte-associated antigen (CTLA-4) on T cells. CD80 is a 45-60 kDa type I transmembrane glycoprotein that contains two extracellular domains, a membrane distal Ig variable-like domain and a membrane proximal Ig constant-like domain. (Soskic et al. Advances in Immunology, Vol. 124, 2014, 95-136).

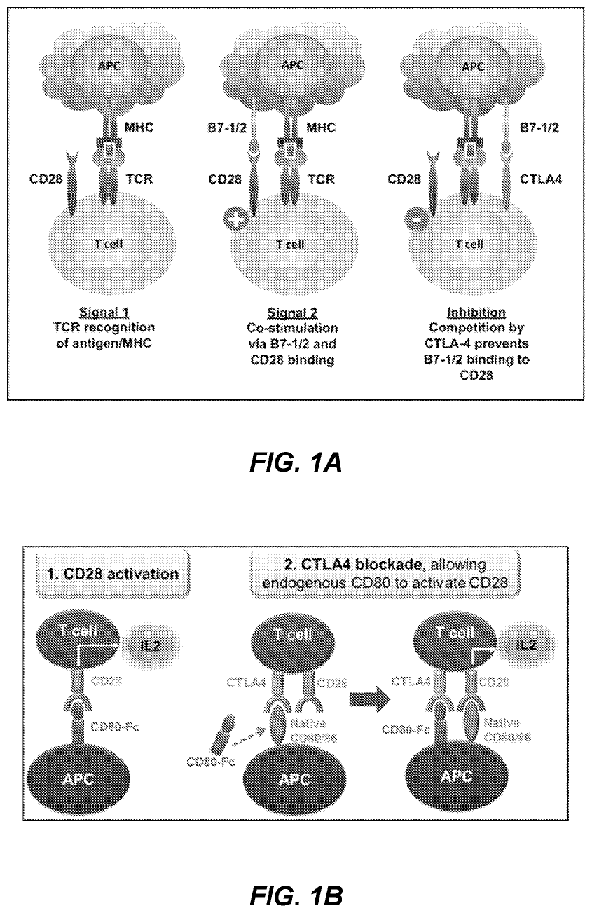

T cells require multiple signals for optimal activation, proliferation and function (FIG. 1A; adapted from Alegre et al, Nat Rev Imm, 2001). Signal 1 is driven by interaction between the T cell receptor (TCR) on T cells and antigen presented on MHC by antigen presenting cells (APCs). Signal 2 is the co-stimulatory signal, and the best described co-stimulatory interactions are CD80 or CD86 on APCs engaging CD28 on T cells after antigen recognition. Signal 2 does not occur in the absence of Signal 1.

The affinity of CD80 for both CD28 and CTLA-4 is substantially greater than CD86 making CD80 a potentially more potent ligand. CD80/CD86 binding to CD28 on both naïve and previously activated T cells in the context of TCR engagement activates downstream signaling pathways resulting in the production of the cytokine IL-2. IL-2 is a key driver of T cell survival, proliferation and differentiation. CTLA-4 is also expressed on T cells and is structurally similar to CD28. CD80/CD86 binding to CTLA-4 on T cells results in reduced T cell co-stimulation. CTLA-4 has a higher affinity for CD80/CD86 compared to CD28 and therefore will outcompete CD28 for binding to CD80/CD86, thus limiting CD28 activation and subsequent IL2 production. CD80 also binds PD-L1 in cis (proteins expressed on the same cell surface interface) which can prevent the interaction of PD-L1 with PD1.

Herein provides for CD80-Fc fusion proteins having an antibody Fc region (e.g. IgG1) fused or linked to a variant CD80 polypeptide (e.g. extracellular domain (ECD) of human CD80). Such CD80-Fc fusion proteins provided herein have enhanced or increased binding affinity to CD28 and promote T cell function by enhancing co-stimulatory signaling without systemic immune activation.

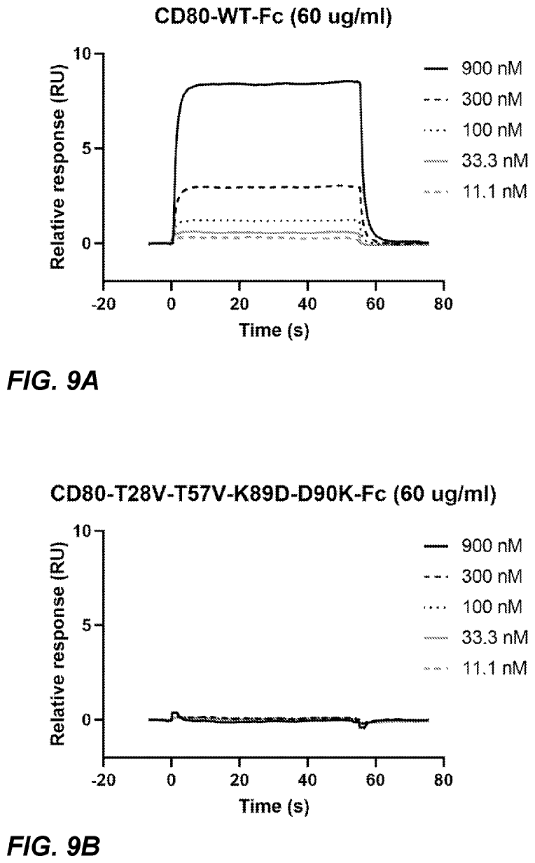

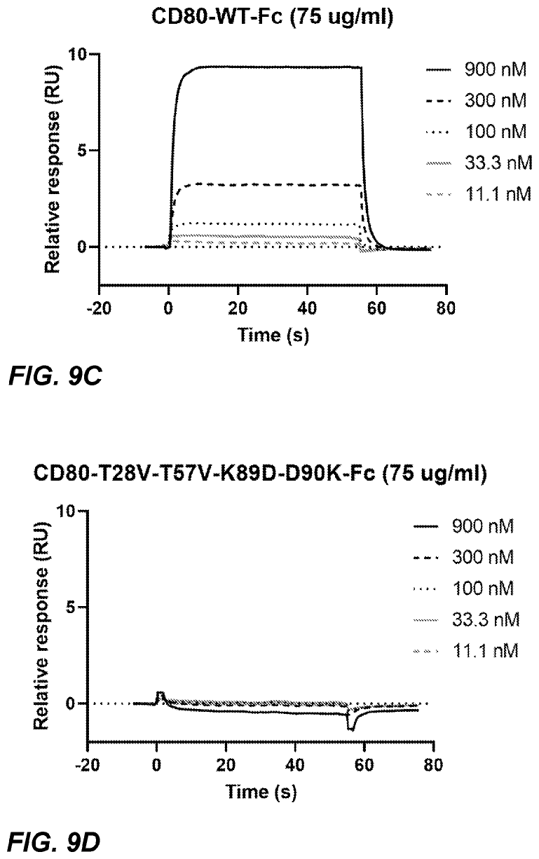

As shown in FIG. 1B, CD80-Fc fusion proteins of the present invention facilitate binding to CD28 on T cells, resulting in T cell priming and CD28 activation, IL-2 production, tumor infiltration of T cells and killing of tumor cells by cytotoxic T cells. In addition, it is demonstrated that the CD80-Fc fusion proteins described herein did not increase or enhance binding to PD-L1, instead no detectable binding to PD-L1 was observed (FIG. 9A-9D). In some aspects, both signal 1 (antigen recognition) and FcγR binding initiate efficacy of CD80-Fc fusion proteins of the present invention. Activity is established at sites comprising both tumor antigen and FcγR-expressing cells, such as tumor-draining lymph nodes and the tumor microenvironment, thus limiting systemic immune activation.

CD80-Fc fusion proteins of the present invention demonstrated enhanced or increased binding to CD28, as compared to wild-type CD80 fusion proteins, see Example 1 (Standard ELISA and Jurkat T cell assay) and Example 7 (Biacore). In some aspects, the CD80-Fc fusion proteins have increased or enhanced binding to CD28, as compared to wild-type CD80 fusion proteins, which enhanced CD28-mediated co-stimulation, see Example 2 (Primary T cell and Jurkat-IL-2-Luc reporter co-stimulation assays), Example 10 (Jurkat IL-2 reporter and Human peripheral blood mononuclear cell (PMBC) assays).

In some aspects, compared to wild-type CD80-Fc fusion proteins, the CD80-Fc fusion proteins of the present invention demonstrated at least at 2%, at least 5%, at least 10%, at least 20%, at least 30%, at least 40%, at least 50%, at least 2-fold, at least 3-fold, at least 4-fold, at least 5-fold, at least 10-fold, at least 15-fold, at least 20-fold, or at least 25-fold increase in binding affinity to CD28. In another aspect, compared to wild-type CD80-Fc fusion proteins, the CD80-Fc fusion proteins of the present invention demonstrated at least at 2%, at least 5%, at least 10%, at least 20%, at least 30%, at least 40%, at least 50%, at least 2-fold, at least 3-fold, at least 4-fold, at least 5-fold, at least 10-fold, at least 15-fold, at least 20-fold, or at least 25-fold increase or enhancement in T cell co stimulation.

CD80-Fc fusion proteins of the present invention demonstrated enhanced efficacy and tumor growth inhibition and/or regression in tumor models in immune-competent mice and increased the levels of tumor-reactive T cells in the spleen and/or tumor-draining lymph nodes (TDLNs) after treatment, see Examples 12, 13, and 14. In some aspects, the CD80-Fc fusion proteins described herein demonstrated enhanced efficacy and tumor growth inhibition when dosed either intravenously (IV) or subcutaneously (SC), see Example 15. In another aspect, CD80-Fc fusion proteins described herein demonstrated an increase in CD8⁺ T cell infiltration in tumors after treatment, see Example 16. Further, CD80-Fc fusion proteins of the present invention demonstrated enhanced efficacy and tumor growth inhibition and/or regression in combination with one or more additional therapeutic agents, see Examples 18 and 19.

In some aspects, treatment with CD80-Fc fusion proteins of the present invention demonstrated at least at 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% tumor growth inhibition. In another aspect, treatment with CD80-Fc fusion proteins of the present invention in combination with one or more additional therapeutic agents demonstrated at least at 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% tumor growth inhibition.

CD80-Fc fusion proteins of the present invention were assessed to demonstrate improved or enhanced molecular stability by identifying disulfide and non-cysteine stabilizing mutation positions, see Example 3. In some aspects, the CD80-Fc fusion proteins described herein demonstrated enhanced thermal stability, as compared to wild-type CD80-Fc fusion proteins, see Example 5. In another aspect, the CD80-Fc fusion proteins described herein demonstrated reduced thermal forced aggregation, as compared to wild-type CD80-Fc fusion proteins, see Example 6. In another aspect, the CD80-Fc fusion proteins described herein demonstrated reduced viscosity and/or improved high concentration viscosity, as compared to wild-type CD80-Fc fusion proteins, see Example 8. In another aspect, the CD80-Fc fusion proteins described herein demonstrated favorable yield and/or purity parameters, as compared to wild-type CD80-Fc fusion proteins, see Example 9.

In some aspects, the CD80-Fc fusion proteins described herein demonstrated favorable pharmacokinetic (PK) assessments which correlate with favorable human PK profiles, see Example 4 (Non-specific binding and Self-interaction assays) and Example 17 (Cynomolgus PK assessment). CD80-Fc fusion proteins of the present invention demonstrated favorable safety parameters and showed no significant induction of cytokine release (IL-2 and IFNγ) and no evidence of superagonism in the absence of TCR stimulation, see Example 11.

Provided herein are CD80-Fc fusion proteins that have increased or enhanced binding to CD28, as compared to wild-type CD80-Fc fusion proteins. Further provided are CD80 fusion proteins that do not increase or enhance binding to PD-L1, as compared to wild-type CD80-Fc fusion proteins. Further provided are CD80-Fc fusion proteins that demonstrate minimal or no detectable binding to PD-L1.

In one aspect, the CD80-Fc fusion proteins have increased or enhanced binding to CD28 and do not increase or enhance binding to PD-L1, as compared to wild-type human CD80-Fc fusion proteins. In another aspect, the CD80-Fc fusion proteins have increased or enhanced binding to CD28, as compared to wild-type CD80-Fc fusion proteins, and demonstrate minimal or no binding to PD-L1.

In one aspect, the CD80-Fc fusion proteins have increased or enhanced binding to CD28 and/or do not increase or enhance binding to PD-L1, as compared to wild-type CD80-Fc fusion proteins. In another aspect, the CD80-Fc fusion proteins have increased or enhanced binding to CD28 and do not increase or enhance binding to PD-L1, as compared to wild-type CD80-Fc fusion proteins.

In one aspect, the CD80-Fc fusion proteins have increased or enhanced binding to CD28, as compared to wild-type CD80-Fc fusion proteins, and/or demonstrate minimal or no binding to PD-L1. In another aspect, the CD80-Fc fusion proteins have increased or enhanced binding to CD28, as compared to wild-type CD80-Fc fusion proteins, and demonstrate minimal or no binding to PD-L1.

Provided herein are CD80-Fc fusion protein comprising: (i) an antibody Fc region and (ii) a variant CD80 polypeptide. In one aspect, the invention provides for a CD80-Fc fusion protein comprising: (i) an antibody Fc region and (ii) a variant CD80 polypeptide comprising a substitution of one or more amino acids of the CD80 extracellular domain (ECD). The variant CD80 polypeptide may be any variant described herein.

Further provided herein is a CD80-Fc fusion protein comprising: (i) an antibody Fc region and (ii) a variant CD80 polypeptide comprising a substitution of one or more amino acids at position V1, I2, H3, V4, T5, K6, E7, V8, K9, E10, V11, A12, T13, L14, S15, C16, G17, H18, N19, V20, S21, V22, E23, E24, L25, A26, Q27, T28, R29, I30, Y31, W32, Q33, K34, E35, K36, K37, M38, V39, L40, T41, M42, M43, S44, G45, D46, M47, N48, I49, W50, P51, E52, Y53, K54, N55, R56, T57, I58, F59, D60, I61, T62, N63, N64, L65, S66, 167, V68, 169, L70, A71, L72, R73, P74, S75, D76, E77, G78, T79, Y80, E81, C82, V83, V84, L85, K86, Y87, E88, K89, D90, A91, F92, K93, R94, E95, H96, L97, A98, E99, V100, T101, L102, S103, V104, K105, A106, D107, F108, P109, T110, P111, S112, I113, S114, D115, F116, E117, I118, P119, T120, S121, N122, I123, R124, R125, I126, I127, C128, S129, T130, S131, G132, G133, F134, P135, E136, P137, H138, L139, S140, W141, L142, E143, N144, G145, E146, E147, L148, N149, A150, I151, N152, T153, T154, V155, S156, Q157, D158, P159, E160, T161, E162, L163, Y164, A165, V166, S167, S168, K169, L170, D171, F172, N173, M174, T175, T176, N177, H178, S179, F180, M181, C182, L183, I184, K185, Y186, G187, H188, L189, R190, V191, N192, Q193, T194, F195, N196, W197, N198, T199, T200, K201, Q202, E203, H204, F205, P206, D207, or N208 of the amino acid sequence of SEQ ID NO: 2.

Further provided herein is a CD80-Fc fusion protein comprising: (i) an antibody Fc region and (ii) a variant CD80 polypeptide comprising a substitution of one or more amino acids at position V11, V22, T28, E23, A26, Y31, Q33, K36, G45, K54, T57, D60, 161, T62, N63, N64, K89, D90, or A91 of the amino acid sequence of SEQ ID NO: 2.

In one aspect, the CD80-Fc fusion protein comprises: (i) an antibody Fc region and (ii) a variant CD80 polypeptide comprising a substitution of one or more amino acids at position K36, K89, D90, and/or A91 of the amino acid sequence of SEQ ID NO: 2. In another aspect, the substitution at position K36 is K36R, the substitution at position K89 is K89D, K89E, or K89Q, the substitution at position D90 is D90K, D90N or D90Q, and the substitution at position A91 is A91S. In another aspect, the CD80-Fc fusion protein comprises: (i) an antibody Fc region and (ii) a variant CD80 polypeptide comprising a substitution of one or more amino acids, wherein the substitution comprises K36R, K89D, K89E, K89Q, D90K, D90N, D90Q, A91S, K36R-K89D, K36R-K89E, K36R-K89Q, K36R-D90K, K36R-D90N, K36R-D90Q, K36R-A91S, K89D-D90K, K89D-D90N, K89D-D90Q, K89D-A91S, K89E-D90K, K89E-D90N, K89E-D90Q, K89E-A91S, K89Q-D90K, K89Q-D90N, K89Q-D90Q, K89Q-A91S, D90K-A91S, D90N-A91S, D90Q-A91S, K36R-K89D-D90K, K36R-K89D-D90N, K36R-K89D-D90Q, K36R-K89D-A91S, K36R-K89E-D90K, K36R-K89E-D90N, K36R-K89E-D90Q, K36R-K89E-A91S, K36R-K89Q-D90K, K36R-K89Q-D90N, K36R-K89Q-D90Q, K36R-K89Q-A91S, K36R-D90K-A91S, K36R-D90N-A91S, K36R-D90Q-A91S, K89D-D90K-A91S, K89D-D90N-A91S, K89D-D90Q-A91S, K89E-D90K-A91S, K89E-D90N-A91S, K89E-D90Q-A91S, K89Q-D90K-A91S, K89Q-D90N-A91S, K89Q-D90Q-A91S, K36R-K89D-D90K-A91S, K36R-K89D-D90N-A91S, K36R-K89D-D90Q-A91S, K36R-K89E-D90K-A91S, K36R-K89E-D90N-A91S, K36R-K89E-D90Q-A91S, K36R-K89Q-D90K-A91S, K36R-K89Q-D90N-A91S, or K36R-K89Q-D90Q-A91S of the amino acid sequence of SEQ ID NO: 2.

In another aspect, the substitution comprises K89D, K89E, K89Q, D90K, D90N, D90Q, A91S, K89D-D90N, K89D-D90Q, K89D-D90K, or K89Q-D90Q of the amino acid sequence of SEQ ID NO: 2.

In some aspects, provided is a CD80-Fc fusion protein comprising: (i) an antibody Fc region and (ii) a variant CD80 polypeptide comprising a substitution of one or more amino acid that increases or enhances the binding affinity of the CD80-Fc fusion protein to CD28 compared to the binding affinity of a wild-type CD80-Fc protein to CD28.

Further provided herein is a CD80-Fc fusion protein comprising: (i) an antibody Fc region and (ii) a variant CD80 polypeptide comprising a substitution of one or more amino acids at position V11, V22, T28, E23, A26, Y31, Q33, G45, K54, T57, D60, I61, T62, N63, and/or N64, of the amino acid sequence of SEQ ID NO: 2.

In one aspect, the substitution at position V11 is V11L; the substitution at position V22 is V22C, V22F or V22M; the substitution at position T28 is T28V; the substitution at position E23 is E23C; the substitution at position A26 is A26C; the substitution at position Y31 is Y31Q; the substitution at position Q33 is Q33E; the substitution at position G45 is G45C; the substitution at position K54 is K54E; the substitution at position T57 is T57V; the substitution at position D60 is D60F, D60Q, D60R, D60T or D60Y; the substitution at position I61 is I61C; the substitution at position T62 is T62F, T62I, T62L or T62Y; the substitution at position N63 is N63D or N63E; and the substitution at position N64 is N64D or N64E of the amino acid sequence of SEQ ID NO: 2.

In another aspect, the substitution comprises V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D, N64E, V11L-V22C, V11L-V22F, V11L-V22M, V11L-T28V, V11L-E23C, V11L-A26C, V11L-Y31Q, V11L-Q33E, V11L-G45C, V11L-K54E, V11L-T57V, V11L-D60F, V11L-D60Q, V11L-D60R, V11L-D60T, V11L-D60Y, V11L-I61C, V11L-T62F, V11L-T62I, V11L-T62L, V11L-T62Y, V11L-N63D, V11L-N63E, V11L-N64D, V11L-N64E, V22C-T28V, V22C-E23C, V22C-A26C, V22C-Y31Q, V22C-Q33E, V22C-G45C, V22C-K54E, V22C-T57V, V22C-I61C, V22C-T62F, V22C-T62I, V22C-T62L, V22C-T62Y, V22C-N63D, V22C-N63E, V22C-N64D, V22C-N64E, V22F-T28V, V22F-E23C, V22F-A26C, V22F-Y31Q, V22F-Q33E, V22F-G45C, V22F-K54E, V22F-T57V, V22F-I61C, V22F-T62F, V22F-T62I, V22F-T62L, V22F-T62Y, V22F-N63D, V22F-N63E, V22F-N64D, V22F-N64E, V22M-T28V, V22M-E23C, V22M-A26C, V22M-Y31Q, V22M-Q33E, V22M-G45C, V22M-K54E, V22M-T57V, V22M-I61C, V22M-T62F, V22M-T62I, V22M-T62L, V22M-T62Y, V22M-N63D, V22M-N63E, V22M-N64D, V22M-N64E, T28V-E23C, T28V-A26C, T28V-Y31Q, T28V-Q33E, T28V-G45C, T28V-K54E, T28V-T57V, T28V-D60F, T28V-D60Q, T28V-D60R, T28V-D60T, T28V-D60Y, T28V-I61C, T28V-T62F, T28V-T62I, T28V-T62L, T28V-T62Y, T28V-N63D, T28V-N63E, T28V-N64D, T28V-N64E, E23C-A26C, E23C-Y31Q, E23C-Q33E, E23C-G45C, E23C-K54E, E23C-T57V, E23C-D60F, E23C-D60Q, E23C-D60R, E23C-D60T, E23C-D60Y, E23C-I61C, E23C-T62F, E23C-T62I, E23C-T62L, E23C-T62Y, E23C-N63D, E23C-N63E, E23C-N64D, E23C-N64E, A26C-Y31Q, A26C-Q33E, A26C-G45C, A26C-K54E, A26C-T57V, A26C-D60F, A26C-D60Q, A26C-D60R, A26C-D60T, A26C-D60Y, A26C-I61C, A26C-T62F, A26C-T62I, A26C-T62L, A26C-T62Y, A26C-N63D, A26C-N63E, A26C-N64D, A26C-N64E, Y31Q-Q33E, Y31Q-G45C, Y31Q-K54E, Y31Q-T57V, Y31Q-D60F, Y31Q-D60Q, Y31Q-D60R, Y31Q-D60T, Y31Q-D60Y, Y31Q-I61C, Y31Q-T62F, Y31Q-T62I, Y31Q-T62L, Y31Q-T62Y, Y31Q-N63D, Y31Q-N63E, Y31Q-N64D, Y31Q-N64E, Q33E-G45C, Q33E-K54E, Q33E-T57V, Q33E-D60F, Q33E-D60Q, Q33E-D60R, Q33E-D60T, Q33E-D60Y, Q33E-I61C, Q33E-T62F, Q33E-T62I, Q33E-T62L, Q33E-T62Y, Q33E-N63D, Q33E-N63E, Q33E-N64D, Q33E-N64E, G45C-K54E, G45C-T57V, G45C-D60F, G45C-D60Q, G45C-D60R, G45C-D60T, G45C-D60Y, G45C-I61C, G45C-T62F, G45C-T62I, G45C-T62L, G45C-T62Y, G45C-N63D, G45C-N63E, G45C-N64D, G45C-N64E, K54E-T57V, K54E-D60F, K54E-D60Q, K54E-D60R, K54E-D60T, K54E-D60Y, K54E-I61C, K54E-T62F, K54E-T62I, K54E-T62L, K54E-T62Y, K54E-N63D, K54E-N63E, K54E-N64D, K54E-N64E, T57V-D60F, T57V-D60Q, T57V-D60R, T57V-D60T, T57V-D60Y, T57V-I61C, T57V-T62F, T57V-T62I, T57V-T62L, T57V-T62Y, T57V-N63D, T57V-N63E, T57V-N64D, T57V-N64E, D60F-I61C, D60F-T62F, D60F-T62I, D60F-T62L, D60F-T62Y, D60F-N63D, D60F-N63E, D60F-N64D, D60F-N64E, D60E-I61C, D60E-T62F, D60E-T62I, D60E-T62L, D60E-T62Y, D60E-N63D, D60E-N63E, D60E-N64D, D60E-N64E, D60R-I61C, D60R-T62F, D60R-T62I, D60R-T62L, D60R-T62Y, D60R-N63D, D60R-N63E, D60R-N64D, D60R-N64E, D60T-I61C, D60T-T62F, D60T-T62I, D60T-T62L, D60T-T62Y, D60T-N63D, D60T-N63E, D60T-N64D, D60T-N64E, D60Y-I61C, D60Y-T62F, D60Y-T62I, D60Y-T62L, D60Y-T62Y, D60Y-N63D, D60Y-N63E, D60Y-N64D, D60Y-N64E, T62F-N63D, T62F-N63E, T62F-N64D, T62F-N64E, T62I-N63D, T62I-N63E, T62I-N64D, T62I-N64E, T62L-N63D, T62L-N63E, T62L-N64D, T62L-N64E, T62Y-N63D, T62Y-N63E, T62Y-N64D, T62Y-N64E, N63D-N64D, N63D-N64E, N63E-N64D, N63E-N64E, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, V22F-T28V-T57V-Y31Q-Q33E-K54E or any other combination of V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D and/or N64E of the amino acid sequence of SEQ ID NO: 2.

In another aspect, the substitution comprises D60Y, I61C, V11L-V22F, V11L-T62Y, V22C-G45C, V22F-D60Y, V22F-T62L, E23C-A26C, T28V-T57V, D60F T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E of the amino acid sequence of SEQ ID NO: 2.

In some aspects, provided is a CD80-Fc fusion protein comprising: (i) an antibody Fc region and (ii) a variant CD80 polypeptide comprising a substitution of one or more amino acids that increases stability of the CD80-Fc fusion protein compared to the stability of a wild-type CD80-Fc fusion protein. In some aspects, the increased stability provides for enhanced thermal stability, reduced thermal forced aggregation and/or reduced viscosity.

Further provided herein is a CD80-Fc fusion protein comprising: (i) an antibody Fc region and (ii) a variant CD80 polypeptide comprising a substitution of one or more amino acids of the amino acid sequence of SEQ ID NO: 2, wherein the substitution comprises K89E-I61C, K89E-D60Y, K89E-E23C-A26C, K89E-V22C-G45C, K89E-T28V-T57V, K89E-V11L-V22F, K89E-V11L-T62Y, K89E-V22F-T62L, K89E-D60Y-T62L, K89E-V22F-K89E-D60Y, K89E-D60F-T62I, K89E-D60R-T62Y, K89E-D60Y-V11L, K89E-D60Y-V22M, K89E-D60T-T62Y, K89E-D60Q-T62F, K89E-V22F-T28V-T57V, K89E-V11L-T62Y-N63D, K89E-D60Y-V11L-N63D, K89E-V22F-T62L-N64E, K89E-D60Y-T62L-N63D, K89E-D60Y-K54E-N63E-N64D, K89E-D60Y-T62L-N63D-N64E, K89E-V22F-D60Y-K54E-N64E, K89E-V22F-T62L-N63D-N64E, K89E-T28V-T57V-Y31Q-Q33E-K54E, K89E-V22F-T28V-T57V-Y31Q-Q33E-K54E, K89Q-I61C, K89Q-D60Y, K89Q-E23C-A26C, K89Q-V22C-G45C, K89Q-T28V-T57V, K89Q-V11L-V22F, K89Q-V11L-T62Y, K89Q-V22F-T62L, K89Q-D60Y-T62L, K89Q-V22F-D60Y, K89Q-D60F-T62I, K89Q-D60R-T62Y, K89Q-D60Y-V11L, K89Q-D60Y-V22M, K89Q-D60T-T62Y, K89Q-D60Q-T62F, K89Q-V22F-T28V-T57V, K89Q-V11L-T62Y-N63D, K89Q-D60Y-V11L-N63D, K89Q-V22F-T62L-N64E, K89Q-D60Y-T62L-N63D, K89Q-D60Y-K54E-N63E-N64D, K89Q-D60Y-T62L-N63D-N64E, K89Q-V22F-D60Y-K54E-N64E, K89Q-V22F-T62L-N63D-N64E, K89Q-T28V-T57V-Y31Q-Q33E-K54E, K89Q-V22F-T28V-T57V-Y31Q-Q33E-K54E, K89D-I61C, K89D-D60Y, K89D-E23C-A26C, K89D-V22C-G45C, K89D-T28V-T57V, K89D-V11L-V22F, K89D-V11L-T62Y, K89D-V22F-T62L, K89D-D60Y-T62L, K89D-V22F-D60Y, K89D-D60F-T62I, K89D-D60R-T62Y, K89D-D60Y-V11L, K89D-D60Y-V22M, K89D-D60T-T62Y, K89D-D60Q-T62F, K89D-V22F-T28V-T57V, K89D-V11L-T62Y-N63D, K89D-D60Y-V11L-N63D, K89D-V22F-T62L-N64E, K89D-D60Y-T62L-N63D, K89D-D60Y-K54E-N63E-N64D, K89D-D60Y-T62L-N63D-N64E, K89D-V22F-D60Y-K54E-N64E, K89D-V22F-T62L-N63D-N64E, K89D-T28V-T57V-Y31Q-Q33E-K54E, K89D-V22F-T28V-T57V-Y31Q-Q33E-K54E, D90K-I61C, D90K-D60Y, D90K-E23C-A26C, D90K-V22C-G45C, D90K-T28V-T57V, D90K-V11L-V22F, D90K-V11L-T62Y, D90K-V22F-T62L, D90K-D60Y-T62L, D90K-V22F-D60Y, D90K-D60F-T62I, D90K-D60R-T62Y, D90K-D60Y-V11L, D90K-D60Y-V22M, D90K-D60T-T62Y, D90K-D60Q-T62F, D90K-V22F-T28V-T57V, D90K-V11L-T62Y-N63D, D90K-D60Y-V11L-N63D, D90K-V22F-T62L-N64E, D90K-D60Y-T62L-N63D, D90K-D60Y-K54E-N63E-N64D, D90K-D60Y-T62L-N63D-N64E, D90K-V22F-D60Y-K54E-N64E, D90K-V22F-T62L-N63D-N64E, D90K-T28V-T57V-Y31Q-Q33E-K54E, D90K-V22F-T28V-T57V-Y31Q-Q33E-K54E, D90N-I61C, D90N-D60Y, D90N-E23C-A26C, D90N-V22C-G45C, D90N-T28V-T57V, D90N-V11L-V22F, D90N-V11L-T62Y, D90N-V22F-T62L, D90N-D60Y-T62L, D90N-V22F-D60Y, D90N-D60F-T62I, D90N-D60R-T62Y, D90N-D60Y-V11L, D90N-D60Y-V22M, D90N-D60T-T62Y, D90N-D60Q-T62F, D90N-V22F-T28V-T57V, D90N-V11L-T62Y-N63D, D90N-D60Y-V11L-N63D, D90N-V22F-T62L-N64E, D90N-D60Y-T62L-N63D, D90N-D60Y-K54E-N63E-N64D, D90N-D60Y-T62L-N63D-N64E, D90N-V22F-D60Y-K54E-N64E, D90N-V22F-T62L-N63D-N64E, D90N-T28V-T57V-Y31Q-Q33E-K54E, D90N-V22F-T28V-T57V-Y31Q-Q33E-K54E, D90Q-I61C, D90Q-D60Y, D90Q-E23C-A26C, D90Q-V22C-G45C, D90Q-T28V-T57V, D90Q-V11L-V22F, D90Q-V11L-T62Y, D90Q-V22F-T62L, D90Q-D60Y-T62L, D90Q-V22F-D60Y, D90Q-D60F-T62I, D90Q-D60R-T62Y, D90Q-D60Y-V11L, D90Q-D60Y-V22M, D90Q-D60T-T62Y, D90Q-D60Q-T62F, D90Q-V22F-T28V-T57V, D90Q-V11L-T62Y-N63D, D90Q-D60Y-V11L-N63D, D90Q-V22F-T62L-N64E, D90Q-D60Y-T62L-N63D, D90Q-D60Y-K54E-N63E-N64D, D90Q-D60Y-T62L-N63D-N64E, D90Q-V22F-D60Y-K54E-N64E, D90Q-V22F-T62L-N63D-N64E, D90Q-T28V-T57V-Y31Q-Q33E-K54E, D90Q-V22F-T28V-T57V-Y31Q-Q33E-K54E, K89Q-D90Q-I61C, K89Q-D90Q-D60Y, K89Q-D90Q-E23C-A26C, K89Q-D90Q-V22C-G45C, K89Q-D90Q-T28V-T57V, K89Q-D90Q-V11L-V22F, K89Q-D90Q-V11L-T62Y, K89Q-D90Q-V22F-T62L, K89Q-D90Q-D60Y-T62L, K89Q-D90Q-V22F-D60Y, K89Q-D90Q-D60F-T62I, K89Q-D90Q-D60R-T62Y, K89Q-D90Q-D60Y-V11L, K89Q-D90Q-D60Y-V22M, K89Q-D90Q-D60T-T62Y, K89Q-D90Q-D60Q-T62F, K89Q-D90Q-V22F-T28V-T57V, K89Q-D90Q-V11L-T62Y-N63D, K89Q-D90Q-D60Y-V11L-N63D, K89Q-D90Q-V22F-T62L-N64E, K89Q-D90Q-D60Y-T62L-N63D, K89Q-D90Q-D60Y-K54E-N63E-N64D, K89Q-D90Q-D60Y-T62L-N63D-N64E, K89Q-D90Q-V22F-D60Y-K54E-N64E, K89Q-D90Q-V22F-T62L-N63D-N64E, K89Q-D90Q-T28V-T57V-Y31Q-Q33E-K54E, K89Q-D90Q-V22F-T28V-T57V-Y31Q-Q33E-K54E, K89D-D90N-I61C, K89D-D90N-D60Y, K89D-D90N-E23C-A26C, K89D-D90N-V22C-G45C, K89D-D90N-T28V-T57V, K89D-D90N-V11L-V22F, K89D-D90N-V11L-T62Y, K89D-D90N-V22F-T62L, K89D-D90N-D60Y-T62L, K89D-D90N-V22F-D60Y, K89D-D90N-D60F-T62I, K89D-D90N-D60R-T62Y, K89D-D90N-D60Y-V11L, K89D-D90N-D60Y-V22M, K89D-D90N-D60T-T62Y, K89D-D90N-D60Q-T62F, K89D-D90N-V22F-T28V-T57V, K89D-D90N-V11L-T62Y-N63D, K89D-D90N-D60Y-V11L-N63D, K89D-D90N-V22F-T62L-N64E, K89D-D90N-D60Y-T62L-N63D, K89D-D90N-D60Y-K54E-N63E-N64D, K89D-D90N-D60Y-T62L-N63D-N64E, K89D-D90N-V22F-D60Y-K54E-N64E, K89D-D90N-V22F-T62L-N63D-N64E, K89D-D90N-T28V-T57V-Y31Q-Q33E-K54E, K89D-D90N-V22F-T28V-T57V-Y31Q-Q33E-K54E, K89D-D90Q-I61C, K89D-D90Q-D60Y, K89D-D90Q-E23C-A26C, K89D-D90Q-V22C-G45C, K89D-D90Q-T28V-T57V, K89D-D90Q-V11L-V22F, K89D-D90Q-V11L-T62Y, K89D-D90Q-V22F-T62L, K89D-D90Q-D60Y-T62L, K89D-D90Q-V22F-D60Y. K89D-D90Q-D60F-T62I, K89D-D90Q-D60R-T62Y, K89D-D90Q-D60Y-V11L, K89D-D90Q-D60Y-V22M, K89D-D90Q-D60T-T62Y, K89D-D90Q-D60Q-T62F, K89D-D90Q-V22F-T28V-T57V, K89D-D90Q-V11L-T62Y-N63D, K89D-D90Q-D60Y-V11L-N63D, K89D-D90Q-V22F-T62L-N64E, K89D-D90Q-D60Y-T62L-N63D, K89D-D90Q-D60Y-K54E-N63E-N64D, K89D-D90Q-D60Y-T62L-N63D-N64E, K89D-D90Q-V22F-D60Y-K54E-N64E, K89D-D90Q-V22F-T62L-N63D-N64E, K89D-D90Q-T28V-T57V-Y31Q-Q33E-K54E, K89D-D90Q-V22F-T28V-T57V-Y31Q-Q33E-K54E, K89D-D90K-I61C, K89D-D90K-D60Y, K89D-D90K-E23C-A26C, K89D-D90K-V22C-G45C, K89D-D90K-T28V-T57V, K89D-D90K-V11L-V22F, K89D-D90K-V11L-T62Y, K89D-D90K-V22F-T62L, K89D-D90K-D60Y-T62L, K89D-D90K-V22F-D60Y, K89D-D90K-D60F-T62I, K89D-D90K-D60R-T62Y, K89D-D90K-D60Y-V11L, K89D-D90K-D60Y-V22M, K89D-D90K-D60T-T62Y, K89D-D90K-D60Q-T62F, K89D-D90K-V22F-T28V-T57V, K89D-D90K-V11L-T62Y-N63D, K89D-D90K-D60Y-V11L-N63D, K89D-D90K-V22F-T62L-N64E, K89D-D90K-D60Y-T62L-N63D, K89D-D90K-D60Y-K54E-N63E-N64D, K89D-D90K-D60Y-T62L-N63D-N64E, K89D-D90K-V22F-D60Y-K54E-N64E, K89D-D90K-V22F-T62L-N63D-N64E, K89D-D90K-T28V-T57V-Y31Q-Q33E-K54E, K89D-D90K-V22F-T28V-T57V-Y31Q-Q33E-K54E, A91S-I61C, A91S-D60Y, A91S-E23C-A26C, A91S-V22C-G45C, A91S-T28V-T57V, A91S-V11L-V22F, A91S-V11L-T62Y, A91S-V22F-T62L, A91S-D60Y-T62L, A91S-V22F-D60Y, A91S-D60F-T62I, A91S-D60R-T62Y, A91S-D60Y-V11L, A91S-D60Y-V22M, A91S-D60T-T62Y, A91S-D60Q-T62F, A91S-V22F-T28V-T57V, A91S-V11L-T62Y-N63D, A91S-D60Y-V11L-N63D, A91S-V22F-T62L-N64E, A91S-D60Y-T62L-N63D, A91S-D60Y-K54E-N63E-N64D, A91S-D60Y-T62L-N63D-N64E, A91S-V22F-D60Y-K54E-N64E, A91S-V22F-T62L-N63D-N64E, A91S-T28V-T57V-Y31Q-Q33E-K54E, or A91S-V22F-T28V-T57V-Y31Q-Q33E-K54E of the amino acid sequence of SEQ ID NO: 2.

Further provided herein is a CD80-Fc fusion protein comprising (i) an antibody Fc region and (ii) a variant CD80 polypeptide comprising i) a first substitution of one or more amino acids at position K36, K89, D90, and/or A91 of the amino acid sequence of SEQ ID NO: 2, and ii) a second substitution of one or more amino acids at position V11, V22, T28, E23, A26, Y31, Q33, G45, K54, T57, D60, I61, T62, N63 and/or N64 of the amino acid sequence of SEQ ID NO: 2.

In some aspects, i) the first substitution comprises K89D, K89E, K89Q, D90K, D90N, D90Q, A91S, K89D-D90N, K89D-D90Q, K89D-D90K, or K89Q-D90Q of the amino acid sequence of SEQ ID NO: 2, and ii) the second substitution comprises D60Y, I61C, V11L-V22F, V11L-T62Y, V22C-G45C, V22F-D60Y, V22F-T62L, E23C-A26C, T28V-T57V, D60F-T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E of the amino acid sequence of SEQ ID NO: 2.

In one aspect, provided is a CD80-Fc fusion protein comprising: (i) an antibody Fc region and (ii) a variant CD80 polypeptide comprising a substitution of one or more amino acids at position V11, V22, T28, E23, A26, Y31, Q33, K36, G45, K54, T57, D60, 161, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of two or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of three or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of four or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of five or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of six or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of seven or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of eight or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of nine or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of ten or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a CD80-Fc fusion protein comprising: (i) an antibody Fc region and (ii) a variant CD80 polypeptide comprising a substitution of eleven or more, twelve or more, thirteen or more, fourteen or more, fifteen or more, sixteen or more, seventeen or more, eighteen or more, nineteen or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

Exemplary CD80-Fc fusion proteins include, but are not limited to, the amino acid sequences set forth in SEQ ID NO: 64-114.

In some aspects, CD80-Fc fusion proteins of the present invention comprise sialic acid residues. In one aspect, CD80-Fc fusion proteins of the present invention may comprise an average of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, or greater than 35 sialic acid residues per molecule. In another aspect, the average sialic acid residues per molecule of CD80-Fc fusion protein may be in the range of 1 to 35, 1 to 30, 1 to 25, 1 to 20, 1 to 15, 1 to 10, 1 to 5, 5 to 35, 5 to 30, 5 to 25, 5 to 20, 5 to 15, 5 to 10, 10 to 35, 10 to 30, 10 to 25, 10 to 20, 10 to 15, 15 to 35, 15 to 30, 20 to 25, 25 to 35, 25 to 30, or 30 to 35.

### Variant CD80 Polypeptides

Provided herein are variant CD80 polypeptides (e.g. extracellular domain (ECD) of human CD80) comprising a substitution of one or more amino acids at position V1, I2, H3, V4, T5, K6, E7, V8, K9, E10, V11, A12, T13, L14, S15, C16, G17, H18, N19, V20, S21, V22, E23, E24, L25, A26, Q27, T28, R29, I30, Y31, W32, Q33, K34, E35, K36, K37, M38, V39, L40, T41, M42, M43, S44, G45, D46, M47, N48, I49, W50, P51, E52, Y53, K54, N55, R56, T57, I58, F59, D60, I61, T62, N63, N64, L65, S66, 167, V68, 169, L70, A71, L72, R73, P74, S75, D76, E77, G78, T79, Y80, E81, C82, V83, V84, L85, K86, Y87, E88, K89, D90, A91, F92, K93, R94, E95, H96, L97, A98, E99, V100, T101, L102, S103, V104, K105, A106, D107, F108, P109, T110, P111, S112, I113, S114, D115, F116, E117, I118, P119, T120, S121, N122, I123, R124, R125, I126, I127, C128, S129, T130, S131, G132, G133, F134, P135, E136, P137, H138, L139, S140, W141, L142, E143, N144, G145, E146, E147, L148, N149, A150, I151, N152, T153, T154, V155, S156, Q157, D158, P159, E160, T161, E162, L163, Y164, A165, V166, S167, S168, K169, L170, D171, F172, N173, M174, T175, T176, N177, H178, S179, F180, M181, C182, L183, I184, K185, Y186, G187, H188, L189, R190, V191, N192, Q193, T194, F195, N196, W197, N198, T199, T200, K201, Q202, E203, H204, F205, P206, D207, or N208 of the amino acid sequence of SEQ ID NO: 2.

Further provided herein are variant CD80 polypeptides comprising a substitution of one or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In some aspects, the substitution at position V11 is V11L; the substitution at position V22 is V22C, V22F or V22M; the substitution at position T28 is T28V; the substitution at position E23 is E23C; the substitution at position A26 is A26C; the substitution at position Y31 is Y31Q; the substitution at position Q33 is Q33E; the substitution at position K36 is K36R, the substitution at position G45 is G45C; the substitution at position K54 is K54E; the substitution at position T57 is T57V; the substitution at position D60 is D60F, D60Q, D60R, D60T or D60Y; the substitution at position I61 is I61C; the substitution at position T62 is T62F, T62I, T62L or T62Y; the substitution at position N63 is N63D or N63E; and the substitution at position N64 is N64D or N64E; the substitution at position K89 is K89D, K89E or K89Q; the substitution at position D90 is D90K, D90N or D90Q; and the substitution at position A91 is A91S.

In some aspects, the substitution comprises K89D, K89E, K89Q, D90K, D90N, D90Q, A91S, K89D-D90N, K89D-D90Q, K89D-D90K, K89Q-D90Q, D60Y, I61C, V11L-V22F, V11L-T62Y, V22C-G45C, V22F-D60Y, V22F-T62L, E23C-A26C, T28V-T57V, D60F-T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E, K89Q-D90Q-I61C, D90Q-E23C-A26C, K89Q-D90Q-E23C-A26C, or K89Q-D90Q-V22C-G45C, or K89D-D90K-T28V-T57V of the amino acid sequence of SEQ ID NO: 2.

Further provided herein are variant CD80 polypeptides comprising (i) a first substitution of one or more amino acids at position K36, K89, D90, or A91, and (ii) a second substitution of one or more amino acids at position V11, V22, T28, E23, A26, Y31, Q33, G45, K54, T57, D60, I61, T62, N63, N64, of the amino acid sequence of SEQ ID NO: 2.

In one aspect, the first substitution comprises K89D, K89E, K89Q, D90K, D90N, D90Q, A91S, K89D-D90N, K89D-D90Q, K89D-D90K or K89Q-D90Q, and (ii) the second substitution comprises D60Y, I61C, V11L-V22F, V11L-T62Y, V22C-G45C, V22F-D60Y, V22F-T62L, E23C-A26C, T28V-T57V, D60F-T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E of the amino acid sequence of SEQ ID NO: 2.

In another aspect, provided is a variant CD80 polypeptide comprising a substitution of one or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of two or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of three or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of four or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of five or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of six or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of seven or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of eight or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of nine or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of ten or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

In another aspect, provided is a variant CD80 polypeptide comprising a substitution of eleven or more, twelve or more, thirteen or more, fourteen or more, fifteen or more, sixteen or more, seventeen or more, eighteen or more, nineteen or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

Exemplary variant CD80 polypeptides include, but are not limited to, the amino acid sequences set forth in SEQ ID NO: 20-63.

### Fc Region

The CD80-Fc fusion proteins of the present invention may comprise an antibody or fragment thereof, such as, monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies. In one aspect, the CD80-Fc fusion protein comprises an antibody fragment, such as an Fc region.

In some aspects, the isotype of an antibody or fragment thereof, is selected from the group consisting of IgG₁, IgG₂, IgG_{2Δa}, IgG₄, IgG_{4Δb}, IgG_{4Δc}, and IgG_{4Δb}. In some aspects, the CD80-Fc fusion proteins as described herein comprise a Fc region of an antibody. In some aspects, the antibody Fc region is a human IgG1, IgG2, or IgG4, having the sequence listed below, with or without a C-terminal lysine (K).

Wild-type Human IgG1 Fc:
(SEQ ID NO: 13)
EPKSCDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFYPSDIAVEWESNGQPENNYKTTP
PVLDSDGSFFLYSKLTVDKSRWQQGNVFSCSVMHEALHNHYTQKS
LSLSPG
Wild-type Human IgG2 Fc
(SEQ ID NO: 14)
ERKCCVECPPCPAPPVAGPSVFLFPPKPKDTLMISRTPEVTCVVV
DVSHEDPEVQFNWYVDGVEVHNAKTKPREEQFNSTFRVVSVLTVV
HQDWLNGKEYKCKVSNKGLPAPIEKTISKTKGQPREPQVYTLPPS
REEMTKNQVSLTCLVKGFYPSDISVEWESNGQPENNYKTTPPMLD
SDGSFFLYSKLTVDKSRWQQGNVFSCSVMHEALHNHYTQKSLSLS
PG
Wild-type Human IgG4 Fc
(SEQ ID NO: 15)
ESKYGPPCPSCPAPEFLGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLG

Exemplary antibody Fc regions used for the present invention include, but are not limited to, the sequences listed herein.

In some aspects, the antibody Fc region as described herein comprises amino acid modifications at position 220 (e.g., C220S) of the human IgG1 (SEQ ID NO: 13). For example, the antibody Fc region as described herein comprises an amino acid sequence of:

EPKSSDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFYPSDIAVEWESNGQPENNYKTTP
PVLDSDGSFFLYSKLTVDKSRWQQGNVFSCSVMHEALHNHYTQKS
LSLSPG
(SEQ ID NO: 16), with or without a
C-terminal lysine (K).

In some aspects, the antibody Fc regions as described herein comprise amino acid modifications at one or more of positions 220, 234, 235, 237, and/or 322 of the human IgG1 (SEQ ID NO: 13). In some aspects, the antibody Fc regions as described herein comprise amino acid modifications at one or more of positions 220 (e.g., C220S), 234 (e.g., L234A), 235 (e.g., L235A), and 237 (e.g., G237A) of the human IgG1 (SEQ ID NO: 13). In some aspects, the antibody Fc region as described herein comprise amino acid modifications at each of positions 220 (e.g., C220S), 234 (e.g., L234A), 235 (e.g., L235A), and 237 (e.g., G237A) of the human IgG1 (SEQ ID NO: 13). For example, the antibody Fc regions as described herein comprise an amino acid sequence of:

EPKSSDKTHTCPPCPAPEAAGAPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFYPSDIAVEWESNGQPENNYKTTP
PVLDSDGSFFLYSKLTVDKSRWQQGNVFSCSVMHEALHNHYTQKS
LSLSPG
(SEQ ID NO: 17), with or without a
C-terminal lysine (K).

In some aspects, the antibody Fc regions as described herein comprise one or more of positions 265 (e.g., D265A), 330 (e.g., A330S), and 331 (e.g., P331S) of the human IgG2 (SEQ ID NO: 14). In some aspects, the antibody Fc region as described herein comprises amino acid modifications at each of positions 265 (e.g., D265A), 330 (e.g., A330S), and 331 (e.g., P331S) of the human IgG2 (SEQ ID NO: 14). For example, the antibody Fc region as described herein comprises an amino acid sequence of:

ERKCCVECPPCPAPPVAGPSVFLFPPKPKDTLMISRTPEVTCVVV
AVSHEDPEVQFNWYVDGVEVHNAKTKPREEQFNSTFRVVSVLTVV
HQDWLNGKEYKCKVSNKGLPSSIEKTISKTKGQPREPQVYTLPPS
REEMTKNQVSLTCLVKGFYPSDISVEWESNGQPENNYKTTPPMLD
SDGSFFLYSKLTVDKSRWQQGNVFSCSVMHEALHNHYTQKSLSLS
PG
(SEQ ID NO: 18), with or without
a C-terminal lysine (K).

In some aspects, a CD80-Fc fusion protein provided herein comprises: (i) an antibody Fc region and (ii) a variant CD80 polypeptide, wherein the variant CD80 polypeptide is covalently linked or fused to the antibody Fc region. In some aspects, the variant CD80 polypeptide is linked or fused to the N-terminus of the antibody Fc region.

In some aspects, the variant CD80 polypeptide is linked or fused to the N-terminus of an antibody Fc region via the human CH1 domain (e.g., EPKSC; SEQ ID NO: 3) of the antibody Fc region (e.g., SEQ ID NO: 13). In other aspects, the variant CD80 polypeptide is linked or fused to the N-terminus of an antibody Fc region via the human CH1 domain having a modification at position 220 (e.g., C220S) (e.g., EPKSS; SEQ ID NO: 4) of the antibody Fc region (e.g., SEQ ID NO: 16).

In some aspects, one or more polypeptides (e.g., heterologous or homologous sequence) can be inserted between the antibody Fc region and variant CD80 polypeptide of the CD80-Fc fusion proteins. In some aspects, the polypeptide can be inserted or conjugated at the amino terminus, at the carboxyl terminus, or both the amino and carboxyl termini of the antibody Fc region. In some aspects, the polypeptide comprises a polypeptide linker conjugating the antibody Fc region and the variant CD80 polypeptide. For example, the polypeptide linker can be a glycine-serine (GS)-linker, including but not limited to, GGGGGTSATATPGA (SEQ ID NO: 5), GGGGSGSGG (SEQ ID NO: 6), GGGGGTSATATPGA (SEQ ID NO: 7), GGSGGGGSGGGSGGGGSGG (SEQ ID NO: 8), and SGGGGSGGGGGGGG (SEQ ID NO: 9).

In some aspects, the polypeptide comprises one or more linker(s) and tag(s). Examples of a polypeptide tag include, but not are not limited to, a FLAG tag, a 6His tag (e.g., HHHHHH; SEQ ID NO: 10), a 8His tag (e.g., HHHHHHHH; SEQ ID NO: 11), or an AVI tag (e.g., GLNDIFEAQKIEWHE; SEQ ID NO: 12).

In some aspects, the antibody Fc regions as described herein may comprise modifications provided in Wang et al. Protein Cell. 2018 January; 9(1):63-73. For example, antibody Fc region (IgG1) modifications include but are not limited to modifications that (i) enhance ADCC, such as F243L/R292P/Y300L/V305l/P396L, S239D/1332E, S298A/E333A/K334A or L234Y/L235Q/G236W/S239M/H268D/D270E/S298A in one heavy chain and D270E/K326D/A330M/K334E in the opposing heavy chain (increased FcγRIIIa binding); S239D/1332E/A330L (increased FcγRIIIa binding, decreased FcγRIIb binding); (ii) enhance ADCP, such as G236A/S239D/1332E (increased FcγRIIa binding, increased FcγRIIIa binding); (iii) enhance CDC, such as K326W/E333S, S267E/H268F/S324T or IgG1/IgG3 cross subclass (increased C1q binding); E345R/E430G/S440Y (hexamerization); (iv) reduce effector function, such as N297A or N297Q or N297G (aglycosylated); L235E or L234A/L235A (reduced FcγR and C1q binding); (v) increase half-life, such as M252Y/S254T/T256E or M428L/N434S (increased FcRn binding at pH 6.0) and (vi) increase coengagement, such as S267E/L328F (increased FcγRIIb binding); N325S/L328F (increased FcγRIIa binding, decreased FcγRIIIa binding).

For example, antibody Fc region (IgG2) modifications include but are not limited to modifications that reduce effector function, such as H268Q/V309L/A330S/P331S, V234A/G237A/P238S/H268A/V309L/A330S/P331S, or IgG2/IgG4 cross isotype (reduced FcγR and C1q binding).

For example, antibody Fc region (IgG4) modifications include but are not limited to modifications that reduce effector function, such as F234A/L235A (reduced FcγR and C1q binding).

In another aspect, the antibody Fc regions as described herein may comprise modifications provided in Shields et al. J Biol Chem. 2001 Mar. 2; 276(9):6591-604. For example, antibody Fc region (IgG1) modifications include but are not limited to modifications that reduce binding to all FcγR (Class 1): E233P, L234V, L235A, G236 deleted, P238A, D265A, N297A, A327Q, P329A; reduce binding to FcγRII and FcγRIIIA (Class 2): D270A, Q295A, A327S; improve binding to FcγRII and FcγRIIIA (Class 3): T256A, A327A; improve binding to FcγRII and no effect on FcγRIIIA (Class 4): R255A, E258A, S267A, E272A, N276A, D280A, H285A, N286A, T307A, L309A, N315A, K326A, P331A, S337A, A378Q, E430A; improve binding to FcγRII and reduced binding to FcγRIIIA (Class 5): H268A, R301A, K322A; reduce binding to FcγRII and no effect on FcγRIIIA (Class 6): R292A, K414A; reduce binding to FcγRII and improved binding to FcγRIIIA (Class 7): S298A; no effect on FcγRII and reduced binding to FcγRIIIA (Class 8): S239A, E269A, E293A, Y296F, V303A, A327G, K338A, D376A; no effect on FcγRII and improved binding to FcγRIIIA (Class 9): E333A, K334A, A339T; and affect only FcRn (Class 10): I253A, S254A, K288A, V305A, Q311A, D312A, K317A, K360A, Q362A, E380A, E382A, S415A, S424A, H433A, N434A, H435A, Y436A.

In some aspects, the antibody Fc regions described herein comprise a modified constant region that have increased or decreased binding affinity to a human Fc gamma receptor, are immunologically inert or partially inert. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some aspects, the constant region is modified as described in Eur. J. Immunol., 1999, 29:2613-2624; PCT Publication No. WO99/058572.

In some aspects, a constant region can be modified to avoid interaction with Fc gamma receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

In still other aspects, the constant region is aglycosylated for N-linked glycosylation. In some aspects, the constant region is aglycosylated for N-linked glycosylation by mutating the oligosaccharide attachment residue and/or flanking residues that are part of the N-glycosylation recognition sequence in the constant region. For example, N-glycosylation site N297 may be mutated to, e.g., A, Q, K, or H. See, Tao et al., J. Immunology 143: 2595-2601, 1989; and Jefferis et al., Immunological Reviews 163:59-76, 1998. In some aspects, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Exemplary antibody Fc regions include, but are not limited to, the amino acid sequences set forth in SEQ ID NO: 13-18.

### Polynucleotides, Vectors, and Host Cells

The invention also provides polynucleotides encoding any of the CD80-Fc fusion proteins and variant CD80 polypeptides as described herein, and vectors and host cells comprising the polynucleotides. In one aspect, a polynucleotide comprises a nucleotide sequence encoding a variant CD80 polypeptides. In another aspect, a polynucleotide comprises a nucleotide sequence encoding an antibody Fc region. In another aspect, a polynucleotide comprises a nucleotide sequence encoding a CD80-Fc fusion protein. Exemplary CD80-Fc fusion proteins include, but are not limited to, the nucleic acid sequences set forth in SEQ ID NOs: 115-119.

The sequence encoding the variant CD80 polypeptide, antibody Fc region and/or CD80-Fc fusion protein of interest may be maintained in a vector in a host cell and the host cell can then be expanded and frozen for future use. Vectors (including expression vectors) and host cells are further described herein.

In one aspect, the invention provides a method of making any of the polynucleotides described herein. Polynucleotides can be made and expressed by procedures known in the art. Typically, the fusion proteins of this invention are made by preparing an expressing a polynucleotide encoding them using recombinant methods described herein, although they may also be prepared by other means known in the art, including, for example, chemical synthesis

In one aspect, the invention provides for compositions (such as a pharmaceutical compositions) comprising any of the polynucleotides of the invention. In some aspects, the composition comprises an expression vector comprising a polynucleotide encoding any of the variant CD80 polypeptides, antibody Fc regions and CD80-Fc fusion proteins described herein. In another aspect, provided is an isolated cell line that produces the variant CD80 polypeptides, antibody Fc regions and CD80-Fc fusion proteins as described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include mature and immature mRNAs, such as precursor mRNAs (pre-mRNA) or heterogeneous nuclear mRNAs (hnRNA) and mature mRNAs. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art (e.g., Sambrook et al., 1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, Atum and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous protein of interest, if present, in the host cells.

### Pharmaceutical Compositions

The invention also provides pharmaceutical compositions comprising an effective amount of CD80-Fc fusion protein or variant CD80 polypeptide as described herein. Examples of such compositions, as well as how to formulate, are also described herein. In some aspects, the composition comprises one or more CD80-Fc fusion proteins. In some aspects, the composition comprises a CD80-Fc fusion protein comprising an antibody Fc region and a variant CD80 polypeptide. In some aspects, the composition comprises a CD80-Fc fusion protein comprising an antibody Fc region and a variant CD80 polypeptide comprising a substitution of one or more amino acids at positions V11, V22, T28, E23, A26, Y31, Q33, K36, G45, K54, T57, D60, I61, T62, N63, N64, K89, D90, or A91, wherein the variant CD80 polypeptide is linked or fused to an antibody Fc region.

It is understood that the compositions can comprise more than one CD80-Fc fusion protein (e.g., a mixture of CD80-Fc fusion proteins comprising different variant CD80 polypeptides and/or different antibody Fc regions).

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The CD80-Fc fusion proteins, and compositions thereof can also be used in conjunction with, or administered separately, simultaneously, or sequentially with other agents that serve to enhance and/or complement the effectiveness of the agents. The invention also provides compositions, including pharmaceutical compositions, comprising any of the polynucleotides of the invention. In some aspects, the composition comprises an expression vector comprising a polynucleotide encoding any of the CD80-Fc fusion proteins and variant CD80 polypeptides as described herein.

### Methods of Treatment

The CD80-Fc fusion proteins and variant CD80 polypeptides of the present invention are useful in various applications including, but are not limited to, therapeutic treatment methods and diagnostic treatment methods.

In one aspect, the invention provides a method for treating a cancer. In some aspects, the method of treating a cancer in a subject comprises administering to the subject in need thereof an effective amount of a composition (e.g., pharmaceutical composition) comprising any of the CD80-Fc fusion proteins as described herein. As used herein, a cancer can be a solid cancer or a liquid cancer. Solid cancers include, but are not limited to, gastric cancer, small intestine cancer, sarcoma, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer (e.g. non-small cell lung cancer), mesothelioma, ovarian cancer, breast cancer, prostate cancer, esophageal cancer, pancreatic cancer, glioma, renal cancer (e.g., renal cell carcinoma), bladder cancer, cervical cancer, uterine cancer, vulvar cancer, penile cancer, testicular cancer, anal cancer, choriocarcinoma, colon cancer, colorectal cancer, oral cancer, skin cancer, Merkel cell carcinoma, glioblastoma, brain tumor, bone cancer, eye cancer, melanoma, and cancer with high microsatellite instability (MSI-H).

Liquid cancers include, but not limited to, multiple myeloma, malignant plasma cell neoplasm, Hodgkin's lymphoma, nodular lymphocyte predominant Hodgkin's lymphoma, Kahler's disease and Myelomatosis, plasma cell leukemia, plasmacytoma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulinemia, diffuse large B cell lymphoma, mucosa-associated lymphatic tissue lymphoma, small cell lymphocytic lymphoma, primary mediastinal (thymic) large B-cell lymphoma, lymphoplasmactyic lymphoma, nodal marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBV positive diffuse large B-cell lymphoma of the elderly, diffuse large B-cell lymphoma associated with inflammation, ALK-positive large B-cell lymphoma, plasmablastic lymphoma, large B-cell lymphoma arising in HHV8-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other hematopoietic cells related cancer.

In some aspects, the cancer is relapsed, resistant, refractory, and/or metastatic. For example, the cancer is resistant and/or refractory to anti-PD-1/anti-PD-L1 therapies.

In some aspects, the cancer is relapsed, resistant, refractory, and/or metastatic. For example, the cancer is resistant and/or refractory to anti-CTLA-4 therapies.

In some aspects, provided is a method of inhibiting tumor growth or progression in a subject, comprising administering to the subject in need thereof an effective amount of a composition comprising the CD80-Fc fusion proteins as described herein. In some aspects, provided is a method of inhibiting metastasis of cancer cells in a subject, comprising administering to the subject in need thereof an effective amount of a composition comprising any of the CD80-Fc fusion proteins as described herein. In other aspects, provided is a method of inducing regression of a tumor in a subject, comprising administering to the subject in need thereof an effective amount of a composition comprising any of the CD80-Fc fusion proteins as described herein.

In another aspect, provided is a method of detecting, diagnosing, and/or monitoring a cancer. For example, the CD80-Fc fusion proteins or variant CD80 polypeptides as described herein can be labeled with a detectable moiety such as an imaging agent and an enzyme-substrate label. The CD80-Fc fusion proteins or variant CD80 polypeptides as described herein can also be used for in vivo diagnostic assays, such as in vivo imaging (e.g., PET or SPECT), or a staining reagent.

In some aspects, the methods described herein further comprise a step of treating a subject with an additional form of therapy. In some aspects, the additional form of therapy is an additional anti-cancer therapy including, but not limited to, chemotherapy, radiation, surgery, hormone therapy, and/or additional immunotherapy.

With respect to all methods described herein, reference to CD80-Fc fusion proteins also includes compositions comprising one or more additional agents. These compositions may further comprise suitable excipients, such as pharmaceutically acceptable excipients including buffers, which are well known in the art. The present invention can be used alone or in combination with other methods of treatment.

The CD80-Fc fusion proteins as described herein can be administered to a subject via any suitable route. It should be apparent to a person skilled in the art that the examples described herein are not intended to be limiting but to be illustrative of the techniques available. Accordingly, in some aspects, the CD80-Fc fusion protein is administered to a subject in accord with known methods, such as intravenous administration, e.g., as a bolus or by continuous infusion over a period of time, by intramuscular, intraperitoneal, intracerebrospinal, transdermal, subcutaneous, intra-articular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, the CD80-Fc fusion proteins can be aerosolized using a fluorocarbon formulation and a metered dose inhaler, or inhaled as a lyophilized and milled powder.

In some aspects, a CD80-Fc fusion protein is administered via site-specific or targeted local delivery techniques. Examples of site-specific or targeted local delivery techniques include various implantable depot sources of the CD80-Fc fusion proteins or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of the CD80-Fc fusion proteins may be used for administration. In some aspects, the CD80-Fc fusion proteins may be administered neat. In some aspects, the CD80-Fc fusion proteins and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000.

In some aspects, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

The CD80-Fc fusion proteins described herein can be administered using any suitable method, including by injection (e.g., intraperitoneally, intravenously, subcutaneously, intramuscularly, etc.). The CD80-Fc fusion proteins can also be administered topically or via inhalation, as described herein. Generally, for administration of CD80-Fc fusion proteins the therapeutic dosage can be administered daily, every week, every other week, every three weeks, every four weeks, every five weeks, every six weeks, every seven weeks, every eight weeks, every ten weeks, every twelve weeks, or more than every twelve weeks. For repeated administrations over several days or longer, depending on the condition, the treatment is sustained until a desired suppression of symptoms occurs or until sufficient therapeutic levels are achieved, for example, to reduce symptoms associated with cancer. The progress of this therapy is easily monitored by conventional techniques and assays. The dosing regimen (including the specific CD80-Fc fusion proteins used) can vary over time.

In some aspects, a therapeutic dosage is administered daily with the dosage ranging from about any of 1 μg/kg to 30 μg/kg to 300 μg/kg to 3 mg/kg, to 30 mg/kg, to 100 mg/kg or more, depending on the factors mentioned above. For example, daily dosage of about 0.01 mg/kg, about 0.03 mg/kg, about 0.1 mg/kg, about 0.3 mg/kg, about 1 mg/kg, about 2.5 mg/kg, about 3 mg/kg, about 5 mg/kg, about 10 mg/kg, about 15 mg/kg, and about 25 mg/kg may be used.

In some aspects, a therapeutic dosage is administered every week (QW) with the dosage ranging from about any of 1 μg/kg to 30 μg/kg to 300 μg/kg to 3 mg/kg, to 30 mg/kg, to 100 mg/kg or more, depending on the factors mentioned above. For example, a weekly dosage of about 0.01 mg/kg, about 0.03 mg/kg, about 0.1 mg/kg, about 0.3 mg/kg, about 0.5 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some aspects, a therapeutic dosage is administered every two weeks (Q2W) with the dosage ranging from about any of 1 μg/kg to 30 μg/kg to 300 μg/kg to 3 mg/kg, to 30 mg/kg, to 100 mg/kg or more, depending on the factors mentioned above. For example, a bi-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some aspects, a therapeutic dosage is administered every three weeks (Q3W) with the dosage ranging from about any of 1 μg/kg to 30 μg/kg to 300 μg/kg to 3 mg/kg, to 30 mg/kg, to 100 mg/kg or more, depending on the factors mentioned above. For example, a tri-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In some aspects, a therapeutic dosage is administered every month or every four weeks (Q4W) with the dosage ranging from about any of 1 μg/kg to 30 μg/kg to 300 μg/kg to 3 mg/kg, to 30 mg/kg, to 100 mg/kg or more, depending on the factors mentioned above. For example, a monthly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In other aspects, a therapeutic dosage is administered daily with the dosage ranging from about 0.01 mg to about 1200 mg or more, depending on the factors mentioned above. For example, daily dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, or about 1200 mg may be used.

In other aspects, a therapeutic dosage is administered every week with the dosage ranging from about 0.01 mg to about 2000 mg or more, depending on the factors mentioned above. For example, weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other aspects, a therapeutic dosage is administered every two weeks with the dosage ranging from about 0.01 mg to about 2000 mg or more, depending on the factors mentioned above. For example, bi-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other aspects, a therapeutic dosage is administered every three weeks with the dosage ranging from about 0.01 mg to about 2500 mg or more, depending on the factors mentioned above. For example, tri-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg may be used.

In other aspects, a therapeutic dosage is administered every four weeks or month with the dosage ranging from about 0.01 mg to about 3000 mg or more, depending on the factors mentioned above. For example, monthly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, about 2600 mg, about 2700 mg, about 2800 mg, about 2900 mg, or about 3000 mg may be used.

In some aspects, a therapeutic of the present invention is administered at a dose ranging from about 1 μg/kg to about 600 μg/kg or more, about 6 μg/kg to about 600 μg/kg, about 6 μg/kg to about 300 μg/kg, about 30 μg/kg to about 600 μg/kg or about 30 μg/kg to about 300 μg/kg. For example, the dose is administered at about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 350 μg/kg, about 400 μg/kg, about 450 μg/kg, about 500 μg/kg, about 550 μg/kg or about 600 μg/kg may be used.

For the purpose of the present invention, the appropriate dosage of a CD80-Fc fusion protein will depend on the CD80-Fc fusion protein (or compositions thereof) employed, the type and severity of symptoms to be treated, whether the agent is administered for preventive or therapeutic purposes, previous therapy, the patient's clinical history and response to the agent, the patient's clearance rate for the administered agent, and the discretion of the attending physician. Typically the clinician will administer a CD80-Fc fusion protein until a dosage is reached that achieves the desired result. Dose and/or frequency can vary over course of treatment. Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of symptoms. Alternatively, sustained continuous release formulations of a CD80-Fc fusion protein may be appropriate. Various formulations and devices for achieving sustained release are known in the art.

In one embodiment, dosages for a CD80-Fc fusion protein may be determined empirically in individuals who have been given one or more administration(s) of a CD80-Fc fusion protein. For example, individuals are given incremental dosages of a CD80-Fc fusion protein. To assess efficacy, an indicator of the disease can be followed.

Administration of a CD80-Fc fusion protein as described herein in accordance with the method in the present invention can be continuous or intermittent, depending, for example, upon the recipient's physiological condition, whether the purpose of the administration is therapeutic or prophylactic, and other factors known to skilled practitioners. The administration of a CD80-Fc fusion protein may be essentially continuous over a preselected period of time or may be in a series of spaced doses.

In some aspects, more than one CD80-Fc fusion protein may be present. At least one, at least two, at least three, at least four, at least five different, or more CD80-Fc fusion proteins can be present. Generally, those CD80-Fc fusion proteins may have complementary activities that do not adversely affect each other.

In some aspects, the CD80-Fc fusion protein or variant CD80 polypeptide may be administered in combination with the administration of one or more additional agents. These include, but are not limited to, the administration of a biotherapeutic agent, a chemotherapeutic agent, a vaccine, immune cell therapy (e.g. CAR-T cell-based therapy), radiotherapy, a cancer vaccine, another cytokine therapy (e.g., immunostimulatory cytokines including various signaling proteins that stimulate immune response, such as interferons, interleukins, and hematopoietic growth factors), a targeted cytokine, an inhibitor of other immunosuppressive pathways, an inhibitors of angiogenesis, a T cell activator, an inhibitor of a metabolic pathway, an mTOR (mechanistic target of rapamycin) inhibitor (e.g., rapamycin, rapamycin derivatives, sirolimus, temsirolimus, everolimus, and deforolimus), an inhibitor of an adenosine pathway, a tyrosine kinase inhibitor including but not limited to inlyta, ALK (anaplastic lymphoma kinase) inhibitors (e.g., crizotinib, ceritinib, alectinib, and sunitinib), a BRAF inhibitor (e.g., vemurafenib and dabrafenib), a PI3K inhibitor, a HPK1 inhibitor, an epigenetic modifier, an inhibitors or depletor of Treg cells and/or of myeloid-derived suppressor cells, a JAK (Janus Kinase) inhibitor (e.g., ruxolitinib and tofacitinb, varicitinib, filgotinib, gandotinib, lestaurtinib, momelotinib, pacritinib, and upadacitinib), a STAT (Signal Transducers and Activators of Transcription) inhibitor (e.g., STAT1, STAT3, and STAT5 inhibitors such as fludarabine), a cyclin-dependent kinase inhibitor, an immunogenic agent (for example, attenuated cancerous cells, tumor antigens, antigen presenting cells such as dendritic cells pulsed with tumor derived antigen or nucleic acids, a MEK inhibitor (e.g., trametinib, cobimetinib, binimetinib, and selumetinib), a GLS1 inhibitor, a PARP inhibitor (e.g. talazoparib, olaparib, rucaparib, niraparib), an oncolytic virus, gene therapies including DNA, RNA delivered directly or by adeno-associated viruses (AAV) or nanoparticles, an innate immune response modulator (e.g., TLRs, KIR, NKG2A), an IDO (Indoleamine-pyrrole 2,3-dioxygenase) inhibitor, a PRR (Pattern Recognition Receptors) agonist, and cells transfected with genes encoding immune stimulating cytokines such as but not limited to GM-CSF).

In some aspects, the biotherapeutic agent is an antibody, including but not limited to, an anti-CTLA-4 antibody, an anti-CD3 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, a BCMA antibody, or an anti-GITR antibody.

In some aspects, other examples of the antibody for the combination use with the CD80-Fc fusion protein of the present invention can be directed to, 5T4; A33; alpha-folate receptor 1 (e.g. mirvetuximab soravtansine); Alk-1; BCMA [e.g. PF-06863135 (see U.S. Pat. No. 9,969,809)]; BTN1A1 (e.g. see WO2018222689); CA-125 (e.g. abagovomab); Carboanhydrase IX; CCR2; CCR4 (e.g. mogamulizumab); CCR5 (e.g. leronlimab); CCR8; CD3 [e.g. blinatumomab (CD3/CD19 bispecific), PF-06671008 (CD3/P-cadherin bispecific), PF-06863135 (CD3/BCMA bispecific)]; CD19 (e.g. blinatumomab, MOR208); CD20 (e.g. ibritumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, isatuximab), CD40; CD-40L; CD44v6; CD47 (e.g. Hu5F9-G4, CC-90002, SRF231, B6H12); CD52 (e.g. alemtuzumab); CD56; CD63; CD79 (e.g. polatuzumab vedotin); CD80; CD86; CD123; CD276/B7-H3 (e.g. omburtamab); CDH17; CEA; CIhCG; CTLA-4 (e.g. ipilimumab, tremelimumab), CXCR4; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO2018/220584); 4-1BB (CD137) [e.g. utomilumab/PF-05082566 (see WO2012/032433) or urelumab/BMS-663513], GD2 (e.g. dinutuximab, 3F8); GD3; GITR (e.g. TRX518); GloboH; GM1; GM2; HER2/neu [e.g. margetuximab, pertuzumab, trastuzumab; ado-trastuzumab emtansine, trastuzumab duocarmazine, PF-06804103 (see U.S. Pat. No. 8,828,401)]; HER3; HER4; ICOS; IL-10; ITG-AvB6; LAG-3 (e.g. relatlimab, IMP701); Lewis-Y; LG; Ly-6; M-CSF [e.g. PD-0360324 (see U.S. Pat. No. 7,326,414)]; MCSP; mesothelin; MUC1; MUC2; MUC3; MUC4; MUC5AC; MUC5B; MUC7; MUC16; Notch1; Notch3; Nectin-4 (e.g. enfortumab vedotin); OX40 [e.g. PF-04518600 (see U.S. Pat. No. 7,960,515)]; P-Cadherein [e.g. PF-06671008 (see WO2016/001810)]; PCDHB2; PD-1 [e.g. BCD-100, camrelizumab, cemiplimab, genolimzumab (CBT-501), MEDI0680, nivolumab, pembrolizumab, pidilizumab, RN888 (see WO2016/092419), sintilimab, spartalizumab, STI-A1110, tislelizumab, TSR-042]; PD-L1 (e.g. atezolizumab, durvalumab, BMS-936559 (MDX-1105), or LY3300054); PDGFRA (e.g. olaratumab); Plasma Cell Antigen; PolySA; PSCA; PSMA; PTK7 [e.g. PF-06647020 (see U.S. Pat. No. 9,409,995)]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g., sacituzumab govitecan); TSPAN8; VEGF (e.g. bevacizumab, brolucizumab); VEGFR1 (e.g. ranibizumab); VEGFR2 (e.g. ramucirumab, ranibizumab); and Wue-1.

In some aspects, the antibody for combination use may be an anti-PD-1 or anti-PD-L1 antibody. The programmed death 1 (PD-1) receptor and PD-1 ligands 1 and 2 (PD-L1 and PD-L2, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al., Nat Med 1999; 5:1365-69; Freeman et al. J Exp Med 2000; 192:1027-34), potentially providing an immune-tolerant environment for tumor development and growth. Conversely, inhibition of this interaction can enhance local T-cell responses and mediate antitumor activity in nonclinical animal models (Iwai Y, et al. Proc Natl Acad Sci USA 2002; 99:12293-97). Examples of anti-PD-L1 antibodies that are useful in the treatment method, medicaments and uses of the present invention include atezolizumab, durvalumab, BMS-936559 (MDX-1105), and LY3300054. Examples of anti-PD-1 antibodies that are useful in the treatment method, medicaments and uses of the present invention include BCD-100, camrelizumab, cemiplimab, genolimzumab (CBT-501), MEDI0680, nivolumab, pembrolizumab, RN888 (see WO2016/092419; U.S. Ser. No. 10/155,037), sintilimab, spartalizumab, STI-A1110, tislelizumab, and TSR-042. In some aspects, the anti-PD-1 antibody is PF-06801591/RN888. In some aspects, the anti-PD-1 antibody comprises a VH CDR1, VH CDR2, and VH CDR3 of a heavy chain variable region set forth as SEQ ID NO: 123 and/or a VL CDR1, VL CDR2, and VL CDR3 of a light chain variable region set forth as SEQ ID NO: 127. In some aspects, the anti-PD-1 antibody comprises a VH CDR1 of SEQ ID NO: 120, a VH CDR2 of SEQ ID NO: 121, and a VH CDR3 of SEQ ID NO: 122, and/or a VL CDR1 of SEQ ID NO: 124, a VL CDR2 of SEQ ID NO: 125, and/a VL CDR3 of SEQ ID NO: 126. In some aspects, the anti-PD-1 antibody comprises a heavy chain variable region set forth as SEQ ID NO: 123 and/or a light chain variable region set forth as SEQ ID NO: 127.

Therapeutic antibodies may have any suitable format. For example, therapeutic antibodies may have any format as described elsewhere herein. In some aspects, a therapeutic antibody may be a naked antibody. In some aspects, a therapeutic antibody may be linked to a drug/agent (also known as an “antibody-drug conjugate” (ADC)). Drugs or agents that can be linked to an antibody in the ADC format can include, for example, cytotoxic agents, immunomodulating agents, imaging agents, therapeutic proteins, biopolymers, or oligonucleotides. Exemplary cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof.

In some aspects, a therapeutic antibody against a particular antigen may incorporated into a multi-specific antibody (e.g. a bispecific antibody). Bispecific antibodies are monoclonal antibodies that have binding specificity for at least two different antigens. In some aspects, a bispecific antibody comprises a first antibody variable domain and a second antibody variable domain, wherein the first antibody variable domain is capable of recruiting the activity of a human immune effector cell by specifically binding to an effector antigen located on the human immune effector cell, and wherein the second antibody variable domain is capable of specifically binding to a target antigen as provided herein. Examples of effector antigens that can be bound by the heterodimeric protein (e.g., a heterodimeric antibody or a bispecific antibody) include, but are not limited to, human CD3 (or CD3 (Cluster of Differentiation) complex), CD16, NKG2D, NKp46, CD2, CD28, CD25, CD64, and CD89. The target antigen is typically expressed on a target cell in a diseased condition (e.g. a cancer cell). Examples of the target antigens of particular interest in bispecific antibodies include, but are not limited to, BCMA, EpCAM (Epithelial Cell Adhesion Molecule), CCR5 (Chemokine Receptor type 5), CD19, HER (Human Epidermal Growth Factor Receptor)-2/neu, HER-3, HER-4, EGFR (Epidermal Growth Factor Receptor), PSMA, CEA, MUC-1 (Mucin), MUC2, MUC3, MUC4, MUC5AC, MUC5B, MUC7, ClhCG, Lewis-Y, CD20, CD33, CD30, ganglioside GD3, 9-O-Acetyl-GD3, GM2, Globo H, fucosyl GM1, Poly SA, GD2, Carboanhydrase IX (MN/CA IX), CD44v6, Shh (Sonic Hedgehog), Wue-1, Plasma Cell Antigen, (membrane-bound) IgE, MCSP (Melanoma Chondroitin Sulfate Proteoglycan), CCR8, TNF-alpha precursor, STEAP, mesothelin, A33 Antigen, PSCA (Prostate Stem Cell Antigen), Ly-6; desmoglein 4, E-cadherin neoepitope, Fetal Acetylcholine Receptor, CD25, CA19-9 marker, CA-125 marker and MIS (Muellerian Inhibitory Substance) Receptor type II, sTn (sialylated Tn antigen; TAG-72), FAP (fibroblast activation antigen), endosialin, EGFRvIII, LG, SAS, PD-L1, CD47, SIRPa, and CD63. In some aspects, the antibody has an IgG1, IgG2, IgG3, or IgG4 isotype. In some aspects, the antibody comprises an immunologically inert Fc region. In some aspects the antibody is a human antibody or humanized antibody.

Immunostimulatory cytokines include various signaling proteins that stimulate immune response, such as interferons, interleukins, and hematopoietic growth factors. In some aspects, exemplary immunostimulatory cytokines include, but are not limited to, GM-CSF, G-CSF, IFNγ, IFNα, IL-2 (e.g. denileukin difitox), IL-6, IL-7, IL-10, IL-11, IL-12, IL-15, IL-18, IL-21, and TNFα. Immunostimulatory cytokines may have any suitable format. In some aspects, an immunostimulatory cytokine may be a recombinant version of a wild-type cytokine. In some aspects, an immunostimulatory cytokine may be a mutein that has one or more amino acid changes as compared to the corresponding wild-type cytokine. In some aspects, an immunostimulatory cytokine may be incorporated into a chimeric protein containing the cytokine and at least one other functional protein (e.g. an antibody). In some aspects, an immunostimulatory cytokine may covalently linked to a drug/agent (e.g. any drug/agent as described elsewhere herein as a possible ADC component). In some aspects, the cytokines are pegylated (e.g., pegylated IL-2, IL-10, IFNγ, and IFNα).

Pattern recognition receptors (PRRs) are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-I-like receptors (RLRs), nucleotide-binding oligomerization domain (NOD)-like receptors (NLRs), C-type lectin receptors (CLRs), and Stimulator of Interferon Genes (STING) protein.

Exemplary TLR agonists provided herein include agonists of TLR2, TLR3, TLR4, TLR5, TLR6, TLR7, TLR8, and TLR9. Examples of RLRs agonists that are useful in the treatment methods, medicaments, and uses of the present invention include, for example, short double-stranded RNA with uncapped 5′ triphosphate (RIG-I agonist); poly I:C (MDA-5 agonist), and BO-112 (MDA-A agonist). Examples of NLR agonists that are useful in the treatment methods, medicaments, and uses of the present invention include, for example, liposomal muramyl tripeptide/mifamurtide (NOD2 agonist). Examples of CLR agonists that are useful in the treatment methods, medicaments, and uses of the present invention include, for example, MD-fraction (a purified soluble beta-glucan extract from Grifola frondosa) and imprime PGG (a beta 1,3/1,6-glucan PAMP derived from yeast). Examples of STING agonists that are useful in the treatment methods, medicaments, and uses of the present invention include various immunostimulatory nucleic acids, such as synthetic double stranded DNA, cyclic di-GMP, cyclic-GMP-AMP (cGAMP), synthetic cyclic dinucleotides (CDN) such as MK-1454 and ADU-S100 (MIW815), and small molecules such as PO-424.

Cancer vaccines include various compositions that contain tumor associated antigens (or which can be used to generate the tumor associated antigen in the subject) and thus can be used to provoke an immune response in a subject that will be directed to tumor cells that contain the tumor associated antigen. Example materials that may be included in a cancer vaccine include, attenuated cancerous cells, tumor antigens, antigen presenting cells such as dendritic cells pulsed with tumor derived antigen or nucleic acids encoding tumor associated antigens. In some aspects, a cancer vaccine may be prepared with a patient's own cancer cells. In some aspects, a cancer vaccine may be prepared with biological material that is not from a patient's own cancer cells. Cancer vaccines include, for example, sipuleucel-T and talimogene laherparepvec (T-VEC).

Immune cell therapy involves treating a patient with immune cells that are capable of targeting cancer cells. Immune cell therapy includes, for example, tumor-infiltrating lymphocytes (TILs) and chimeric antigen receptor T cells (CAR-T cells).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1l and calicheamicin phil1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, s, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, fluridil, apalutamide, enzalutamide, cimetidine and goserelin; KRAS inhibitors; MCT4 inhibitors; MAT2a inhibitors; tyrosine kinase inhibitors/vascular endothelial growth factor (VEGF) receptor such as sunitinib, axitinib, sorafenib, tivozanib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600, abemaciclib and ribociclib; erb inhibitors such as dacomitinib; PARP inhibitors such as talazoparib, olaparib, rucaparib, niraparib; SMO inhibitors such as glasdegib, PF-5274857; EGFR T790M inhibitors such as PF-06747775; EZH2 inhibitors such as PF-06821497; PRMT5 inhibitors such as PF-06939999; TGFRBr1 inhibitors such as PF-06952229; and pharmaceutically acceptable salts, acids or derivatives of any of the above.

Poly (ADP-ribose) polymerase (PARP) engages in the naturally occurring process of DNA repair in a cell. PARP inhibition has been shown to be an effective therapeutic strategy against tumors associated with germline mutation in double-strand DNA repair genes by inducing synthetic lethality (Sonnenblick, A., et al., Nat Rev Clin Oncol, 2015. 12(1), 27-4).

Talazoparib is a potent, orally available PARP inhibitor, which is cytotoxic to human cancer cell lines harboring gene mutations that compromise deoxyribonucleic acid (DNA) repair, an effect referred to as synthetic lethality, and by trapping PARP protein on DNA thereby preventing DNA repair, replication, and transcription. The compound, talazoparib, which is “(8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one” and “(8S,9R)-5-fluoro-8-(4-fluorophenyl)-9-(1-methyl-1H-1,2,4-triazol-5-yl)-2,7,8,9-tetrahydro-3H-pyrido[4,3,2-de]phthalazin-3-one” (also referred to as “PF-06944076”, “MDV3800”, and “BMN673”) is a PARP inhibitor, having the structure,

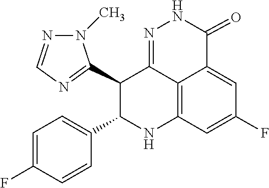 [SMILES: Cn1ncnc1[C@H]1c2n[nH]c(=O)c3cc(F)cc(c23)N[C@@H]1c1ccc(F)cc1]

Talazoparib, and pharmaceutically acceptable salts thereof, including the tosylate salt, are disclosed in International Publication Nos. WO 2010/017055 and WO 2012/054698. Additional methods of preparing talazoparib, and pharmaceutically acceptable salts thereof, including the tosylate salt, are described in International Publication Nos. WO 2011/097602, WO 2015/069851, and WO 2016/019125. Additional methods of treating cancer using talazoparib, and pharmaceutically acceptable salts thereof, including the tosylate salt, are disclosed in International Publication Nos. WO 2011/097334 and WO 2017/075091.

Talazoparib, as a single agent, has demonstrated efficacy, as well as an acceptable toxicity profile in patients with multiple types of solid tumors with DNA repair pathway abnormalities. There are also data supporting the efficacy of talazoparib in combination with chemotherapy in solid tumor types.

In some aspects, a CD80-Fc fusion protein is used in conjunction with one or more other therapeutic agents targeting an immune checkpoint modulator, such as, for example without limitation, an agent targeting PD-1, PD-L1, CTLA-4, LAG-3, B7-H3, B7-H4, B7-DC (PD-L2), B7-H5, B7-H6, B7-H8, B7-H2, B7-1, B7-2, ICOS, ICOS-L, TIGIT, CD2, CD47, CD80, CD86, CD48, CD58, CD226, CD155, CD112, LAIR1, 2B4, BTLA, CD160, TIM1, TIM-3, TIM4, VISTA (PD-H1), OX40, OX40L, GITRL, CD70, CD27, 4-1BB, 4-BBL, DR3, TL1A, CD40, CD40L, CD30, CD30L, LIGHT, HVEM, SLAM (SLAMF1, CD150), SLAMF2 (CD48), SLAMF3 (CD229), SLAMF4 (2B4, CD244), SLAMF5 (CD84), SLAMF6 (NTB-A), SLAMCF7 (CS1), SLAMF8 (BLAME), SLAMF9 (CD2F), CD28, CEACAM1(CD66a), CEACAM3, CEACAM4, CEACAM5, CEACAM6, CEACAM7, CEACAM8, CEACAM1-3AS CEACAM3C2, CEACAM1-15, PSG1-11, CEACAM1-4C1, CEACAM1-4S, CEACAM1-4L, IDO, TDO, CCR2, CD39-CD73-adenosine pathway (A2AR), BTKs, TIKs, CXCR2, CCR4, CCR8, CCR5, VEGF pathway, CSF-1, or an innate immune response modulator.

In some aspects, a CD80-Fc fusion protein composition comprises one or more additional therapeutic agents selected from talazoparib, crizotinib, palbociclib, gemcitabine, cyclophosphamide, fluorouracil, FOLFOX, folinic acid, oxaliplatin, axitinib, sunitinib malate, tofacitinib, bevacizumab, rituximab, and trastuzumab.

In some aspects, a CD80-Fc fusion protein is used in conjunction with a biotherapeutic agent and a chemotherapeutic agent. For example, provided is a method for treating cancer in a subject in need thereof comprising administering to the subject an effective amount of the CD80-Fc fusion protein as described herein, an anti-PD-1 antibody (e.g., RN888 (see WO2016/092419), nivolumab, or pembrolizumab, and a chemotherapeutic agent (e.g., gemcitabine, methotrexate, or a platinum analog).

In some aspects, provided is a method for treating cancer in a subject in need thereof comprising administering to the subject an effective amount of the CD80-Fc fusion protein as described wherein, a PARP inhibitor (e.g., talazoparib, olaparib, rucaparib, niraparib, and a chemotherapeutic agent (e.g., gemcitabine, methotrexate, or a platinum analog).

In some aspects, provided is a method for treating cancer in a subject in need thereof comprising administering to the subject an effective amount of the CD80-Fc fusion protein as described wherein, an anti-CTLA-4 antagonist antibody (e.g., ipilimumab, tremelimumab), and a chemotherapeutic agent (e.g., gemcitabine, methotrexate, or a platinum analog).

In some aspects, a CD80-Fc fusion protein composition is combined with a treatment regimen further comprising a traditional therapy selected from the group consisting of: surgery, radiation therapy, chemotherapy, targeted therapy, immunotherapy, hormonal therapy, angiogenesis inhibition and palliative care.

In some aspects, the CD80-Fc fusion protein therapy may precede or follow the other agent treatment by intervals ranging from minutes to weeks. In aspects where the other agents and/or a proteins or polynucleotides are administered separately, one would generally ensure that a significant period of time did not expire between each delivery, such that the agent and the composition of the present invention would still be able to exert an advantageously combined effect on the subject. In such instances, it is contemplated that one may administer both modalities within about 12-24 hours of each other and, more preferably, within about 6-12 hours of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

### Formulations

Therapeutic formulations of the CD80-Fc fusion protein or variant CD80 polypeptide used in accordance with the present invention are prepared for storage by mixing the protein having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the CD80-Fc fusion protein are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic CD80-Fc fusion protein compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a CD80-Fc fusion protein with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some aspects, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Kits

The invention also provides kits comprising any or all of the CD80-Fc fusion and variant CD80 polypeptides proteins described herein. Kits of the invention include one or more containers comprising a CD80-Fc fusion protein described herein and instructions for use in accordance with any of the methods of the invention described herein. Generally, these instructions comprise a description of administration of the CD80-Fc fusion protein for the above described therapeutic treatments. In some aspects, kits are provided for producing a single-dose administration unit. In certain aspects, the kit can contain both a first container having a dried protein and a second container having an aqueous formulation. In certain aspects, kits containing single and multi-chambered pre-filled syringes (e.g., liquid syringes and lyosyringes) are included.

The instructions relating to the use of a CD80-Fc fusion protein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a CD80-Fc fusion protein. The container may further comprise a second pharmaceutically active agent.

Kits may optionally provide additional components such as buffers and interpretive information. Normally, the kit comprises a container and a label or package insert(s) on or associated with the container.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1

### CD80-Fc Fusion Protein Generation and Binding Activity

### A. Expression and Purification

Gene syntheses were performed using endogenous codons of CD80 (from ATUM, formerly DNA2.0, Newark, CA). CD80-Fc fusion proteins having wild-type (WT) and variant CD80 extracellular domain were expressed as N-terminal fusion proteins of human IgG1 Fc (fragment crystallizable). The endogenous nucleotide sequences of human CD80 (Refseq: NM_005191.3, UniProtKB P33681) extracellular domain were genetically fused in frame with germline nucleotide sequences of human IgG1 Fc (UniProtKB P01857) with full hinge (with C220S mutation, EU numbering) and subcloned into the mammalian expression vector pDT5 (from ATUM, formerly DNA2.0, Newark, CA).

Fusion proteins were expressed by transient transfection using either Expi293 or ExpiCHO expression systems (from ThermoFisher Scientific USA) following supplier's instructions. CD80-Fc fusion proteins were purified on prepacked Protein A column and on size exclusion chromatography column to high purity. Purified fusion proteins were filter sterilized and stored at −80° C. before use.

The purity and homogeneity of the CD80-Fc fusion proteins were tested by analytical size exclusion chromatography (aSEC), capillary gel electrophoresis and mass spectromtery. The intact mass of the purified fusion proteins was confirmed by Xevo G2-XS QTof Quadrupole Time-of-Flight Mass Spectrometry (from WATERS) coupled to an Acquity UPLC Protein BEH C4 (300 Å 1.7 μm). CD80-Fc fusion proteins were deglycosylated first in non-reducing and reducing conditions using rapid PNGase F enzyme (from New England Biolabs, P0710S and P0711S) to determine mass of the intact proteins (non-reduced) and reduced proteins.

### B. ELISA Binding Affinity

Binding affinity of purified CD80-Fc fusion proteins (WT and variant) to recombinant soluble CD28 and CTLA-4 proteins was determined by standard ELISA. Recombinant soluble CD28 and CTLA-4 proteins (from Creative BioMart, Product #CD28-3910H, CTLA-4-2232H) were immobilized on 96 well microtiter plate (Thermo Scientific, Product #436110) in bicarbonate buffer at 4° C. overnight. After washing with 1×PBS containing 0.05% Tween 20 (wash buffer) plates were incubated with blocking buffer, 5% BSA in PBS, for 1 hour at room temperature. A serial dilution of CD80-Fc fusion proteins and human IgG1 isotype control antibody was prepared in blocking buffer, added into the plate and incubated at room temperature for 1 hour. Following washing 3× with wash buffer, 1:4000 dilution of HRP conjugated secondary anti-human IgG1 antibody (R&D Systems, MAB110-100) was added into the plate and incubated at room temperature for 1 hour. Plates were washed 3× with wash buffer and a 100 μl of LumiGlo (SeraCare, Product #5430-0040) reagent added into each well, incubated for 5 minutes at room temperature before reading luminescence signal in EnSight™ plate reader (PerkinElmer).

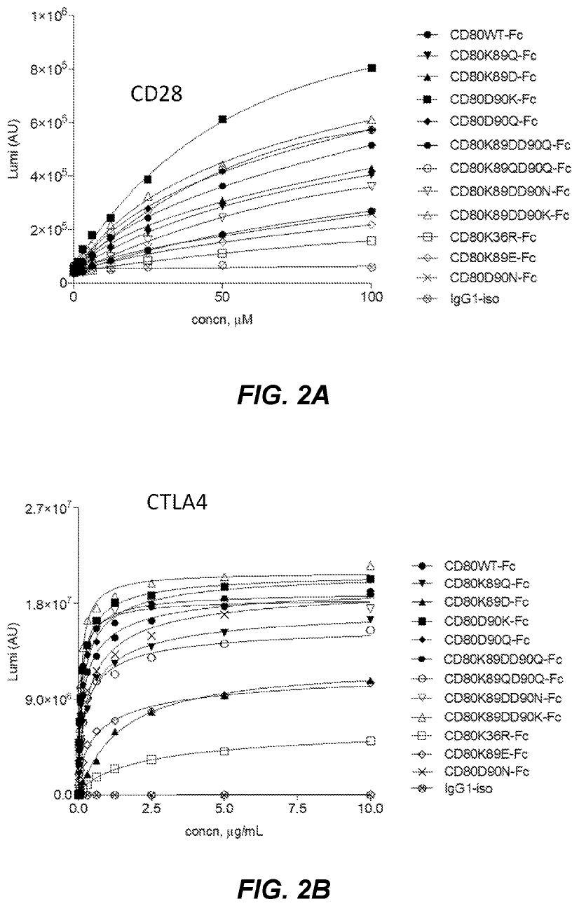

FIGS. 2A and 2B show binding activity of WT and variant CD80-Fc fusion proteins against soluble CD28 and CTLA-4 proteins, respectively, by standard ELISA. FIG. 2A shows variant CD80-Fc fusion proteins increased binding affinity to CD28, as compared to WT CD80-Fc, for example, CD80-D90K-Fc, CD80-K89D-D90K-Fc, CD80-D90Q-Fc, CD80-K89Q-D90Q-Fc, CD80-K89D-D90Q-Fc, CD80-K89D-Fc, K89Q-Fc, and CD80-K89D-D90N-Fc. FIG. 2B shows the binding affinity of WT and variant CD80-Fc fusion proteins to CTLA-4.

### C. Jurkat Cell Binding Affinity

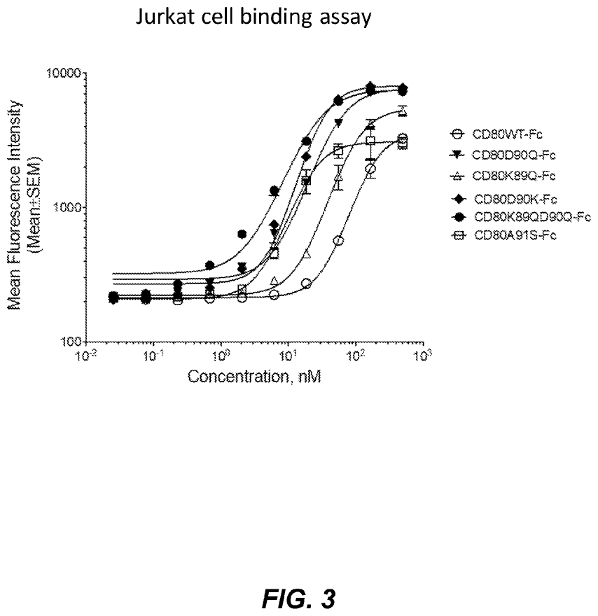

Binding affinity of CD80-Fc fusion proteins (WT and variant) against CD28 expressed on Jurkat T cell line (ATCC TIB-152) was measured by flow cytometry. Jurkat cells (100,000 cells suspended in 100 μL PBS supplemented with 0.5% BSA, 2 mM EDTA, and 10% normal goat serum) were incubated with CD80-Fc fusion proteins at concentrations ranging 0.025-500 nM on ice for 30 minutes. Cells were then washed with PBS supplemented with 0.5% BSA and 2 mM EDTA twice. Next, cells were stained with PE-conjugated goat anti-human Fc secondary antibody (Jackson ImmunoResearch 109-116-170) diluted 1:200 in 50 μL PBS supplemented with 0.5% BSA and 2 mM EDTA on ice for 30 minutes. Afterward, cells were washed with PBS supplemented with 0.5% BSA and 2 mM EDTA twice. Finally, cells were suspended in PBS containing 0.5 μg/mL 7-amino-actinomycin D and acquired on BD LSRFortessa X-20 flow cytometer. Data were analyzed by Flowjo v10 software (Flowjo, LLC). FIG. 3 and Table 1 show the binding affinity of CD80-Fc fusion proteins (WT and variants) against CD28 expressed on Jurkat cells.

TABLE 1
Binding affinity of WT and variant CD80-Fc
fusion proteins on Jurkat cells.

| | KD |
|---|---|
| CD80-Fc fusion proteins | (nM) |
| CD80-WT-Fc | 162 |
| CD80-K89Q-Fc | 91 |
| CD80-D90Q-Fc | 49 |
| CD80-D90K-Fc | 29 |
| CD80-K89Q-D90Q-Fc | 23 |
| CD80-A91S-Fc | 20 |

### Example 2

### Co-Stimulation Assays

### A. Primary T Cell Assay for Co-Stimulation Bioactivity Measurements

A primary T cell co-stimulation assay was designed to measure IL-2 production in culture media upon T cell activation and co-stimulation. Co-engagement of T cell antigen receptor (TCR/CD3) and CD28 co-stimulatory receptor induces activation of T cells and downstream intracellular signaling pathways that lead to the regulation of transcriptional factor NFAT, NF-kB and AP-1, which bind to IL-2 promoter and induce IL-2 expression.

Human primary T cells were isolated from fresh peripheral blood obtained from Stem Cell Technologies (Leuko Pak, Product #70500.2) using EasySep Human T cell isolation kit (Stem Cell Technologies Product #17951). A 1 mL aliquot of Leuko Pak cells was thawed and resuspended in 25 mL Lymphocyte medium. Cells were centrifuged and resuspended in 2 mL RoboSep medium (Stem Cell Technologies, Catalog #20104). Cell density was adjusted and transferred to a 15 mL Falcon tube. Isolation cocktail, 50 μL/mL cells was added to the tube and the cells incubated before adding 40 μL/mL Rapid spheres and mixing gently. The tube with cells was placed in an EasySep Magnet (Stem Cell Technologies, Product #18001), incubated and collected transferred cell supernatant in a separate tube. Enriched lymphocytes were centrifuged and resuspended in growth media (RPMI, Gibco-11875, with 10% HI FBS, 100 μg/mL Pen-strep). A stock of 4×10⁵ untouched pan T cells per mL was prepared in growth media for further use.

A human colon cancer cell line, HCT116, that expresses CDH3 (P-Cadherin) was used for TCR engagement through a CDH3×CD3 bispecific, which binds to CD3 on human primary T cells and CDH3 on tumor cells. HCT116 cells were genetically engineered by lentiviral transduction to overexpress human FCγRI (CD64, GenBank:AAA58414.1, RefSeq:P12314.1) on the cell surface for the assembly and display of CD80-Fc fusion proteins.

Tissue culture treated 96 well flat bottom plates (Corning, USA) was seeded with HCT116 parental and HCT116-CD64 cells, 4,000 cells per well in 100 μL growth media (RPMI, Gibco-11875, with 10% HI FBS, 100 μg/mL Pen-strep) and grown overnight at 37 ºC in CO₂ incubator. To each well 50 μL of pan T cells (20,000 cells per well) and 25 μL of the CDH3×CD3 bispecific (20 ng/ml final concentration per well) were added. 25 μL serial dilutions of CD80-Fc fusion proteins with concentrations ranging from 0-1000 ng/ml (final concentrations per well) were added with appropriate controls. Cells were cultured for 72 hours at 37° C. in CO₂ incubator before harvesting culture supernatants and measuring human IL-2 release.

IL-2 release in culture supernatants from T cell co-stimulation assay was determined by standard ELISA. Commercial Human IL-2 ELISA kit from Biolegend (Product #431803) was used to determine IL-2 release levels. Anti-human IL-2 capture antibody was immobilized on Maxisorp 96 well microtiter plate (Thermo Scientific, Product #436110) in bicarbonate buffer at 4° C. overnight. After washing with 1×PBS, plates were incubated with blocking buffer. Growth media from assay plates were diluted 5× in blocking buffer and 100 μL of diluted media was added into ELISA plate. The plates were incubated and washed with washing buffer. A 1:1000 dilution of biotin conjugated anti-human IL-2 detection antibody was added into the plate and incubated. Following washing with wash buffer a 1:4000 dilution of Avidin-HRP was added into the plate and incubated. Plates were washed and LumiGlo (SeraCare, Product #5430-0040) reagent was added into each well, incubated for 5 minutes before reading luminescence in EnSight™ plate reader (PerkinElmer).

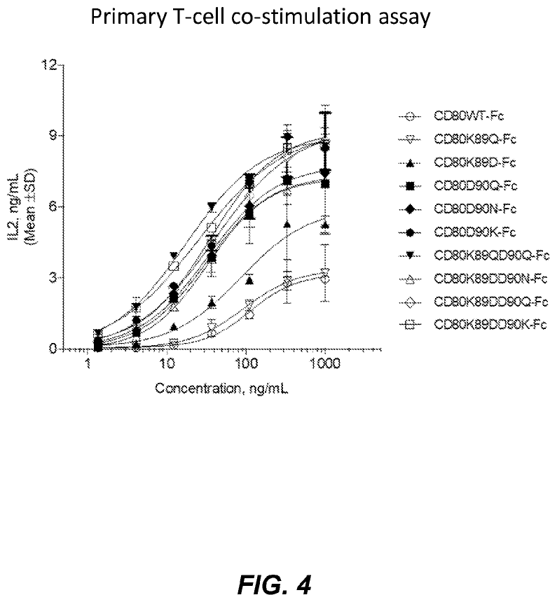

FIG. 4 shows IL-2 production levels and Table 2 shows EC₅₀ from the primary T cell and HCT116-CD64 cell co-stimulation assay. As shown in Table 2, variant CD80-Fc fusion proteins showed varying degree of enhanced T cell activation and co-stimulation compared to that of CD80-WT-Fc fusion proteins (EC₅₀ 1.2 nM; Table 2). CD80-D90K-Fc and CD80-K89D-D90K-Fc fusion proteins showed enhanced T cell co-stimulation by about a 8-10-fold compared that of WT CD80-Fc fusion proteins. CD80-D90N-Fc, CD80-D90Q-Fc, CD80-K89Q-D90Q-Fc, CD80-K89D-D90N-Fc and CD80-K89D-D90Q-Fc fusion proteins showed enhanced T cell co-stimulation by about a 3-fold compared to WT CD80-Fc fusion proteins. CD80-K89D-Fc and CD80-K89Q-Fc showed enhanced T cell co-stimulation by about a 1.2-1.5-fold compared to CD80-WT-Fc fusion proteins. Further, variant CD80-Fc fusion proteins that had higher binding affinity compared to CD80-WT-Fc fusion proteins (see Example 1) showed higher T cell co-stimulation.

TABLE 2
EC₅₀ from primary T cell and HCT116-
CD64 cells co-culture assay

| | EC₅₀ | EC₅₀ |
|---|---|---|
| CD80-Fc fusion proteins | ng/mL | nM |
| CD80-WT-Fc | 113.8 | 1.15 |
| CD80-K89Q-Fc | 92.9 | 0.94 |
| CD80-K89D-Fc | 86.0 | 0.86 |
| CD80-D90Q-Fc | 31.9 | 0.32 |
| CD80-D90N-Fc | 33.0 | 0.33 |
| CD80-D90K-Fc | 17.9 | 0.18 |
| CD80-K89Q-D90Q-Fc | 43.3 | 0.46 |
| CD80-K89D-D90N-Fc | 40.4 | 0.41 |
| CD80-K89D-D90Q-Fc | 35.8 | 0.36 |
| CD80-K89D-D90K-Fc | 15.3 | 0.16 |

### B. Jurkat-IL-2-Luc Reporter Assay for Co-Stimulation Bioactivity Measurements

A T cell co-stimulation assay was designed based on T Cell Activation Bioassay (IL-2) Kit from Promega (Product #J1631). The assay consisted of a genetically engineered Jurkat T cell line that expresses luciferase reporter driven by an IL-2 promoter. Co-engagement of T cell antigen receptor (TCR/CD3) and CD28 co-stimulatory receptor induces activation of T cells and downstream intracellular signaling pathways that in turn regulate the expression of luciferase reporter. As described above, HCT116 human colon cancer cell line was used for TCR engagement using the CDH3×CD3 bispecific and genetically engineered by lentiviral transduction to express human FCγRI.

A 96 well culture plate was seeded with HCT116 parental or HCT116-CD64 cells at 40,000 cells per well in 100 μL RPMI with 10% HI FBS and grown overnight at 37° C. in CO₂ incubator. To each well, Jurkat reporter cells (100,000 per well) and 10 μL of the CDH3×CD3 bispecific (8 ng/ml final concentration) were added. 10 μL of serial dilutions of variant CD80-Fc fusion proteins with concentrations ranging from 0-1000 ng/ml (final concentrations per well) was added with appropriate controls. Assay plates were incubated for 6 hours at 37° C. in CO₂ incubator for before harvesting culture supernatants and transferring to new plates. Following equilibration, 80 μL of BioGlo luminescence reagent (BioGlo Luciferase assay kit, Promega, G7940) was added into each well. Plates were incubated for 5 minutes before reading luminescence on the EnSight™ plate reader (PerkinElmer).

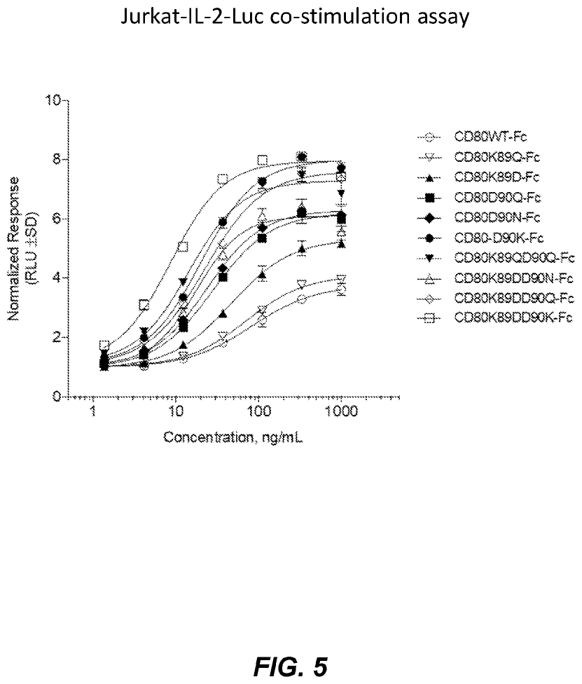

FIG. 5 shows the normalized responses for luciferase reporter activity and Table 3 shows the EC50 from Jurkat-IL-2-Luc and HCT116-CD64 cells co-stimulation assay. The variant CD80-Fc fusion proteins exhibited similar enhancement of co-stimulatory activity as observed in the primary T cell-tumor cell assay described above. As shown in Table 3, compared to the CD80-WT-Fc fusion protein (EC50 0.894 nM), CD80-K89D-D90K-Fc and CD80-D90K-Fc fusion proteins enhanced co-stimulation by about an 8-fold and CD80-K89Q-D90Q-Fc, CD80-K89D-D90N-Fc and CD80-K89D-D90Q-Fc fusion proteins enhanced co-stimulation by about 4-fold. In a further experiment, CD80-A91S-Fc was determined to have an EC50 (nM) of 0.50.

TABLE 3
EC₅₀ from Jurkat-IL-2-Luc and HCT116-
CD64 cells co-culture assay

| | EC₅₀ | EC₅₀ |
|---|---|---|
| CD80-Fc fusion proteins | ng/mL | nM |
| CD80-WT-Fc | 82.5 | 0.836 |
| CD80-K89Q-Fc | 70.2 | 0.709 |
| CD80-K89D-Fc | 46.8 | 0.470 |
| CD80-D90Q-Fc | 28.2 | 0.285 |
| CD80-D90N-Fc | 23.9 | 0.240 |
| CD80-D90K-Fc | 14.4 | 0.145 |
| CD80-K89Q-D90Q-Fc | 17.7 | 0.179 |
| CD80-K89D-D90N-Fc | 22.7 | 0.228 |
| CD80-K89D-D90Q-Fc | 20.3 | 0.204 |
| CD80-K89D-D90K-Fc | 8.8 | 0.088 |
| CD80-A91S-Fc | — | 0.500 |

### C. Jurkat-IL-2-Luc Cell Co-Stimulation Assay by Endogenous TCR (NY-ESO1-MHCI) Engagement

Jurkat-IL-2-Luc reporter cells (Promega, Product #J1631) were engineered with T cell receptor, TCR (Wargo J. Cancer Immunol Immunother 2009; Zhao Y. J Immunol 2005, Li Y, Nat Biotechnol 2005) against HLA-A2 restricted NY-ESO1 (SLLMWITQC-SEQ ID NO: 19) epitope using lentiviral transduction system. Human melanoma cells A375, which express human HLA-A*0201, were transduced with lentiviral vector containing endogenous codon for NY-ESO1 gene (Refseq accession NM_001327.2, CCDS14758.1). Upon expression, the construct underwent proteolytic cleavage at the ubiquitin/epitope junction, thus generating free, cytoplasmic epitope, which can be translocated into the endoplasmic reticulum and loaded onto class I HLA. A375-NY-ESO1 positive cells were treated with IFNγ to induce HLA expression and NY-ESO1 antigen presentation. The HLA expression and the antigen presentation was confirmed by Western Blot. The NY-ESO1⁺-A375 cells were further engineered to express human FCγRI (CD64, GenBank:AAA58414.1, RefSeq:P12314.1) for cell surface assembly and displaying of CD80-Fc fusion proteins. The expression of FCγRI was confirmed by flow cytometry analysis.

A 96 well culture plate was seeded with NY-ESO1⁺-A375 cells or NY-ESO1⁺/FCγRI⁺-A375 at 40,000 cells per well in 100 UL growth media (RPMI, 10% Heat Inactivated FBS, 200 μg/mL Hygromycin B, 1 mM sodium pyruvate, 0.1 mM MEM NEAA, 1 μg/mL puromycin) containing 10 ng/ml of human IFNγ (R&D Systems, product #285-IF) to induce MHC-A2 expression and grown overnight at 37° C. in CO₂ incubator. To each well, NY-ESO1-TCR⁺ Jurkat reporter cells, 100,000 (in 20 μL media), were added. 20 μL of serial dilutions of variant CD80-Fc fusion proteins with concentrations ranging from 0-1000 ng/ml (final concentrations per well) was added with appropriate controls. Assay plates were incubated for 6 hours at 37° C. in CO₂ incubator. Following equilibration, 80 μL BioGlo luminescence reagent (BioGlo Luciferase assay kit, Promega, G7940) was added into each well. Plates were incubated for 5 minutes before reading luminescence on the EnSight™ plate reader (PerkinElmer).

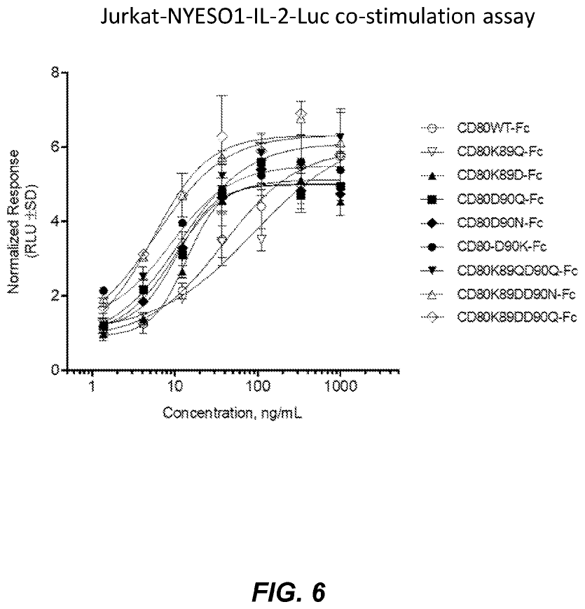

FIG. 6 shows the normalized responses for luciferase reporter activity and Table 4 shows the EC₅₀ from the Jurkat-NYESO1-IL-2-Luc and A375-CD64 cells co-stimulation assay. As shown in Table 4, variant CD80-Fc fusion proteins showed varying degree of enhanced T cell co-stimulation compared to CD80-WT-Fc fusion proteins (EC₅₀ 0.394 nM). Compared to CD80-WT-Fc fusion protein, CD80-K89D-D90N-Fc and CD80-K89D-D90Q-Fc fusion proteins enhanced co-stimulation by about 8-fold and CD80-D90Q-Fc, CD80-D90K-Fc, CD80-D90N-Fc and CD80-K89Q-D90Q-Fc fusion proteins enhanced T cell co-stimulation by about 4-fold. In a further experiment, CD80-A91S-Fc was determined to have an EC50 (nM) of 1.1.

TABLE 4
EC₅₀ from Jurkat-NYESO1-IL-2-Luc and
A375-CD64 cells co-culture assay

| | EC₅₀ | EC₅₀ |
|---|---|---|
| CD80-Fc fusion proteins | ng/mL | nM |
| CD80-WT-Fc | 38.9 | 0.394 |
| CD80-K89Q-Fc | 89.6 | 0.906 |
| CD80-K89D-Fc | 13.7 | 0.138 |
| CD80-D90Q-Fc | 9.8 | 0.099 |
| CD80-D90N-Fc | 10.5 | 0.105 |
| CD80-D90K-Fc | 9.1 | 0.092 |
| CD80-K89Q-D90Q-Fc | 11.5 | 0.116 |
| CD80-K89D-D90N-Fc | 5.8 | 0.059 |
| CD80-K89D-D90Q-Fc | 5.5 | 0.055 |
| CD80-A91S-Fc | — | 1.1 |

### D. Jurkat-CTLA-4-IL-2-Luc Report Assay for Co-Stimulation Bioactivity Measurements

A T cell co-stimulation assay to interrogate the activity of CD80-Fc molecules at the presence of both CD28 and CTLA-4 was designed based on CTLA-4 Blockade Bioassay Kit from Promega (Product #JA3001). This assay uses a genetically engineered Jurkat T cell line that constitutively expresses human CTLA-4 and expresses luciferase reporter driven by an IL-2 promoter. Co-engagement of T cell receptor (TCR/CD3) and CD28 co-stimulatory receptor activates T cells and induces downstream signaling pathways that regulate the expression of luciferase reporter. However, this activation can be attenuated by CTLA-4 competing away CD80-Fc molecules from co-stimulatory receptor CD28. As described above, HCT116 human colon cancer cell line was used for TCR engagement using a CDH3×CD3 bispecific and genetically engineered by lentiviral transduction to express human FCγRI.

A 96 well culture plate was seeded with HCT116-CD64 cells at 40,000 cells per well in 100 μL RPMI-1640 medium supplemented with 10% heat-inactivated FBS and grown overnight at 37 ºC in 5% CO₂ incubator. To each well, Jurkat reporter cells (100,000 per well) and 10 μL of PF-06671008 (8 ng/ml final concentration) were added. 10 μL of serial dilutions of variant CD80-Fc fusion proteins with concentrations ranging 0.01-200 nM (final concentrations per well) was added with appropriate controls. Assay plates were incubated for 6 hours at 37ºC in 5% CO₂ incubator before harvesting culture supernatants and transferring to new plates. Following equilibration, 80 μL of BioGlo luminescence reagent (BioGlo Luciferase assay kit, Promega, G7940) was added into each well. Plates were incubated before reading luminescence on the EnSight™ plate reader (PerkinElmer).

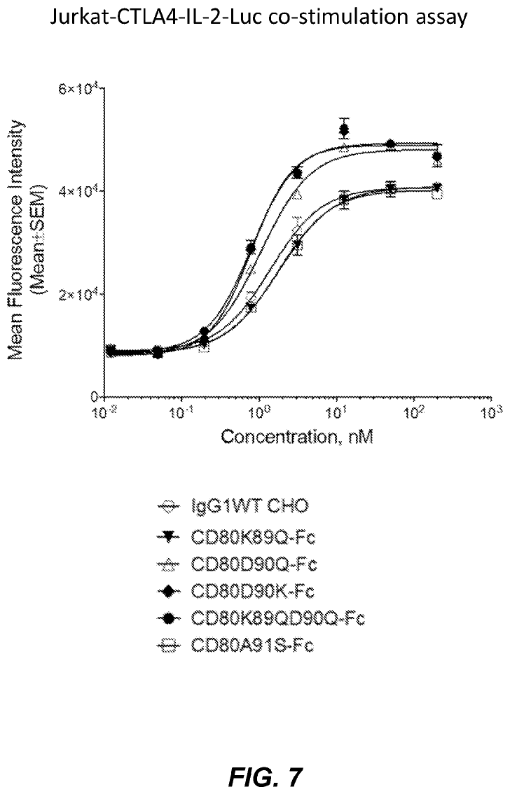

FIG. 7 shows luciferase reporter activity and Table 5 shows the EC₅₀ from Jurkat-CTLA-4-IL-2-Luc and HCT116-CD64 cells co-stimulation assay. As shown in Table 5, variant CD80-Fc fusion proteins, CD80-D90Q-Fc, CD80-D89Q-D90Q-Fc, and CD80-D90K-Fc, exhibited similar enhancement of co-stimulatory activity as observed for primary T cell-tumor cell assay (described above).

TABLE 5
EC₅₀ from Jurkat-CTLA-4-IL-2-Luc and
HCT116-CD64 cells co-culture assay

| | EC₅₀ |
|---|---|
| CD80-Fc fusion proteins | nM |
| CD80-WT-Fc | 1.4 |
| CD80-K89Q-Fc | 1.8 |
| CD80-D90Q-Fc | 1.0 |
| CD80-D90K-Fc | 0.80 |
| CD80-K89Q-D90Q-Fc | 0.80 |
| CD80-A91S-Fc | 1.8 |

### Example 3

### Stabilized Variant CD80-Fc Fusion Proteins

Increasing the temperature by a few degrees higher than the normal functioning temperature of protein therapeutics can lead to unfolding and structural changes that can impact their function. A structure-based engineering approach was utilized to improve the thermostability of CD80-Fc fusion proteins described herein.

### A. Disulfide Stabilized

To identify disulfide stabilizing mutations the crystal structure of dimeric CD80-ECD was analyzed to identify mechanically fragile regions of the protein that are most likely to unfold at higher temperatures. This region was located at the dimeric interface between the two CD80 ECDs. The interface was loosely packed and may contribute to instability. To strengthen the identified dimeric interface and improve stability, disulfide bridges were engineered through the introduction of cysteine mutations. Locations for engineering new disulfide bridges were evaluated using a computational customized tool based on MODELLER (B. Webb, A. Sali. Comparative Protein Structure Modeling Using Modeller. Current Protocols in Bioinformatics 54, John Wiley & Sons, Inc., 5.6.1-5.6.37, 2016). Residues having alpha and beta carbons closer than the following values: maximum Cα-Cα distance=7.0 Å and maximum Cβ-Cβ distance=5.5 Å were considered for engineering disulfides. Three disulfide stabilized variants were produced as CD80-Fc fusion proteins and scaled up in transient HEK and stable CHO cell lines for further characterization and profiling: CD80-K89Q-D90Q-V22C-G45C-Fc, CD80-K89Q-D90Q-161C-Fc and K89Q-D90Q-E23C-A26C-Fc.

### B. Non-Cysteine Stabilized

To identify non-cysteine stabilizing mutations analysis of the crystal structure of CD80-ECD further identified regions with polar amino acids facing hydrophobic amino acids in the interior of one CD80-ECD monomer as well as at the dimer CD80-ECD interface. FoldX (O. Buß, J. Rudat, K. Ochsenreither. FoldX as Protein Engineering Tool: Better Than Random Based Approaches? Computational and Structural Biotechnology Journal 16 (2018) 25-33) was used to evaluate single and double mutations in the CD80 interface followed by evaluation of complex energy and total system energy. Based on most favorable stability (internal structure stabilization and ECD interface stabilization), 24 non-cysteine stabilized variants were produced as CD80-Fc fusion proteins and scaled up in transient HEK and stable CHO cell lines for further characterization and profiling: CD80-V11L-V22F-Fc, CD80-V11L-T62Y-Fc, CD80-V11L-T62Y-N63D-Fc, CD80-V22F-T62L-Fc, CD80-T28V-T57V-Fc, CD80-T28V-T57V-Y31Q-Q33E-K54E-Fc, CD80-D60Y-Fc, CD80-D60Y-K54E-N63E-N64D-Fc, CD80-D60Y-T62L-Fc, CD80-D60Y-T62L-N63D-N64E-Fc, CD80-V22F-D60Y-Fc, CD80-V22F-D60Y-K54E-N64E-Fc, CD80-D60F-T62I-Fc, CD80-D60R-T62Y-Fc, CD80-D60Y-V11L-Fc, CD80-D60Y-V11L-N63D-Fc, CD80-D60Y-V22M-Fc, CD80-D60T-T62Y-Fc, CD80-D60Q-T62F-Fc, CD80-V22F-T28V-T57V-Fc, CD80-V22F-T28V-T57V-Y31Q-Q33E-K54E-Fc, CD80-V22F-T62L-N64E-Fc, CD80-V22F-T62L-N63D-N64E-Fc and CD80-D60Y-T62L-N63D-Fc.

### Example 4

### Non-Specific Binding and Self-Interaction

Variant CD80-Fc fusion proteins were assessed by measuring non-specific binding using a DNA- and insulin-binding ELISA (Avery et al., MAbs. 2018 February/March; 10(2): 244-255) and for self-interaction in an AC-SINS assay (affinity-capture self-interaction nanoparticle spectroscopy; Liu et al., 2014, mAbs 6:483-92). DNA- and insulin-binding scores were calculated as the signal ratio of the ELISA signal of the CD80-Fc fusion protein at 10 μg/ml to the ELISA signal in the absence of the Fc-fusion protein (buffer only). For the AC-SINS assay, proteins are captured by anti-human Fc antibodies coated on gold nanoparticles. Proteins that self-interact exhibit a clustering of nanoparticles which leads to a shift in absorbance maximum (AC-SINS score). The score ranges obtained from these in vitro assays correlate well with in vivo clearance using huFcRn transgenic (Tg32) mouse. Therapeutic proteins with high scores are at high risk for rapid clearance and unfavorable PK and therapeutics that score low are at low risk and favorable PK. As shown in Tables 6 and 7, low AC-SINS and DNA/insulin scores were observed for variant CD80-Fc fusion proteins.

TABLE 6
Non-specific binding and self-interactions
of variant CD80-Fc fusion proteins

| | AC-SINS | DNA | Insulin |
|---|---|---|---|
| Treatment | Score | Score | Score |
| Positive Control | 23 | 20 | 20 |
| Negative Control | 2 | 3 | 3 |
| CD80-WT-Fc | 0 | 1 | 1 |
| CD80-D90Q-Fc | 0 | 2 | 1 |
| CD80-K89Q-D90Q-Fc | 0 | 3 | 2 |
| CD80-K89Q-D90Q- | −1 | 1 | 1 |
| E23C-A26C-Fc | | | |
| CD80-K89Q-D90Q- | −1 | 1 | 1 |
| V22C-G45C -Fc | | | |
| CD80-K89Q-D90Q-161C -Fc | −1 | 2 | 1 |

TABLE 7
Non-specific binding and self-interactions
of variant CD80-Fc fusion proteins

| | AC-SINS | DNA | Insulin |
|---|---|---|---|
| Treatment | Score | Score | Score |
| Positive control | 17 | 36 | 32 |
| Negative control | 2 | 4 | 5 |
| CD80-WT-Fc | 0 | 1 | 1 |
| CD80-K89D-D90K- | −1 | 1 | 1 |
| T28V-T57V-Fc | | | |

### Example 5

### Thermal Stability

There is a correlation between the thermal stability of a protein with the overall stability of the protein. Enhanced thermal stability often provides improved manufacturability and longer shelf life/stability. Thermal stability of variant CD80-Fc fusion proteins was assessed by Differential Scanning calorimetry (DSC). Variant CD80-Fc fusion proteins were analyzed using a MicroCal VP-DSC instrument. Protein concentration was 0.03 mM in PBS, and sample and reference cells were heated from 10° ° C. to 100° C. at a scan rate of 100° C. per hour. Tables 8-10 show the first thermal transition temperature (Tm1) of CD80-Fc fusion proteins and the enhanced thermal stability of variant CD80-Fc fusion proteins compared to CD80-WT-Fc fusion proteins.

TABLE 8
Thermal stability of variant CD80-Fc fusion proteins

| CD80-Fc Fusion Proteins | DSC Tm1 [° C.] |
|---|---|
| CD80-WT-Fc | 59.52 ± 0.07 |
| CD80-D90Q-Fc | 59.83 ± 0.08 |
| CD80-K89Q-D90Q-Fc | 59.67 ± 0.10 |
| CD80-K89Q-D90Q-E23C-A26C-Fc | 67.28 ± 0.2 |
| CD80-K89Q-D90Q-V22C-G45C -Fc | 68.67 ± 0.22 |
| CD80-K89Q-D90Q-161C -Fc | 64.10 ± 0.17 |

TABLE 9
Thermal stability of variant CD80-Fc fusion proteins

| CD80-Fc Fusion Proteins | DSC Tm1 [° C.] |
|---|---|
| CD80-WT-Fc | 59.52 ± 0.07 |
| CD80-V11L-V22F-Fc | 61.52 ± 0.12 |
| CD80-V11L-T62Y-Fc | 58.76 ± 0.14 |
| CD80-T28V-T57V-Fc | 64 |
| CD80-V22F-T28V-T57V-Fc | 64 |

TABLE 10
Thermal stability of variant CD80-Fc fusion proteins

| CD80-FcFusion Proteins | Formulation | DSC Tm1 [° C.] |
|---|---|---|
| CD80-K89D-D90K-T28V-T57V-Fc | PBS | 63.70 ± 0.07 |
| | Tris pH 7.5 | 66.94 ± 1.6 |
| | His pH 5.8 | 68.57 ± 0.74 |
| | Glu pH 4.5 | 65.11 ± 0.23 |
| CD80-WT-Fc | PBS | 59.52 ± 0.07 |
| | Tris pH 7.5 | 64.77 ± 0.14 |
| | His pH 5.8 | 63.40 ± 0.35 |
| | Glu pH 4.5 | 62.78 ± 0.03 |

### Example 6

### Thermal Forced Aggregation

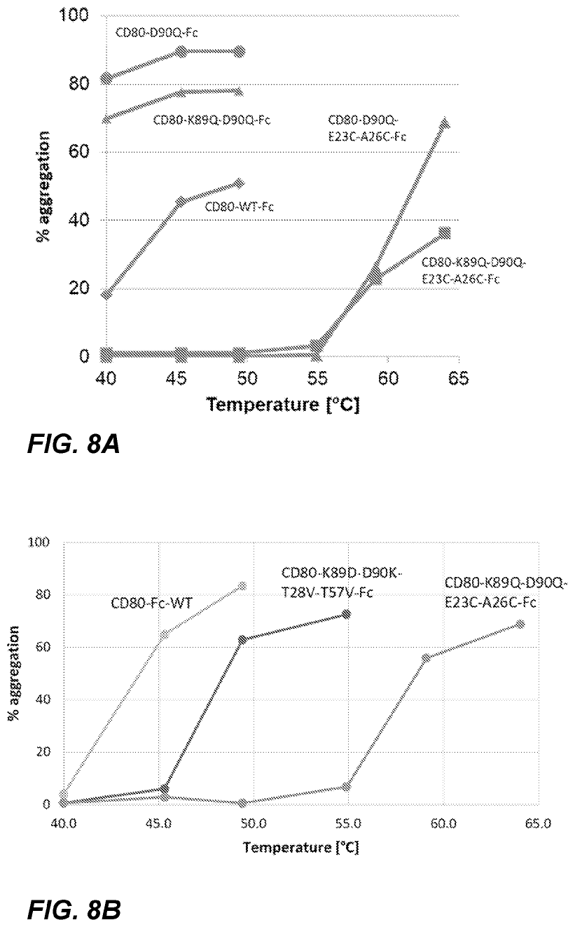

To assess the aggregation propensity of variant CD80-Fc fusion proteins, 1 mg/ml of CD80-Fc fusion protein was incubated in PBS at increasing temperatures for 24 hours. The samples were analyzed by SEC on an Agilent 1100 (Agilent Technologies, Germany) HPLC system using a YMC-Pack Diol-200 (YMC, Germany) analytical size exclusion chromatography column and PBS supplemented to 400 mM NaCl as running buffer. % aggregation was calculated from the loss of intact peak area. As shown in Table 11 and FIG. 8A, and Table 12 and FIG. 8B, variant CD80-Fc fusion proteins demonstrated a significantly reduced aggregation propensity compared to WT CD80-Fc fusion proteins. ND=Not Determined.

TABLE 11
Thermal forced aggregation of variant CD80-Fc fusion proteins

| CD80-Fc | Temperature | | | | | |
|---|---|---|---|---|---|---|
| Fusion Protein | 40° C. | 45.3° C. | 49.4° C. | 54.9° C. | 59.1° C. | 64° C. |
| CD80-WT-Fc | 18.0% | 45.4% | 50.8% | ND | ND | ND |
| CD80-K89Q- | 1.0% | 1.0% | 1.0% | 3.2% | 22.8% | 36.2% |
| D90Q-E23C- | | | | | | |
| A26C-Fc | | | | | | |
| CD80-D90Q- | 0.0% | 0.1% | 0.1% | 0.5% | 26.2% | 68.8% |
| E23C-A26C-Fc | | | | | | |
| CD80-D90Q-Fc | 81.6% | 89.5% | 89.5% | ND | ND | ND |
| CD80-K89Q- | 69.9% | 77.7% | 78.0% | ND | ND | ND |
| D90Q-Fc | | | | | | |

TABLE 12
Thermal forced aggregation of variant CD80-Fc fusion proteins

| | Temperature | | | | | |
|---|---|---|---|---|---|---|
| CD80-Fc Fusion Protein | 40° C. | 45.3° C. | 49.4° C. | 54.9° C. | 59.1° C. | 64° C. |
| CD80-K89D-D90K-T28V-T57V-Fc | 0.6% | 6.0% | 62.8% | 72.5% | ND | ND |
| CD80-K89Q-D90Q-E23C-A26C-Fc | 0.6% | 2.9% | 0.5% | 6.7% | 55.9% | 68.8% |
| CD80-WT-Fc | 0.6% | 64.9% | 83.4% | ND | ND | ND |

### Example 7

### Binding Affinity

Binding affinity of CD80-Fc fusion proteins against recombinant soluble CD28 and CTLA-4 proteins was determined by surface plasmon resonance (SPR) using a Biacore 8K+ instrument at 37° C. (physiologic temperature) with a collection rate of 10 Hz. Purified soluble ligands were covalently coupled onto a CM5 sensor chip using an Amine coupling Kit (GE Healthcare, Product #BR100050) following the manufacturer's recommendations. Three-fold serial dilutions of the CD80-Fc fusion protein analytes in HBS-EP+ running buffer (10 mM HEPES pH 7.4, 0.15 M NaCl, 3 mM EDTA, 0.05% P-20), ranging in concentration from 900 nM to 11.1 nM, were injected for 55 sec over the directly immobilized ligands at a flow rate of 50 μLs/min. The flow was again returned to running buffer and dissociation was monitored for 300 sec. Binding affinities and rate constants were determined by fitting the resulting double referenced (Myszka, D. G. J. Mol. Recognit. 12, 279-284 (1999)) sensorgram data to a 1:1 Langmuir model with Biacore Insight Evaluation software version 2.0 (GE Healthcare). Tables 13 and 14 shows the bivalent apparent K_{D}±SE, where either CD28 or CTLA-4 was immobilized to the sensor chip, and binding of variant CD80-Fc fusion proteins or WT CD80-Fc was performed at 37° C. As shown in Table 13, affinity of variant CD80-Fc fusion proteins to CD28 was enhanced or increased compared to WT CD80-Fc fusion proteins, while binding to CTLA-4 remained unchanged. Variant CD80-Fc fusion proteins has similar affinity to CD28 and CTLA-4, in contrast to WT CD80-Fc which demonstrated higher CTLA-4 affinity.

TABLE 13
Binding of CD80-Fc fusion protein to mouse, rat and human CD28 and CTLA-4

| | CD28 *K_{D} (nM) | | CTLA-4 *K_{D} (nM) | |
|---|---|---|---|---|
| | | CD80-K89Q-D90Q- | | CD80-K89Q-D90Q- |
| | CD80-WT-Fc | E23C-A26C-Fc | CD80-WT-Fc | E23C-A26C-Fc |
| Human | 2308.48 ± 46.05 | 1100.76 ± 6.45 | 96.14 ± 0.97 | 291.2 ± 5.5 |
| Rat | 823.34 ± 16.17 | 855.99 ± 21.74 | 50.43 ± 4.56 | 204.51 ± 14.19 |
| Mouse | 1151.69 ± 278.62 | 955.49 ± 185.81 | 131.51 ± 14.39 | 406.74 ± 29.38 |

As shown in Table 14, affinity of variant CD80-Fc fusion protein to human CD28 was increased 18-fold over WT CD80-Fc fusion protein, while binding to human CTLA-4 was unchanged. Variant CD80-Fc fusion protein had similar affinity to human CD28 and human CTLA-4, in contrast to WT CD80-Fc which demonstrated higher human CTLA-4 affinity. Binding of variant CD80-Fc to PD-L1 was not detected. FIG. 9A-9D show the SPR sensorgrams depicting binding of varying concentrations of CD80-WT-Fc and CD80-K89D-D90K-T28V-T57V-Fc to two different concentrations of immobilized human PD-L1 (60 μg/ml and 75 μg/ml). CD80-WT-Fc has detectable binding to human PD-L1, however binding of CD80-K89D-D90K-T28V-T57V-Fc to PD-L1 is undetectable.

TABLE 14
Binding of CD80-Fc to mouse, rat and human CD28, CTLA-4 and PD-L1

| | CD28 *K_{D} (nM) | | CTLA-4 *K_{D} (nM) | | PD-L1 *K_{D} (nM) | |
|---|---|---|---|---|---|---|
| | | CD80-K89D- | | CD80-K89D- | | CD80-K89D- |
| | CD80- | D90K-T28V- | CD80- | D90K-T28V- | CD80-WT- | D90K-T28V- |
| | WT-Fc | T57V-Fc | WT-Fc | T57V-Fc | Fc | T57V-Fc |
| Human | 1685.2 ± 6.3 | 92.4 ± 2.3 | 108.5 ± 1.36 | 115.45 ± 2.45 | >1 μM | No binding |
| | | | | | | detected |
| Rat | 827.34 ± 25.81 | 375.1 ± 0.91 | 62.57 ± 0.96 | 126.06 ± 2.88 | Not | Not |
| | | | | | determined | determined |
| Mouse | 1370 ± 80 | 478 ± 5 | 125.5 ± 5.5 | 211.5 ± 1.5 | Not | Not |
| | | | | | determined | determined |

### Example 8

### Viscosity

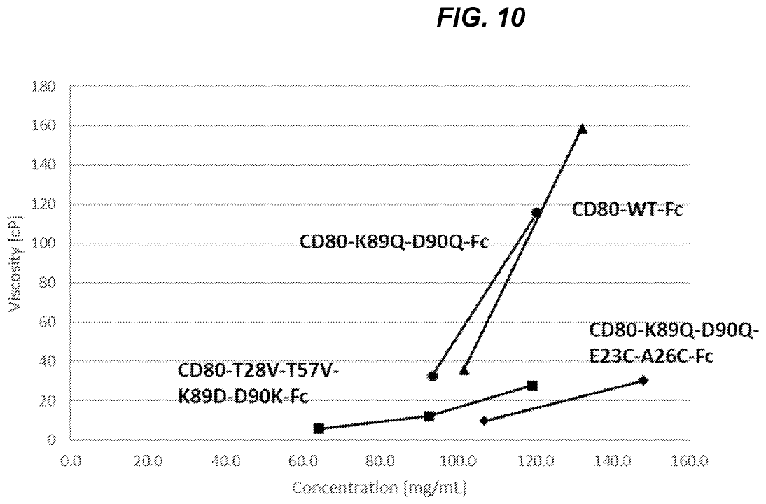

The viscosity of CD80-Fc fusion proteins was assessed at various concentrations. Lower viscosity is desired for subcutaneous administration, and providing optimal syringeability and minimal pain to patients. Viscosity was measured using an Anton Paar rheometer and a CP-25 measuring system at 150 rpm. The sample comprised CD80-Fc fusion proteins in 20 mM histidine and 8.5% sucrose at pH 5.8. As shown in Table 15 and FIG. 10, variant CD80-Fc fusion proteins demonstrated lower viscosity compared to WT CD80-Fc fusion proteins.

TABLE 15
Viscosity of variant CD80-Fc fusion proteins

| | Concentration | Viscosity |
|---|---|---|
| CD80-Fc fusion proteins | [mg/ml] | [cP] |
| CD80-WT-Fc | 132.25 | 158.8 |
| | 101.65 | 35.7 |
| CD80-K89Q-D90Q-E23C-A26C-Fc | 148.0 | 30.16 |
| | 107.0 | 9.878 |
| CD80-K89Q-D90Q-Fc | 120.6 | 115.8 |
| | 93.5 | 32.8 |
| CD80-K89D-D90K-T28V-T57V-Fc | 119 | 27.8 |
| | 92.7 | 12.1 |

### Example 9

### Yield and Purity

Upon harvest, the media was clarified by centrifugation and filtered using a 0.22 μm filter. Each variant was loaded onto a 5 ml Protein A (MabSelect SuRe) column equilibrated with PBS, PH 7.2. Columns were washed with 10 CVs PBS, PH 7.2 prior to the product being eluted with 150 mM Glycine, 40 mM NaCl, pH 3.5. Eluted product was immediately neutralized with 10% (v/v) 2 M HEPES, pH 8.0. Protein concentration of each variant were determined via Nanodrop readings at absorbance of 280 nm (A280). Each A280 value was divided by each of the variant's extinction coefficient to obtain a mg/ml concentration. Total protein for each variant was calculated by multiplying concentration by elution volume. Yield (mg/L) for each variant was then calculated by dividing the total protein by the volume of conditioned media generated. Size exclusion chromatography was used to determine purity following Protein A capture. Each variant was injected onto an Agilent 1200 equipped with a YMC Diol-300 column (300×8 mm I.D. S-5 μm, 30 nm) equilibrated with 20 mM Sodium Phosphate, 400 mM NaCl, pH 7.2. Manual integration of the main peak was performed to determine percent protein of interest. Table 16 shows the improved yield and purity of various variant CD80-Fc fusion proteins having non-cysteine stabilizing mutations. Four variants (*) were selected to move forward to test stability. ND=Not determined, Aggregate=High Molecular Mass Species (HMMS), Distortion=Main peak demonstrated speciation with low resolution; unable to obtain accurate integration of protein of interest.

TABLE 16
Non-cysteine stabilizing mutations.

| | ProA Yield | |
|---|---|---|
| CD80-Fc fusion protein | (mg/L) | ProA % Purity |
| CD80-WT-Fc | 164 | 94.4 |
| CD80-V11L-V22F-Fc | 177 | 94* |
| CD80-V11L-T62Y-Fc | 186 | 93* |
| CD80-V11L-T62Y-N63D-Fc | 184 | Aggregate |
| CD80-V22F-T62L-Fc | 145 | 86.7 |
| CD80-T28V-T57V-Fc | 194 | 94.7* |
| CD80-T28V-T57V- | 185 | 76 + Distortion |
| Y31Q-Q33E-K54E-Fc | | |
| CD80-D60Y-Fc | 157 | Aggregate |
| CD80-D60Y-K54E- | 68 | Aggregate |
| N63E-N64D-Fc | | |
| CD80-D60Y-T62L-Fc | 171 | Aggregate |
| CD80-D60Y-T62L- | 226 | Aggregate |
| N63D-N64E-Fc | | |
| CD80-V22F-D60Y-Fc | 73 | Aggregate |
| CD80-V22F-D60Y-K54E-N64E-Fc | 173 | Aggregate |
| CD80-D60F-T621-Fc | ND | ND |
| CD80-D60R-T62Y-Fc | 142 | 70.5 |
| CD80-D60Y-V11L-Fc | 195 | Aggregate |
| CD80-D60Y-V11L-N63D-Fc | 159 | Aggregate |
| CD80-D60Y-V22M-Fc | 152 | 50 |
| CD80-D60T-T62Y-Fc | 210 | Aggregate |
| CD80-D60Q-T62F-Fc | 235 | Aggregate |
| CD80-V22F-T28V-T57V-Fc | 210 | 92.8* |
| CD80-V22F-T28V-T57V- | 125 | 78.7 |
| Y31Q-Q33E-K54E-Fc | | |
| CD80-V22F-T62L-N64E-Fc | 157 | 83 |
| CD80-V22F-T62L-N63D-N64E-Fc | 161 | 73.5 |
| CD80-D60Y-T62L-N63D-Fc | 137 | Aggregate |

### Example 10

### Functional Assays Measuring IL-2 Production

When CD28 is engaged by CD80 following TCR stimulation, a signaling cascade results in the production of IL-2. Thus, IL-2 is a direct readout of CD28-mediated co-stimulation. Two IL-2-based in vitro assays were used to measure potency and functionality of the CD80-Fc fusion proteins: A. Jurkat IL-2 reporter and B. human peripheral blood mononuclear cells (PBMCs).

### A. Jurkat IL-2-Reporter Assay

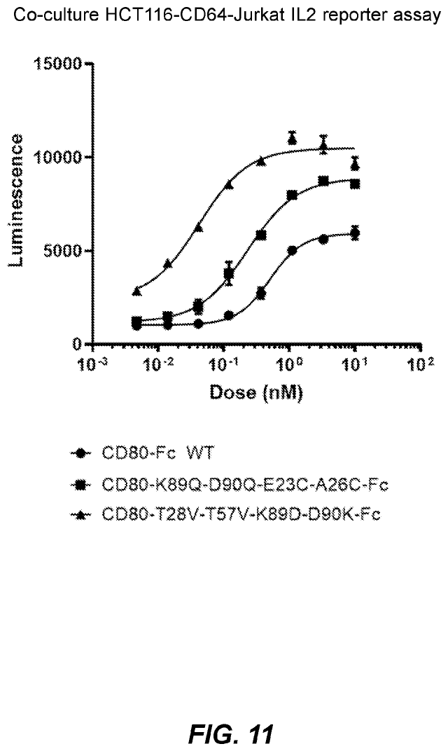

Jurkat is an immortalized human T cell line, and the Jurkat IL-2-reporter assay assesses the CD28-mediated signaling resulting in activation of the IL-2 promoter. The activity of CD80-Fc fusion proteins was measured using the co-culture HCT116-CD64-Jurkat IL-2 reporter assay, as described in Example 2, but using 20 ng/ml CDH3×CD3 bispecific. Table 17 (n=2) EC₅₀±SEM and fold-change over background (CD3 stimulation alone) and FIG. 11 IL-2 reporter activity shows that variant CD80-Fc fusion proteins demonstrated enhanced IL-2 production and potency compared to WT CD80-Fc.

TABLE 17
EC₅₀ of variant CD80-Fc fusion proteins
in Jurkat-IL-2 reporter assay

| | | Fold- |
|---|---|---|
| | | increase over |
| CD80-Fc fusion protein | EC₅₀ (nM) | background |
| CD80-WT-Fc | 0.58 ± 0.074 | 3.60 ± 0.45 |
| CD80-K89Q-D90Q- | 0.27 ± 0.044 | 6.00 ± 1.40 |
| E23C-A26C-Fc | | |
| CD80-K89D-D90K- | 0.047 ± 0.004 | 7.75 ± 1.92 |
| T28V-T57V-Fc | | |

### B. Human Peripheral Blood Mononuclear Cell (PBMC) Assay

The ability of the variant CD80-Fc fusion proteins to promote IL-2 production in the context of anti-CD3 (to stimulate T cell receptor signaling) in human peripheral blood mononuclear cells (PBMCs) was tested. To bind anti-CD3 antibody on the plate, the anti-human-CD3 (clone HIT3a, BD Pharmingen) was incubated in 96-well plates at 4° C. overnight at 1 μg/ml unless otherwise noted. Human PBMCs were isolated from freshly collected human whole blood using Ficoll-Paque Plus. Human PBMCs (100,000 per well) were incubated with plate-bound anti-CD3 and the indicated concentrations of soluble CD80-Fc fusion protein in RPMI+10% HI-FBS, pen/strep, sodium pyruvate and non-essential amino acids. After 48 hours incubation, supernatants were collected and IL-2 measured by the Quantikine human IL-2 ELISA kit from R&D.

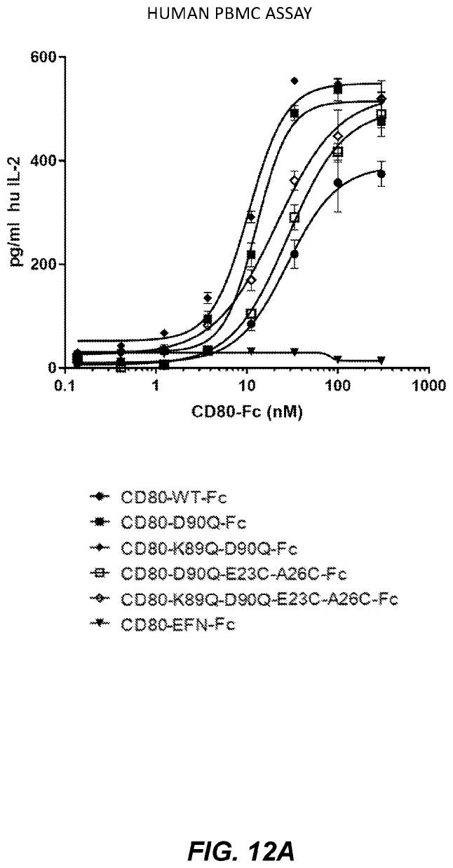

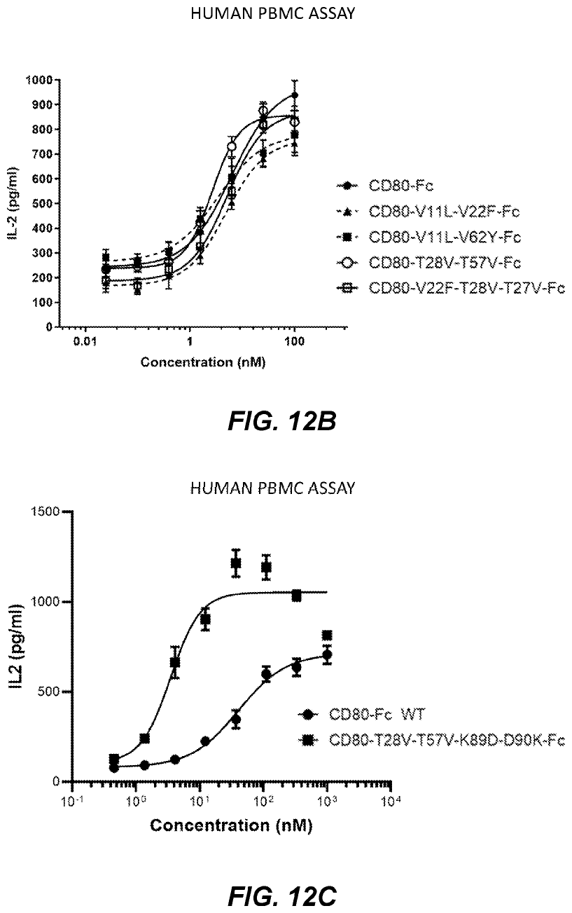

Table 18 shows the EC₅₀ of WT and variant CD80-Fc fusion proteins. Values shown are the average EC₅₀ from 5 different PBMC donors. FIG. 12A depicts the dose-response of IL-2 production from 1 individual PBMC donor. The CD80-Fc variants, CD80-D90Q-Fc, CD80-K89Q-D90Q-Fc and CD80-K89Q-D90Q-E23C-A26C-Fc, were more potent at promoting IL-2 production compared to WT CD80-Fc in human PBMCs in the presence of anti-CD3.

TABLE 18
EC₅₀ of variant CD80-Fc fusion proteins in human PBMC assay

| | IL-2 EC₅₀ |
|---|---|
| CD80-Fc fusion proteins | (nM) |
| CD80-WT-Fc | 23.56 ± 4.60 |
| CD80-D90Q-Fc | 7.45 ± 1.75 |
| CD80-K89Q-D90Q-Fc | 6.12 ± 1.45 |
| CD80-D90Q-E23C-A26C-Fc | 24.19 ± 10.01 |
| CD80-K89Q-D90Q-E23C-A26C-Fc | 11.53 ± 2.98 |

Additional CD80-Fc fusion proteins were tested in the same PBMC assay, at the concentration of anti-human-CD3 (clone HIT3a, BD Pharmingen) noted. Table 19 shows the EC₅₀ of WT and variant CD80-Fc fusion proteins from individual PBMC donors and FIG. 12B (Donor 418) and FIG. 12C (Donor 379) show the dose-response of IL-2 production. The variant CD80-Fc fusion proteins were more potent for IL-2 production compared to WT CD80-Fc fusion proteins.

TABLE 19
EC₅₀ of WT and variant CD80-Fc fusion
proteins in human PBMC assay.

| | | Dose of | | IL-2 EC₅₀ |
|---|---|---|---|---|
| CD80-Fc fusion protein | Donor | anti-CD3 | | (nM) |
| CD80-WT-Fc | 418 | 0.3 | ug/ml | 6.24 |
| CD80-V11L-V22F-Fc | 418 | 0.3 | ug/ml | 4.95 |
| CD80-V11L-T62Y-Fc | 418 | 0.3 | ug/ml | 3.34 |
| CD80-T28V-T57V-Fc | 418 | 0.3 | ug/ml | 2.64 |
| CD80-V22F-T28V-T57V-Fc | 418 | 0.3 | ug/ml | 5.13 |
| CD80-WT-Fc | 379 | 1 | ug/ml | 40.95 |
| CD80-K89D-D90K-T28V-T57V-Fc | 379 | 1 | ug/ml | 3.52 |

CD80-Fc effector function null (EFN) contained mutations that rendered the Fc portion unable to bind to Fcγ receptors, which are expressed by monocytes/macrophages present in human PBMCs. CD80-Fc EFN did not promote IL-2 production in the context of anti-CD3 in either the Jurkat or PMBC assay, demonstrating the importance of Fcγ receptor binding for optimal IL-2 production.

### Example 11

### Cytokine Release Assessment

The ability of variant CD80-Fc fusion proteins to promote cytokine release in the absence of TCR stimulation was assessed in the RESTORE (RESetting T cells to Original Reactivity) assay (Römer et al, Blood, 2011). Freshly isolated peripheral blood mononuclear cells (PBMC) from whole blood of several healthy donors were cultured for 48 hours at 107/mL within RPMI1640 media supplemented with 10% autologous plasma, 10 mM HEPES, 1× GlutaMAX, 1× PenStrep, beta2-mercaptoethanol, NEAA and sodium pyruvate. Following the 48-hour incubation, PBMC were pelleted and resuspended at a final concentration of 10\hat{ }6/ml with indicated soluble drug. Treatment was allowed for 48 hours then supernatant was harvested and assessed in IL-2 and IFNγ ELISA according to manufacturer's protocol (BioLegend).

Positive controls included CD28 superagonist anti-CD28 clone TGN1412 as IgG1 and IgG4 isotypes and anti-CD3. Table 20 shows there was no significant induction of cytokines by any of the variant CD80-Fc fusion proteins up to 10 μg/ml among 4 different donors in the high density PBMC assay, whereas positive controls anti-CD3 and superagonist TGN1412 promoted cytokine production. Values shown are the average of 3 technical replicates. D=Donor.

TABLE 20
IL-2 and IFNγ production in absence of TCR stimulation.

| | | IL-2 | | | | IFNγ | | | |
|---|---|---|---|---|---|---|---|---|---|
| Treatment | Dose μg/ml | D222 | D281 | D462 | D547 | D222 | D281 | D462 | D547 |
| Media | — | 11.3 | 3.3 | 2.7 | 9.3 | 24.3 | 5.3 | 8.3 | 8.7 |
| anti-CD3 | 5 | 128.0 | 148.3 | 9.0 | 241.0 | 1059.0 | 1015.0 | 492.0 | 814.7 |
| CD80-WT-Fc | 10 | 9.3 | 2.0 | 2.0 | 5.3 | 3.3 | 3.3 | 7.3 | 3.3 |
| | 1 | 7.7 | 3.0 | 1.7 | 9.0 | 4.3 | 3.0 | 6.0 | 4.0 |
| CD80-D90Q-Fc | 10 | 9.0 | 2.3 | 1.7 | 3.3 | 4.0 | 3.3 | 10.3 | 7.7 |
| | 1 | 3.0 | 2.3 | 1.7 | 9.0 | 3.3 | 3.3 | 8.3 | 3.3 |
| CD80-K89Q- | 10 | 4.0 | 2.0 | 1.7 | 3.3 | 4.0 | 6.7 | 15.0 | 8.0 |
| D90Q-Fc | 1 | 4.3 | 2.3 | 2.0 | 6.3 | 3.7 | 3.3 | 7.0 | 4.0 |
| CD80-K89Q- | 10 | 6.7 | 5.7 | 3.0 | 6.0 | 4.3 | 3.7 | 7.3 | 3.0 |
| D90Q-E23C- | 1 | 6.0 | 2.7 | 2.0 | 13.3 | 7.3 | 3.0 | 7.0 | 4.3 |
| A26C-Fc | | | | | | | | | |
| CD80-D90Q- | 10 | 11.0 | 2.0 | 2.0 | 12.0 | 4.7 | 6.3 | 6.0 | 5.0 |
| E23C-A26C-Fc | 1 | 7.0 | 2.0 | 1.7 | 4.3 | 4.0 | 5.0 | 7.3 | 3.3 |
| TGN1412 IgG4 | 10 | 390.0 | 240.0 | 206.7 | 243.0 | 241.0 | 206.3 | 186.7 | 61.3 |
| | 1 | 235.0 | 88.3 | 137.7 | 73.3 | 162.7 | 78.7 | 145.7 | 31.0 |

Additional variant CD80-Fc fusion proteins were tested up to 50 μg/ml in 6 different PBMC donors. As shown in Tables 21 and 22, no significant induction of cytokines was observed. Values shown are the average of 3 technical replicates. D=Donor.

TABLE 21
IL-2 production in absence of TCR stimulation.

| | Dose | IL-2 | | | | | |
|---|---|---|---|---|---|---|---|
| Treatment | μg/ml | D379 | D417 | D399 | D518 | D132 | D149 |
| Media | — | 4.0 | 0.3 | 0.0 | 1.3 | 0.0 | 0.0 |
| anti-CD3 | 5 | 144.0 | 490.0 | 121.7 | 35.7 | 67.0 | 118.0 |
| CD80-WT-Fc | 50 | 0.3 | 0.3 | 3.0 | 2.7 | 1.0 | 0.0 |
| | 16.7 | 1.0 | 0.3 | 0.0 | 1.3 | 0.3 | 0.0 |
| | 5.6 | 1.0 | 10.7 | 0.0 | 4.7 | 0.0 | 4.3 |
| | 1.9 | 3.3 | 5.7 | 0.0 | 4.7 | 0.0 | 0.3 |
| CD80-K89Q-D90Q- | 50 | 0.0 | 0.0 | 0.0 | 2.7 | 1.0 | 0.0 |
| E23C-A26C-Fc | 16.7 | 0.0 | 0.3 | 0.0 | 1.7 | 0.3 | 0.0 |
| | 5.6 | 2.3 | 0.0 | 2.0 | 2.0 | 0.0 | 0.0 |
| | 1.9 | 29.0 | 3.0 | 0.0 | 0.0 | 0.0 | 0.0 |
| CD80-K89D-D90K- | 50 | 0.0 | 0.0 | 0.0 | 3.0 | 3.7 | 0.3 |
| T28V-T57V-Fc | 16.7 | 0.0 | 1.0 | 0.0 | 0.0 | 0.7 | 3.7 |
| | 5.6 | 0.0 | 0.0 | 0.0 | 0.3 | 0.3 | 0.0 |
| | 1.9 | 2.3 | 0.0 | 0.0 | 1.3 | 0.0 | 0.0 |
| TGN1412 IgG4 | 10 | 537.0 | 535.3 | 422.7 | 588.0 | 443.3 | 577.0 |
| | 1 | 518.0 | 428.3 | 373.3 | 356.7 | 207.7 | 577.0 |
| | 0.1 | 19.0 | 4.7 | 1.0 | 9.7 | 0.0 | 2.3 |
| TGN1412 IgG1 | 10 | 31.3 | 194.3 | 91.3 | 134.3 | 396.3 | 122.3 |
| | 1 | 44.7 | 256.3 | 157.3 | 133.3 | 447.0 | 232.3 |
| | 0.1 | 7.7 | 7.0 | 1.7 | 3.0 | 23.0 | 9.7 |

TABLE 22
IFNγ production in absence of TCR stimulation.

| | Dose | IFNγ | | | | | |
|---|---|---|---|---|---|---|---|
| Treatment | μg/ml | D379 | D417 | D399 | D518 | D132 | D149 |
| Media | — | 0.0 | 0.0 | 0.0 | 0.0 | 0.0 | 0.0 |
| anti-CD3 | 5 | 1317.0 | 1672.7 | 868.3 | 598.7 | 1600.7 | 1013.3 |
| CD80-WT-Fc | 50 | 0.0 | 0.0 | 18.3 | 0.0 | 12.3 | 5.7 |
| | 16.7 | 0.0 | 13.3 | 0.0 | 0.0 | 0.0 | 0.0 |
| | 5.6 | 0.0 | 146.7 | 0.0 | 0.0 | 0.0 | 0.0 |
| | 1.9 | 0.3 | 0.0 | 0.0 | 0.0 | 14.3 | 1.0 |
| CD80-K89Q- | 50 | 0.0 | 0.0 | 4.3 | 0.0 | 0.0 | 41.3 |
| D90Q- | 16.7 | 0.0 | 0.0 | 0.0 | 20.7 | 0.0 | 0.0 |
| E23C- | 5.6 | 0.0 | 0.0 | 0.0 | 0.0 | 78.0 | 0.0 |
| A26C-Fc | 1.9 | 0.0 | 0.0 | 0.0 | 0.0 | 0.0 | 0.0 |
| CD80- | 50 | 0.0 | 0.0 | 13.7 | 0.0 | 0.0 | 12.3 |
| K89D-D90K- | 16.7 | 1.0 | 0.0 | 0.0 | 0.0 | 41.3 | 0.0 |
| T28V- | 5.6 | 0.0 | 94.3 | 0.0 | 0.0 | 0.0 | 0.0 |
| T57V-Fc | 1.9 | 2.0 | 0.0 | 0.0 | 0.0 | 0.0 | 0.0 |
| TGN1412 | 10 | 1316.7 | 1668.7 | 475.3 | 1943.0 | 1439.7 | 690.0 |
| IgG4 | 1 | 1124.0 | 1536.3 | 359.7 | 1232.0 | 794.3 | 621.0 |
| | 0.1 | 5.0 | 0.0 | 2.7 | 0.0 | 0.0 | 32.3 |
| TGN1412 | 10 | 12.0 | 605.3 | 538.3 | 346.7 | 1694.3 | 203.3 |
| IgG1 | 1 | 25.7 | 945.7 | 2.7 | 351.3 | 1674.3 | 286.0 |
| | 0.1 | 3.3 | 0.0 | 0.0 | 0.0 | 604.0 | 0.0 |

### Example 12

### Efficacy of CD80-Fc Fusion Proteins in Renal Carcinoma Model

Renca murine renal carcinoma tumor cells (1 million) were subcutaneously implanted in the hind flank of female Balb/c mice. Five days after tumor implantation, PBS vehicle control or variant CD80-Fc fusion proteins were dosed intravenously at 0.3 and 1 mg/kg (on Day 0 and Day 3) or 3 mg/kg (Day 0, Day 3 and Day 6). Tumor volume was determined by caliper measurements obtained in 2 dimensions and calculated as width²×length/2. Error bars are depicted as ±SEM.

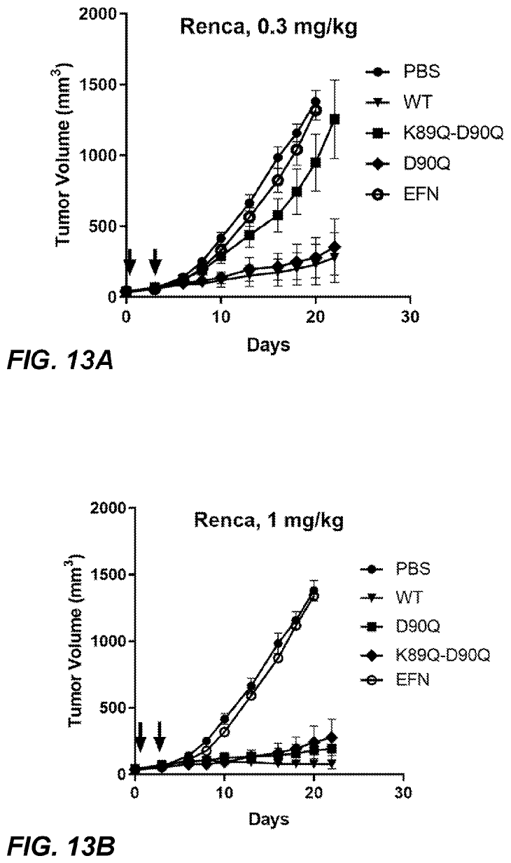

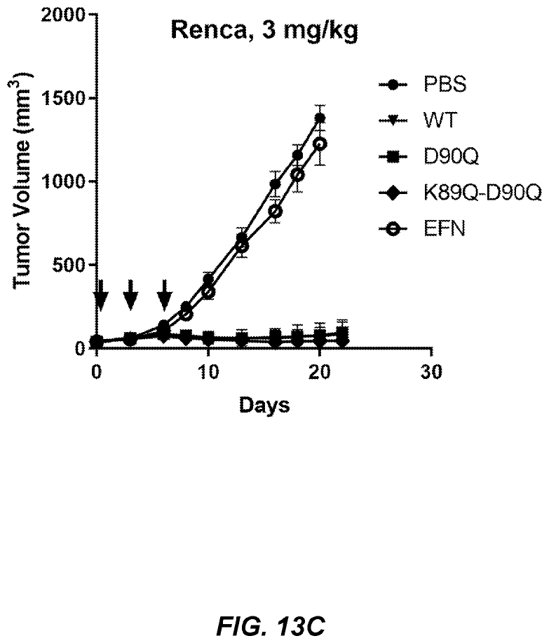

As shown in Table 23 and FIG. 13A-13C, treatment with the variant CD80-Fc fusion proteins significantly inhibited tumor growth, with 50% or more animals remaining tumor-free after treatment with 3 mg/kg of the variant CD80-Fc fusion proteins through the duration of the study. CD80-EFN-Fc with EFN mutations did not inhibit tumor growth.

TABLE 23
Efficacy of variant CD80-Fc fusion
proteins in renal carcinoma model

| | | | | | Number |
|---|---|---|---|---|---|
| | | | Day 18 | % | of |
| | | | tumor | tumor | tumor- |
| | | | volume | growth | free |
| Treatment | Dose | | (mm³) | inhibition | mice |
| PBS | — | | 1157.7 ± 63.4 | 0.00 | 0/10 |
| CD80-WT-Fc | 0.3 | mg/kg | 199.0 ± 114.0 | 85.6 | 5/10 |
| | 1 | mg/kg | 79.6 ± 25.5 | 96.2 | 5/10 |
| | 3 | mg/kg | 73.0 ± 69.0 | 93.7 | 6/7 |
| CD80-D90Q-Fc | 0.3 | mg/kg | 248.2 ± 126.1 | 81.4 | 4/10 |
| | 1 | mg/kg | 157.6 ± 64.2 | 89.6 | 4/10 |
| | 3 | mg/kg | 72.0 ± 41.6 | 97.1 | 8/10 |
| CD80-K89Q- | 0.3 | mg/kg | 743.2 ± 159.5 | 37.4 | 2/10 |
| D90Q-Fc | 1 | mg/kg | 192.6 ± 89.3 | 86.1 | 3/10 |
| | 3 | mg/kg | 43.2 ± 21.8 | 99.8 | 8/10 |
| CD80-EFN-Fc | 0.3 | mg/kg | 1040.4 ± 109.0 | 11.0 | 0/10 |
| | 1 | mg/kg | 1118.4 ± 40.2 | 3.6 | 0/10 |
| | 3 | mg/kg | 1040.4 ± 103.3 | 11.1 | 0/10 |

Pharmacodynamic (PD) modulation with variant CD80-Fc fusion proteins was assessed by measuring tumor-reactive T cells in the spleen using IFNγ ELIspot. Single-cell suspensions were generated from dissociated spleens taken at Day 9 after the first dose of CD80-Fc. Splenocytes were incubated with irradiated tumor cells overnight at 37° C., and the number of IFNγ-producing spots were measured using the IFNγ ELIspot Kit (BD Biosciences #551083). As shown in Table 24, treatment with variant CD80-Fc fusion proteins led to an increase in the amount of tumor-reactive T cells compared to PBS control.

TABLE 24
Splenic tumor-reactive T cells measured in renal carcinoma model

| | # IFNγ-producing spots |
|---|---|
| Treatment | (average of n = 4 mice per group) |
| Naïve (tumor-free mice) | 0.92 |
| PBS | 26.83 |
| CD80-WT-Fc, 0.3 mg/kg | 373.67 |
| CD80-WT-Fc, 1 mg/kg | 271.08 |
| CD80-WT-Fc, 3 mg/kg | 288.5 |
| CD80-D90Q-Fc, 3 mg/kg | 334.75 |
| CD80-K89Q-D90Q-Fc, 3 mg/kg | 152.17 |
| CD80-EFN-Fc, 3 mg/kg | 93.75 |

### Example 13

### Efficacy of CD80-Fc Fusion Proteins in Colotectal Carcinoma Model

CT26 murine colorectal carcinoma tumor cells (1×10⁶) were subcutaneously implanted in the hind flank of female Balb/c mice. When tumors reached ˜100 mm3 (Day 8), mice were randomized. PBS vehicle control or variant CD80-Fc fusion proteins were dosed intravenously at 0.1 and 1.0 mg/kg on Days 0 and 3. Tumor volume was determined by caliper measurements obtained in 2 dimensions and calculated as width²×length/2. Error bars are depicted as ±SEM.

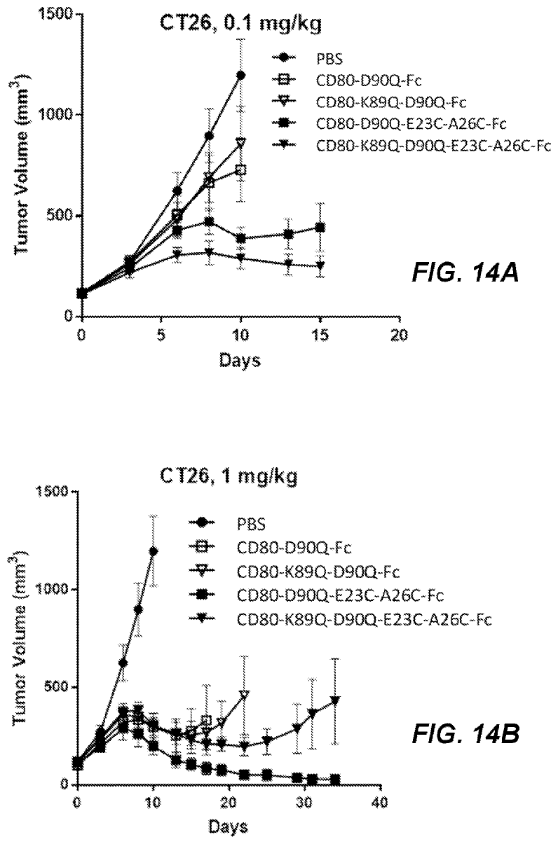

Shown in FIGS. 14A and 14B, treatment with disulfide-stabilized variant CD80-Fc fusion proteins resulted in improved tumor growth control compared to variant CD80-Fc fusion proteins without cysteine mutations at low (0.1 mg/kg) and high (1 mg/kg) doses. Table 25 shows tumor volumes and % tumor growth inhibition at Day 10, and tumor volumes continue to decrease after Day 10, with 50% or more animals experiencing tumor regressions at the 1 mg/kg doses with all variant CD80-Fc fusion proteins.

TABLE 25
Efficacy of variant CD80-Fc fusion proteins
in colorectal carcinoma model

| | | Day 10 | | % | Number |
|---|---|---|---|---|---|
| | | tumor | | tumor | of |
| | Dose | volume | | growth | tumor |
| Treatment | (mg/kg) | (mm³) | SEM | inhibition | regressions |
| PBS | | 1197.9 | 178.9 | 0.00 | 0/10 |
| CD80-D90Q-Fc | 0.1 | 728.3 | 157.3 | 39.20 | 0/10 |
| CD80-K89Q- | 0.1 | 857.6 | 185.3 | 28.41 | 1/10 |
| D90Q-Fc | | | | | |
| CD80-D90Q-E23C- | 0.1 | 388.9 | 54.7 | 67.53 | 2/10 |
| A26C-Fc | | | | | |
| CD80-K89Q-D90Q- | 0.1 | 289.9 | 52.2 | 75.80 | 5/10 |
| E23C-A26C-Fc | | | | | |
| CD80-D90Q-Fc | 1 | 295.8 | 70.4 | 75.31 | 6/10 |
| CD80-K89Q- | 1 | 299.0 | 44.7 | 75.04 | 5/10 |
| D90Q-Fc | | | | | |
| CD80-D90Q-E23C- | 1 | 197.8 | 44.0 | 83.49 | 9/10 |
| A26C-Fc | | | | | |
| CD80-K89Q-D90Q- | 1 | 305.3 | 29.4 | 74.51 | 6/10 |
| E23C-A26C-Fc | | | | | |

IFNγ ELIspot was used to measure tumor-reactive T cells in the spleen in mice treated with CD80-Fc fusion proteins at a 1 mg/kg dose. As shown in Table 26, treatment with 1 mg/kg of variant CD80-Fc fusion proteins led to an increase in the amount of tumor-reactive T cells in the spleen compared to PBS control in the CT26 model.

TABLE 26
Tumor-reactive T cells in spleens after
treatment with CD80-Fc fusion protein

| | # IFNγ-producing spots |
|---|---|
| Treatment | (average of n = 4 mice per group) |
| Naïve (tumor-free mice) | 0.0625 |
| PBS | 4.9375 |
| CD80-D90Q-Fc | 341.6875 |
| CD80-K89Q-D90Q-Fc | 569.5 |
| CD80-D90Q-E23C-A26C-Fc | 523.1875 |
| CD80-K89Q-D90Q-E23C-A26C-Fc | 547.375 |

### Example 14

### Efficacy of CD80-Fc Fusion Proteins in Breast Cancer Model

EMT6 murine breast cancer cells (3×10⁵) were orthotopically implanted in the mammary fat pad of female Balb/c mice. When tumors reached ˜90 mm³ (day 5 after implantation), mice were randomized. PBS vehicle control or variant CD80-Fc fusion proteins were dosed intravenously at 0.01, 0.1, 1.0 and 3.0 mg/kg on Days 0 and 3. Tumor volume was determined by caliper measurements obtained in 2 dimensions and calculated as width²×length/2. Error bars are depicted as ±SEM.

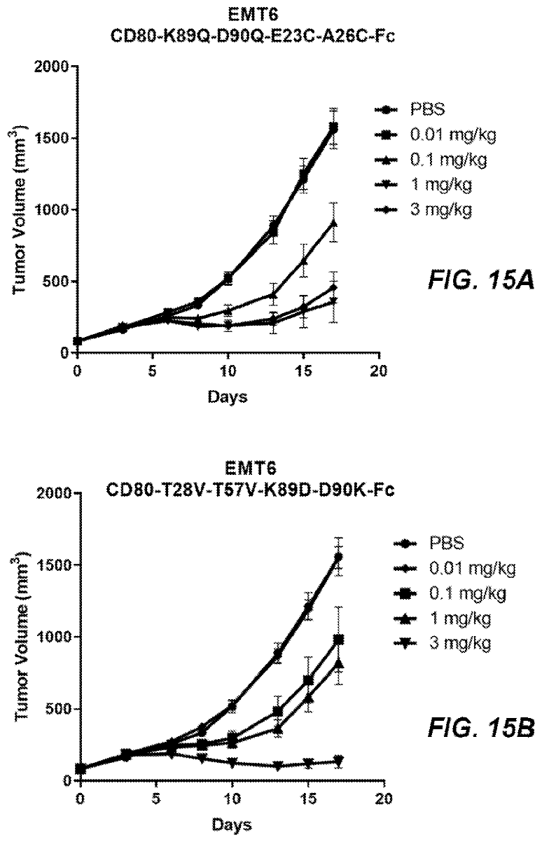

As shown in FIGS. 15A and 15B (Table 27), treatment with both variant CD80-Fc fusion proteins, respectively, significantly inhibited tumor growth starting at 0.1 mg/kg.

TABLE 27
Efficacy of CD80-Fc fusion proteins in breast cancer model

| | | | Day 17 | | % |
|---|---|---|---|---|---|
| | | | tumor | | tumor |
| | | | volume | | growth |
| Treatment | Dose | | (mm³) | SEM | inhibition |
| PBS | — | | 1557.8 | 131.4 | 0 |
| CD80-K89Q-D90Q- | 0.01 | mg/kg | 1581.7 | 125.8 | −1.7 |
| E23C-A26C-Fc | 0.1 | mg/kg | 912.2 | 135.5 | 43.7 |
| | 1 | mg/kg | 357.2 | 145.5 | 81.4 |
| | 3 | mg/kg | 457.5 | 108.2 | 74.6 |
| CD80-K89D-D90K- | 0.01 | mg/kg | 1552.0 | 76.7 | 0.4 |
| T28V-T57V-Fc | 0.1 | mg/kg | 981.2 | 226.1 | 39.0 |
| | 1 | mg/kg | 818.8 | 150.7 | 50.1 |
| | 3 | mg/kg | 133.4 | 44.5 | 96.6 |

Tumor-reactive T cells in the spleen and tumor-draining lymph nodes (TDLNs) were measured by IFNγ ELIspot in mice treated with CD80-K89D-D90K-T28V-T57V-Fc. As shown in Table 28, treatment with variant CD80-Fc fusion proteins led to a dose-dependent increase in the amount of tumor-reactive T cells in both the spleen and TDLNs compared to PBS control in the EMT6 model.

TABLE 28
Tumor-reactive T cells in spleen and TDLNs
with variant CD80-Fc fusion proteins

# IFNγ-producing spots
(average of n = 4 mice per group)

| Treatment | Spleen | TDLN |
|---|---|---|
| Naïve (tumor-free mice) | 0.3 | 0.0 |
| PBS | 16.1 | 14.2 |

| | | | |
|---|---|---|---|
| 0.01 | mg/kg | 123.1 | 51.9 |
| 0.1 | mg/kg | 117.9 | 132.3 |
| 1 | mg/kg | 336.4 | 475.2 |
| 3 | mg/kg | 427.7 | 540.9 |

### Example 15

### Efficacy of CD80-Fc Fusion Proteins by Subcutaneous Dosing

MC38 murine colorectal carcinoma cells (5×10⁵) were subcutaneously implanted in the hind flank of female Balb/c mice. When tumors reach ˜50 mm³ (day 6 after implantation), mice were randomized. PBS vehicle control or variant CD80-Fc fusion proteins were dosed intravenously (IV) or subcutaneously (SC) with doses ranging from 0.01-3.0 mg/kg on Days 0 and 3. Tumor volume was determined by caliper measurements obtained in 2 dimensions and calculated as width²×length/2. Error bars are depicted as ±SEM.

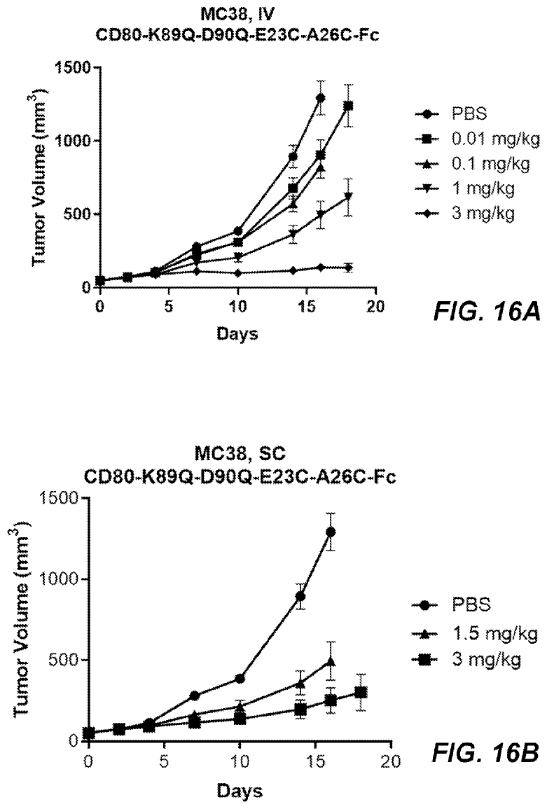

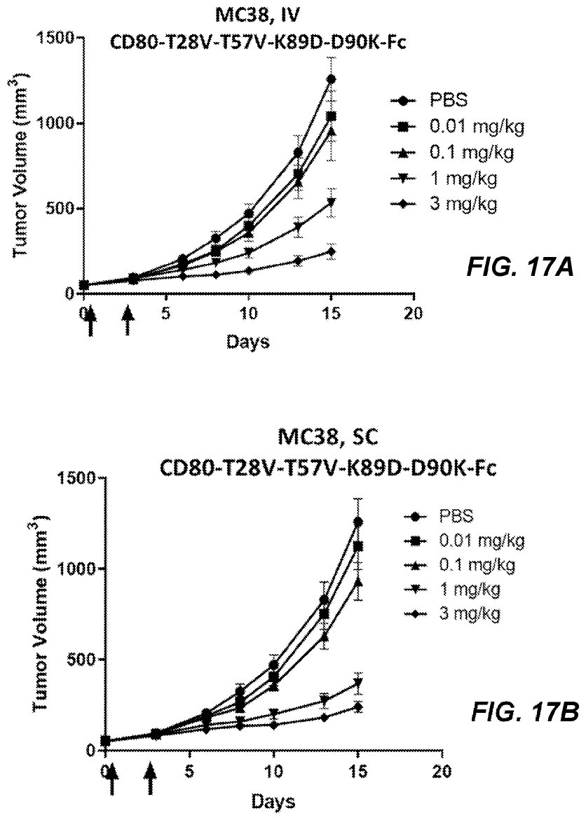

As shown in FIGS. 16A and 16B (Table 29) and FIGS. 17A and 17B (Table 30), treatment with variant CD80-Fc fusion proteins significantly inhibited tumor growth starting at 0.1 mg/kg. Additionally, subcutaneous dosing of variant CD80-Fc fusion proteins (FIGS. 16B and 17B) displayed similar efficacy as intravenous dosing (FIGS. 16A and 17A).

TABLE 29
Efficacy of variant CD80-Fc fusion proteins by IV and SC dosing

| | | | Day 16 | | % |
|---|---|---|---|---|---|
| | | | tumor | | tumor |
| Treatment and | | | volume | | growth |
| dosing route | Dose | | (mm³) | SEM | inhibition |
| PBS | — | | 1293.2 | 114.1 | 0 |
| CD80- K89Q-D90Q- | 0.01 | mg/kg | 905.1 | 102.1 | 31.2 |
| E23C-A26C -Fc | 0.1 | mg/kg | 824.7 | 77.3 | 37.7 |
| Intravenous | 1 | mg/kg | 495.3 | 93.0 | 64.2 |
| | 3 | mg/kg | 138.9 | 26.4 | 93.0 |
| CD80- K89Q-D90Q- | 1.5 | mg/kg | 495.6 | 117.6 | 64.2 |
| E23C-A26C -Fc | 3 | mg/kg | 252.2 | 79.2 | 83.7 |
| Subcutaneous | | | | | |

TABLE 30
Efficacy of variant CD80-Fc fusion proteins by IV and SC dosing

| | | | Day 15 | | % |
|---|---|---|---|---|---|
| | | | tumor | | tumor |
| Treatment and | | | volume | | growth |
| dosing route | Dose | | (mm³) | SEM | inhibition |
| PBS | — | | 1257.8 | 128.2 | 0 |
| CD80-K89D-D90K- | 0.01 | mg/kg | 1042.1 | 147.8 | 17.9 |
| T28V-T57V-Fc | 0.1 | mg/kg | 954.8 | 175.1 | 25.2 |
| Intravenous | 1 | mg/kg | 535.3 | 82.0 | 59.9 |
| | 3 | mg/kg | 248.1 | 44.3 | 83.8 |
| CD80-K89D-D90K- | 0.01 | mg/kg | 1125.4 | 129.1 | 11.1 |
| T28V-T57V-Fc | 0.1 | mg/kg | 931.7 | 103.7 | 27.1 |
| Subcutaneous | 1 | mg/kg | 369.7 | 59.5 | 73.7 |
| | 3 | mg/kg | 240.9 | 30.0 | 84.4 |

### Example 16

### Analysis of T Cell Infiltration in Tumors

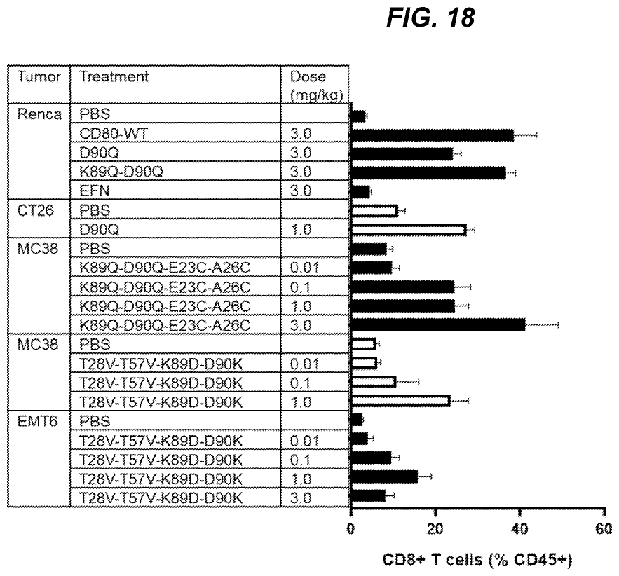

Cytotoxic CD8⁺ T cells are responsible for killing cancer cells and the measurement of CD8⁺ T cells that have infiltrated tumors commonly correlates with efficacy for immune-oncology therapies. For all in vivo studies, tumors were collected ˜1 week after the first dose and dissociated by mincing, followed by incubation in an enzymatic digestion cocktail and homogenization. The resulting cell suspension was filtered and washed, and single cells were stained with antibodies to CD45, CD8, and a viability dye to detect tumor-infiltrating lymphocytes (TILs) by flow cytometry. Data were acquired on a BD LSRFortessa flow cytometer or a Cytek Aurora spectral flow cytometer. Table 31 and FIG. 18 show the amount of tumor-infiltrating CD8+ T cells expressed as a percentage of total immune cells (CD45+). Each value is the average of n=4 mice per group. Treatment with all variant CD80-Fc fusion proteins led to increase in CD8+ TILs in various tumor models.

TABLE 31
Tumor infiltration of CD8+ T cells with variant CD80-Fc fusion protein treatment

| Tumor | | | | CD8⁺ T cell | |
|---|---|---|---|---|---|
| Model | Treatment | Dose | | infiltrates (% CD45) | SEM |
| Renca | PBS | | | 3.5 | 0.28 |
| | CD80-WT-Fc | 3 | mg/kg | 38.6 | 5.3 |
| | CD80-D90Q-Fc | 3 | mg/kg | 24.2 | 1.9 |
| | CD80-K89Q-D90Q-Fc | 3 | mg/kg | 36.7 | 2.3 |
| | CD80-EFN-Fc | 3 | mg/kg | 4.5 | 0.4 |
| CT26 | PBS | | | 11.2 | 1.6 |
| | CD80-D90Q-Fc | 1 | mg/kg | 27.4 | 1.9 |
| MC38 | PBS | | | 8.5 | 1.4 |
| | CD80-K89Q-D90Q-E23C-A26C- | 0.01 | mg/kg | 9.8 | 1.7 |
| | Fc | | | | |
| | CD80-K89Q-D90Q-E23C-A26C- | 0.1 | mg/kg | 24.5 | 3.9 |
| | Fc | | | | |
| | CD80-K89Q-D90Q-E23C-A26C- | 1 | mg/kg | 24.6 | 3.2 |
| | Fc | | | | |
| | CD80-K89Q-D90Q-E23C-A26C- | 3 | mg/kg | 41.3 | 7.8 |
| | Fc | | | | |
| MC38 | PBS | | | 5.98 | 0.68 |
| | CD80-K89D-D90K-T28V-T57V-Fc | 0.01 | mg/kg | 6.26 | 0.77 |
| | CD80-K89D-D90K-T28V-T57V-Fc | 0.1 | mg/kg | 10.81 | 5.24 |
| | CD80-K89D-D90K-T28V-T57V-Fc | 1 | mg/kg | 23.58 | 4.22 |
| EMT6 | PBS | | | 2.66 | 0.31 |
| | CD80-K89D-D90K-T28V-T57V-Fc | 0.01 | mg/kg | 3.98 | 1.32 |
| | CD80-K89D-D90K-T28V-T57V-Fc | 0.1 | mg/kg | 9.59 | 1.81 |
| | CD80-K89D-D90K-T28V-T57V-Fc | 1 | mg/kg | 15.91 | 3.13 |
| | CD80-K89D-D90K-T28V-T57V-Fc | 3 | mg/kg | 8.19 | 2.03 |

### Example 17

### Pharmacokinetics (PK)

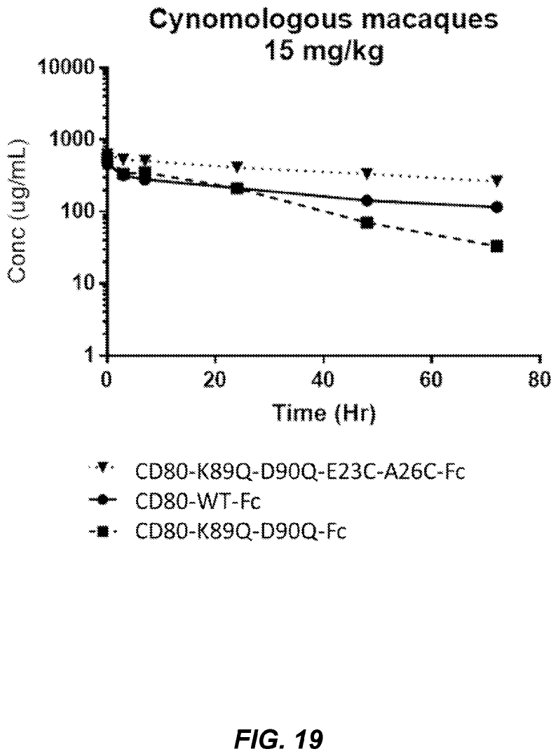

PK of variant CD80-Fc fusion proteins was assessed in non-human primates. Female cynomolgus monkeys were dosed intravenously with variant CD80-Fc fusion proteins at 3, 15 and 50 mg/kg, and blood was collected at the timepoints indicated on the graph. A ligand binding assay using the Gyrolab Immunoassay platform was used to quantitate CD80-Fc molecules in cynomolgus monkey serum following dose administration. CD80-Fc constructs were captured onto the Gyrolab Bioaffy CD using a biotinylated monoclonal anti-human CD80 antibody (Thermo Fisher Cat #13-0809-82). Bound CD80-Fc constructs were detected using a mouse anti-human Fc antibody (SouthernBiotech Cat #9040-01) that was labeled with Alexa Fluor 647. Sample concentrations were determined by interpolation from a calibration curve that were fit using a 5-parameter logistic regression model. The range of quantitation of the assay was 100-15000 ng/ml in 100% serum. As shown in Table 32 and FIG. 19, stabilized variant CD80-Fc fusion proteins demonstrated improved PK over the WT CD80-Fc, with a higher Cmax and higher AUC at all doses. AUC (Area Under the Curve); Cmax (Maximum Concentration observed); Tmax (Time of Maximum concentration observed)

TABLE 32
PK assessment of variant CD80-Fc fusion proteins.

| | | | | | AUC | AUC fold- |
|---|---|---|---|---|---|---|
| | | Dose | Cmax | Tmax | (μg*Hours/ | increase |
| Treatment | Subject | (mpk) | (μg/mL) | (Hours) | mL) | over WT |
| CD90-WT-Fc | 002F | 3 | 90.7 | 0.083 | 2480 | 1.00 |
| | 003F | 15 | 460 | 0.083 | 13900 | 1.00 |
| | 004F | 50 | 1150 | 0.083 | 37000 | 1.00 |
| CD80-K89Q- | 005F | 3 | 89.4 | 0.083 | 1230 | 0.50 |
| D90Q-Fc | 006F | 15 | 570 | 0.083 | 12100 | 0.87 |
| | 007F | 50 | 1420 | 0.083 | 27300 | 0.74 |
| CD80-K89Q- | 008F | 3 | 107 | 0.083 | 3680 | 1.48 |
| D90Q-E23C- | 009F | 15 | 627 | 0.083 | 27800 | 2.00 |
| A26C-Fc | 010F | 50 | 1790 | 0.083 | 72200 | 1.95 |

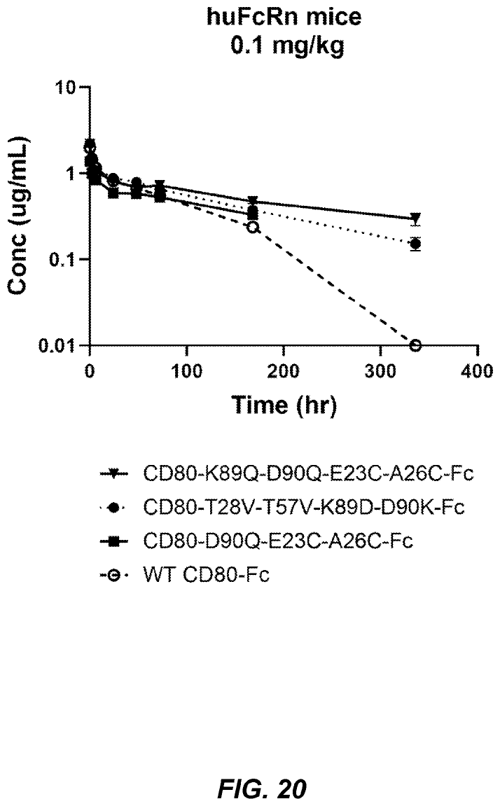

PK of CD80-Fc fusion proteins was also assessed in transgenic mice expressing the human neonatal Fc receptor (huFcRn) a-chain transgene under the control of its natural human promoter. Female mice were dosed intravenously with CD80-Fc fusion proteins at 0.1 mg/kg, and blood was collected at the timepoints indicated on the graph. Levels of CD80-Fc in mouse plasma were measured as described above. Table 33 and FIG. 20 shows the PK of CD80-Fc fusion proteins (average of 3 animals per group), with all variants tested showing a similar or improved PK compared to WT CD80-Fc fusion proteins. CL (Clearance); Vdss (Volume of Distribution); T½ (half-life).

TABLE 33
PK assessment of CD80-Fc fusion proteins
in huFcRn transgenic mice

| | | | CL | | |
|---|---|---|---|---|---|
| | Cmax | AUC | minutes | Vdss | |
| | (μg/ | (μg*Hours/ | (mL/ | (mL/ | T1/2 |
| Treatment | mL) | mL) | min/kg) | kg) | (Hours) |
| CD80-D90Q-E23C- | 1.4 | 88 | 0.011 | 131 | 148 |
| A26C-Fc | | | | | |
| CD80-K89Q-D90Q- | 2.2 | 182 | 0.006 | 109 | 211 |
| E23C-A26C-Fc | | | | | |
| CD80-K89D-D90K- | 2.3 | 147 | 0.01 | 82 | 106 |
| T28V-T57V-Fc | | | | | |
| CD80-WT-Fc | 2 | 97 | 0.014 | 87 | 80 |

### Example 18

Combination Efficacy of CD80-Fc Fusion Proteins with αPD1 Antibody

### A. CT26 Colorectal Carcinoma Model

CT26 murine colorectal carcinoma cells (1×10⁶) were subcutaneously implanted in the hind flank of female Balb/c mice. When tumors reached ˜100 mm3 (day 8 after implantation), mice were randomized and dosed with PBS vehicle control, 0.1 mg/kg CD80-D90Q-Fc, and/or 10 mg/kg murine αPD1 antibody. CD80-D90Q-Fc was dosed on Days 0 and 3, αPD1 antibody was dosed on Days 0, 3 and 6. Tumor volume was determined by caliper measurements obtained in 2 dimensions and calculated as width²×length/2. Error bars are depicted as ±SEM

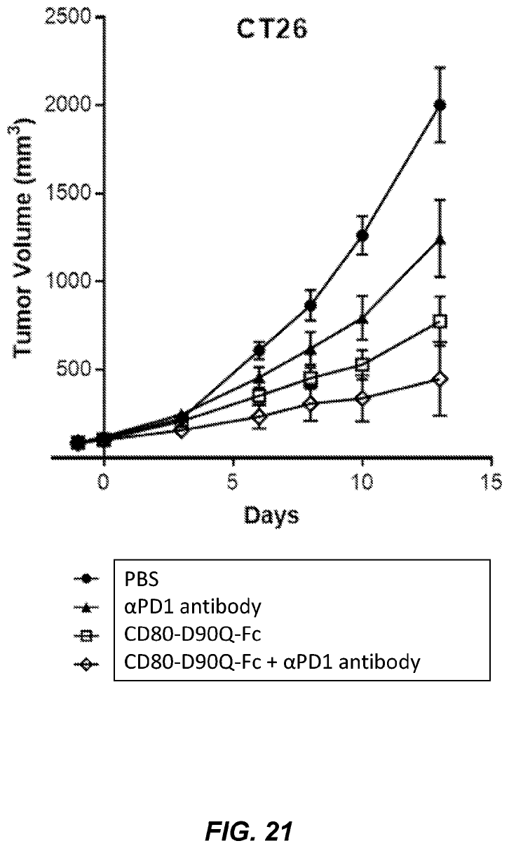

Shown in FIG. 21 (Table 34), treatment with CD80-D90Q-Fc or αPD1 Ab exhibited single-agent efficacy, with 61.2% tumor growth inhibition for 0.1 mg/kg of CD80-D90Q-Fc and 37.9% for αPD1 at Day 13. The combination of low dose CD80-D90Q-Fc and αPD1 antibody resulted in improved efficacy with 77.6% tumor growth inhibition at Day 13.

TABLE 34
Efficacy of CD80-Fc fusion proteins
in combination with αPD1 antibody

| | | Day 13 | | % |
|---|---|---|---|---|
| | CD80-Fc | tumor | | tumor |
| | dose | volume | | growth |
| Treatment | (mg/kg) | (mm³) | SEM | inhibition |
| PBS | — | 2002.5 | 211.0 | 0.00 |
| CD80-D90Q-Fc | 0.1 | 776.1 | 139.4 | 61.2 |
| CD80-D90Q-Fc | 1 | 270.4 | 44.4 | 86.5 |
| αPD1 Ab (10 mg/kg) | — | 1244.5 | 218.7 | 37.9 |
| CD80-D90Q-Fc + αPD1 Ab | 0.1 | 448.7 | 208.5 | 77.6 |
| CD80-D90Q-Fc + αPD1 Ab | 1 | 244.4 | 99.9 | 87.80 |

IFNγ ELIspot was used to measure tumor-reactive T cells in the spleen and TDLNs in mice treated with 1 mg/kg D90Q, αPD1 Ab, or the combination. As shown in Table 35, treatment with either agent alone led to increase in the amount of tumor-reactive T cells in both the spleen and tumor-draining lymph nodes compared to PBS control (n=4 per group). The combination led to an even higher amount of tumor-reactive T cells in the TDLN compared to either agent alone.

TABLE 35
Tumor-reactive T cells measured in spleens and TDLNs

| Treatment | Spleen | TDLN |
|---|---|---|
| Naive | 0 | 0.083 |
| PBS | 49.00 | 39.75 |
| CD80-D90Q-Fc (1 mg/kg) | 881.42 | 361.08 |
| αPD1 Ab | 70.00 | 220.58 |
| αPD1 Ab + CD80-D90Q-Fc (1 mg/kg) | 508.92 | 836.42 |

### B. B16F10 Melanoma Model

B16F10 murine melanoma cells (0.5 million) were subcutaneously implanted in the hind flank of female C57BL/6J mice. When tumors reached ˜100 mm3, mice were randomized and dosed with PBS vehicle control, 3 mg/kg CD80-D90Q-E23C-A26C-Fc, and or 10 mg/kg murine αPD1 antibody. CD80-D90Q-E23C-A26C-Fc was dosed on Days 0 and 3, αPD1 antibody was dosed on Days 0, 3 and 6. Tumor volume was determined by caliper measurements obtained in 2 dimensions and calculated as width²×length/2. Error bars are depicted as ±SEM

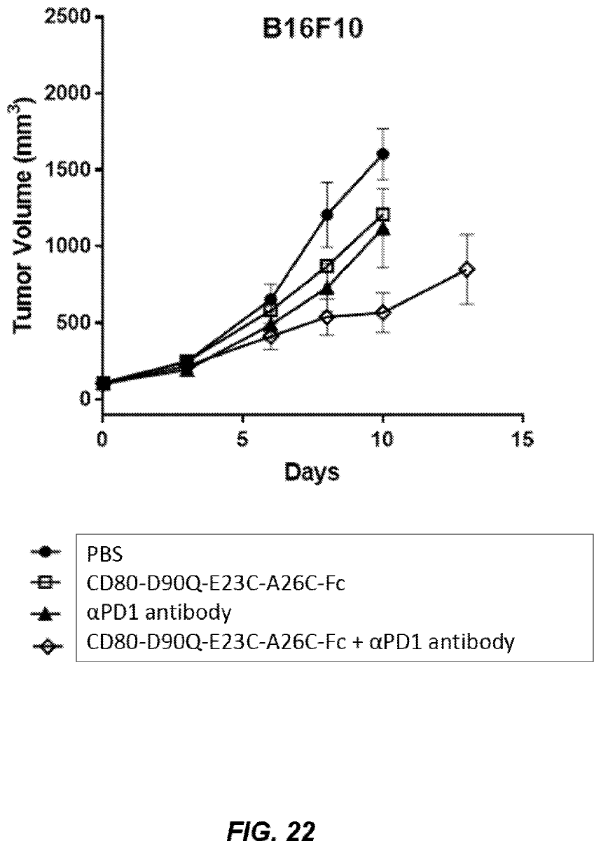

Shown in FIG. 22 (Table 36), single-agent treatment with CD80-D90Q-E23C-A26C-Fc exhibited 26.3% tumor growth inhibition and αPD1 Ab exhibited 32.3% tumor growth inhibition at Day 10. The combination of CD80-D90Q-E23C-A26C-Fc and αPD1 antibody resulted in improved efficacy, with 69.0% tumor growth inhibition at Day 10.

TABLE 36
Efficacy of CD80-Fc fusion proteins
in combination with αPD1 antibody

| | Day 10 | | % |
|---|---|---|---|
| | tumor | | tumor |
| | volume | | growth |
| Treatment | (mm³) | SEM | inhibition |
| PBS | 1604.0 | 166.5 | 0.00 |
| CD80-D90Q- | 1208.8 | 161.2 | 26.3 |
| E23C-A26C-Fc | | | |
| αPD1 Ab (10 mg/kg) | 1118.7 | 259.4 | 32.3 |
| CD80-D90Q-E23C- | 567.2 | 130.1 | 69.0 |
| A26C-Fc + αPD1 Ab | | | |

### Example 19

Combination Efficacy of CD80-Fc Fusion Protein with Talazoparib

A. EMT6 Breast Cancer Model

EMT6murine breast cancer cells (3×10⁵) were orthotopically implanted in the mammary fat pad of female Balb/c mice. When tumors reached ˜90 mm³ (day 5 after implantation), mice were randomized and dosed with PBS vehicle control, CD80-K89Q-D90Q-E23C-A26C-Fc (0.1 or 1 mg/kg), and/or 0.33 mg/kg talazoparib (Tala). CD80-D90Q-Fc was dosed on Days 0 and 3, talazoparib was dosed daily. Tumor volume was determined by caliper measurements obtained in 2 dimensions and calculated as width²×length/2. Error bars are depicted as ±SEM

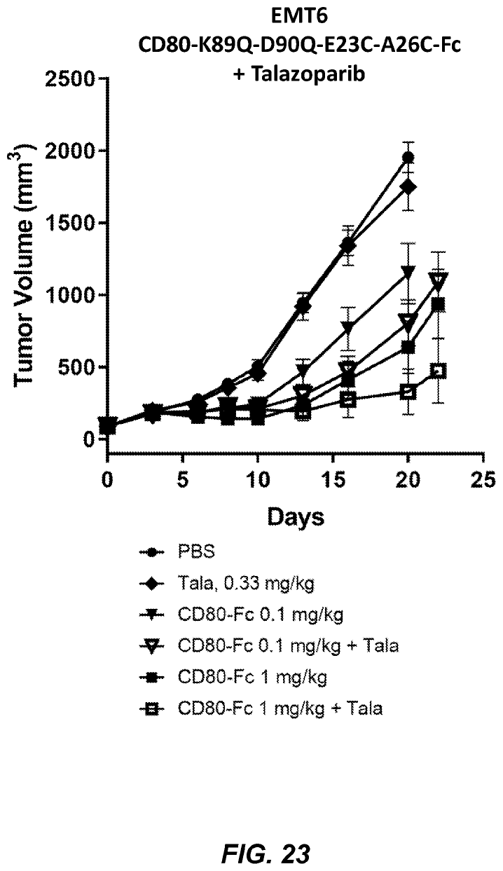

As shown in Table 37 and FIG. 23, treatment with CD80-K89Q-D90Q-E23C-A26C-Fc exhibited single-agent efficacy, with 43.1% and 70.5% tumor growth inhibition for 0.1 mg/kg and 1 mg/kg of CD80-K89Q-D90Q-E23C-A26C-Fc, respectively. No single agent efficacy of talazoparib was observed in this model, but the combination of CD80-Fc K89Q-D90Q-E23C-A26C and talazoparib resulted in improved efficacy over CD80-Fc K89Q-D90Q-E23C-A26C alone.

TABLE 37
Efficacy of CD80-Fc fusion proteins
in combination with talazoparib.

| | | Day 20 | | % |
|---|---|---|---|---|
| | CD80-Fc | tumor | | tumor |
| | dose | volume | | growth |
| Treatment | (mg/kg) | (mm³) | SEM | inhibition |
| PBS | — | 1953.9 | 107.3 | 0 |
| CD80-K89Q-D90Q- | 0.1 | 1149.2 | 210.1 | 43.1 |
| E23C-A26C-Fc | | | | |
| CD80-K89Q-D90Q- | 1 | 637.5 | 181.8 | 70.5 |
| E23C-A26C-Fc | | | | |
| Talazoparib (0.33 mg/kg) | — | 1750.1 | 164.6 | 10.9 |
| CD80-K89Q-D90Q- | 0.1 | 805.0 | 160.5 | 61.7 |
| E23C-A26C-Fc + | | | | |
| Talazoparib | | | | |
| CD80-K89Q-D90Q- | 1 | 329.1 | 157.5 | 87.1 |
| E23C-A26C-Fc + | | | | |
| Talazoparib | | | | |

### Example 20

### Co-Stimulation of Primary Human T Cells

Serial, 3-fold dilutions of CD80-K89D-D90K-T28V-T57V-Fc and CD80-WT-Fc were prepared in complete IMDM medium (10% FBS, 1% Pen/Strep). CEFT peptide (PM-CEFT, JPT Peptide Technologies) was reconstituted in the same medium to the final concentration of 1 μg/ml. Frozen human PBMCs were thawed following a standard protocol and 250,000 were seeded per well of flat bottom 96-wells plates. CEFT peptide and appropriate concentration of the CD80-Fc fusion proteins were added to the cell suspension. Plates were sealed with breathable plate sealers and incubated for 72 hours at 37° C. in a CO₂ incubator. Six hours prior to cell harvest, culture supernatants were collected and stored at −20° C. until used for cytokine analysis by MSD (V-Plex pro-inflammatory panel human kit, Meso Scale Discovery). At the same time brefeldin A solution was added to the cells, to enable intracellular accumulation of cytokines for evaluation by flow cytometry. Upon harvest, cells were stained for surface (among others CD25) and intracellular markers (among others Ki-67 and IFNγ) following a standard flow cytometry staining protocol. Data was collected using BD LSR Fortessa and analyzed using FlowJo v10 software.

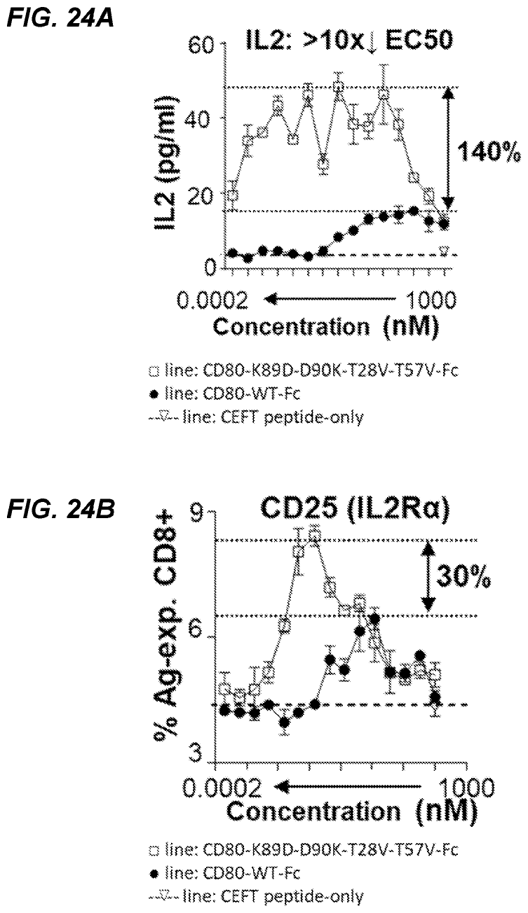

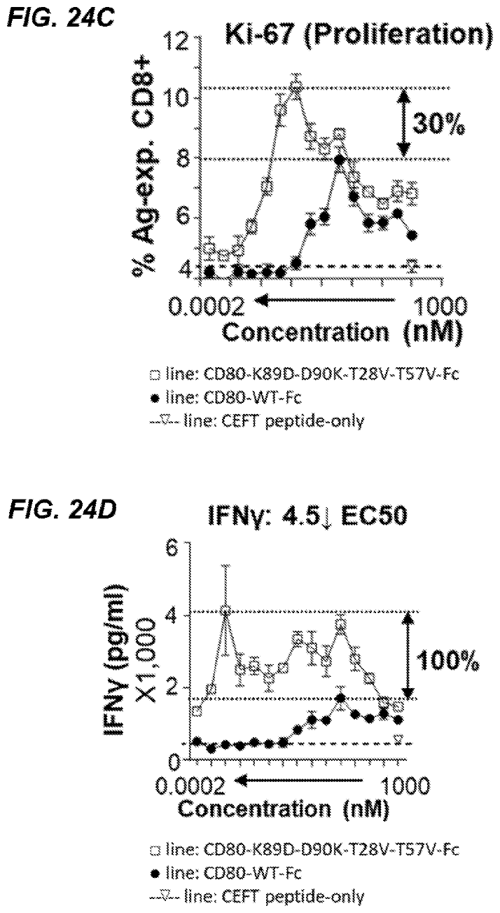

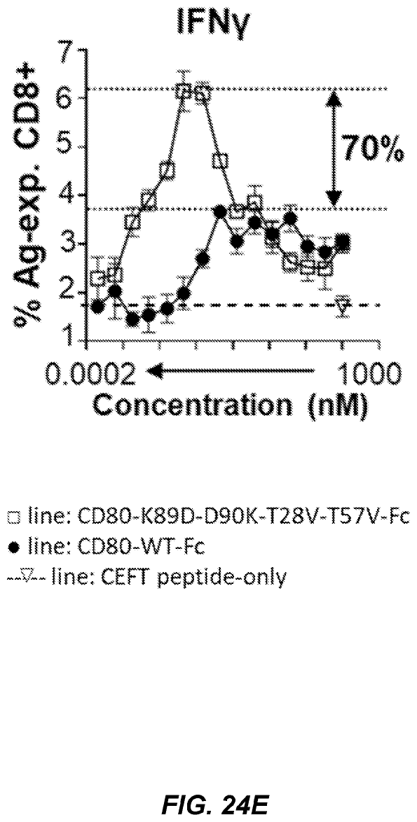

As shown in FIGS. 24A-24E, CD80-K89D-D90K-T28V-T57V-Fc demonstrates superior in vitro potency and max responses in human T cells compared to CD80-WT-Fc. FIG. 24A depicts production of IL-2 by T cells stimulated by CEFT peptide combined with increasing concentrations of CD80-K89D-D90K-T28V-T57V-Fc (square line) compared to CD80-WT-Fc (circle line). Co-stimulation of T cells by CD80-K89D-D90K-T28V-T57V-Fc is associated with an increase in maximum level of IL-2 production (˜140%) and >10× decrease in EC50 relative to CD80-WT-Fc. Dashed line (triangle) represents CEFT peptide-only baseline response.

FIGS. 24B and 24C depict expression of CD25 (IL2Rα, high-affinity IL-2 receptor subunit) and Ki-67 (proliferation marker) respectively, on the surface of T cells (gated on CD8+ T cells) stimulated by CEFT peptide combined with increasing concentrations of CD80-K89D-D90K-T28V-T57V-Fc (square line) and CD80-WT-Fc (circle line) fusion proteins. Co-stimulation using CD80-K89D-D90K-T28V-T57V-Fc is associated with an increase in CD25 expression (˜30%) and proliferative capacity (as depicted by increase in Ki-67 staining, ˜30%) of CD8+ T cells.

FIGS. 24D and 24E depict production of IFNγ expressed by pg/ml of cytokine measured by MSD in cell supernatant (FIG. 24D) and relative abundance (%) of IFNγ+CD8+ T cells measured by flow cytometry (FIG. 24E) upon stimulation by CEFT peptide combined with increasing concentrations of CD80-K89D-D90K-T28V-T57V-Fc (square line) and CD80-WT-Fc (circle line). Stimulation with CD80-K89D-D90K-T28V-T57V-Fc induced enhanced IFNγ production and higher % of IFNγ+CD8+ T cells relative to CD80-WT-Fc.

### Example 21

### Gene Expression Pattern in Primary Human T Cells

96-well plates were coated overnight with 1 μg/ml of anti-human CD3 antibody (HIT3a clone, BioLegend). The following day, plates were washed 2× with PBS and blocked for 1 hour with 200 μl of complete IMDM (10% FBS, 1% Pen/Strep)/well. Upon removal of the blocking medium, plates were seeded with 250,000 PBMCs derived from three different donors and the following proteins: 1) 1.2 μg/ml CD80-K89D-D90K-T28V-T57V-Fc (concentration associated with max IL-2 production in similar assays), 2) 12 μg/ml CD80-WT-Fc (concentration associated with max IL-2 production in similar assay), and 3) 5 μg/ml anti-human CD28 antibody.

After 24 hour incubation at 37° C. in a CO₂ incubator, cells were harvested and stained with viability dye and anti-human CD3 (UCHT1 clone, Biolegend)). Viable CD3+ T cells were flow sorted and used for RNA extraction following Qiagen RNeasy protocol. Isolated RNA was used for a gene expression analysis utilizing Nanostring platform.

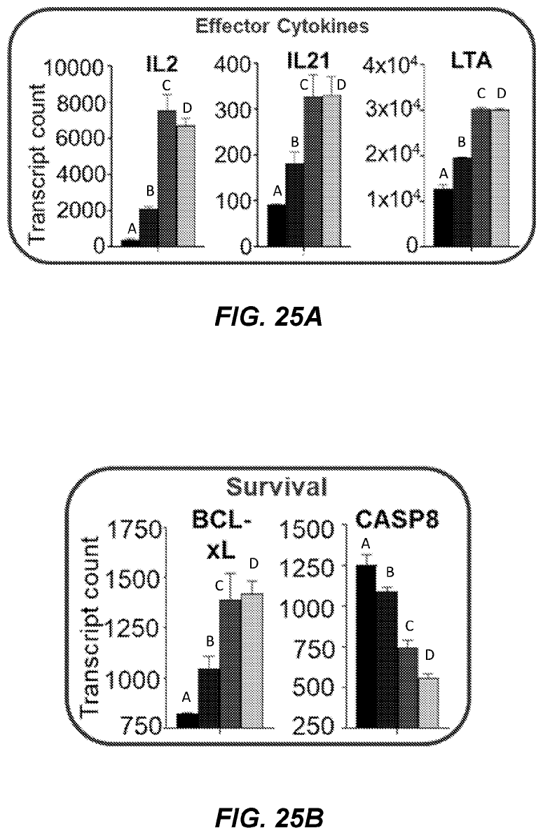

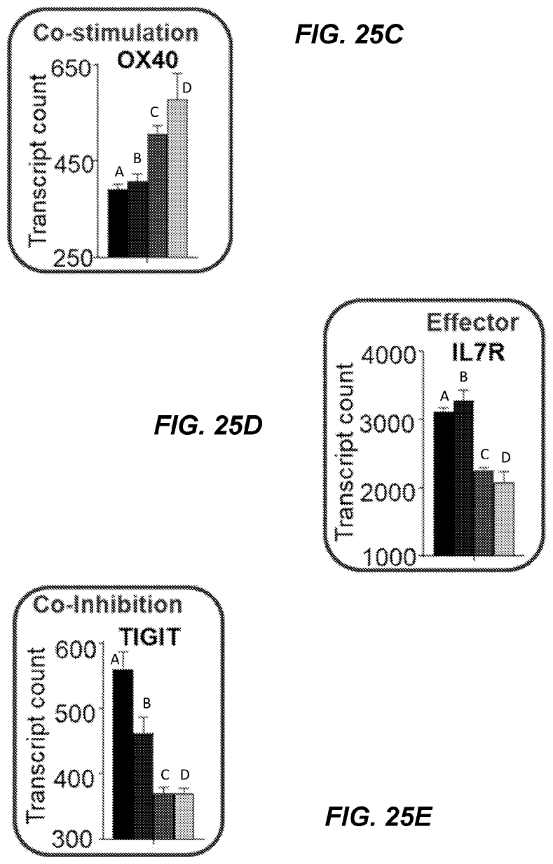

FIGS. 25A-25E show expression levels of selected genes depicted as transcript counts (representative PBMC donors are shown). In each graph of FIGS. 25A-25E, A=anti-human CD3 antibody, B=CD80-WT-Fc, C=CD80-K89D-D90K-T28V-T57V-Fc, and D=anti-human CD28 antibody.

FIG. 25A represents gene expression level of key effector cytokines: IL-2, IL-21 and Lymphotoxin Alpha (LTA). Treatment with anti-human CD3 antibody in combination with 1.2 μg/ml CD80-K89D-D90K-T28V-T57V-Fc (C) or 5 μg/ml anti-human CD28 antibody (D) is associated with enhanced gene expression of all three cytokines compared to co-stimulation with 12 μg/ml CD80-WT-Fc (B).

FIGS. 25B-25E show the expression level of genes encoding survival-defining molecules (BCL-XL and CASP8, FIG. 25B), co-stimulatory molecules (OX-40, FIG. 25C), molecules negatively correlated with effector T cells (IL-7Rα, FIG. 25D) and co-inhibitory molecules (TIGIT, FIG. 25E). As shown in FIG. 25B, co-stimulation with CD80-K89D-D90K-T28V-T57V-Fc (C) and anti-human CD28 antibody (D) is associated with enhanced expression of anti-apoptotic BCL-XL and decreased expression of pro-apoptotic CASP8 compared to co-stimulation with CD80-WT-Fc (B). FIG. 25C shows the expression level of co-stimulatory OX-40-encoding gene is enhanced upon CD80-K89D-D90K-T28V-T57V-Fc (C) and anti-human CD28 antibody-mediated (D) co-stimulation relative to CD80-WT-Fc (B) treatment. FIGS. 25D and 25E show CD80-K89D-D90K-T28V-T57V-Fc and anti-human CD28 antibody co-stimulation is associated with greater decrease relative to anti-human CD3-only baseline of IL7Rα- and TIGIT-encoding genes compared to CD80-WT-Fc treatment.

| SEQ | | |
|---|---|---|
| ID | | |
| NO: | NAME | SEQUENCES |
| 20 | CD80-K36R | VIHVTKEVKEVATLSCGHNVSVEELAQTRIYWQKERKMVLTMMSGDMNIWPE |
| | | YKNRTIFDITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 21 | CD80-K89D | VIHVTKEVKEVATLSCGHNVSVEELAQTRIYWQKEKKMVLTMMSGDMNIWPE |
| | | YKNRTIFDITNNLSIVILALRPSDEGTYECVVLKYEDDAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 22 | CD80-K89E | VIHVTKEVKEVATLSCGHNVSVEELAQTRIYWQKEKKMVLTMMSGDMNIWPE |
| | | YKNRTIFDITNNLSIVILALRPSDEGTYECVVLKYEEDAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 23 | CD80-K89Q | VIHVTKEVKEVATLSCGHNVSVEELAQTRIYWQKEKKMVLTMMSGDMNIWPE |
| | | YKNRTIFDITNNLSIVILALRPSDEGTYECVVLKYEQDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 24 | CD80-D90K | VIHVTKEVKEVATLSCGHNVSVEELAQTRIYWQKEKKMVLTMMSGDMNIWPE |
| | | YKNRTIFDITNNLSIVILALRPSDEGTYECVVLKYEKKAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 25 | CD80-D90N | VIHVTKEVKEVATLSCGHNVSVEELAQTRIYWQKEKKMVLTMMSGDMNIWPE |
| | | YKNRTIFDITNNLSIVILALRPSDEGTYECVVLKYEKNAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 26 | CD80-D90Q | VIHVTKEVKEVATLSCGHNVSVEELAQTRIYWQKEKKMVLTMMSGDMNIWPE |
| | | YKNRTIFDITNNLSIVILALRPSDEGTYECVVLKYEKQAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 27 | CD80-A91S | VIHVTKEVKEVATLSCGHNVSVEELAQTRIYWQKEKKMVLTMMSGDMNIWPE |
| | | YKNRTIFDITNNLSIVILALRPSDEGTYECVVLKYEKDSFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 28 | CD80-K89D- | VIHVTKEVKEVATLSCGHNVSVEELAQTRIYWQKEKKMVLTMMSGDMNIWPE |
| | D90K | YKNRTIFDITNNLSIVILALRPSDEGTYECVVLKYEDKAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 29 | CD80-K89D- | VIHVTKEVKEVATLSCGHNVSVEELAQTRIYWQKEKKMVLTMMSGDMNIWPE |
| | D90Q | YKNRTIFDITNNLSIVILALRPSDEGTYECVVLKYEDQAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 30 | CD80-K89D- | VIHVTKEVKEVATLSCGHNVSVEELAQTRIYWQKEKKMVLTMMSGDMNIWPE |
| | D90N | YKNRTIFDITNNLSIVILALRPSDEGTYECVVLKYEDNAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTENWNTTKQEHFPDN |
| 31 | CD80-K89Q- | VIHVTKEVKEVATLSCGHNVSVEELAQTRIYWQKEKKMVLTMMSGDMNIWPE |
| | D90Q | YKNRTIFDITNNLSIVILALRPSDEGTYECVVLKYEQQAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 32 | CD80-161C | VIHVTKEVKEVATLSCGHNVSVEELAQTRIYWQKEKKMVLTMMSGDMNIWPE |
| | | YKNRTIFDCTNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 33 | CD80-E23C- | VIHVTKEVKEVATLSCGHNVSVCELCQTRIYWQKEKKMVLTMMSGDMNIWPE |
| | A26C | YKNRTIFDITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 34 | CD80-V22C- | VIHVTKEVKEVATLSCGHNVSCEELAQTRIYWQKEKKMVLTMMSCDMNIWPE |
| | G45C | YKNRTIFDITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 35 | CD80-V11L- | VIHVTKEVKELATLSCGHNVSFEELAQTRIYWQKEKKMVLTMMSGDMNIWPE |
| | V22F | YKNRTIFDITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 36 | CD80-V11L- | VIHVTKEVKELATLSCGHNVSVEELAQTRIYWQKEKKMVLTMMSGDMNIWPE |
| | T62Y | YKNRTIFDIYNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 37 | CD80-V11L- | VIHVTKEVKELATLSCGHNVSVEELAQTRIYWQKEKKMVLTMMSGDMNIWPE |
| | T62Y-N63D | YKNRTIFDIYDNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 38 | CD80-V22F- | VIHVTKEVKEVATLSCGHNVSFEELAQTRIYWQKEKKMVLTMMSGDMNIWPE |
| | T62L | YKNRTIFDILNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 39 | CD80-T28V- | VIHVTKEVKEVATLSCGHNVSVEELAQVRIYWQKEKKMVLTMMSGDMNIWPE |
| | T57V | YKNRVIFDITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 40 | CD80-T28V- | VIHVTKEVKEVATLSCGHNVSVEELAQVRIQWEKEKKMVLTMMSGDMNIWPE |
| | T57V-Y31Q- | YENRVIFDITNNLSIVILALRPSDEGTYECVVLKYEKDAFKREHLAEVTLSV |
| | Q33E-K54E | KADFPTPSISDFEIPTSNIRRIICSTSGGFPEPHLSWLENGEELNAINTTVS |
| | | QDPETELYAVSSKLDFNMTTNHSFMCLIKYGHLRVNQTENWNTTKQEHFPDN |
| 41 | CD80-D60Y | VIHVTKEVKEVATLSCGHNVSVEELAQTRIYWQKEKKMVLTMMSGDMNIWPE |
| | | YKNRTIFYITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 42 | CD80-D60Y- | VIHVTKEVKEVATLSCGHNVSVEELAQTRIYWQKEKKMVLTMMSGDMNIWPE |
| | K54E-N63E- | YENRTIFYITEDLSIVILALRPSDEGTYECVVLKYEKDAFKREHLAEVTLSV |
| | N64D | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 43 | CD80-D60Y- | VIHVTKEVKEVATLSCGHNVSVEELAQTRIYWQKEKKMVLTMMSGDMNIWPE |
| | T62L | YKNRTIFYILNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 44 | CD80-D60Y- | VIHVTKEVKEVATLSCGHNVSVEELAQTRIYWQKEKKMVLTMMSGDMNIWPE |
| | T62L-N63D- | YKNRTIFYILDELSIVILALRPSDEGTYECVVLKYEKDAFKREHLAEVTLSV |
| | N64E | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 45 | CD80-V22F- | VIHVTKEVKEVATLSCGHNVSFEELAQTRIYWQKEKKMVLTMMSGDMNIWPE |
| | D60Y | YKNRTIFYITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 46 | CD80-V22F- | VIHVTKEVKEVATLSCGHNVSFEELAQTRIYWQKEKKMVLTMMSGDMNIWPE |
| | D60Y-K54E- | YENRTIFYITNELSIVILALRPSDEGTYECVVLKYEKDAFKREHLAEVTLSV |
| | N64E | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 47 | CD80-D60F- | VIHVTKEVKEVATLSCGHNVSVEELAQTRIYWQKEKKMVLTMMSGDMNIWPE |
| | T62\| | YKNRTIFFIINNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 48 | CD80-D60R- | VIHVTKEVKEVATLSCGHNVSVEELAQTRIYWQKEKKMVLTMMSGDMNIWPE |
| | T62Y | YKNRTIFRIYNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 49 | CD80-D60Y- | VIHVTKEVKELATLSCGHNVSVEELAQTRIYWQKEKKMVLTMMSGDMNIWPE |
| | V11L | YKNRTIFYITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 50 | CD80-D60Y- | VIHVTKEVKELATLSCGHNVSVEELAQTRIYWQKEKKMVLTMMSGDMNIWPE |
| | V11L-N63D | YKNRTIFYITDNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 51 | CD80-D60Y- | VIHVTKEVKEVATLSCGHNVSMEELAQTRIYWQKEKKMVLTMMSGDMNIWPE |
| | V22M | YKNRTIFYITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 52 | CD80-D60T- | VIHVTKEVKEVATLSCGHNVSVEELAQTRIYWQKEKKMVLTMMSGDMNIWPE |
| | T62Y | YKNRTIFTIYNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTENWNTTKQEHFPDN |
| 53 | CD80-D60Q- | VIHVTKEVKEVATLSCGHNVSVEELAQTRIYWQKEKKMVLTMMSGDMNIWPE |
| | T62F | YKNRTIFQIFNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 54 | CD80- V22F- | VIHVTKEVKEVATLSCGHNVSFEELAQVRIYWQKEKKMVLTMMSGDMNIWPE |
| | T28V-T57V | YKNRVIFDITNNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 55 | CD80-T28V- | VIHVTKEVKEVATLSCGHNVSVEELAQVRIQWEKEKKMVLTMMSGDMNIWPE |
| | T57V-Y31Q- | YENRVIFDITNNLSIVILALRPSDEGTYECVVLKYEKDAFKREHLAEVTLSV |
| | Q33E-K54E | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 56 | CD80-V22F- | VIHVTKEVKEVATLSCGHNVSFEELAQTRIYWQKEKKMVLTMMSGDMNIWPE |
| | T62L-N64E | YKNRTIFDILNELSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 57 | CD80-V22F- | VIHVTKEVKEVATLSCGHNVSFEELAQTRIYWQKEKKMVLTMMSGDMNIWPE |
| | T62L-N63D- | YKNRTIFDILDELSIVILALRPSDEGTYECVVLKYEKDAFKREHLAEVTLSV |
| | N64E | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 58 | CD80-D60Y- | VIHVTKEVKEVATLSCGHNVSVEELAQTRIYWQKEKKMVLTMMSGDMNIWPE |
| | T62L-N63D | YKNRTIFYILDNLSIVILALRPSDEGTYECVVLKYEKDAFKREHLAEVTLSV |
| | | KADFPTPSISDFEIPTSNIRRIICSTSGGFPEPHLSWLENGEELNAINTTVS |
| | | QDPETELYAVSSKLDENMTTNHSFMCLIKYGHLRVNQTFNWNTTKQEHFPDN |
| 59 | CD80-K89Q- | VIHVTKEVKEVATLSCGHNVSVEELAQTRIYWQKEKKMVLTMMSGDMNIWPE |
| | D90Q-161C | YKNRTIFDCTNNLSIVILALRPSDEGTYECVVLKYEQQAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 60 | CD80-D90Q- | VIHVTKEVKEVATLSCGHNVSVCELCQTRIYWQKEKKMVLTMMSGDMNIWPE |
| | E23C-A26C | YKNRTIFDITNNLSIVILALRPSDEGTYECVVLKYEKQAFKREHLAEVTLSV |
| | | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 61 | CD80-K89Q- | VIHVTKEVKEVATLSCGHNVSVCELCQTRIYWQKEKKMVLTMMSGDMNIWPE |
| | D90Q-E23C- | YKNRTIFDITNNLSIVILALRPSDEGTYECVVLKYEQQAFKREHLAEVTLSV |
| | A26C | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 62 | CD80-K89Q- | VIHVTKEVKEVATLSCGHNVSCEELAQTRIYWQKEKKMVLTMMSCDMNIWPE |
| | D90Q-V22C- | YKNRTIFDITNNLSIVILALRPSDEGTYECVVLKYEQQAFKREHLAEVTLSV |
| | G45C | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 63 | CD80-K89D- | VIHVTKEVKEVATLSCGHNVSVEELAQVRIYWQKEKKMVLTMMSGDMNIWPE |
| | D90K-T28V- | YKNRVIFDITNNLSIVILALRPSDEGTYECVVLKYEDKAFKREHLAEVTLSV |
| | T57V | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| 64 | CD80-WT-Fc | VIHVTKEVKEVATLSCGHNVSVEELAQTRIYWQKEKKMVLTMMSGDMNIWPE |
| | (WT Human | YKNRTIFDITNNLSIVILALRPSDEGTYECVVLKYEKDAFKREHLAEVTLSV |
| | IgG1) | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| | | EPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWINGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 65 | CD80-D90Q- | VIHVTKEVKEVATLSCGHNVSVEELAQTRIYWQKEKKMVLTMMSGDMNIWPE |
| | Fc (WT | YKNRTIFDITNNLSIVILALRPSDEGTYECVVLKYEKQAFKREHLAEVTLSV |
| | Human IgG1) | KADFPTPSISDFEIPTSNIRRIICSTSGGFPEPHLSWLENGEELNAINTTVS |
| | | QDPETELYAVSSKLDFNMTTNHSFMCLIKYGHLRVNQTFNWNTTKQEHFPDN |
| | | EPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 66 | CD80-K89Q- | VIHVTKEVKEVATLSCGHNVSVEELAQTRIYWQKEKKMVLTMMSGDMNIWPE |
| | D90Q-Fc | YKNRTIFDITNNLSIVILALRPSDEGTYECVVLKYEQQAFKREHLAEVTLSV |
| | (WT Human | KADFPTPSISDFEIPTSNIRRIICSTSGGFPEPHLSWLENGEELNAINTTVS |
| | IgG1 | QDPETELYAVSSKLDFNMTTNHSFMCLIKYGHLRVNQTFNWNTTKQEHFPDN |
| | | EPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 67 | CD80-D90Q- | VIHVTKEVKEVATLSCGHNVSVCELCQTRIYWQKEKKMVLTMMSGDMNIWPE |
| | E23C-A26C- | YKNRTIFDITNNLSIVILALRPSDEGTYECVVLKYEKQAFKREHLAEVTLSV |
| | Fc (WT | KADFPTPSISDFEIPTSNIRRIICSTSGGFPEPHLSWLENGEELNAINTTVS |
| | Human IgG1) | QDPETELYAVSSKLDENMTTNHSFMCLIKYGHLRVNQTFNWNTTKQEHFPDN |
| | | EPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 68 | CD80-K89Q- | VIHVTKEVKEVATLSCGHNVSVCELCQTRIYWQKEKKMVLTMMSGDMNIWPE |
| | D90Q-E23C- | YKNRTIFDITNNLSIVILALRPSDEGTYECVVLKYEQQAFKREHLAEVTLSV |
| | A26C-Fc (WT | KADFPTPSISDFEIPTSNIRRIICSTSGGFPEPHLSWLENGEELNAINTTVS |
| | Human IgG1) | QDPETELYAVSSKLDFNMTTNHSFMCLIKYGHLRVNQTFNWNTTKQEHFPDN |
| | | EPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 69 | CD80-K89D- | VIHVTKEVKEVATLSCGHNVSVEELAQVRIYWQKEKKMVLTMMSGDMNIWPE |
| | D90K-T28V- | YKNRVIFDITNNLSIVILALRPSDEGTYECVVLKYEDKAFKREHLAEVTLSV |
| | T57V-Fc (WT | KADFPTPSISDFEIPTSNIRRIICSTSGGFPEPHLSWLENGEELNAINTTVS |
| | Human IgG1) | QDPETELYAVSSKLDFNMTTNHSFMCLIKYGHLRVNQTFNWNTTKQEHFPDN |
| | | EPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 70 | CD80-WT-Fc | VIHVTKEVKEVATLSCGHNVSVEELAQTRIYWQKEKKMVLTMMSGDMNIWPE |
| | (Human IgG1 | YKNRTIFDITNNLSIVILALRPSDEGTYECVVLKYEKDAFKREHLAEVTLSV |
| | with C220S | KADFPTPSISDFEIPTSNIRRIICSTSGGFPEPHLSWLENGEELNAINTTVS |
| | mutation) | QDPETELYAVSSKLDENMTTNHSFMCLIKYGHLRVNQTENWNTTKQEHFPDN |
| | | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWINGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 71 | CD80-K36R- | VIHVTKEVKEVATLSCGHNVSVEELAQTRIYWQKERKMVLTMMSGDMNIWPE |
| | Fc (Human | YKNRTIFDITNNLSIVILALRPSDEGTYECVVLKYEKDAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 72 | CD80-K89D- | VIHVTKEVKEVATLSCGHNVSVEELAQTRIYWQKEKKMVLTMMSGDMNIWPE |
| | Fc (Human | YKNRTIFDITNNLSIVILALRPSDEGTYECVVLKYEDDAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 73 | CD80-K89E- | VIHVTKEVKEVATLSCGHNVSVEELAQTRIYWQKEKKMVLTMMSGDMNIWPE |
| | Fc (Human | YKNRTIFDITNNLSIVILALRPSDEGTYECVVLKYEEDAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 74 | CD80-K89Q- | VIHVTKEVKEVATLSCGHNVSVEELAQTRIYWQKEKKMVLTMMSGDMNIWPE |
| | Fc (Human | YKNRTIFDITNNLSIVILALRPSDEGTYECVVLKYEQDAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 75 | CD80-D90K- | VIHVTKEVKEVATLSCGHNVSVEELAQTRIYWQKEKKMVLTMMSGDMNIWPE |
| | Fc (Human | YKNRTIFDITNNLSIVILALRPSDEGTYECVVLKYEKKAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDENMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 76 | CD80-D90N- | VIHVTKEVKEVATLSCGHNVSVEELAQTRIYWQKEKKMVLTMMSGDMNIWPE |
| | Fc (Human | YKNRTIFDITNNLSIVILALRPSDEGTYECVVLKYEKNAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDENMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWINGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 77 | CD80-D90Q- | VIHVTKEVKEVATLSCGHNVSVEELAQTRIYWQKEKKMVLTMMSGDMNIWPE |
| | Fc (Human | YKNRTIFDITNNLSIVILALRPSDEGTYECVVLKYEKQAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTENWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 78 | CD80-A91S- | VIHVTKEVKEVATLSCGHNVSVEELAQTRIYWQKEKKMVLTMMSGDMNIWPE |
| | Fc (Human | YKNRTIFDITNNLSIVILALRPSDEGTYECVVLKYEKDSFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 79 | CD80-K89D- | VIHVTKEVKEVATLSCGHNVSVEELAQTRIYWQKEKKMVLTMMSGDMNIWPE |
| | D90K-Fc | YKNRTIFDITNNLSIVILALRPSDEGTYECVVLKYEDKAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 80 | CD80-K89D- | VIHVTKEVKEVATLSCGHNVSVEELAQTRIYWQKEKKMVLTMMSGDMNIWPE |
| | D90Q-Fc | YKNRTIFDITNNLSIVILALRPSDEGTYECVVLKYEDQAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 81 | CD80-K89D- | VIHVTKEVKEVATLSCGHNVSVEELAQTRIYWQKEKKMVLTMMSGDMNIWPE |
| | D90N-Fc | YKNRTIFDITNNLSIVILALRPSDEGTYECVVLKYEDNAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 82 | CD80-K89Q- | VIHVTKEVKEVATLSCGHNVSVEELAQTRIYWQKEKKMVLTMMSGDMNIWPE |
| | D90Q-Fc | YKNRTIFDITNNLSIVILALRPSDEGTYECVVLKYEQQAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 83 | CD80-161C- | VIHVTKEVKEVATLSCGHNVSVEELAQTRIYWQKEKKMVLTMMSGDMNIWPE |
| | Fc (Human | YKNRTIFDCTNNLSIVILALRPSDEGTYECVVLKYEKDAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 84 | CD80-E23C- | VIHVTKEVKEVATLSCGHNVSVCELCQTRIYWQKEKKMVLTMMSGDMNIWPE |
| | A26C-Fc | YKNRTIFDITNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 85 | CD80-V22C- | VIHVTKEVKEVATLSCGHNVSCEELAQTRIYWQKEKKMVLTMMSCDMNIWPE |
| | G45C-Fc | YKNRTIFDITNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 86 | CD80-V11L- | VIHVTKEVKELATLSCGHNVSFEELAQTRIYWQKEKKMVLTMMSGDMNIWPE |
| | V22F-Fc | YKNRTIFDITNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 87 | CD80-V11L- | VIHVTKEVKELATLSCGHNVSVEELAQTRIYWQKEKKMVLTMMSGDMNIWPE |
| | T62Y-Fc | YKNRTIFDIYNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 88 | CD80-V11L- | VIHVTKEVKELATLSCGHNVSVEELAQTRIYWQKEKKMVLTMMSGDMNIWPE |
| | T62Y-N63D- | YKNRTIFDIYDNLSIVILALRPSDEGTYECVVLKYEKDAFKREHLAEVTLSV |
| | Fc (Human | KADFPTPSISDFEIPTSNIRRIICSTSGGFPEPHLSWLENGEELNAINTTVS |
| | IgG1 with | QDPETELYAVSSKLDENMTTNHSFMCLIKYGHLRVNQTFNWNTTKQEHFPDN |
| | C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWINGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 89 | CD80-V22F- | VIHVTKEVKEVATLSCGHNVSFEELAQTRIYWQKEKKMVLTMMSGDMNIWPE |
| | T62L-Fc | YKNRTIFDILNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTENWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 90 | CD80-T28V- | VIHVTKEVKEVATLSCGHNVSVEELAQVRIYWQKEKKMVLTMMSGDMNIWPE |
| | T57V-Fc | YKNRVIFDITNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 91 | CD80-T28V- | VIHVTKEVKEVATLSCGHNVSVEELAQVRIQWEKEKKMVLTMMSGDMNIWPE |
| | T57V-Y31Q- | YENRVIFDITNNLSIVILALRPSDEGTYECVVLKYEKDAFKREHLAEVTLSV |
| | Q33E-K54E- | KADFPTPSISDFEIPTSNIRRIICSTSGGFPEPHLSWLENGEELNAINTTVS |
| | Fc (Human | QDPETELYAVSSKLDFNMTTNHSFMCLIKYGHLRVNQTFNWNTTKQEHFPDN |
| | IgG1 with | EPKSSDKTHTCPPCPAPELLGGPSVFLFPPKPKDTLMISRTPEVTCVVVDVS |
| | C220S | HEDPEVKFNWYVDGVEVHNAKTKPREEQYNSTYRVVSVLTVLHQDWINGKEY |
| | mutation) | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 92 | CD80-D60Y- | VIHVTKEVKEVATLSCGHNVSVEELAQTRIYWQKEKKMVLTMMSGDMNIWPE |
| | Fc (Human | YKNRTIFYITNNLSIVILALRPSDEGTYECVVLKYEKDAFKREHLAEVTLSV |
| | IgG1 with | KADFPTPSISDFEIPTSNIRRIICSTSGGFPEPHLSWLENGEELNAINTTVS |
| | C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 93 | CD80-D60Y- | VIHVTKEVKEVATLSCGHNVSVEELAQTRIYWQKEKKMVLTMMSGDMNIWPE |
| | K54E-N63E- | YENRTIFYITEDLSIVILALRPSDEGTYECVVLKYEKDAFKREHLAEVTLSV |
| | N64D-Fc | KADFPTPSISDFEIPTSNIRRIICSTSGGFPEPHLSWLENGEELNAINTTVS |
| | (Human IgG1 | QDPETELYAVSSKLDFNMTTNHSFMCLIKYGHLRVNQTFNWNTTKQEHFPDN |
| | with C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 94 | CD80-D60Y- | VIHVTKEVKEVATLSCGHNVSVEELAQTRIYWQKEKKMVLTMMSGDMNIWPE |
| | T62L-Fc | YKNRTIFYILNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDENMTTNHSFMCLIKYGHLRVNQTENWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWINGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 95 | CD80-D60Y- | VIHVTKEVKEVATLSCGHNVSVEELAQTRIYWQKEKKMVLTMMSGDMNIWPE |
| | T62L-N63D- | YKNRTIFYILDELSIVILALRPSDEGTYECVVLKYEKDAFKREHLAEVTLSV |
| | N64E-Fc | KADFPTPSISDFEIPTSNIRRIICSTSGGFPEPHLSWLENGEELNAINTTVS |
| | (Human IgG1 | QDPETELYAVSSKLDFNMTTNHSFMCLIKYGHLRVNQTFNWNTTKQEHFPDN |
| | with C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 96 | CD80-V22F- | VIHVTKEVKEVATLSCGHNVSFEELAQTRIYWQKEKKMVLTMMSGDMNIWPE |
| | D60Y-Fc | YKNRTIFYITNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 97 | CD80-V22F- | VIHVTKEVKEVATLSCGHNVSFEELAQTRIYWQKEKKMVLTMMSGDMNIWPE |
| | D60Y-K54E- | YENRTIFYITNELSIVILALRPSDEGTYECVVLKYEKDAFKREHLAEVTLSV |
| | N64E-Fc | KADFPTPSISDFEIPTSNIRRIICSTSGGFPEPHLSWLENGEELNAINTTVS |
| | (Human IgG1 | QDPETELYAVSSKLDENMTTNHSFMCLIKYGHLRVNQTFNWNTTKQEHFPDN |
| | with C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWINGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 98 | CD80-D60F- | VIHVTKEVKEVATLSCGHNVSVEELAQTRIYWQKEKKMVLTMMSGDMNIWPE |
| | T621-Fc | YKNRTIFFIINNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 99 | CD80-D60R- | VIHVTKEVKEVATLSCGHNVSVEELAQTRIYWQKEKKMVLTMMSGDMNIWPE |
| | T62Y-Fc | YKNRTIFRIYNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 100 | CD80-D60Y- | VIHVTKEVKELATLSCGHNVSVEELAQTRIYWQKEKKMVLTMMSGDMNIWPE |
| | V11L-Fc | YKNRTIFYITNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDENMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 101 | CD80-D60Y- | VIHVTKEVKELATLSCGHNVSVEELAQTRIYWQKEKKMVLTMMSGDMNIWPE |
| | V11L-N63D- | YKNRTIFYITDNLSIVILALRPSDEGTYECVVLKYEKDAFKREHLAEVTLSV |
| | Fc (Human | KADFPTPSISDFEIPTSNIRRIICSTSGGFPEPHLSWLENGEELNAINTTVS |
| | IgG1 with | QDPETELYAVSSKLDFNMTTNHSFMCLIKYGHLRVNQTENWNTTKQEHFPDN |
| | C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 102 | CD80-D60Y- | VIHVTKEVKEVATLSCGHNVSMEELAQTRIYWQKEKKMVLTMMSGDMNIWPE |
| | V22M-Fc | YKNRTIFYITNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 103 | CD80-D60T- | VIHVTKEVKEVATLSCGHNVSVEELAQTRIYWQKEKKMVLTMMSGDMNIWPE |
| | T62Y-Fc | YKNRTIFTIYNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWINGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 104 | CD80-D60Q- | VIHVTKEVKEVATLSCGHNVSVEELAQTRIYWQKEKKMVLTMMSGDMNIWPE |
| | T62F-Fc | YKNRTIFQIFNNLSIVILALRPSDEGTYECVVLKYEKDAFKREHLAEVTLSV |
| | (Human IgG1 | KADFPTPSISDFEIPTSNIRRIICSTSGGFPEPHLSWLENGEELNAINTTVS |
| | with C220S | QDPETELYAVSSKLDFNMTTNHSFMCLIKYGHLRVNQTFNWNTTKQEHFPDN |
| | mutation) | EPKSSDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 105 | CD80- V22F- | VIHVTKEVKEVATLSCGHNVSFEELAQVRIYWQKEKKMVLTMMSGDMNIWPE |
| | T28V-T57V- | YKNRVIFDITNNLSIVILALRPSDEGTYECVVLKYEKDAFKREHLAEVTLSV |
| | Fc (Human | KADFPTPSISDFEIPTSNIRRIICSTSGGFPEPHLSWLENGEELNAINTTVS |
| | IgG1 with | QDPETELYAVSSKLDENMTTNHSFMCLIKYGHLRVNQTFNWNTTKQEHFPDN |
| | C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 106 | CD80-T28V- | VIHVTKEVKEVATLSCGHNVSVEELAQVRIQWEKEKKMVLTMMSGDMNIWPE |
| | T57V-Y31Q- | YENRVIFDITNNLSIVILALRPSDEGTYECVVLKYEKDAFKREHLAEVTLSV |
| | Q33E-K54E- | KADFPTPSISDFEIPTSNIRRIICSTSGGFPEPHLSWLENGEELNAINTTVS |
| | Fc (Human | QDPETELYAVSSKLDENMTTNHSFMCLIKYGHLRVNQTFNWNTTKQEHFPDN |
| | IgG1 with | EPKSSDKTHTCPPCPAPELLGGPSVFLFPPKPKDTLMISRTPEVTCVVVDVS |
| | C220S | HEDPEVKFNWYVDGVEVHNAKTKPREEQYNSTYRVVSVLTVLHQDWLNGKEY |
| | mutation) | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 107 | CD80-V22F- | VIHVTKEVKEVATLSCGHNVSFEELAQTRIYWQKEKKMVLTMMSGDMNIWPE |
| | T62L-N64E- | YKNRTIFDILNELSIVILALRPSDEGTYECVVLKYEKDAFKREHLAEVTLSV |
| | Fc (Human | KADFPTPSISDFEIPTSNIRRIICSTSGGFPEPHLSWLENGEELNAINTTVS |
| | IgG1 with | QDPETELYAVSSKLDFNMTTNHSFMCLIKYGHLRVNQTFNWNTTKQEHFPDN |
| | C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 108 | CD80-V22F- | VIHVTKEVKEVATLSCGHNVSFEELAQTRIYWQKEKKMVLTMMSGDMNIWPE |
| | T62L-N63D- | YKNRTIFDILDELSIVILALRPSDEGTYECVVLKYEKDAFKREHLAEVTLSV |
| | N64E-Fc | KADFPTPSISDFEIPTSNIRRIICSTSGGFPEPHLSWLENGEELNAINTTVS |
| | (Human IgG1 | QDPETELYAVSSKLDFNMTTNHSFMCLIKYGHLRVNQTFNWNTTKQEHFPDN |
| | with C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 109 | CD80-D60Y- | VIHVTKEVKEVATLSCGHNVSVEELAQTRIYWQKEKKMVLTMMSGDMNIWPE |
| | T62L-N63D- | YKNRTIFYILDNLSIVILALRPSDEGTYECVVLKYEKDAFKREHLAEVTLSV |
| | Fc (Human | KADFPTPSISDFEIPTSNIRRIICSTSGGFPEPHLSWLENGEELNAINTTVS |
| | IgG1 with | QDPETELYAVSSKLDFNMTTNHSFMCLIKYGHLRVNQTFNWNTTKQEHFPDN |
| | C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 110 | CD80-K89Q- | VIHVTKEVKEVATLSCGHNVSVEELAQTRIYWQKEKKMVLTMMSGDMNIWPE |
| | D90Q-161C- | YKNRTIFDCTNNLSIVILALRPSDEGTYECVVLKYEQQAFKREHLAEVTLSV |
| | Fc (Human | KADFPTPSISDFEIPTSNIRRIICSTSGGFPEPHLSWLENGEELNAINTTVS |
| | IgG1 with | QDPETELYAVSSKLDFNMTTNHSFMCLIKYGHLRVNQTENWNTTKQEHFPDN |
| | C220S | EPKSSDKTHTCPPCPAPELLGGPSVFLFPPKPKDTLMISRTPEVTCVVVDVS |
| | mutation) | HEDPEVKENWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 111 | CD80-D90Q- | VIHVTKEVKEVATLSCGHNVSVCELCQTRIYWQKEKKMVLTMMSGDMNIWPE |
| | E23C-A26C- | YKNRTIFDITNNLSIVILALRPSDEGTYECVVLKYEKQAFKREHLAEVTLSV |
| | Fc (Human | KADFPTPSISDFEIPTSNIRRIICSTSGGFPEPHLSWLENGEELNAINTTVS |
| | IgG1 with | QDPETELYAVSSKLDFNMTTNHSFMCLIKYGHLRVNQTFNWNTTKQEHFPDN |
| | C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVF |
| | | SCSVMHEALHNHYTQKSLSLSPG |
| 112 | CD80-K89Q- | VIHVTKEVKEVATLSCGHNVSVCELCQTRIYWQKEKKMVLTMMSGDMNIWPE |
| | D90Q-E23C- | YKNRTIFDITNNLSIVILALRPSDEGTYECVVLKYEQQAFKREHLAEVTLSV |
| | A26C-Fc | KADFPTPSISDFEIPTSNIRRIICSTSGGFPEPHLSWLENGEELNAINTTVS |
| | (Human IgG1 | QDPETELYAVSSKLDENMTTNHSFMCLIKYGHLRVNQTFNWNTTKQEHFPDN |
| | with C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 113 | CD80-K89Q- | VIHVTKEVKEVATLSCGHNVSCEELAQTRIYWQKEKKMVLTMMSCDMNIWPE |
| | D90Q-V22C- | YKNRTIFDITNNLSIVILALRPSDEGTYECVVLKYEQQAFKREHLAEVTLSV |
| | G45C-Fc | KADFPTPSISDFEIPTSNIRRIICSTSGGFPEPHLSWLENGEELNAINTTVS |
| | (Human IgG1 | QDPETELYAVSSKLDENMTTNHSFMCLIKYGHLRVNQTFNWNTTKQEHFPDN |
| | with C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 114 | CD80-K89D- | VIHVTKEVKEVATLSCGHNVSVEELAQVRIYWQKEKKMVLTMMSGDMNIWPE |
| | D90K-T28V- | YKNRVIFDITNNLSIVILALRPSDEGTYECVVLKYEDKAFKREHLAEVTLSV |
| | T57V-Fc | KADFPTPSISDFEIPTSNIRRIICSTSGGFPEPHLSWLENGEELNAINTTVS |
| | (Human IgG1 | QDPETELYAVSSKLDFNMTTNHSFMCLIKYGHLRVNQTFNWNTTKQEHFPDN |
| | with C220S | EPKSSDKTHTCPPCPAPELLGGPSVFLFPPKPKDTLMISRTPEVTCVVVDVS |
| | mutation) | HEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVE |
| | | SCSVMHEALHNHYTQKSLSLSPG |
| 115 | CD80-K89Q- | GTGATCCACGTCACCAAGGAAGTCAAGGAAGTGGCCACCCTCTCCTGCGGTC |
| | D90Q-161C- | ACAATGTGTCCGTGGAAGAACTGGCCCAGACGCGGATCTACTGGCAAAAGGA |
| | Fc (Human | GAAGAAGATGGTGCTGACCATGATGAGCGGAGACATGAACATCTGGCCCGAG |
| | IgG1 with | TACAAGAACCGGACTATCTTTGACTGCACCAACAACTTGTCGATCGTCATCC |
| | C220S | TGGCACTCCGGCCTAGCGATGAGGGAACCTATGAGTGCGTGGTGTTGAAATA |
| | mutation) | CGAACAGCAAGCCTTCAAGAGAGAGCACCTCGCCGAAGTGACCCTGAGCGTG |
| | Nucleic acid | AAGGCCGACTTCCCCACCCCGAGCATTTCGGACTTCGAGATTCCGACCTCCA |
| | | ACATTCGCCGCATTATCTGTTCAACCTCCGGCGGATTCCCGGAGCCACATCT |
| | | GTCCTGGCTGGAGAACGGCGAAGAACTGAACGCGATTAACACTACCGTGTCC |
| | | CAAGACCCTGAAACTGAGCTGTACGCCGTGTCATCGAAGCTCGACTTCAACA |
| | | TGACTACCAACCACTCCTTCATGTGCCTGATCAAATACGGGCATCTCCGGGT |
| | | CAACCAGACCTTCAACTGGAACACTACCAAGCAGGAGCACTTTCCCGACAAT |
| | | GAGCCTAAGTCCTCGGACAAGACCCACACCTGTCCTCCATGTCCGGCGCCGG |
| | | AATTGCTTGGCGGTCCGAGCGTGTTCCTGTTCCCACCGAAGCCAAAGGACAC |
| | | CCTGATGATTAGCAGGACTCCCGAAGTCACTTGCGTGGTCGTGGATGTGTCT |
| | | CACGAGGACCCGGAAGTCAAGTTCAATTGGTACGTGGATGGCGTGGAAGTCC |
| | | ATAACGCCAAGACGAAACCCCGCGAGGAACAGTACAACAGCACCTACCGCGT |
| | | GGTGTCAGTGCTGACCGTGCTGCACCAGGATTGGCTCAACGGAAAGGAGTAC |
| | | AAGTGCAAAGTGTCGAACAAAGCCCTGCCTGCTCCCATCGAAAAGACAATCT |
| | | CGAAGGCCAAGGGACAACCCCGGGAACCTCAGGTCTACACCCTGCCTCCTTC |
| | | CCGGGAGGAAATGACCAAGAACCAAGTGTCCCTCACTTGCCTTGTGAAGGGA |
| | | TTCTACCCGTCCGACATCGCCGTGGAGTGGGAATCCAACGGTCAACCCGAGA |
| | | ACAACTACAAGACCACCCCTCCGGTGCTCGACTCGGATGGGTCATTCTTCCT |
| | | GTACTCCAAGCTCACCGTGGACAAGTCCAGATGGCAGCAGGGAAACGTGTTC |
| | | TCCTGCTCGGTCATGCACGAGGCCCTGCACAACCATTACACTCAGAAGTCCC |
| | | TGTCCCTGAGCCCGGGAAAA |
| 116 | CD80-D90Q- | GTGATCCACGTCACCAAGGAAGTCAAGGAAGTGGCCACCCTCTCCTGCGGTC |
| | E23C-A26C- | ACAATGTGTCCGTGTGCGAACTGTGCCAGACGCGGATCTACTGGCAAAAGGA |
| | Fc (Human | GAAGAAGATGGTGCTGACCATGATGAGCGGAGACATGAACATCTGGCCCGAG |
| | IgG1 with | TACAAGAACCGGACTATCTTTGACATCACCAACAACTTGTCGATCGTCATCC |
| | C220S | TGGCACTCCGGCCTAGCGATGAGGGAACCTATGAGTGCGTGGTGTTGAAATA |
| | mutation) | CGAAAAGCAAGCCTTCAAGAGAGAGCACCTCGCCGAAGTGACCCTGAGCGTG |
| | Nucleic acid | AAGGCCGACTTCCCCACCCCGAGCATTTCGGACTTCGAGATTCCGACCTCCA |
| | | ACATTCGCCGCATTATCTGTTCAACCTCCGGCGGATTCCCGGAGCCACATCT |
| | | GTCCTGGCTGGAGAACGGCGAAGAACTGAACGCGATTAACACTACCGTGTCC |
| | | CAAGACCCTGAAACTGAGCTGTACGCCGTGTCATCGAAGCTCGACTTCAACA |
| | | TGACTACCAACCACTCCTTCATGTGCCTGATCAAATACGGGCATCTCCGGGT |
| | | CAACCAGACCTTCAACTGGAACACTACCAAGCAGGAGCACTTTCCCGACAAT |
| | | GAGCCTAAGTCCTCGGACAAGACCCACACCTGTCCTCCATGTCCGGCGCCGG |
| | | AATTGCTTGGCGGTCCGAGCGTGTTCCTGTTCCCACCGAAGCCAAAGGACAC |
| | | CCTGATGATTAGCAGGACTCCCGAAGTCACTTGCGTGGTCGTGGATGTGTCT |
| | | CACGAGGACCCGGAAGTCAAGTTCAATTGGTACGTGGATGGCGTGGAAGTCC |
| | | ATAACGCCAAGACGAAACCCCGCGAGGAACAGTACAACAGCACCTACCGCGT |
| | | GGTGTCAGTGCTGACCGTGCTGCACCAGGATTGGCTCAACGGAAAGGAGTAC |
| | | AAGTGCAAAGTGTCGAACAAAGCCCTGCCTGCTCCCATCGAAAAGACAATCT |
| | | CGAAGGCCAAGGGACAACCCCGGGAACCTCAGGTCTACACCCTGCCTCCTTC |
| | | CCGGGAGGAAATGACCAAGAACCAAGTGTCCCTCACTTGCCTTGTGAAGGGA |
| | | TTCTACCCGTCCGACATCGCCGTGGAGTGGGAATCCAACGGTCAACCCGAGA |
| | | ACAACTACAAGACCACCCCTCCGGTGCTCGACTCGGATGGGTCATTCTTCCT |
| | | GTACTCCAAGCTCACCGTGGACAAGTCCAGATGGCAGCAGGGAAACGTGTTC |
| | | TCCTGCTCGGTCATGCACGAGGCCCTGCACAACCATTACACTCAGAAGTCCC |
| | | TGTCCCTGAGCCCGGGAAAA |
| 117 | CD80-K89Q- | GTGATCCACGTCACCAAGGAAGTCAAGGAAGTGGCCACCCTCTCCTGCGGTC |
| | D90Q-E23C- | ACAATGTGTCCGTGTGCGAACTGTGCCAGACGCGGATCTACTGGCAAAAGGA |
| | A26C-Fc | GAAGAAGATGGTGCTGACCATGATGAGCGGAGACATGAACATCTGGCCCGAG |
| | (Human IgG1 | TACAAGAACCGGACTATCTTTGACATCACCAACAACTTGTCGATCGTCATCC |
| | with C220S | TGGCACTCCGGCCTAGCGATGAGGGAACCTATGAGTGCGTGGTGTTGAAATA |
| | mutation) | CGAACAGCAAGCCTTCAAGAGAGAGCACCTCGCCGAAGTGACCCTGAGCGTG |
| | Nucleic acid | AAGGCCGACTTCCCCACCCCGAGCATTTCGGACTTCGAGATTCCGACCTCCA |
| | | ACATTCGCCGCATTATCTGTTCAACCTCCGGCGGATTCCCGGAGCCACATCT |
| | | GTCCTGGCTGGAGAACGGCGAAGAACTGAACGCGATTAACACTACCGTGTCC |
| | | CAAGACCCTGAAACTGAGCTGTACGCCGTGTCATCGAAGCTCGACTTCAACA |
| | | TGACTACCAACCACTCCTTCATGTGCCTGATCAAATACGGGCATCTCCGGGT |
| | | CAACCAGACCTTCAACTGGAACACTACCAAGCAGGAGCACTTTCCCGACAAT |
| | | GAGCCTAAGTCCTCGGACAAGACCCACACCTGTCCTCCATGTCCGGCGCCGG |
| | | AATTGCTTGGCGGTCCGAGCGTGTTCCTGTTCCCACCGAAGCCAAAGGACAC |
| | | CCTGATGATTAGCAGGACTCCCGAAGTCACTTGCGTGGTCGTGGATGTGTCT |
| | | CACGAGGACCCGGAAGTCAAGTTCAATTGGTACGTGGATGGCGTGGAAGTCC |
| | | ATAACGCCAAGACGAAACCCCGCGAGGAACAGTACAACAGCACCTACCGCGT |
| | | GGTGTCAGTGCTGACCGTGCTGCACCAGGATTGGCTCAACGGAAAGGAGTAC |
| | | AAGTGCAAAGTGTCGAACAAAGCCCTGCCTGCTCCCATCGAAAAGACAATCT |
| | | CGAAGGCCAAGGGACAACCCCGGGAACCTCAGGTCTACACCCTGCCTCCTTC |
| | | CCGGGAGGAAATGACCAAGAACCAAGTGTCCCTCACTTGCCTTGTGAAGGGA |
| | | TTCTACCCGTCCGACATCGCCGTGGAGTGGGAATCCAACGGTCAACCCGAGA |
| | | ACAACTACAAGACCACCCCTCCGGTGCTCGACTCGGATGGGTCATTCTTCCT |
| | | GTACTCCAAGCTCACCGTGGACAAGTCCAGATGGCAGCAGGGAAACGTGTTC |
| | | TCCTGCTCGGTCATGCACGAGGCCCTGCACAACCATTACACTCAGAAGTCCC |
| | | TGTCCCTGAGCCCGGGAAAA |
| 118 | CD80-K89Q- | GTGATCCACGTCACCAAGGAAGTCAAGGAAGTGGCCACCCTCTCCTGCGGTC |
| | D90Q-V22C- | ACAATGTGTCCTGCGAAGAACTGGCCCAGACGCGGATCTACTGGCAAAAGGA |
| | G45C-Fc | GAAGAAGATGGTGCTGACCATGATGAGCTGCGACATGAACATCTGGCCCGAG |
| | (Human IgG1 | TACAAGAACCGGACTATCTTTGACATCACCAACAACTTGTCGATCGTCATCC |
| | with C220S | TGGCACTCCGGCCTAGCGATGAGGGAACCTATGAGTGCGTGGTGTTGAAATA |
| | mutation) | CGAACAGCAAGCCTTCAAGAGAGAGCACCTCGCCGAAGTGACCCTGAGCGTG |
| | Nucleic acid | AAGGCCGACTTCCCCACCCCGAGCATTTCGGACTTCGAGATTCCGACCTCCA |
| | | ACATTCGCCGCATTATCTGTTCAACCTCCGGCGGATTCCCGGAGCCACATCT |
| | | GTCCTGGCTGGAGAACGGCGAAGAACTGAACGCGATTAACACTACCGTGTCC |
| | | CAAGACCCTGAAACTGAGCTGTACGCCGTGTCATCGAAGCTCGACTTCAACA |
| | | TGACTACCAACCACTCCTTCATGTGCCTGATCAAATACGGGCATCTCCGGGT |
| | | CAACCAGACCTTCAACTGGAACACTACCAAGCAGGAGCACTTTCCCGACAAT |
| | | GAGCCTAAGTCCTCGGACAAGACCCACACCTGTCCTCCATGTCCGGCGCCGG |
| | | AATTGCTTGGCGGTCCGAGCGTGTTCCTGTTCCCACCGAAGCCAAAGGACAC |
| | | CCTGATGATTAGCAGGACTCCCGAAGTCACTTGCGTGGTCGTGGATGTGTCT |
| | | CACGAGGACCCGGAAGTCAAGTTCAATTGGTACGTGGATGGCGTGGAAGTCC |
| | | ATAACGCCAAGACGAAACCCCGCGAGGAACAGTACAACAGCACCTACCGCGT |
| | | GGTGTCAGTGCTGACCGTGCTGCACCAGGATTGGCTCAACGGAAAGGAGTAC |
| | | AAGTGCAAAGTGTCGAACAAAGCCCTGCCTGCTCCCATCGAAAAGACAATCT |
| | | CGAAGGCCAAGGGACAACCCCGGGAACCTCAGGTCTACACCCTGCCTCCTTC |
| | | CCGGGAGGAAATGACCAAGAACCAAGTGTCCCTCACTTGCCTTGTGAAGGGA |
| | | TTCTACCCGTCCGACATCGCCGTGGAGTGGGAATCCAACGGTCAACCCGAGA |
| | | ACAACTACAAGACCACCCCTCCGGTGCTCGACTCGGATGGGTCATTCTTCCT |
| | | GTACTCCAAGCTCACCGTGGACAAGTCCAGATGGCAGCAGGGAAACGTGTTC |
| | | TCCTGCTCGGTCATGCACGAGGCCCTGCACAACCATTACACTCAGAAGTCCC |
| | | TGTCCCTGAGCCCGGGAAAA |
| 119 | CD80-K89D- | GTGATCCACGTCACCAAGGAAGTCAAGGAAGTGGCCACCCTCTCCTGCGGTC |
| | D90K-T28V- | ACAATGTGTCCGTGGAAGAACTGGCCCAGGTGCGGATCTACTGGCAAAAGGA |
| | T57V-Fc | GAAGAAGATGGTGCTGACCATGATGAGCGGAGACATGAACATCTGGCCCGAG |
| | (Human IgG1 | TACAAGAACCGGGTGATCTTTGACATCACCAACAACTTGTCGATCGTCATCC |
| | with C220S | TGGCACTCCGGCCTAGCGATGAGGGAACCTATGAGTGCGTGGTGTTGAAATA |
| | mutation) | CGAAGACAAAGCCTTCAAGAGAGAGCACCTCGCCGAAGTGACCCTGAGCGTG |
| | Nucleic acid | AAGGCCGACTTCCCCACCCCGAGCATTTCGGACTTCGAGATTCCGACCTCCA |
| | | ACATTCGCCGCATTATCTGTTCAACCTCCGGCGGATTCCCGGAGCCACATCT |
| | | GTCCTGGCTGGAGAACGGCGAAGAACTGAACGCGATTAACACTACCGTGTCC |
| | | CAAGACCCTGAAACTGAGCTGTACGCCGTGTCATCGAAGCTCGACTTCAACA |
| | | TGACTACCAACCACTCCTTCATGTGCCTGATCAAATACGGGCATCTCCGGGT |
| | | CAACCAGACCTTCAACTGGAACACTACCAAGCAGGAGCACTTTCCCGACAAT |
| | | GAGCCTAAGTCCTCGGACAAGACCCACACCTGTCCTCCATGTCCGGCGCCGG |
| | | AATTGCTTGGCGGTCCGAGCGTGTTCCTGTTCCCACCGAAGCCAAAGGACAC |
| | | CCTGATGATTAGCAGGACTCCCGAAGTCACTTGCGTGGTCGTGGATGTGTCT |
| | | CACGAGGACCCGGAAGTCAAGTTCAATTGGTACGTGGATGGCGTGGAAGTCC |
| | | ATAACGCCAAGACGAAACCCCGCGAGGAACAGTACAACAGCACCTACCGCGT |
| | | GGTGTCAGTGCTGACCGTGCTGCACCAGGATTGGCTCAACGGAAAGGAGTAC |
| | | AAGTGCAAAGTGTCGAACAAAGCCCTGCCTGCTCCCATCGAAAAGACAATCT |
| | | CGAAGGCCAAGGGACAACCCCGGGAACCTCAGGTCTACACCCTGCCTCCTTC |
| | | CCGGGAGGAAATGACCAAGAACCAAGTGTCCCTCACTTGCCTTGTGAAGGGA |
| | | TTCTACCCGTCCGACATCGCCGTGGAGTGGGAATCCAACGGTCAACCCGAGA |
| | | ACAACTACAAGACCACCCCTCCGGTGCTCGACTCGGATGGGTCATTCTTCCT |
| | | GTACTCCAAGCTCACCGTGGACAAGTCCAGATGGCAGCAGGGAAACGTGTTC |
| | | TCCTGCTCGGTCATGCACGAGGCCCTGCACAACCATTACACTCAGAAGTCCC |
| | | TGTCCCTGAGCCCGGGAAAA |
| 120 | PD-1 Ab | SYWIN |
| | VH CDR1 | |
| 121 | PD-1 Ab | NIYPGSSLTNYNEKFKN |
| | VH CDR2 | |
| 122 | PD-1 Ab | LSTGTFAY |
| | VH CDR3 | |
| 123 | PD-1 Ab VH | QVQLVQSGAEVKKPGASVKVSCKASGYTFTSYWINWVRQAPGQGLEWMGNIY |
| | | PGSSLTNYNEKFKNRVTMTRDTSTSTVYMELSSLRSEDTAVYYCARLSTGTF |
| | | AYWGQGTLVTVSS |
| 124 | PD-1 Ab | KSSQSLWDSGNQKNFLT |
| | VL CDR1 | |
| 125 | PD-1 Ab | WTSYRES |
| | VL CDR2 | |
| 126 | PD-1 Ab | QNDYFYPHT |
| | VL CDR3 | |
| 127 | PD-1 Ab VL | DIVMTQSPDSLAVSLGERATINCKSSQSLWDSGNQKNFLTWYQQKPGQPPKL |
| | | LIYWTSYRESGVPDRFSGSGSGTDFTLTISSLQAEDVAVYYCQNDYFYPHTF |
| | | GGGTKVEIK |

It is claimed:

1. A CD80-Fc fusion protein comprising (i) an antibody Fc region and (ii) a variant CD80 polypeptide comprising a substitution of one or more amino acids at position V11, V22, T28, E23, A26, Y31, Q33, K36, G45, K54, T57, D60, I61, T62, N63, N64, K89, D90, or A91 of the amino acid sequence of SEQ ID NO: 2.

2. The CD80-Fc fusion protein of claim 1, wherein the substitution is at position K36, K89, D90, and/or A91.

3. The CD80-Fc fusion protein of claim 2, wherein the substitution at position K36 is K36R, the substitution at position K89 is K89D, K89E or K89Q, the substitution at position D90 is D90K, D90N or D90Q, and the substitution at position A91 is A91S.

4. The CD80-Fc fusion protein of claim 3, wherein the substitution comprises K36R, K89D, K89E, K89Q, D90K, D90N, D90Q, A91S, K36R-K89D, K36R-K89E, K36R-K89Q, K36R-D90K, K36R-D90N, K36R-D90Q, K36R-A91S, K89D-D90K, K89D-D90N, K89D-D90Q, K89D-A91S, K89E-D90K, K89E-D90N, K89E-D90Q, K89E-A91S, K89Q-D90K, K89Q-D90N, K89Q-D90Q, K89Q-A91S, D90K-A91S, D90N-A91S, D90Q-A91S, K36R-K89D-D90K, K36R-K89D-D90N, K36R-K89D-D90Q, K36R-K89D-A91S, K36R-K89E-D90K, K36R-K89E-D90N, K36R-K89E-D90Q, K36R-K89E-A91S, K36R-K89Q-D90K, K36R-K89Q-D90N, K36R-K89Q-D90Q, K36R-K89Q-A91S, K36R-D90K-A91S, K36R-D90N-A91S, K36R-D90Q-A91S, K89D-D90K-A91S, K89D-D90N-A91S, K89D-D90Q-A91S, K89E-D90K-A91S, K89E-D90N-A91S, K89E-D90Q-A91S, K89Q-D90K-A91S, K89Q-D90N-A91S, K89Q-D90Q-A91S, K36R-K89D-D90K-A91S, K36R-K89D-D90N-A91S, K36R-K89D-D90Q-A91S, K36R-K89E-D90K-A91S, K36R-K89E-D90N-A91S, K36R-K89E-D90Q-A91S, K36R-K89Q-D90K-A91S, K36R-K89Q-D90N-A91S, or K36R-K89Q-D90Q-A91S.

5. The CD80-Fc fusion protein of claim 4, wherein the substitution comprises K89D, K89E, K89Q, D90K, D90N, D90Q, A91S, K89D-D90N, K89D-D90Q, K89D-D90K, or K89Q-D90Q.

6. The CD80-Fc fusion protein of any one of claims 1-5, wherein the substitution increases the binding affinity of a CD80-Fc fusion protein to CD28 compared to the binding affinity of a wild-type CD80-Fc fusion protein to CD28.

7. The CD80-Fc fusion protein of claim 1, wherein the substitution is at position V11, V22, T28, E23, A26, Y31, Q33, G45, K54, T57, D60, I61, T62, N63 and/or N64.

8. The CD80-Fc fusion protein of claim 7, wherein the substitution at position V11 is V11L; the substitution at position V22 is V22C, V22F or V22M; the substitution at position T28 is T28V; the substitution at position E23 is E23C; the substitution at position A26 is A26C; the substitution at position Y31 is Y31Q; the substitution at position Q33 is Q33E; the substitution at position G45 is G45C; the substitution at position K54 is K54E; the substitution at position T57 is T57V; the substitution at position D60 is D60F, D60Q, D60R, D60T or D60Y; the substitution at position I61 is I61C; the substitution at position T62 is T62F, T62I, T62L or T62Y; the substitution at position N63 is N63D or N63E; and the substitution at position N64 is N64D or N64E.

9. The CD80-Fc fusion protein of claim 8, wherein the substitution comprises V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D, N64E, V11L-V22C, V11L-V22F, V11L-V22M, V11L-T28V, V11L-E23C, V11L-A26C, V11L-Y31Q, V11L-Q33E, V11L-G45C, V11L-K54E, V11L-T57V, V11L-D60F, V11L-D60Q, V11L-D60R, V11L-D60T, V11L-D60Y, V11L-I61C, V11L-T62F, V11L-T62I, V11L-T62L, V11L-T62Y, V11L-N63D, V11L-N63E, V11L-N64D, V11L-N64E, V22C-T28V, V22C-E23C, V22C-A26C, V22C-Y31Q, V22C-Q33E, V22C-G45C, V22C-K54E, V22C-T57V, V22C-I61C, V22C-T62F, V22C-T62I, V22C-T62L, V22C-T62Y, V22C-N63D, V22C-N63E, V22C-N64D, V22C-N64E, V22F-T28V, V22F-E23C, V22F-A26C, V22F-Y31Q, V22F-Q33E, V22F-G45C, V22F-K54E, V22F-T57V, V22F-I61C, V22F-T62F, V22F-T62I, V22F-T62L, V22F-T62Y, V22F-N63D, V22F-N63E, V22F-N64D, V22F-N64E, V22M-T28V, V22M-E23C, V22M-A26C, V22M-Y31Q, V22M-Q33E, V22M-G45C, V22M-K54E, V22M-T57V, V22M-I61C, V22M-T62F, V22M-T62I, V22M-T62L, V22M-T62Y, V22M-N63D, V22M-N63E, V22M-N64D, V22M-N64E, T28V-E23C, T28V-A26C, T28V-Y31Q, T28V-Q33E, T28V-G45C, T28V-K54E, T28V-T57V, T28V-D60F, T28V-D60Q, T28V-D60R, T28V-D60T, T28V-D60Y, T28V-I61C, T28V-T62F, T28V-T62I, T28V-T62L, T28V-T62Y, T28V-N63D, T28V-N63E, T28V-N64D, T28V-N64E, E23C-A26C, E23C-Y31Q, E23C-Q33E, E23C-G45C, E23C-K54E, E23C-T57V, E23C-D60F, E23C-D60Q, E23C-D60R, E23C-D60T, E23C-D60Y, E23C-I61C, E23C-T62F, E23C-T62I, E23C-T62L, E23C-T62Y, E23C-N63D, E23C-N63E, E23C-N64D, E23C-N64E, A26C-Y31Q, A26C-Q33E, A26C-G45C, A26C-K54E, A26C-T57V, A26C-D60F, A26C-D60Q, A26C-D60R, A26C-D60T, A26C-D60Y, A26C-I61C, A26C-T62F, A26C-T62I, A26C-T62L, A26C-T62Y, A26C-N63D, A26C-N63E, A26C-N64D, A26C-N64E, Y31Q-Q33E, Y31Q-G45C, Y31Q-K54E, Y31Q-T57V, Y31Q-D60F, Y31Q-D60Q, Y31Q-D60R, Y31Q-D60T, Y31Q-D60Y, Y31Q-I61C, Y31Q-T62F, Y31Q-T62I, Y31Q-T62L, Y31Q-T62Y, Y31Q-N63D, Y31Q-N63E, Y31Q-N64D, Y31Q-N64E, Q33E-G45C, Q33E-K54E, Q33E-T57V, Q33E-D60F, Q33E-D60Q, Q33E-D60R, Q33E-D60T, Q33E-D60Y, Q33E-I61C, Q33E-T62F, Q33E-T62I, Q33E-T62L, Q33E-T62Y, Q33E-N63D, Q33E-N63E, Q33E-N64D, Q33E-N64E, G45C-K54E, G45C-T57V, G45C-D60F, G45C-D60Q, G45C-D60R, G45C-D60T, G45C-D60Y, G45C-I61C, G45C-T62F, G45C-T62I, G45C-T62L, G45C-T62Y, G45C-N63D, G45C-N63E, G45C-N64D, G45C-N64E, K54E-T57V, K54E-D60F, K54E-D60Q, K54E-D60R, K54E-D60T, K54E-D60Y, K54E-I61C, K54E-T62F, K54E-T62I, K54E-T62L, K54E-T62Y, K54E-N63D, K54E-N63E, K54E-N64D, K54E-N64E, T57V-D60F, T57V-D60Q, T57V-D60R, T57V-D60T, T57V-D60Y, T57V-I61C, T57V-T62F, T57V-T62I, T57V-T62L, T57V-T62Y, T57V-N63D, T57V-N63E, T57V-N64D, T57V-N64E, D60F-I61C, D60F-T62F, D60F-T62I, D60F-T62L, D60F-T62Y, D60F-N63D, D60F-N63E, D60F-N64D, D60F-N64E, D60E-I61C, D60E-T62F, D60E-T62I, D60E-T62L, D60E-T62Y, D60E-N63D, D60E-N63E, D60E-N64D, D60E-N64E, D60R-I61C, D60R-T62F, D60R-T62I, D60R-T62L, D60R-T62Y, D60R-N63D, D60R-N63E, D60R-N64D, D60R-N64E, D60T-I61C, D60T-T62F, D60T-T62I, D60T-T62L, D60T-T62Y, D60T-N63D, D60T-N63E, D60T-N64D, D60T-N64E, D60Y-I61C, D60Y-T62F, D60Y-T62I, D60Y-T62L, D60Y-T62Y, D60Y-N63D, D60Y-N63E, D60Y-N64D, D60Y-N64E, T62F-N63D, T62F-N63E, T62F-N64D, T62F-N64E, T62I-N63D, T62I-N63E, T62I-N64D, T62I-N64E, T62L-N63D, T62L-N63E, T62L-N64D, T62L-N64E, T62Y-N63D, T62Y-N63E, T62Y-N64D, T62Y-N64E, N63D-N64D, N63D-N64E, N63E-N64D, N63E-N64E, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, V22F-T28V-T57V-Y31Q-Q33E-K54E or any other combination of V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D and/or N64E.

10. The CD80-Fc fusion protein of claim 9, wherein the substitution comprises D60Y, I61C, V11L-V22F, V11L-T62Y, V22C-G45C, V22F-D60Y, V22F-T62L, E23C-A26C, T28V-T57V, D60F T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E.

11. The CD80-Fc fusion protein of any one of claims 1-10, wherein the substitution increases stability of a CD80-Fc fusion protein compared to the stability of a wild-type CD80-Fc fusion protein.

12. The CD80-Fc fusion protein of claim 11, wherein the increased stability provides for enhanced thermal stability, reduced thermal forced aggregation and/or reduced viscosity.

13. The CD80-Fc fusion protein of any one of claims 1-12, wherein the substitution comprises K89E-I61C, K89E-D60Y, K89E-E23C-A26C, K89E-V22C-G45C, K89E-T28V-T57V, K89E-V11L-V22F, K89E-V11L-T62Y, K89E-V22F-T62L, K89E-D60Y-T62L, K89E-V22F-K89E-D60Y, K89E-D60F-T62I, K89E-D60R-T62Y, K89E-D60Y-V11L, K89E-D60Y-V22M, K89E-D60T-T62Y, K89E-D60Q-T62F, K89E-V22F-T28V-T57V, K89E-V11L-T62Y-N63D, K89E-D60Y-V11L-N63D, K89E-V22F-T62L-N64E, K89E-D60Y-T62L-N63D, K89E-D60Y-K54E-N63E-N64D, K89E-D60Y-T62L-N63D-N64E, K89E-V22F-D60Y-K54E-N64E, K89E-V22F-T62L-N63D-N64E, K89E-T28V-T57V-Y31Q-Q33E-K54E, K89E-V22F-T28V-T57V-Y31Q-Q33E-K54E, K89Q-I61C, K89Q-D60Y, K89Q-E23C-A26C, K89Q-V22C-G45C, K89Q-T28V-T57V, K89Q-V11L-V22F, K89Q-V11L-T62Y, K89Q-V22F-T62L, K89Q-D60Y-T62L, K89Q-V22F-D60Y, K89Q-D60F-T62I, K89Q-D60R-T62Y, K89Q-D60Y-V11L, K89Q-D60Y-V22M, K89Q-D60T-T62Y, K89Q-D60Q-T62F, K89Q-V22F-T28V-T57V, K89Q-V11L-T62Y-N63D, K89Q-D60Y-V11L-N63D, K89Q-V22F-T62L-N64E, K89Q-D60Y-T62L-N63D, K89Q-D60Y-K54E-N63E-N64D, K89Q-D60Y-T62L-N63D-N64E, K89Q-V22F-D60Y-K54E-N64E, K89Q-V22F-T62L-N63D-N64E, K89Q-T28V-T57V-Y31Q-Q33E-K54E, K89Q-V22F-T28V-T57V-Y31Q-Q33E-K54E, K89D-I61C, K89D-D60Y, K89D-E23C-A26C, K89D-V22C-G45C, K89D-T28V-T57V, K89D-V11L-V22F, K89D-V11L-T62Y, K89D-V22F-T62L, K89D-D60Y-T62L, K89D-V22F-D60Y, K89D-D60F-T62I, K89D-D60R-T62Y, K89D-D60Y-V11L, K89D-D60Y-V22M, K89D-D60T-T62Y, K89D-D60Q-T62F, K89D-V22F-T28V-T57V, K89D-V11L-T62Y-N63D, K89D-D60Y-V11L-N63D, K89D-V22F-T62L-N64E, K89D-D60Y-T62L-N63D, K89D-D60Y-K54E-N63E-N64D, K89D-D60Y-T62L-N63D-N64E, K89D-V22F-D60Y-K54E-N64E, K89D-V22F-T62L-N63D-N64E, K89D-T28V-T57V-Y31Q-Q33E-K54E, K89D-V22F-T28V-T57V-Y31Q-Q33E-K54E,D90K-I61C, D90K-D60Y, D90K-E23C-A26C, D90K-V22C-G45C, D90K-T28V-T57V, D90K-V11L-V22F, D90K-V11L-T62Y, D90K-V22F-T62L, D90K-D60Y-T62L, D90K-V22F-D60Y, D90K-D60F-T62I, D90K-D60R-T62Y, D90K-D60Y-V11L, D90K-D60Y-V22M, D90K-D60T-T62Y, D90K-D60Q-T62F, D90K-V22F-T28V-T57V, D90K-V11L-T62Y-N63D, D90K-D60Y-V11L-N63D, D90K-V22F-T62L-N64E, D90K-D60Y-T62L-N63D, D90K-D60Y-K54E-N63E-N64D, D90K-D60Y-T62L-N63D-N64E, D90K-V22F-D60Y-K54E-N64E, D90K-V22F-T62L-N63D-N64E, D90K-T28V-T57V-Y31Q-Q33E-K54E, D90K-V22F-T28V-T57V-Y31Q-Q33E-K54E, D90N-I61C, D90N-D60Y, D90N-E23C-A26C, D90N-V22C-G45C, D90N-T28V-T57V, D90N-V11L-V22F, D90N-V11L-T62Y, D90N-V22F-T62L, D90N-D60Y-T62L, D90N-V22F-D60Y, D90N-D60F-T62I, D90N-D60R-T62Y, D90N-D60Y-V11L, D90N-D60Y-V22M, D90N-D60T-T62Y, D90N-D60Q-T62F, D90N-V22F-T28V-T57V, D90N-V11L-T62Y-N63D, D90N-D60Y-V11L-N63D, D90N-V22F-T62L-N64E, D90N-D60Y-T62L-N63D, D90N-D60Y-K54E-N63E-N64D, D90N-D60Y-T62L-N63D-N64E, D90N-V22F-D60Y-K54E-N64E, D90N-V22F-T62L-N63D-N64E, D90N-T28V-T57V-Y31Q-Q33E-K54E, D90N-V22F-T28V-T57V-Y31Q-Q33E-K54E, D90Q-I61C, D90Q-D60Y, D90Q-E23C-A26C, D90Q-V22C-G45C, D90Q-T28V-T57V, D90Q-V11L-V22F, D90Q-V11L-T62Y, D90Q-V22F-T62L, D90Q-D60Y-T62L, D90Q-V22F-D60Y, D90Q-D60F-T62I, D90Q-D60R-T62Y, D90Q-D60Y-V11L, D90Q-D60Y-V22M, D90Q-D60T-T62Y, D90Q-D60Q-T62F, D90Q-V22F-T28V-T57V, D90Q-V11L-T62Y-N63D, D90Q-D60Y-V11L-N63D, D90Q-V22F-T62L-N64E, D90Q-D60Y-T62L-N63D, D90Q-D60Y-K54E-N63E-N64D, D90Q-D60Y-T62L-N63D-N64E, D90Q-V22F-D60Y-K54E-N64E, D90Q-V22F-T62L-N63D-N64E, D90Q-T28V-T57V-Y31Q-Q33E-K54E, D90Q-V22F-T28V-T57V-Y31Q-Q33E-K54E, K89Q-D90Q-I61C, K89Q-D90Q-D60Y, K89Q-D90Q-E23C-A26C, K89Q-D90Q-V22C-G45C, K89Q-D90Q-T28V-T57V, K89Q-D90Q-V11L-V22F, K89Q-D90Q-V11L-T62Y, K89Q-D90Q-V22F-T62L, K89Q-D90Q-D60Y-T62L, K89Q-D90Q-V22F-D60Y, K89Q-D90Q-D60F-T62I, K89Q-D90Q-D60R-T62Y, K89Q-D90Q-D60Y-V11L, K89Q-D90Q-D60Y-V22M, K89Q-D90Q-D60T-T62Y, K89Q-D90Q-D60Q-T62F, K89Q-D90Q-V22F-T28V-T57V, K89Q-D90Q-V11L-T62Y-N63D, K89Q-D90Q-D60Y-V11L-N63D, K89Q-D90Q-V22F-T62L-N64E, K89Q-D90Q-D60Y-T62L-N63D, K89Q-D90Q-D60Y-K54E-N63E-N64D, K89Q-D90Q-D60Y-T62L-N63D-N64E, K89Q-D90Q-V22F-D60Y-K54E-N64E, K89Q-D90Q-V22F-T62L-N63D-N64E, K89Q-D90Q-T28V-T57V-Y31Q-Q33E-K54E, K89Q-D90Q-V22F-T28V-T57V-Y31Q-Q33E-K54E, K89D-D90N-I61C, K89D-D90N-D60Y, K89D-D90N-E23C-A26C, K89D-D90N-V22C-G45C, K89D-D90N-T28V-T57V, K89D-D90N-V11L-V22F, K89D-D90N-V11L-T62Y, K89D-D90N-V22F-T62L, K89D-D90N-D60Y-T62L, K89D-D90N-V22F-D60Y, K89D-D90N-D60F-T62I, K89D-D90N-D60R-T62Y, K89D-D90N-D60Y-V11L, K89D-D90N-D60Y-V22M, K89D-D90N-D60T-T62Y, K89D-D90N-D60Q-T62F, K89D-D90N-V22F-T28V-T57V, K89D-D90N-V11L-T62Y-N63D, K89D-D90N-D60Y-V11L-N63D, K89D-D90N-V22F-T62L-N64E, K89D-D90N-D60Y-T62L-N63D, K89D-D90N-D60Y-K54E-N63E-N64D, K89D-D90N-D60Y-T62L-N63D-N64E, K89D-D90N-V22F-D60Y-K54E-N64E, K89D-D90N-V22F-T62L-N63D-N64E, K89D-D90N-T28V-T57V-Y31Q-Q33E-K54E, K89D-D90N-V22F-T28V-T57V-Y31Q-Q33E-K54E, K89D-D90Q-I61C, K89D-D90Q-D60Y, K89D-D90Q-E23C-A26C, K89D-D90Q-V22C-G45C, K89D-D90Q-T28V-T57V, K89D-D90Q-V11L-V22F, K89D-D90Q-V11L-T62Y, K89D-D90Q-V22F-T62L, K89D-D90Q-D60Y-T62L, K89D-D90Q-V22F-D60Y, K89D-D90Q-D60F-T62I, K89D-D90Q-D60R-T62Y, K89D-D90Q-D60Y-V11L, K89D-D90Q-D60Y-V22M, K89D-D90Q-D60T-T62Y, K89D-D90Q-D60Q-T62F, K89D-D90Q-V22F-T28V-T57V, K89D-D90Q-V11L-T62Y-N63D, K89D-D90Q-D60Y-V11L-N63D, K89D-D90Q-V22F-T62L-N64E, K89D-D90Q-D60Y-T62L-N63D, K89D-D90Q-D60Y-K54E-N63E-N64D, K89D-D90Q-D60Y-T62L-N63D-N64E, K89D-D90Q-V22F-D60Y-K54E-N64E, K89D-D90Q-V22F-T62L-N63D-N64E, K89D-D90Q-T28V-T57V-Y31Q-Q33E-K54E, K89D-D90Q-V22F-T28V-T57V-Y31Q-Q33E-K54E, K89D-D90K-I61C, K89D-D90K-D60Y, K89D-D90K-E23C-A26C, K89D-D90K-V22C-G45C, K89D-D90K-T28V-T57V, K89D-D90K-V11L-V22F, K89D-D90K-V11L-T62Y, K89D-D90K-V22F-T62L, K89D-D90K-D60Y-T62L, K89D-D90K-V22F-D60Y, K89D-D90K-D60F-T62I, K89D-D90K-D60R-T62Y, K89D-D90K-D60Y-V11L, K89D-D90K-D60Y-V22M, K89D-D90K-D60T-T62Y, K89D-D90K-D60Q-T62F, K89D-D90K-V22F-T28V-T57V, K89D-D90K-V11L-T62Y-N63D, K89D-D90K-D60Y-V11L-N63D, K89D-D90K-V22F-T62L-N64E, K89D-D90K-D60Y-T62L-N63D, K89D-D90K-D60Y-K54E-N63E-N64D, K89D-D90K-D60Y-T62L-N63D-N64E, K89D-D90K-V22F-D60Y-K54E-N64E, K89D-D90K-V22F-T62L-N63D-N64E, K89D-D90K-T28V-T57V-Y31Q-Q33E-K54E, K89D-D90K-V22F-T28V-T57V-Y31Q-Q33E-K54E, A91S-I61C, A91S-D60Y, A91S-E23C-A26C, A91S-V22C-G45C, A91S-T28V-T57V, A91S-V11L-V22F, A91S-V11L-T62Y, A91S-V22F-T62L, A91S-D60Y-T62L, A91S-V22F-D60Y, A91S-D60F-T62I, A91S-D60R-T62Y, A91S-D60Y-V11L, A91S-D60Y-V22M, A91S-D60T-T62Y, A91S-D60Q-T62F, A91S-V22F-T28V-T57V, A91S-V11L-T62Y-N63D, A91S-D60Y-V11L-N63D, A91S-V22F-T62L-N64E, A91S-D60Y-T62L-N63D, A91S-D60Y-K54E-N63E-N64D, A91S-D60Y-T62L-N63D-N64E, A91S-V22F-D60Y-K54E-N64E, A91S-V22F-T62L-N63D-N64E, A91S-T28V-T57V-Y31Q-Q33E-K54E, or A91S-V22F-T28V-T57V-Y31Q-Q33E-K54E.

14. The CD80-Fc fusion protein of any one of claims 1-13, wherein the substitution comprises D90Q.

15. The CD80-Fc fusion protein of any one of claims 1-13, wherein the substitution comprises K89Q-D90Q.

16. The CD80-Fc fusion protein of any one of claims 1-13, wherein the substitution comprises K89Q-D90Q-E23C-A26C.

17. The CD80-Fc fusion protein of any one of claims 1-13, wherein the substitution comprises K89D-D90K-T28V-T57V.

18. A CD80-Fc fusion protein comprising (i) an antibody Fc region and (ii) a variant CD80 polypeptide comprising a variant CD80 polypeptide comprises i) a first substitution at position K36, K89, D90, and/or A91 of the amino acid sequence of SEQ ID NO: 2, and ii) a second substitution at position V11, V22, T28, E23, A26, Y31, Q33, G45, K54, T57, D60, I61, T62, N63 and/or N64 of the amino acid sequence of SEQ ID NO: 2.

19. The CD80-Fc fusion protein of claim 18, wherein i) the first substitution at position K36 is K36R, the first substitution at position K89 is K89D, K89E or K89Q, and the first substitution at position D90 is D90K or D90Q, and the first substitution at position A91 is A91S, and ii) the second substitution at position V11 is V11L; the second substitution at position V22 is V22C, V22F or V22M; the second substitution at position T28 is T28V; the second substitution at position E23 is E23C; the second substitution at position A26 is A26C; the second substitution at position Y31 is Y31Q; the second substitution at position Q33 is Q33E; the second substitution at position G45 is G45C; the second substitution at position K54 is K54E; the second substitution at position T57 is T57V; the second substitution at position D60 is D60F, D60Q, D60R, D60T or D60Y; the second substitution at position I61 is I61C; the second substitution at position T62 is T62F, T62I, T62L or T62Y; the second substitution at position N63 is N63D or N63E; and the second substitution at position N64 is N64D or N64E.

20. The CD80-Fc fusion protein of claim 18 or 19, wherein i) the first substitution comprises K36R, K89D, K89E, K89Q, D90K, D90N, D90Q, A91S, K36R-K89D, K36R-K89E, K36R-K89Q, K36R-D90K, K36R-D90N, K36R-D90Q, K36R-A91S, K89D-D90K, K89D-D90N, K89D-D90Q, K89D-A91S, K89E-D90K, K89E-D90N, K89E-D90Q, K89E-A91S, K89Q-D90K, K89Q-D90N, K89Q-D90Q, K89Q-A91S, D90K-A91S, D90N-A91S, D90Q-A91S, K36R-K89D-D90K, K36R-K89D-D90N, K36R-K89D-D90Q, K36R-K89D-A91S, K36R-K89E-D90K, K36R-K89E-D90N, K36R-K89E-D90Q, K36R-K89E-A91S, K36R-K89Q-D90K, K36R-K89Q-D90N, K36R-K89Q-D90Q, K36R-K89Q-A91S, K36R-D90K-A91S, K36R-D90N-A91S, K36R-D90Q-A91S, K89D-D90K-A91S, K89D-D90N-A91S, K89D-D90Q-A91S, K89E-D90K-A91S, K89E-D90N-A91S, K89E-D90Q-A91S, K89Q-D90K-A91S, K89Q-D90N-A91S, K89Q-D90Q-A91S, K36R-K89D-D90K-A91S, K36R-K89D-D90N-A91S, K36R-K89D-D90Q-A91S, K36R-K89E-D90K-A91S, K36R-K89E-D90N-A91S, K36R-K89E-D90Q-A91S, K36R-K89Q-D90K-A91S, K36R-K89Q-D90N-A91S, or K36R-K89Q-D90Q-A91S, and ii) the second substitution comprises V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D, N64E, V11L-V22C, V11L-V22F, V11L-V22M, V11L-T28V, V11L-E23C, V11L-A26C, V11L-Y31Q, V11L-Q33E, V11L-G45C, V11L-K54E, V11L-T57V, V11L-D60F, V11L-D60Q, V11L-D60R, V11L-D60T, V11L-D60Y, V11L-I61C, V11L-T62F, V11L-T62I, V11L-T62L, V11L-T62Y, V11L-N63D, V11L-N63E, V11L-N64D, V11L-N64E, V22C-T28V, V22C-E23C, V22C-A26C, V22C-Y31Q, V22C-Q33E, V22C-G45C, V22C-K54E, V22C-T57V, V22C-I61C, V22C-T62F, V22C-T62I, V22C-T62L, V22C-T62Y, V22C-N63D, V22C-N63E, V22C-N64D, V22C-N64E, V22F-T28V, V22F-E23C, V22F-A26C, V22F-Y31Q, V22F-Q33E, V22F-G45C, V22F-K54E, V22F-T57V, V22F-I61C, V22F-T62F, V22F-T62I, V22F-T62L, V22F-T62Y, V22F-N63D, V22F-N63E, V22F-N64D, V22F-N64E, V22M-T28V, V22M-E23C, V22M-A26C, V22M-Y31Q, V22M-Q33E, V22M-G45C, V22M-K54E, V22M-T57V, V22M-I61C, V22M-T62F, V22M-T62I, V22M-T62L, V22M-T62Y, V22M-N63D, V22M-N63E, V22M-N64D, V22M-N64E, T28V-E23C, T28V-A26C, T28V-Y31Q, T28V-Q33E, T28V-G45C, T28V-K54E, T28V-T57V, T28V-D60F, T28V-D60Q, T28V-D60R, T28V-D60T, T28V-D60Y, T28V-I61C, T28V-T62F, T28V-T62I, T28V-T62L, T28V-T62Y, T28V-N63D, T28V-N63E, T28V-N64D, T28V-N64E, E23C-A26C, E23C-Y31Q, E23C-Q33E, E23C-G45C, E23C-K54E, E23C-T57V, E23C-D60F, E23C-D60Q, E23C-D60R, E23C-D60T, E23C-D60Y, E23C-I61C, E23C-T62F, E23C-T62I, E23C-T62L, E23C-T62Y, E23C-N63D, E23C-N63E, E23C-N64D, E23C-N64E, A26C-Y31Q, A26C-Q33E, A26C-G45C, A26C-K54E, A26C-T57V, A26C-D60F, A26C-D60Q, A26C-D60R, A26C-D60T, A26C-D60Y, A26C-I61C, A26C-T62F, A26C-T62I, A26C-T62L, A26C-T62Y, A26C-N63D, A26C-N63E, A26C-N64D, A26C-N64E, Y31Q-Q33E, Y31Q-G45C, Y31Q-K54E, Y31Q-T57V, Y31Q-D60F, Y31Q-D60Q, Y31Q-D60R, Y31Q-D60T, Y31Q-D60Y, Y31Q-I61C, Y31Q-T62F, Y31Q-T62I, Y31Q-T62L, Y31Q-T62Y, Y31Q-N63D, Y31Q-N63E, Y31Q-N64D, Y31Q-N64E, Q33E-G45C, Q33E-K54E, Q33E-T57V, Q33E-D60F, Q33E-D60Q, Q33E-D60R, Q33E-D60T, Q33E-D60Y, Q33E-I61C, Q33E-T62F, Q33E-T62I, Q33E-T62L, Q33E-T62Y, Q33E-N63D, Q33E-N63E, Q33E-N64D, Q33E-N64E, G45C-K54E, G45C-T57V, G45C-D60F, G45C-D60Q, G45C-D60R, G45C-D60T, G45C-D60Y, G45C-I61C, G45C-T62F, G45C-T62I, G45C-T62L, G45C-T62Y, G45C-N63D, G45C-N63E, G45C-N64D, G45C-N64E, K54E-T57V, K54E-D60F, K54E-D60Q, K54E-D60R, K54E-D60T, K54E-D60Y, K54E-I61C, K54E-T62F, K54E-T62I, K54E-T62L, K54E-T62Y, K54E-N63D, K54E-N63E, K54E-N64D, K54E-N64E, T57V-D60F, T57V-D60Q, T57V-D60R, T57V-D60T, T57V-D60Y, T57V-I61C, T57V-T62F, T57V-T62I, T57V-T62L, T57V-T62Y, T57V-N63D, T57V-N63E, T57V-N64D, T57V-N64E, D60F-I61C, D60F-T62F, D60F-T62I, D60F-T62L, D60F-T62Y, D60F-N63D, D60F-N63E, D60F-N64D, D60F-N64E, D60E-I61C, D60E-T62F, D60E-T62I, D60E-T62L, D60E-T62Y, D60E-N63D, D60E-N63E, D60E-N64D, D60E-N64E, D60R-I61C, D60R-T62F, D60R-T62I, D60R-T62L, D60R-T62Y, D60R-N63D, D60R-N63E, D60R-N64D, D60R-N64E, D60T-I61C, D60T-T62F, D60T-T62I, D60T-T62L, D60T-T62Y, D60T-N63D, D60T-N63E, D60T-N64D, D60T-N64E, D60Y-I61C, D60Y-T62F, D60Y-T62I, D60Y-T62L, D60Y-T62Y, D60Y-N63D, D60Y-N63E, D60Y-N64D, D60Y-N64E, T62F-N63D, T62F-N63E, T62F-N64D, T62F-N64E, T62I-N63D, T62I-N63E, T62I-N64D, T62I-N64E, T62L-N63D, T62L-N63E, T62L-N64D, T62L-N64E, T62Y-N63D, T62Y-N63E, T62Y-N64D, T62Y-N64E, N63D-N64D, N63D-N64E, N63E-N64D, N63E-N64E, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, V22F-T28V-T57V-Y31Q-Q33E-K54E or any other combination of V11L, V22C, V22F, V22M, T28V, E23C, A26C, Y31Q, Q33E, G45C, K54E, T57V, D60F, D60Q, D60R, D60T, D60Y, I61C, T62F, T62I, T62L, T62Y, N63D, N63E, N64D and/or N64E.

21. The CD80-Fc fusion protein of any one of claims 18-20, wherein i) the first substitution comprises K89D K89E, K89Q, D90K, D90N, D90Q, A91S, K89D-D90N, K89D-D90Q, K89D-D90K, or K89Q-D90Q, and ii) the second substitution comprises D60Y, I61C, V11L-V22F, V11L-T62Y, V22C-G45C, V22F-D60Y, V22F-T62L, E23C-A26C, T28V-T57V, D60F T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E.

22. The CD80-Fc fusion protein of claim 21, wherein i) the first substitution comprises K89Q-D90Q and ii) the second substitution comprises E23C-A26C.

23. The CD80-Fc fusion protein of claim 21, wherein i) the first substitution comprises K89D-D90K and ii) the second substitution comprises T28V-T57V.

24. The CD80-Fc fusion protein of any one of claims 18-23, wherein i) the first substitution increases the binding affinity of a CD80-Fc fusion protein to CD28 compared to the binding affinity of a wild-type CD80-Fc fusion protein to CD28, and ii) the second substitution increases stability of a CD80-Fc fusion protein compared to the stability of a wild-type CD80-Fc fusion protein.

25. The CD80-Fc fusion protein of any one of claim 24, wherein the increased stability provides for enhanced thermal stability, reduced thermal forced aggregation and/or reduced viscosity.

26. The CD80-Fc fusion protein of any one of claims 1-25, wherein the CD80-Fc fusion protein i) does not increase or enhance binding to PD-L1, or ii) demonstrates minimal or no detectable binding to PD-L1.

27. The CD80-Fc fusion protein of any one of claims 1-26, wherein the variant CD80 polypeptide comprises the amino acid sequence of any of SEQ ID NO: 20-63.

28. The CD80-Fc fusion protein of any one of claims 1-27, wherein the antibody Fc region is derived from IgG1, IgG2 or IgG4.

29. The CD80-Fc fusion protein of claim 28, wherein the antibody Fc region comprises the amino acid sequence of any of SEQ ID NO. 13-18.

30. The CD80-Fc fusion protein of any one of claims 1-29, wherein the antibody Fc region is linked to the variant CD80 polypeptide.

31. The CD80-Fc fusion protein of any one of claims 1-30, wherein the CD80-Fc fusion protein comprises the amino acid sequence of any of SEQ ID NO: 64-114.

32. An isolated cell line that produces the CD80-Fc fusion protein of any one of claims 1-31.

33. An isolated nucleic acid encoding the CD80-Fc fusion protein of any one of claims 1-31.

34. A vector comprising the nucleic acid of claim 33.

35. A host cell comprising the nucleic acid of claim 33 or the vector of claim 34.

36. A method of producing a CD80-Fc fusion protein, comprising culturing the host cell of claim 35 under conditions that result in the production of the CD80-Fc fusion protein of any one of claims 1-31, and purifying the produced CD80-Fc fusion protein.

37. A pharmaceutical composition comprising the CD80-Fc fusion protein of any one of claims 1-31 and a pharmaceutically acceptable carrier.

38. A method for treating cancer in a subject in need thereof, the method comprising administering to the subject an effective amount of the CD80-Fc fusion protein of any one of claims 1-31 or the pharmaceutical composition of claim 37.

39. The method of claim 38, wherein the cancer is gastric cancer, small intestine cancer, sarcoma, lymphoma, Hodgkin's lymphoma, leukemia, multiple myeloma, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer (e.g., non-small-cell lung cancer), mesothelioma, ovarian cancer, breast cancer, prostate cancer, esophageal cancer, pancreatic cancer, glioma, renal cancer (e.g., renal cell carcinoma), bladder cancer, cervical cancer, uterine cancer, vulvar cancer, penile cancer, testicular cancer, anal cancer, choriocarcinoma, colon cancer, colorectal cancer, oral cancer, skin cancer, Merkel cell carcinoma, glioblastoma, brain tumor, bone cancer, eye cancer, melanoma, or cancer with high microsatellite instability (MSI-H).

40. The method of claim 38 or 39, wherein the cancer is relapsed, resistant, refractory, and/or metastatic.

41. The method of any one of claims 38-40, wherein the cancer is resistant and/or refractory to anti-PD-1 and/or anti-PD-L1 therapies

42. A method of enhancing an immune response in a subject in need thereof, the method comprising administering to the subject an effective amount of the CD80-Fc fusion protein of any one of claims 1-31 or the pharmaceutical composition of claim 37.

43. The method of any one of claims 38-42, wherein the method further comprises administering an effective amount of one or more additional agents.

44. The method of claim 43, wherein the one or more additional agents is an antibody selected from the group consisting of an anti-CTLA-4 antibody, an anti-CD3 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, and an anti-GITR antibody.

45. The method of claim 43, wherein the one or more additional agents is a cytokine, an immunocytokine, a targeted cytokine, TNFα, a PARP inhibitor, an oncolytic virus, a kinase inhibitor, an ALK inhibitor, a MEK inhibitor, an IDO inhibitor, a GLS1 inhibitor, a tyrosine kinase inhibitor, a CART cell or T cell therapy, a TLR agonist, cancer vaccine, KRAS inhibitor, BRAF inhibitor, PI3K inhibitor, EGFR inhibitor, HPK1 inhibitor, CDK or other cell cycle inhibitor, EZH2 inhibitor or other epigenetic modifier, anti-estrogen or anti-androgen therapy, radiation therapy, chemotherapy, a PRR agonist, a bispecific or multispecific antibody, an antibody-drug conjugate or other innate immune modulator.

46. The method of any one of claims 43-45, wherein the one or more additional agents is an anti-PD-1 antibody, a bispecific antibody, a CDK inhibitor and/or chemotherapy.

47. Use of the CD80-Fc fusion protein on any one of claims 1-31 or the pharmaceutical composition of claim 37, the isolate nucleic acid of claim 33, the vector of claim 34, or the host cell of claim 35 in the manufacture of a medicament.

48. The CD80-Fc fusion protein of any one of claims 1-31 or the pharmaceutical composition of claim 37, for use as a medicament.

49. The CD80-Fc fusion protein or pharmaceutical composition of claim 48, wherein the medicament is for use in the treatment of cancer.

50. A variant CD80 polypeptide comprising a substitution of one or more amino acids at position V11, V22, T28, E23, A26, Y31, Q33, K36, G45, K54, T57, D60, 161, T62, N63, N64, K89, D90, or A91 of the amino acid sequence of SEQ ID NO: 2.

51. The variant CD80 polypeptide of claim 50, wherein the substitution at position V11 is V11L; the substitution at position V22 is V22C, V22F or V22M; the substitution at position T28 is T28V; the substitution at position E23 is E23C; the substitution at position A26 is A26C; the substitution at position Y31 is Y31Q; the substitution at position Q33 is Q33E; the substitution at position K36 is K36R, the substitution at position G45 is G45C; the substitution at position K54 is K54E; the substitution at position T57 is T57V; the substitution at position D60 is D60F, D60Q, D60R, D60T or D60Y; the substitution at position I61 is I61C; the substitution at position T62 is T62F, T62I, T62L or T62Y; the substitution at position N63 is N63D or N63E; and the substitution at position N64 is N64D or N64E; the substitution at position K89 is K89D, K89E or K89Q; the substitution at position D90 is D90K, D90N or D90Q; and the substitution at position A91 is A91S.

52. The variant CD80 polypeptide of claim 50 or 51, wherein the substitution comprises K89D, K89E, K89Q, D90K, D90N, D90Q, A91S, K89D-D90N, K89D-D90Q, K89D-D90K, K89Q-D90Q, D60Y, I61C, V11L-V22F, V11L-T62Y, V22C-G45C, V22F-D60Y, V22F-T62L, E23C-A26C, T28V-T57V, D60F-T62I, D60Q-T62F, D60R-T62Y, D60T-T62Y, D60Y-V11L, D60Y-V22M, D60Y-T62L, V11L-T62Y-N63D, V22F-T28V-T57V, V22F-T62L-N64E, D60Y-V11L-N63D, D60Y-T62L-N63D, V22F-D60Y-K54E-N64E, V22F-T62L-N63D-N64E, D60Y-K54E-N63E-N64D, D60Y-T62L-N63D-N64E, T28V-T57V-Y31Q-Q33E-K54E, or V22F-T28V-T57V-Y31Q-Q33E-K54E, K89Q-D90Q-I61C, D90Q-E23C-A26C, K89Q-D90Q-E23C-A26C, or K89Q-D90Q-V22C-G45C, or K89D-D90K-T28V-T57V of the amino acid sequence of SEQ ID NO: 2.


SEQUENCE LISTING (127 sequences)

SEQ ID NO: 1 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 2 - protein, 208 aa, Homo sapiens [sequence omitted]

SEQ ID NO: 3 - protein, 5 aa, Artificial Sequence
  Synthetic Peptide Sequence
EPKSC

SEQ ID NO: 4 - protein, 5 aa, Artificial Sequence
  Synthetic Peptide Sequence
EPKSS

SEQ ID NO: 5 - protein, 14 aa, Artificial Sequence
  Synthetic Peptide Sequence
GGGGGTSATATPGA

SEQ ID NO: 6 - protein, 9 aa, Artificial Sequence
  Synthetic Peptide Sequence
GGGGSGSGG

SEQ ID NO: 7 - protein, 14 aa, Artificial Sequence
  Synthetic Peptide Sequence
GGGGGTSATATPGA

SEQ ID NO: 8 - protein, 19 aa, Artificial Sequence
  Synthetic Peptide Sequence
GGSGGGGSGGGSGGGGSGG

SEQ ID NO: 9 - protein, 15 aa, Artificial Sequence
  Synthetic Peptide Sequence
SGGGGSGGGGSGGGG

SEQ ID NO: 10 - protein, 6 aa, Artificial Sequence
  Synthetic Peptide Sequence
HHHHHH

SEQ ID NO: 11 - protein, 8 aa, Artificial Sequence
  Synthetic Peptide Sequence
HHHHHHHH

SEQ ID NO: 12 - protein, 15 aa, Artificial Sequence
  Synthetic Peptide Sequence
GLNDIFEAQKIEWHE

SEQ ID NO: 13 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 227 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 15 - protein, 228 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 231 aa, Artificial Sequence
  Synthetic Peptide Sequence
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 231 aa, Artificial Sequence
  Synthetic Peptide Sequence
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 227 aa, Artificial Sequence
  Synthetic Peptide Sequence
ERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 19 - protein, 9 aa, Artificial Sequence
  Synthetic Peptide Sequence
SLLMWITQC

SEQ ID NO: 20 - protein, 208 aa, Artificial Sequence
  Synthetic Peptide Sequence
VIHVTKEVKEVATLSCGHNVSVEELAQTRIYWQKERKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 21 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 22 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEEDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 23 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEQDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 24 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKKAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 25 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKNAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 26 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 27 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKDSFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 28 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDKAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 29 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 30 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDNAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 31 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 32 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDCTNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 33 - protein, 208 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 34 - protein, 208 aa, Artificial Sequence
  Synthetic Peptide Sequence
VIHVTKEVKEVATLSCGHNVSCEELAQTRIYWQKEKKMVLTMMSCDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 35 - protein, 208 aa, Artificial Sequence
  Synthetic Peptide Sequence
VIHVTKEVKELATLSCGHNVSFEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 36 - protein, 208 aa, Artificial Sequence
  Synthetic Peptide Sequence
VIHVTKEVKELATLSCGHNVSVEELAQTRIYWQKEKKMVLTMMSGDMNIWPEYKNRTIFDIYNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 37 - protein, 208 aa, Artificial Sequence
  Synthetic Peptide Sequence
VIHVTKEVKELATLSCGHNVSVEELAQTRIYWQKEKKMVLTMMSGDMNIWPEYKNRTIFDIYDNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 38 - protein, 208 aa, Artificial Sequence
  Synthetic Peptide Sequence
VIHVTKEVKEVATLSCGHNVSFEELAQTRIYWQKEKKMVLTMMSGDMNIWPEYKNRTIFDILNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 39 - protein, 208 aa, Artificial Sequence
  Synthetic Peptide Sequence
VIHVTKEVKEVATLSCGHNVSVEELAQVRIYWQKEKKMVLTMMSGDMNIWPEYKNRVIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 40 - protein, 208 aa, Artificial Sequence
  Synthetic Peptide Sequence
VIHVTKEVKEVATLSCGHNVSVEELAQVRIQWEKEKKMVLTMMSGDMNIWPEYENRVIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 41 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 42 - protein, 208 aa, Artificial Sequence
  Synthetic Peptide Sequence
VIHVTKEVKEVATLSCGHNVSVEELAQTRIYWQKEKKMVLTMMSGDMNIWPEYENRTIFYITEDLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 43 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFYILNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 44 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFYILDELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 45 - protein, 208 aa, Artificial Sequence
  Synthetic Peptide Sequence
VIHVTKEVKEVATLSCGHNVSFEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 46 - protein, 208 aa, Artificial Sequence
  Synthetic Peptide Sequence
VIHVTKEVKEVATLSCGHNVSFEELAQTRIYWQKEKKMVLTMMSGDMNIWPEYENRTIFYITNELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 47 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFFIINNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 48 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFRIYNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 49 - protein, 208 aa, Artificial Sequence
  Synthetic Peptide Sequence
VIHVTKEVKELATLSCGHNVSVEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 50 - protein, 208 aa, Artificial Sequence
  Synthetic Peptide Sequence
VIHVTKEVKELATLSCGHNVSVEELAQTRIYWQKEKKMVLTMMSGDMNIWPEYKNRTIFYITDNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 51 - protein, 208 aa, Artificial Sequence
  Synthetic Peptide Sequence
VIHVTKEVKEVATLSCGHNVSMEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 52 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFTIYNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 53 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFQIFNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 54 - protein, 208 aa, Artificial Sequence
  Synthetic Peptide Sequence
VIHVTKEVKEVATLSCGHNVSFEELAQVRIYWQKEKKMVLTMMSGDMNIWPEYKNRVIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 55 - protein, 208 aa, Artificial Sequence; identical to SEQ ID NO: 40
  Synthetic Peptide Sequence

SEQ ID NO: 56 - protein, 208 aa, Artificial Sequence
  Synthetic Peptide Sequence
VIHVTKEVKEVATLSCGHNVSFEELAQTRIYWQKEKKMVLTMMSGDMNIWPEYKNRTIFDILNELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 57 - protein, 208 aa, Artificial Sequence
  Synthetic Peptide Sequence
VIHVTKEVKEVATLSCGHNVSFEELAQTRIYWQKEKKMVLTMMSGDMNIWPEYKNRTIFDILDELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 58 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFYILDNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 59 - protein, 208 aa, Artificial Sequence
  Synthetic Peptide Sequence
VIHVTKEVKEVATLSCGHNVSVEELAQTRIYWQKEKKMVLTMMSGDMNIWPEYKNRTIFDCTNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 60 - protein, 208 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEKQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 61 - protein, 208 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 62 - protein, 208 aa, Artificial Sequence
  Synthetic Peptide Sequence
VIHVTKEVKEVATLSCGHNVSCEELAQTRIYWQKEKKMVLTMMSCDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 63 - protein, 208 aa, Artificial Sequence
  Synthetic Peptide Sequence
VIHVTKEVKEVATLSCGHNVSVEELAQVRIYWQKEKKMVLTMMSGDMNIWPEYKNRVIFDITNNLSIVILALRPSDEGTYECVVLKYEDKAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 64 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 439 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEKQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 439 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 439 aa, Artificial Sequence
  Synthetic Peptide Sequence
VIHVTKEVKEVATLSCGHNVSVEELAQVRIYWQKEKKMVLTMMSGDMNIWPEYKNRVIFDITNNLSIVILALRPSDEGTYECVVLKYEDKAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 439 aa, Artificial Sequence
  Synthetic Peptide Sequence
VIHVTKEVKEVATLSCGHNVSVEELAQTRIYWQKERKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEEDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEQDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKKAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKNAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEKDSFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDKAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEDNAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDCTNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 439 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 439 aa, Artificial Sequence
  Synthetic Peptide Sequence
VIHVTKEVKEVATLSCGHNVSCEELAQTRIYWQKEKKMVLTMMSCDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 439 aa, Artificial Sequence
  Synthetic Peptide Sequence
VIHVTKEVKELATLSCGHNVSFEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 439 aa, Artificial Sequence
  Synthetic Peptide Sequence
VIHVTKEVKELATLSCGHNVSVEELAQTRIYWQKEKKMVLTMMSGDMNIWPEYKNRTIFDIYNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 439 aa, Artificial Sequence
  Synthetic Peptide Sequence
VIHVTKEVKELATLSCGHNVSVEELAQTRIYWQKEKKMVLTMMSGDMNIWPEYKNRTIFDIYDNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 439 aa, Artificial Sequence
  Synthetic Peptide Sequence
VIHVTKEVKEVATLSCGHNVSFEELAQTRIYWQKEKKMVLTMMSGDMNIWPEYKNRTIFDILNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 439 aa, Artificial Sequence
  Synthetic Peptide Sequence
VIHVTKEVKEVATLSCGHNVSVEELAQVRIYWQKEKKMVLTMMSGDMNIWPEYKNRVIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 439 aa, Artificial Sequence
  Synthetic Peptide Sequence
VIHVTKEVKEVATLSCGHNVSVEELAQVRIQWEKEKKMVLTMMSGDMNIWPEYENRVIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 439 aa, Artificial Sequence
  Synthetic Peptide Sequence
VIHVTKEVKEVATLSCGHNVSVEELAQTRIYWQKEKKMVLTMMSGDMNIWPEYENRTIFYITEDLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFYILNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFYILDELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 439 aa, Artificial Sequence
  Synthetic Peptide Sequence
VIHVTKEVKEVATLSCGHNVSFEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 439 aa, Artificial Sequence
  Synthetic Peptide Sequence
VIHVTKEVKEVATLSCGHNVSFEELAQTRIYWQKEKKMVLTMMSGDMNIWPEYENRTIFYITNELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFFIINNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFRIYNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 439 aa, Artificial Sequence
  Synthetic Peptide Sequence
VIHVTKEVKELATLSCGHNVSVEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 439 aa, Artificial Sequence
  Synthetic Peptide Sequence
VIHVTKEVKELATLSCGHNVSVEELAQTRIYWQKEKKMVLTMMSGDMNIWPEYKNRTIFYITDNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 439 aa, Artificial Sequence
  Synthetic Peptide Sequence
VIHVTKEVKEVATLSCGHNVSMEELAQTRIYWQKEKKMVLTMMSGDMNIWPEYKNRTIFYITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFTIYNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFQIFNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 439 aa, Artificial Sequence
  Synthetic Peptide Sequence
VIHVTKEVKEVATLSCGHNVSFEELAQVRIYWQKEKKMVLTMMSGDMNIWPEYKNRVIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 439 aa, Artificial Sequence; identical to SEQ ID NO: 91
  Synthetic Peptide Sequence

SEQ ID NO: 107 - protein, 439 aa, Artificial Sequence
  Synthetic Peptide Sequence
VIHVTKEVKEVATLSCGHNVSFEELAQTRIYWQKEKKMVLTMMSGDMNIWPEYKNRTIFDILNELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 439 aa, Artificial Sequence
  Synthetic Peptide Sequence
VIHVTKEVKEVATLSCGHNVSFEELAQTRIYWQKEKKMVLTMMSGDMNIWPEYKNRTIFDILDELSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFYILDNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 439 aa, Artificial Sequence
  Synthetic Peptide Sequence
VIHVTKEVKEVATLSCGHNVSVEELAQTRIYWQKEKKMVLTMMSGDMNIWPEYKNRTIFDCTNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 439 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEKQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 439 aa, Artificial Sequence
  Synthetic Peptide Sequence
VIHVTKEVKEVATLSCGHNVSVCELCQTRIYWQKEKKMVLTMMSGDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 439 aa, Artificial Sequence
  Synthetic Peptide Sequence
VIHVTKEVKEVATLSCGHNVSCEELAQTRIYWQKEKKMVLTMMSCDMNIWPEYKNRTIFDITNNLSIVILALRPSDEGTYECVVLKYEQQAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 439 aa, Artificial Sequence
  Synthetic Peptide Sequence
VIHVTKEVKEVATLSCGHNVSVEELAQVRIYWQKEKKMVLTMMSGDMNIWPEYKNRVIFDITNNLSIVILALRPSDEGTYECVVLKYEDKAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - DNA, 1320 bp, Artificial Sequence
  Synthetic Nucleotide Sequence
gtgatccacgtcaccaaggaagtcaaggaagtggccaccctctcctgcggtcacaatgtgtccgtggaagaactggcccagacgcggatctactggcaaaaggagaagaagatggtgctgaccatgatgagcggagacatgaacatctggcccgagtacaagaaccggactatctttgactgcaccaacaacttgtcgatcgtcatcctggcactccggcctagcgatgagggaacctatgagtgcgtggtgttgaaatacgaacagcaagccttcaagagagagcacctcgccgaagtgaccctgagcgtgaaggccgacttccccaccccgagcatttcggacttcgagattccgacctccaacattcgccgcattatctgttcaacctccggcggattcccggagccacatctgtcctggctggagaacggcgaagaactgaacgcgattaacactaccgtgtcccaagaccctgaaactgagctgtacgccgtgtcatcgaagctcgacttcaacatgactaccaaccactccttcatgtgcctgatcaaatacgggcatctccgggtcaaccagaccttcaactggaacactaccaagcaggagcactttcccgacaatgagcctaagtcctcggacaagacccacacctgtcctccatgtccggcgccggaattgcttggcggtccgagcgtgttcctgttcccaccgaagccaaaggacaccctgatgattagcaggactcccgaagtcacttgcgtggtcgtggatgtgtctcacgaggacccggaagtcaagttcaattggtacgtggatggcgtggaagtccataacgccaagacgaaaccccgcgaggaacagtacaacagcacctaccgcgtggtgtcagtgctgaccgtgctgcaccaggattggctcaacggaaaggagtacaagtgcaaagtgtcgaacaaagccctgcctgctcccatcgaaaagacaatctcgaaggccaagggacaaccccgggaacctcaggtctacaccctgcctccttcccgggaggaaatgaccaagaaccaagtgtccctcacttgccttgtgaagggattctacccgtccgacatcgccgtggagtgggaatccaacggtcaacccgagaacaactacaagaccacccctccggtgctcgactcggatgggtcattcttcctgtactccaagctcaccgtggacaagtccagatggcagcagggaaacgtgttctcctgctcggtcatgcacgaggccctgcacaaccattacactcagaagtccctgtccctgagcccgggaaaa

SEQ ID NO: 116 - DNA, 1320 bp, Artificial Sequence
  Synthetic Nucleotide Sequence
gtgatccacgtcaccaaggaagtcaaggaagtggccaccctctcctgcggtcacaatgtgtccgtgtgcgaactgtgccagacgcggatctactggcaaaaggagaagaagatggtgctgaccatgatgagcggagacatgaacatctggcccgagtacaagaaccggactatctttgacatcaccaacaacttgtcgatcgtcatcctggcactccggcctagcgatgagggaacctatgagtgcgtggtgttgaaatacgaaaagcaagccttcaagagagagcacctcgccgaagtgaccctgagcgtgaaggccgacttccccaccccgagcatttcggacttcgagattccgacctccaacattcgccgcattatctgttcaacctccggcggattcccggagccacatctgtcctggctggagaacggcgaagaactgaacgcgattaacactaccgtgtcccaagaccctgaaactgagctgtacgccgtgtcatcgaagctcgacttcaacatgactaccaaccactccttcatgtgcctgatcaaatacgggcatctccgggtcaaccagaccttcaactggaacactaccaagcaggagcactttcccgacaatgagcctaagtcctcggacaagacccacacctgtcctccatgtccggcgccggaattgcttggcggtccgagcgtgttcctgttcccaccgaagccaaaggacaccctgatgattagcaggactcccgaagtcacttgcgtggtcgtggatgtgtctcacgaggacccggaagtcaagttcaattggtacgtggatggcgtggaagtccataacgccaagacgaaaccccgcgaggaacagtacaacagcacctaccgcgtggtgtcagtgctgaccgtgctgcaccaggattggctcaacggaaaggagtacaagtgcaaagtgtcgaacaaagccctgcctgctcccatcgaaaagacaatctcgaaggccaagggacaaccccgggaacctcaggtctacaccctgcctccttcccgggaggaaatgaccaagaaccaagtgtccctcacttgccttgtgaagggattctacccgtccgacatcgccgtggagtgggaatccaacggtcaacccgagaacaactacaagaccacccctccggtgctcgactcggatgggtcattcttcctgtactccaagctcaccgtggacaagtccagatggcagcagggaaacgtgttctcctgctcggtcatgcacgaggccctgcacaaccattacactcagaagtccctgtccctgagcccgggaaaa

SEQ ID NO: 117 - DNA, 1320 bp, Artificial Sequence
  Synthetic Nucleotide Sequence
gtgatccacgtcaccaaggaagtcaaggaagtggccaccctctcctgcggtcacaatgtgtccgtgtgcgaactgtgccagacgcggatctactggcaaaaggagaagaagatggtgctgaccatgatgagcggagacatgaacatctggcccgagtacaagaaccggactatctttgacatcaccaacaacttgtcgatcgtcatcctggcactccggcctagcgatgagggaacctatgagtgcgtggtgttgaaatacgaacagcaagccttcaagagagagcacctcgccgaagtgaccctgagcgtgaaggccgacttccccaccccgagcatttcggacttcgagattccgacctccaacattcgccgcattatctgttcaacctccggcggattcccggagccacatctgtcctggctggagaacggcgaagaactgaacgcgattaacactaccgtgtcccaagaccctgaaactgagctgtacgccgtgtcatcgaagctcgacttcaacatgactaccaaccactccttcatgtgcctgatcaaatacgggcatctccgggtcaaccagaccttcaactggaacactaccaagcaggagcactttcccgacaatgagcctaagtcctcggacaagacccacacctgtcctccatgtccggcgccggaattgcttggcggtccgagcgtgttcctgttcccaccgaagccaaaggacaccctgatgattagcaggactcccgaagtcacttgcgtggtcgtggatgtgtctcacgaggacccggaagtcaagttcaattggtacgtggatggcgtggaagtccataacgccaagacgaaaccccgcgaggaacagtacaacagcacctaccgcgtggtgtcagtgctgaccgtgctgcaccaggattggctcaacggaaaggagtacaagtgcaaagtgtcgaacaaagccctgcctgctcccatcgaaaagacaatctcgaaggccaagggacaaccccgggaacctcaggtctacaccctgcctccttcccgggaggaaatgaccaagaaccaagtgtccctcacttgccttgtgaagggattctacccgtccgacatcgccgtggagtgggaatccaacggtcaacccgagaacaactacaagaccacccctccggtgctcgactcggatgggtcattcttcctgtactccaagctcaccgtggacaagtccagatggcagcagggaaacgtgttctcctgctcggtcatgcacgaggccctgcacaaccattacactcagaagtccctgtccctgagcccgggaaaa

SEQ ID NO: 118 - DNA, 1320 bp, Artificial Sequence
  Synthetic Nucleotide Sequence
gtgatccacgtcaccaaggaagtcaaggaagtggccaccctctcctgcggtcacaatgtgtcctgcgaagaactggcccagacgcggatctactggcaaaaggagaagaagatggtgctgaccatgatgagctgcgacatgaacatctggcccgagtacaagaaccggactatctttgacatcaccaacaacttgtcgatcgtcatcctggcactccggcctagcgatgagggaacctatgagtgcgtggtgttgaaatacgaacagcaagccttcaagagagagcacctcgccgaagtgaccctgagcgtgaaggccgacttccccaccccgagcatttcggacttcgagattccgacctccaacattcgccgcattatctgttcaacctccggcggattcccggagccacatctgtcctggctggagaacggcgaagaactgaacgcgattaacactaccgtgtcccaagaccctgaaactgagctgtacgccgtgtcatcgaagctcgacttcaacatgactaccaaccactccttcatgtgcctgatcaaatacgggcatctccgggtcaaccagaccttcaactggaacactaccaagcaggagcactttcccgacaatgagcctaagtcctcggacaagacccacacctgtcctccatgtccggcgccggaattgcttggcggtccgagcgtgttcctgttcccaccgaagccaaaggacaccctgatgattagcaggactcccgaagtcacttgcgtggtcgtggatgtgtctcacgaggacccggaagtcaagttcaattggtacgtggatggcgtggaagtccataacgccaagacgaaaccccgcgaggaacagtacaacagcacctaccgcgtggtgtcagtgctgaccgtgctgcaccaggattggctcaacggaaaggagtacaagtgcaaagtgtcgaacaaagccctgcctgctcccatcgaaaagacaatctcgaaggccaagggacaaccccgggaacctcaggtctacaccctgcctccttcccgggaggaaatgaccaagaaccaagtgtccctcacttgccttgtgaagggattctacccgtccgacatcgccgtggagtgggaatccaacggtcaacccgagaacaactacaagaccacccctccggtgctcgactcggatgggtcattcttcctgtactccaagctcaccgtggacaagtccagatggcagcagggaaacgtgttctcctgctcggtcatgcacgaggccctgcacaaccattacactcagaagtccctgtccctgagcccgggaaaa

SEQ ID NO: 119 - DNA, 1320 bp, Artificial Sequence
  Synthetic Nucleotide Sequence
gtgatccacgtcaccaaggaagtcaaggaagtggccaccctctcctgcggtcacaatgtgtccgtggaagaactggcccaggtgcggatctactggcaaaaggagaagaagatggtgctgaccatgatgagcggagacatgaacatctggcccgagtacaagaaccgggtgatctttgacatcaccaacaacttgtcgatcgtcatcctggcactccggcctagcgatgagggaacctatgagtgcgtggtgttgaaatacgaagacaaagccttcaagagagagcacctcgccgaagtgaccctgagcgtgaaggccgacttccccaccccgagcatttcggacttcgagattccgacctccaacattcgccgcattatctgttcaacctccggcggattcccggagccacatctgtcctggctggagaacggcgaagaactgaacgcgattaacactaccgtgtcccaagaccctgaaactgagctgtacgccgtgtcatcgaagctcgacttcaacatgactaccaaccactccttcatgtgcctgatcaaatacgggcatctccgggtcaaccagaccttcaactggaacactaccaagcaggagcactttcccgacaatgagcctaagtcctcggacaagacccacacctgtcctccatgtccggcgccggaattgcttggcggtccgagcgtgttcctgttcccaccgaagccaaaggacaccctgatgattagcaggactcccgaagtcacttgcgtggtcgtggatgtgtctcacgaggacccggaagtcaagttcaattggtacgtggatggcgtggaagtccataacgccaagacgaaaccccgcgaggaacagtacaacagcacctaccgcgtggtgtcagtgctgaccgtgctgcaccaggattggctcaacggaaaggagtacaagtgcaaagtgtcgaacaaagccctgcctgctcccatcgaaaagacaatctcgaaggccaagggacaaccccgggaacctcaggtctacaccctgcctccttcccgggaggaaatgaccaagaaccaagtgtccctcacttgccttgtgaagggattctacccgtccgacatcgccgtggagtgggaatccaacggtcaacccgagaacaactacaagaccacccctccggtgctcgactcggatgggtcattcttcctgtactccaagctcaccgtggacaagtccagatggcagcagggaaacgtgttctcctgctcggtcatgcacgaggccctgcacaaccattacactcagaagtccctgtccctgagcccgggaaaa

SEQ ID NO: 120 - protein, 5 aa, Artificial Sequence
  Synthetic Peptide Sequence
SYWIN

SEQ ID NO: 121 - protein, 17 aa, Artificial Sequence
  Synthetic Peptide Sequence
NIYPGSSLTNYNEKFKN

SEQ ID NO: 122 - protein, 8 aa, Artificial Sequence
  Synthetic Peptide Sequence
LSTGTFAY

SEQ ID NO: 123 - protein, 117 aa, Artificial Sequence
  Synthetic Peptide Sequence
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 124 - protein, 17 aa, Artificial Sequence
  Synthetic Peptide Sequence
KSSQSLWDSGNQKNFLT

SEQ ID NO: 125 - protein, 7 aa, Artificial Sequence
  Synthetic Peptide Sequence
WTSYRES

SEQ ID NO: 126 - protein, 9 aa, Artificial Sequence
  Synthetic Peptide Sequence
QNDYFYPHT

SEQ ID NO: 127 - protein, 113 aa, Artificial Sequence
  Synthetic Peptide Sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK